(19) United States
(12) Patent Application Publication
(10) Pub. No.: US 2017/0002011 A1
(43) Pub. Date: Jan. 5, 2017

(54) BENZENE SULFONAMIDES AS CCR9 INHIBITORS

(71) Applicant: NORGINE B.V., Amsterdam Zuid-Ost (NL)

(72) Inventors: Rajagopal Bakthavatchalam, Bangalore (IN); Manas Kumar Basu, Ghaziabad, Uttar Pradesh (IN); Ajit Kumar Behera, Dist-nayagarth, Orissa (IN); Chandregowda Venkateshappa, Bangalore (IN); Christopher Alexander Hewson, Middlesex (GB); Sanjay Venkatachalapathi Kadnur, Bangalore (IN); Sarkis Barret Kalindjian, Middlesex (GB); Bheemashankar Kulkarni, Bangalore (IN); Rohit Saxena, Lucknow, Uttar Pradesh (IN); Juluri Suresh, Hyderabad, Andhra Pradesh (IN); Vellarkad Viswanathan, Bangalore, Karnataka (IN); Mohd Zainuddin, Kanpur, Uttar Pradesh (IN); Akila Parvathy Dharshinis, Thruneveli, Tamil Nadu (IN); Rajenda Kristam, Bangalore (IN)

(73) Assignee: Norgine B.V., Amsterdam Zuid-Ost (NL)

(21) Appl. No.: 15/107,374

(22) PCT Filed: Dec. 22, 2014
(86) PCT No.: PCT/EP2014/078945

§ 371 Date: Jun. 22, 2016

(30) Foreign Application Priority Data
Dec. 23, 2013 (IN) 5985/CHE/2013

(51) Int. Cl. C07D 487/04; C07D 471/04

(52) U.S. Cl. CPC C07D 487/04; C07D 471/04

(57) ABSTRACT

The present invention relates to compounds useful as CCR9 modulators, to compositions containing them, to methods of making them, and to methods of using them. In particular, the present invention relates to compounds capable of modulating the function of the CCR9 receptor by acting as partial agonists, antagonists or inverse agonists. Such compounds may be useful to treat, prevent or ameliorate a disease or condition associated with CCR9 activation, including inflammatory and immune disorder diseases or conditions such as inflammatory bowel diseases (IBD).

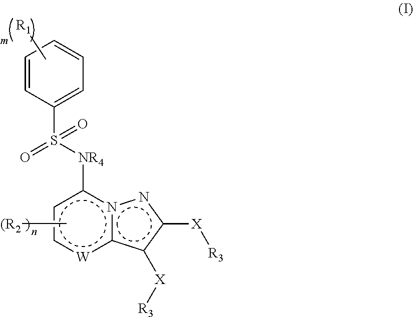

The present invention relates to compounds useful as CCR9 modulators, to compositions containing them, to methods of making them, and to methods of using them. In particular, the present invention relates to compounds capable of modulating the function of the CCR9 receptor by acting as partial agonists, antagonists or inverse agonists. Such compounds may be useful to treat, prevent or ameliorate a disease or condition associated with CCR9 activation, including inflammatory and immune disorder diseases or conditions such as inflammatory bowel diseases (IBD).

### BACKGROUND OF THE INVENTION

Chemokines are a family of structurally related small proteins released from a variety of different cells within the body (reviewed in Vinader et al, 2012, Future Med Chem, 4(7): 845-52). The name derives from their primary ability to induce chemotaxis and thereby attract multiple cells of the immune system to sites of inflammation or as a part of normal immune function homeostasis. Examples of the types of cells attracted by chemokines include monocytes, T and B lymphocytes, dendritic cells, natural killer cells, eosinophils, basophils and neutrophils. Chemokines, in addition to their primary role in inducing chemotaxis, are also able to cause activation of leukocytes at the site of inflammation—for example, but not limited to, causing degranulation of granulocytes, generation of super-oxide anions (oxidative burst) and up-regulation of integrins to cause extravasation. Chemokines initiate their biological activity through binding to and activation of cell surface receptors—chemokine receptors. Chemokine receptors belong to the G-coupled protein receptor (GPCR), 7-trans-membrane (7-TM) superfamily—comprising an extracellular N-terminus with 7 helical trans-membrane domains and an intracellular C-terminus. Traditionally, chemokines are considered to bind to their receptors in the 7-TM region—this binding leading to activation of the receptor and resulting in G-protein activation (and subsequent secondary messenger transmission) by the intracellular portion of the receptor.

CCR9 is a chemokine receptor shown to be expressed on circulating T lymphocytes (Zabel et al, 1999, J Exp Med, 190:1241-56) and, in contrast to the majority of human chemokine receptors, CCR9 currently has only a single ligand identified: CCL25, otherwise known as thymus-expressed chemokine (TECK) (Zabalos et al, 1999, J Immunol, 162: 5671-5). As CCL25 expression is limited to intestinal epithelium and the thymus (Kunkel et al, 2000, J Exp Med, 192(5): 761-8), this interaction has been demonstrated to be the key chemokine receptor involved in targeting of T lymphocytes to the intestine (Papadakis et al, 2000, J Immunol, 165(9): 5069-76). The infiltration of T lymphocytes into tissues has been implicated in a broad range of diseases, including, but not limited to, such diseases as asthma, rheumatoid arthritis and inflammatory bowel disease (IBD). Specific to IBD, it has been observed that CCR9+CD4 and CD8 T lymphocytes are increased in disease alongside an increased expression of CCL25 that correlates with disease severity (Papadakis et al, 2001, Gastroenterology, 121(2): 246-54). Indeed, disruption of the CCR9/CCL25 interaction by antibody and small molecule antagonists of CCR9 has been demonstrated to be effective in preventing the inflammation observed in small animal models of IBD (Rivera-Nieves et al, 2006, Gastroenterology, 131(5): 1518-29 and Walters et al, 2010, J Pharmacol Exp Ther, 335(1):61-9). In addition to the IBD specific role for CCR9, recent data also implicates the CCR9/CCL25 axis in liver inflammation and fibrosis where increased expression of CCL25 has been observed in the inflamed liver of primary sclerosing cholangitis patients along with a concomitant increase in the numbers of CCR9+ T lymphocytes (Eksteen et al, 2004, J Exp Med, 200(11):1511-7). CCR9+macrophages have also been observed in in vivo models of liver disease and their function proven with CCL25 neutralising antibodies and CCR9-knockout mice exhibiting a reduction in CCR9+ macrophage number, hepatitis and liver fibrosis (Nakamoto et al, 2012, Gastroenterol, 142:366-76 and Chu et al, 2012, 63^{rd} Annual Meeting of the American Association for the Study of Liver Diseases, abstract 1209). Therefore, modulation of the function of CCR9 represents an attractive target for the treatment of inflammatory, immune disorder and other conditions and diseases associated with CCR9 activation, including IBD and liver disease.

In addition to inflammatory conditions, there is increasing evidence for the role of CCR9 in cancer. Certain types of cancer are caused by T lymphocytes expressing CCR9. For example, in thymoma and thymic carcinoma (where cancer cells are found in the thymus), the developing T lymphocytes (thymocytes) are known to express high levels of CCR9 and CCL25 is highly expressed in the thymus itself. In the thymus, there is evidence that the CCR9/CCL25 interaction is important for thymocyte maturation (Svensson et al, 2008, J Leukoc Biol, 83(1): 156-64). In another example, T lymphocytes from acute lymphocytic leukaemia (ALL) patients express high levels of CCR9 (Qiuping et al, 2003, Cancer Res, 63(19): 6469-77). While the role for chemokine receptors is not clear in the pathogenesis of cancer, recent work has indicated that chemokine receptors, including CCR9, are important in metastasis of tumours—with a potential therapeutic role suggested for chemokine receptor antagonists (Fusi et al, 2012, J Transl Med, 10:52). Therefore, blocking the CCR9/CCL25 interaction may help to prevent or treat cancer expansion and/or metastasis.

Inflammatory bowel diseases (IBD) are chronic inflammatory disorders of the gastrointestinal tract in which tissue damage and inflammation lead to long-term, often irreversible impairment of the structure and function of the gastrointestinal tract (Bouma and Strober, 2003, Nat Rev Immunol, 3(7):521-533). Inflammatory bowel diseases may include collagenous colitis, lymphocytic colitis, ischaemic colitis, diversion colitis, Behçet's disease (also known as Behçet's syndrome), indeterminate colitis, ileitis and enteritis, but Crohn's disease and ulcerative colitis are the most common forms of IBD. Crohn's disease and ulcerative colitis both involve chronic inflammation and ulceration in the intestines, the result of an abnormal immune response. Chronic and abnormal activation of the immune system leads to tissue destruction in both diseases, although ulcerative colitis is generally limited to the rectum and colon, whereas Crohn's disease (also known as regional ileitis) extends deeper in the intestinal wall and can involve the entire digestive tract, from the mouth to the anus.

Up to one million Americans have inflammatory bowel disease, according to an estimate by the Crohn's and Colitis Foundation of America. The incidence of IBD is highest in Western countries. In North America and Europe, both ulcerative colitis and Crohn's disease have an estimated prevalence of 10-20 cases per 100,000 populations (Bouma and Strober, 2003).

The primary goal when treating a patient with IBD is to control active disease until a state of remission is obtained; the secondary goal is to maintain this state of remission (Kamm, 2004, Aliment Pharmacol Ther, 20(4):102). Most treatments for IBD are either medical or surgical (typically only used after all medical options have failed). Some of the more common drugs used to treat IBD include 5-aminosalicylic acid (5-ASA) compounds (such as sulfasalazine, mesalamine, and olsazine), immunosuppressants (such as azathioprine, 6-mercaptopurine (6-MP), cyclosporine A and methotrexate), corticosteroids (such as prednisone, methylprednisolone and budesonide), infliximab (an anti-TNFα antibody) and other biologics (such as adilumumab, certolizumab and natalizumab). None of the currently available drugs provides a cure, although they can help to control disease by suppressing destructive immune processes, promoting healing of intestinal tissues and relieving symptoms (diarrhoea, abdominal pain and fever).

There is a need to develop alternative drugs for the treatment of IBD, with increased efficacy and/or improved safety profile (such as reduced side effects) and/or improved pharmacokinetic properties. Treatment of IBD includes control or amelioration of the active disease, maintenance of remission and prevention of recurrence.

Various new drugs have been in development, including the aryl sulfonamide compound N-{4-chloro-2-[(1-oxidopyridin-4-yl)carbonyl]phenyl}-4-(1,1-dimethylethyl) benzenesulfonamide, also known as Vercirnon or GSK1605786 (CAS Registry number 698394-73-9), and Vercirnon sodium. Vercirnon was taken into Phase III clinical development for the treatment of patients with moderate-to-severe Crohn's disease. Vercirnon is the compound claimed in U.S. Pat. No. 6,939,885 (Chemocentryx) and is described as an antagonist of the CCR9 receptor. Various other aryl sulfonamide compounds have also been disclosed as CCR9 antagonists that may be useful for the treatment of CCR9-mediated diseases such as inflammatory and immune disorder conditions and diseases; for example, see the following Chemocentryx patent applications, WO2004/046092 which includes Vercirnon, WO2004/085384, WO2005/112916, WO2005/112925, WO2005/113513, WO2008/008374, WO2008/008375, WO2008/008431, WO2008/010934, WO2009/038847; also WO2003/099773 (Millennium Pharmaceuticals), WO2007/071441 (Novartis) and US2010/0029753 (Pfizer).

Thus a number of CCR9-modulating compounds are known and some are being developed for medical uses (see, for example, the review of CCR9 and IBD by Koenecke and Førster, 2009, Expert Opin Ther Targets, 13 (3):297-306, or the review of CCR antagonists by Proudfoot, 2010, Expert Opin Investig Drugs, 19(3): 345-55). Different classes of compounds may have different degrees of potency and selectivity for modulating CCR9. There is a need to develop alternative CCR9 modulators with improved potency and/or beneficial activity profiles and/or beneficial selectivity profiles and/or increased efficacy and/or improved safety profiles (such as reduced side effects) and/or improved pharmacokinetic properties.

Other classes of compounds with different biological targets have been suggested for different uses. For example, pyrazolo[1,5-a]pyrimidine derivatives said to be useful as analgesic compounds are disclosed in European patent publication number 0714898 (Otsuka Pharmaceutical Factory, Inc); for example, see compounds 127 and 128 in Table 4 of EP0714898.

We now provide a new class of compounds that are useful as CCR9 modulators, and in particular as partial agonists, antagonists or inverse agonists of CCR9. The compounds of the invention may have improved potency and/or beneficial activity profiles and/or beneficial selectivity profiles and/or increased efficacy and/or improved safety profiles (such as reduced side effects) and/or improved pharmacokinetic properties. Some of the preferred compounds may show selectivity for CCR9 over other receptors, such as other chemokine receptors.

Such compounds may be useful to treat, prevent or ameliorate a disease or condition associated with CCR9 activation, including inflammatory and immune disorder diseases or conditions such as inflammatory bowel diseases (IBD).

### SUMMARY OF THE INVENTION

The present invention provides a compound of Formula (I) or a salt or solvate thereof, including a solvate of such a salt:

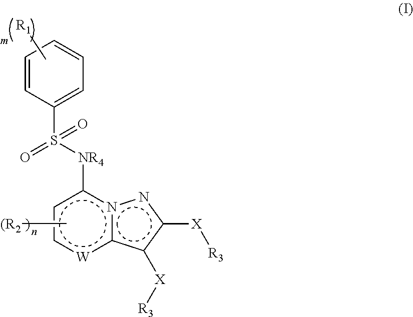

in which:

each R₁ is Z_{q1}B;

m is 0, 1, 2 or 3;

q₁ is 0, 1, 2, 3, 4, 5 or 6;

each Z is independently selected from CR₅R₆, O, C═O, SO₂, and NR₇;

each R₅ is independently selected from hydrogen, methyl, ethyl, and halo;

each R₆ is independently selected from hydrogen, methyl, ethyl, and halo;

each R₇ is independently selected from hydrogen, methyl, and ethyl;

each B is independently selected from hydrogen, halo, cyano (CN), optionally substituted aryl,

optionally substituted heteroaryl, optionally substituted cycloalkyl, and A;

A is

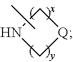

Q is selected from CH₂, O, NH, and NCH₃;

x is 0, 1, 2, 3 or 4, and y is 1, 2, 3, 4 or 5, the total of x and y being greater or equal to 1 and less than or equal to 5 (1≦x+y≦5);

each R₂ is independently selected from halo, cyano (CN), C₁₋₆ alkyl, C₁₋₆alkoxy, haloalkyl, haloalkoxy, and C₃₋₇ cycloalkyl;

n is 0, 1 or 2;

each X is independently selected from a direct bond and (CR₈R₉)ₚ;

each R₈ is independently selected from hydrogen, methyl, and fluoro;

each R₉ is independently selected from hydrogen, methyl, and fluoro;

p is 1, 2, 3, 4, or 5;

each R₃ is independently selected from hydrogen, cyano (CN), C₃₋₇ cycloalkyl, optionally substituted C₅₋₆ heterocycloalkyl, optionally substituted aryl, and optionally substituted heteroaryl;

R₄ is selected from hydrogen, methyl, and ethyl;

W is selected from N, and CRio;

R₁₀ is selected from hydrogen, halo, cyano (CN), methyl sulfonyl (SO₂CH₃), C₁₋₆ alkyl, C₁₋₆alkoxy, haloalkyl, haloalkoxy, and C₃₋₇ cycloalkyl;

provided that when W is N and n is 1 and R₂ is butyl, at least one of the XR₃ groups is not hydrogen.

It will be appreciated that the compounds of the invention may contain one or more asymmetrically substituted carbon atoms. The presence of one or more of these asymmetric centres (chiral centres) in a compound of Formula (I) can give rise to stereoisomers, and in each case the invention is to be understood to extend to all such stereoisomers, including enantiomers and diastereomers, and mixtures thereof (including racemic mixtures thereof).

Where tautomers exist in the compounds of Formula (I), we disclose all individual tautomeric forms and combinations of these as individual specific embodiments of the invention.

In addition, the invention is to be understood to extend to all isomers which are compounds with one or more isotopic substitutions. For example, H may be in any isotopic form, including ¹H, ²H(D), and ³H(T); C may be in any isotopic form, including ¹²C, ¹³C, and ¹⁴C; O may be in any isotopic form, including ¹⁶O and ¹⁸O; and the like.

It will be appreciated that the particular groups or substituents, the number of groups or substituents, and the position of substitution in compounds of Formula (I) are selected so as to avoid sterically undesirable combinations.

When present, each of the R₁ and R₂ groups may be attached at any suitable position. An R₁ group may be para, meta or ortho to the sulfonamide, especially para. For example, when m is 1, then R₁ is preferably meta or para to the sulfonamide, and most preferably para to the sulfonamide; and when m is 2, then most preferably one R₁ group is meta to the sulfonamide and the other R₁ group is para to the sulfonamide. An R₂ group may be ortho or meta to the sulfonamide, especially ortho. For example, when W is N or CH, and n is 1, then R₂ is most preferably ortho to the sulfonamide.

Certain compounds of the invention may act as prodrugs, or may be converted into prodrugs by known methods, and in each case the invention is to be understood to extend to all such prodrugs.

Except where otherwise stated, throughout this specification and claims, any of the following groups present in a compound of the invention or in an intermediate used for the preparation of a compound of the invention, is as defined below:
an alkyl group is any branched or unbranched (straight chain) hydrocarbon, and may for example contain from 1 to 7 carbon atoms, especially from 1 to 6 carbon atoms;
a cycloalkyl group is any monocyclic saturated hydrocarbon ring structure, and may for example contain from 3 to 7 carbon atoms, especially 3, 4, 5 or 6 carbon atoms;
a heteroalkyl group is any alkyl group wherein any one or more carbon atoms is replaced by a heteroatom independently selected from N, O, S;
a heterocycloalkyl group is any cycloalkyl group wherein any one or more carbon atoms is replaced by a heteroatom independently selected from N, O, S;
an aryl group is any polyunsaturated, aromatic hydrocarbon group having a single ring or multiple rings which are fused together or linked covalently; aryl groups with up to 10 carbon atoms are preferred, particularly a monocyclic aryl group having 6 carbon atoms; examples of aryl groups include phenyl, biphenyl and naphthalene;
a heteroaryl group is any aryl group wherein any one or more carbon atoms is replaced by a heteroatom independently selected from N, O, S; heteroaryl groups with 5 to 10 ring atoms are preferred, particularly a monocyclic heteroaryl group having 5 or 6 ring atoms; examples of heteroaryl groups include pyridyl, pyrazolyl, pyridazinyl, pyrrolyl, oxazolyl, quinolinyl and isoquinolinyl;
a halo group is any halogen atom, and may for example be fluorine (F), chlorine (Cl) or bromine (Br), and especially fluorine or chlorine;
a haloalkyl group is any alkyl group substituted with one or more halogen atoms, particularly 1, 2 or 3 halogen atoms, especially fluorine or chlorine;

an alkoxy group is any Oalkyl group, especially OC₁₋₆ alkyl;
a haloalkoxy group is any Ohaloalkyl group, especially OC₁₋₆ haloalkyl.

Except where otherwise stated, throughout this specification and claims, the phrase “optionally substituted” means unsubstituted or substituted by up to three groups (“optional substituents”) independently selected from OH, ═O or O⁻, NO₂, CF₃, CN, halo (such as Cl or F or Br), CHO, CO₂H, C₁₋₄alkyl (such as methyl), C₃₋₇cycloalkyl, C₁₋₄alkoxy (such as —O-methyl, —O-ethyl), COC₁₋₄alkyl (such as —(CO)-methyl), COC₁₋₄alkoxy (such as —(CO)—O-methyl), and C₁₋₄haloalkoxy.

Except where otherwise stated, throughout this specification and claims, the term “prodrug” means a compound which, upon administration to the recipient, has very low activity or is inactive in its administered state but is capable of providing (directly or indirectly) an active compound or an active metabolite thereof. A prodrug is converted within the body into its active form which has medical effects.

### DETAILED DESCRIPTION OF THE INVENTION

The compounds as defined above are useful as CCR9 modulators, and in particular as partial agonists, antagonists or inverse agonists of CCR9. Such compounds may be useful to treat, prevent or ameliorate a disease or condition associated with CCR9 activation, including inflammatory and immune disorder diseases or conditions. Such diseases or conditions include inflammatory bowel diseases (IBD). In particular, the compounds as defined above may be useful to treat, prevent or ameliorate Crohn's disease and/or ulcerative colitis, and most particularly Crohn's disease.

The compounds as defined above are novel. Accordingly, the present invention provides a compound of Formula (I) as defined above or a salt or solvate thereof, including a solvate of such a salt, per se. In particular, the present invention provides a compound of Formula (I) as defined above or a pharmaceutically acceptable salt or solvate thereof, including a solvate of such a salt, per se. Most particularly, the present invention provides a compound of Formula (I) or a pharmaceutically acceptable salt thereof, per se.

In order to use a compound of Formula (I) or a salt or solvate thereof for therapy, it is normally formulated in accordance with standard practice as a composition.

Thus the invention also provides a composition comprising a compound of Formula (I) or a salt or solvate thereof, including a solvate of such a salt, together with an acceptable carrier. In particular, the invention provides a pharmaceutical composition comprising a compound of Formula (I) or a salt or solvate thereof, including a solvate of such a salt, together with a pharmaceutically acceptable carrier.

The invention further provides a compound according to the invention for use in therapy, specifically, for use in the treatment, prevention or amelioration of a disease or condition associated with CCR9 activation, including inflammatory and immune disorder diseases or conditions. Such diseases or conditions include: (1) Inflammatory bowel diseases (IBD) such as Crohn's disease, ulcerative colitis, collagenous colitis, lymphocytic colitis, ischaemic colitis, diversion colitis, Behget's disease, indeterminate colitis, ileitis and enteritis; (2) allergic diseases such as systemic anaphylaxis or hypersensitivity responses, drug allergies, insect sting allergies and food allergies; (3) immune-mediated food allergies such as Coeliac (Celiac) disease; (4) autoimmune diseases, such as rheumatoid arthritis, fibromyalagia, scleroderma, ankylosing spondylitis, juvenile RA, Still's disease, polyarticular juvenile RA, pauclarticular juvenile RA, polymyalgia rheumatica, psoriatic arthritis, osteoarthritis, polyarticular arthritis, multiple scerlosis, systemic lupus erythematosus, type I diabetes, type II diabetes, glomerulonephritis, and the like; (5) psoriasis and inflammatory dermatoses such as dermatitis, eczema, atopic dermatitis, allergic contact dermatitis, urticaria and pruritus; (6) asthma and respiratory allergic diseases such as allergic asthma, allergic rhinitis, hypersensitivity lung diseases and the like; (7) vaginitis; (8) vasculitis; (9) spondyloarthropathies; (10) scleroderma; (11) graft rejection (including allograft rejection); (12) graft-v-host disease (including both acute and chronic); (13) other diseases in which undesired inflammatory responses are to be inhibited, such as atherosclerosis, myositis, neurodegenerative diseases (such as Alzheimer's disease), encephalitis, meningitis, liver diseases (such as liver inflammation, liver fibrosis, hepatitis, NASH), nephritis, sepsis, sarcoidosis, allergic conjunctivitis, otitis, chronic obstructive pulmonary disease, sinusitis, Behcet's disease and gout; (14) cancers, such as thymoma and thymic carcinoma, and acute lymphocytic leukemia (ALL, also known as acute lymphoblastic leukemia).

In particular, the invention provides a compound according to the invention for use to treat, prevent or ameliorate Crohn's disease and/or ulcerative colitis, and most particularly Crohn's disease.

The invention further provides the use of a compound of the invention for the treatment, prevention or amelioration of diseases or conditions as mentioned above; the use of a compound of the invention for the manufacture of a medicament for the treatment, prevention or amelioration of diseases or conditions as mentioned above; and a method of treating, preventing or ameliorating a disease or condition as mentioned above in a subject, which comprises administering an effective amount of a compound or a composition according to the invention to said subject. The subject to be treated according to the present invention is typically a mammal. The mammal is generally a human but may for example be a commercially reared animal or a companion animal.

A compound of Formula (I) may also be used as an intermediate in a method to synthesise another chemical compound, including but not limited to another compound of Formula (I); as a reagent in an analytical method; as a research tool—for example, as a comparator compound in an assay, or during compound screening to assist in identifying and/or profiling a compound with similar or differing activity in the test conditions applied, or as a control in cell based, in vitro and/or in vivo test assays.

In preferred compounds of Formula (I), n is 0 or 1, and in particularly preferred compounds of Formula (I), n is 0 (so there is no R₂ group present).

In preferred compounds of Formula (I), at least one of the XR₃ groups is not hydrogen; most especially, either one of the XR₃ groups is not hydrogen and the other XR₃ group is hydrogen (ie X is a direct bond and R₃ is H). Particularly preferred compounds of Formula (I) are compounds of Formula (II):

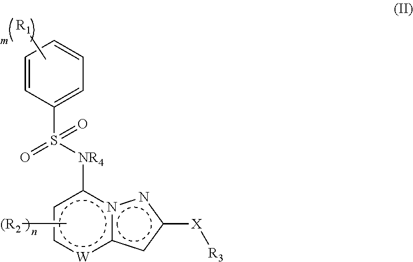

wherein the definitions of R₁, R₂, R₃, R₄, X, m and n are as given above for Formula (I).

In preferred compounds of Formula (II), n is 0 or 1, and in particularly preferred compounds of Formula (II), n is 0 (so there is no R₂ group present). In preferred compounds of Formula (II), the XR₃ group is not hydrogen. In particularly preferred compounds of Formula (II), n is 0 and the XR₃ group is not hydrogen, or n is 0 and W is C-halo (particularly C-chloro) or C-cyano.

In most particularly preferred compounds of Formula (II), n is 0, the XR₃ group is not hydrogen, and W is C-halo (particularly C-chloro) or C-cyano.

Preferred compounds of Formula (I) include those wherein any one or more of the following apply; particularly preferred compounds are compounds of Formula (II) wherein any one or more of the following apply:
R₁ is Z_{q1}B and q₁ is 0, each B is independently selected from halo, CN, optionally substituted aryl, optionally substituted heteroaryl, and A; especially each B is independently selected from halo, optionally substituted C₅₋₆heteroaryl (particularly unsubstituted C₅₋₆heteroaryl), and C₅₋₆heterocycloalkyl (where B is A, and the total of x and y is 3 or 4, and Q is CH₂ or O); more especially each B is independently selected from bromo, chloro, fluoro, pyridyl, pyrazolyl, methyl-pyrazolyl, oxazolyl, isoxazolyl, dimethyl-isoxazolyl, imidazolyl, thiophenyl, pyrrolyl, piperidinyl, pyrrolidinyl, and morpholinyl; most especially each B is independently selected from bromo, chloro, fluoro, and oxazolyl; particularly B is oxazolyl; and/or
R₁ is Z_{q1}B and q₁ is 1, 2 or 3, each Z is independently selected from C₁₋₃alkyl, each B is independently selected from halo, CN, optionally substituted aryl, optionally substituted heteroaryl, and A; especially each B is independently selected from halo, optionally substituted C₅₋₆heteroaryl (particularly unsubstituted C₅₋₆heteroaryl), and C₅₋₆heterocycloalkyl (where B is A, and the total of x and y is 3 or 4, and Q is CH₂ or O); more especially each B is independently selected from bromo, chloro, fluoro, pyridyl, pyrazolyl, methyl-pyrazolyl, oxazolyl, isoxazolyl, dimethyl-isoxazolyl, imidazolyl, thiophenyl, pyrrolyl, piperidinyl, pyrrolidinyl, and morpholinyl; most especially each B is independently selected from bromo, chloro, fluoro, and oxazolyl; particularly B is oxazolyl; and/or
R₁ is Z_{q1}B and q₁ is 1, 2, 3, 4, 5 or 6, particularly q₁ is 1 or 2, each Z is independently selected from CR₅R₆, O, C═O, and SO₂, each R₅ is independently selected from hydrogen, methyl, and halo (particularly fluoro), each R₆ is independently selected from hydrogen, methyl, and halo (particularly fluoro), B is selected from hydrogen, halo (particularly fluoro), and cyano; most especially each R₁ is independently selected from butyl (particularly tert-butyl), propyl (particularly isopropyl), methyl, trifluoromethyl, trifluoromethoxy, difluoromethoxy, methoxy, carboxy-methyl (CO)CH₃, methyl sulfonyl (SO₂CH₃), (CH₂)₃OCH₃, and C(CH₃)(CH₃)CN; particularly each R₁ is independently selected from butyl (particularly tert-butyl), propyl (particularly isopropyl), trifluoromethyl, trifluoromethoxy, and C(CH₃)(CH₃)CN; most particularly each R₁ is independently selected from butyl (particularly tert-butyl), and/or

m is 0, 1 or 2; especially m is 1 or 2; most especially m is 1; when m is 1, then R₁ is preferably meta or para to the sulfonamide, and most preferably para to the sulfonamide; and when m is 2, then most preferably one R₁ group is meta to the sulfonamide and the other R₁ group is para to the sulfonamide; for example when m is 1, R₁ may be meta or para to the sulfonamide (especially para) and may be tert-butyl, isopropyl, methyl, trifluoromethyl, trifluoromethoxy, difluoromethoxy, or methoxy (especially R₁ may be tert-butyl or trifluoromethyl); for example when m is 2, one R₁ group is meta to the sulfonamide and the other R₁ group is para to the sulfonamide, and the two R₁ groups may be trifluoromethyl and chloro or the two R₁ groups may be trifluoromethyl and fluoro; and/or

each R₂ is independently selected from halo, cyano (CN), C₁₋₃alkyl, C₁₋₃alkoxy, C₁₋₃haloalkyl, and cyclopropyl; especially each R₂ is independently selected from bromo, chloro, cyano, methyl, methoxy (CH₃O), propoxy particularly isopropoxy (Oisopropyl), trifluoromethyl, and cyclopropyl; especially R₂ is chloro, bromo or cyano; most especially R₂ is chloro or cyano; and/or
n is 0 or 1; especially n is 0 when W is N or when W is C-halo or C-cyano; especially n is 1 when W is CH; when n is 1, the R₂ group may be ortho or meta to the sulfonamide, preferably ortho; for example, when n is 1 and W is N or CH, then R₂ is most preferably ortho to the sulfonamide; and/or
each X is independently selected from a direct bond, CH₂, CH₂CH₂, C(CH₃)(CH₃), and C(CH₃)(CH₃)CH₂; especially X is selected from a direct bond, CH₂, and CH₂CH₂; most especially X is a direct bond or CH₂; and/or
p is 1, 2, or 3 (particularly 1); and/or
each R₃ is independently selected from hydrogen, C₃₋₇cycloalkyl, optionally substituted C₅-6heterocycloalkyl, optionally substituted aryl, and optionally substituted heteroaryl; especially each R₃ is selected from hydrogen, cyclopropyl, optionally substituted piperidinyl, optionally substituted phenyl, optionally substituted pyridyl, optionally substituted thiophenyl, optionally substituted pyrazolyl, optionally substituted pyridonyl, optionally substituted pyrimidinyl, optionally substituted imidazolyl, optionally substituted pyridazinyl, optionally substituted pyrazinyl, optionally substituted thiazolyl, optionally substituted oxazolyl, optionally substituted pyrrolyl, and optionally substituted isoquinoline, including piperidinyl, phenyl, chloro-phenyl, methyl-phenyl, cyano-phenyl, pyridyl, cyano-pyridyl, chloro-pyridyl, fluoro-pyridyl, methoxy-pyridyl, pyridyl-N oxide, methoxy-pyridyl-N oxide, ethoxy-pyridyl, ethoxy-pyridyl N-oxide, methyl-pyridyl and methyl-pyridyl N-oxide, thiophenyl, thiophenyl-CO₂H, thiophenyl-CO₂CH₃, pyrazolyl, methyl-pyrazolyl, dimethyl-pyrazolyl, pyrimidinyl, pyrazinyl, imidazolyl, methyl-imidazolyl, pyridazinyl, thiazolyl, oxazolyl, pyrrolyl, methyl-pyrrolyl, methyl-pyridonyl and isoquinoline; preferred optional substituents are selected from halo (particularly chloro or fluoro), cyano (CN), methyl, ethyl, isopropyl, methoxy (CH₃O), acetyl (CH₃CO), CO₂H, CO₂CH₃, OH, and O—; more especially each R₃ is selected from hydrogen, cyclopropyl, optionally substituted pyridyl, optionally substituted thiophenyl, optionally substituted pyrazolyl, optionally substituted pyridazinyl, optionally substituted oxazolyl, and optionally substituted pyrrolyl, including pyridyl, cyano-pyridyl, fluoro-pyridyl, methoxy-pyridyl, pyridyl-N oxide, methoxy-pyridyl-N oxide, ethoxy-pyridyl, ethoxy-pyridyl N-oxide, methyl-pyridyl and methyl-pyridyl N-oxide, thiophenyl-CO₂H, pyrazolyl, methyl-pyrazolyl, dimethyl-pyrazolyl, pyridazinyl, oxazolyl, and methyl-pyrrolyl; most preferably each R₃ is selected from hydrogen, optionally substituted pyridyl, optionally substituted thiophenyl, optionally substituted pyrazolyl, and optionally substituted pyrrolyl, including pyridyl, fluoro-pyridyl, methoxy-pyridyl, pyridyl-N oxide, methoxy-pyridyl-N oxide, methyl-pyridyl, methyl-pyridyl N-oxide, thiophenyl-CO₂H, pyrazolyl, methyl-pyrazolyl, and methyl-pyrrolyl; and/or
at least one of the XR₃ groups is not hydrogen; most especially, one of the XR₃ groups is not hydrogen and the other XR₃ group (if present) is hydrogen; and/or
R₄ is hydrogen; and/or
W is selected from N, CH, C-halo, and C-cyano; especially W is selected from C-halo (particularly C-chloro) and C-cyano; most particularly W is C-cyano.

In preferred compounds of the invention, optionally substituted groups are those that are unsubstituted or substituted by one or two groups independently selected from OH, ═O or O⁻, NO₂, CF₃, CN, halo (such as Cl or F or Br), CHO, CO₂H, C₁₋₄alkyl (such as methyl, ethyl, isopropyl), C₃₋₇cycloalkyl, C₁₋₄alkoxy (such as —O-methyl, —O-ethyl), COC₁₋₄alkyl (such as —(CO)-methyl), COC₁₋₄alkoxy (such as —(CO)—O-methyl), and C₁₋₄haloalkoxy. Preferred substituents (particularly for R₃) are selected from O⁻, CN, CO₂H, methyl, methoxy (—O— methyl), ethyl, ethoxy (—O-ethyl), and CO₂methyl. When R₃ is an optionally substituted aryl, each substituent may be ortho, meta or para to the point of attachment to X. When R₃ is an optionally substituted heteroaryl, each substituent may be ortho, meta or para to the point of attachment to X, or may be attached to a heteroatom.

For compounds of Formula (I), examples of preferred XR₃ groups include those shown below plus XR₃ groups wherein the aryl or heteroaryl groups shown below are further optionally substituted (preferably, in a compound of Formula (I), one XR₃ group is selected from such preferred XR₃ groups, and one XR₃ group is H; most preferably, in a compound of Formula (II), the XR₃ group is selected from such preferred XR₃ groups):

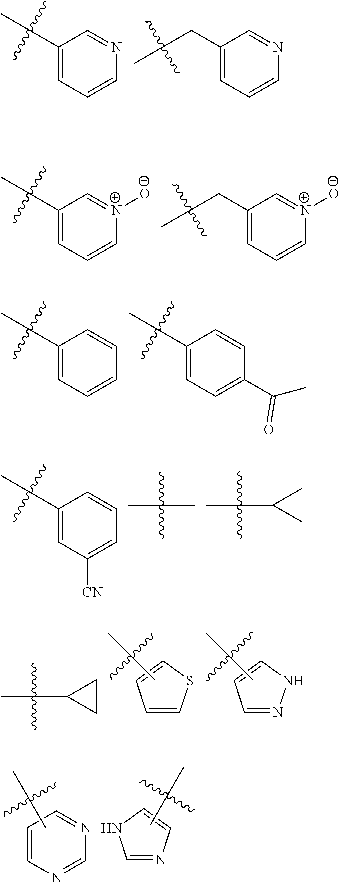

In certain preferred compounds of Formula (II), X is selected from a direct bond, CH₂, CH₂CH₂, C(CH₃)(CH₃) and C(CH₃)(CH₃)CH₂, and R₃ is hydrogen, so that XR₃ is selected from H, methyl, ethyl, isopropyl, and tert-butyl. In particular, XR₃ is selected from methyl and ethyl.

In other preferred compounds of Formula (II), X is a direct bond and R₃ is selected from cyano (CN), C₃₋₇cycloalkyl, optionally substituted C₅₋₆heterocycloalkyl, optionally substituted aryl, and optionally substituted heteroaryl.

For compounds of Formula (I), when R₁ is A (ie q₁ is 0 and B is A), R₁ is a C₃₋₇heterocycloalkyl containing one heteroatom (N) or two heteroatoms (N plus O or N, where the second N may be substituted with methyl). For example, A may be pyrrolidinyl, piperidinyl, or morpholinyl. The group A is attached through any of its carbon or nitrogen atoms, for example as follows:

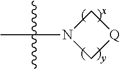

Particular compounds of Formula (I) and Formula (II) include those wherein:
m is 2; and
one R₁ group is halo (particularly bromo, chloro or fluoro, most particularly chloro,), and the other R₁ group is trifluoromethyl; and
one R₁ group is meta to the sulfonamide and the other R₁ group is para to the sulfonamide; and
n is 0 (so there is no R₂ group present); and
X is CH₂CH₂; and
R₃ is hydrogen; and
R₄ is hydrogen; and
W is N.
An example of such a compound is shown below:

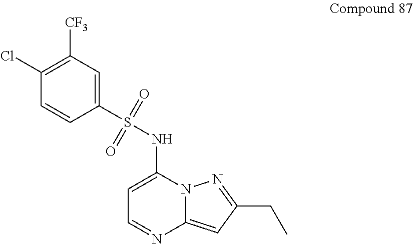 [SMILES: CCc1cc2nccc(NS(=O)(=O)c3ccc(Cl)c(C(F)(F)F)c3)n2n1]

Further particular compounds of Formula (I) and Formula (II) include those wherein:
R₁ is C₅₋₆heterocycloalkyl, particularly pyrrolidinyl or morpholinyl; and
m is 1; and
R₁ is meta or para to the sulfonamide, preferably para to the sulfonamide; and
n is 0 (so there is no R₂ group present); and
X is CH₂; and
R₃ is hydrogen; and
R₄ is hydrogen; and
W is N.

Examples of such compounds are shown below:

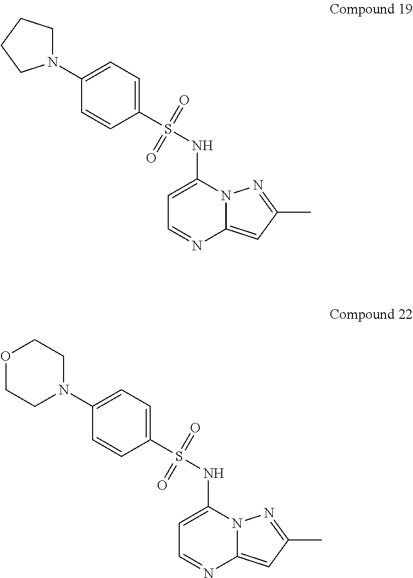 [SMILES: Cc1cc2nccc(NS(=O)(=O)c3ccc(N4CCCC4)cc3)n2n1 ; Cc1cc2nccc(NS(=O)(=O)c3ccc(N4CCOCC4)cc3)n2n1]

Other particular compounds of Formula (I) and Formula (II) include those wherein:
R₁ is optionally substituted heteroaryl, particularly unsubstituted heteroaryl, most preferably oxazolyl; and
m is 1; and
R₁ is meta or para to the sulfonamide, preferably para to the sulfonamide; and
n is 0 (so there is no R₂ group present); and
X is CH₂; and
R₃ is hydrogen; and
R₄ is hydrogen; and
W is N.

An example of such a compound is Compound 1 shown below:

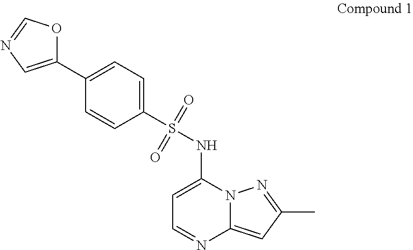 [SMILES: Cc1cc2nccc(NS(=O)(=O)c3ccc(-c4cnco4)cc3)n2n1]

Other particular compounds of Formula (I) and Formula (II) include those wherein:
R₁ is butyl (particularly tert-butyl); and
m is 1; and
R₁ is meta or para to the sulfonamide, preferably para to the sulfonamide; and
n is 0 (so there is no R₂ group present); and
X is a direct bond; and
R₃ is optionally substituted heteroaryl, particularly unsubstituted heteroaryl such as pyridyl; and
R₄ is hydrogen; and
W is N.
An example of such a compound is shown below:

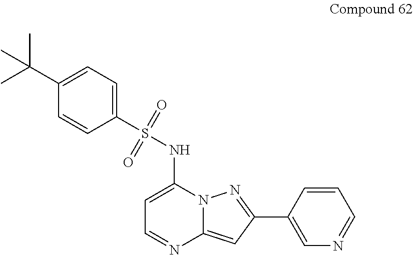 [SMILES: CC(C)(C)c1ccc(S(=O)(=O)Nc2ccnc3cc(-c4cccnc4)nn23)cc1]

Further particular compounds of Formula (I) and Formula (II) include those wherein:
R₁ is tert-butyl, trifluoromethyl, trifluoromethoxy, difluoromethoxy (, or methoxy; and
m is 1; and
R₁ is meta or para to the sulfonamide, preferably para to the sulfonamide; and
n is 0 (so there is no R₂ group present); and
X is a direct bond; and
R₃ is cyclopropyl; and
R₄ is hydrogen; and
W is N.
Examples of such compounds are shown below:

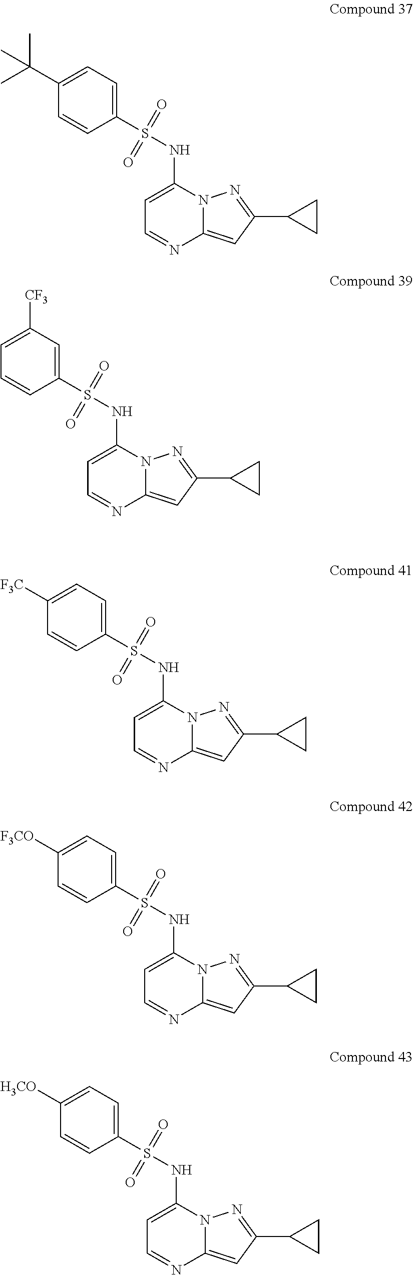 [SMILES: CC(C)(C)c1ccc(S(=O)(=O)Nc2ccnc3cc(C4CC4)nn23)cc1 ; O=S(=O)(Nc1ccnc2cc(C3CC3)nn12)c1cccc(C(F)(F)F)c1 ; O=S(=O)(Nc1ccnc2cc(C3CC3)nn12)c1ccc(C(F)(F)F)cc1 ; O=S(=O)(Nc1ccnc2cc(C3CC3)nn12)c1ccc(OC(F)(F)F)cc1 ; COc1ccc(S(=O)(=O)Nc2ccnc3cc(C4CC4)nn23)cc1]

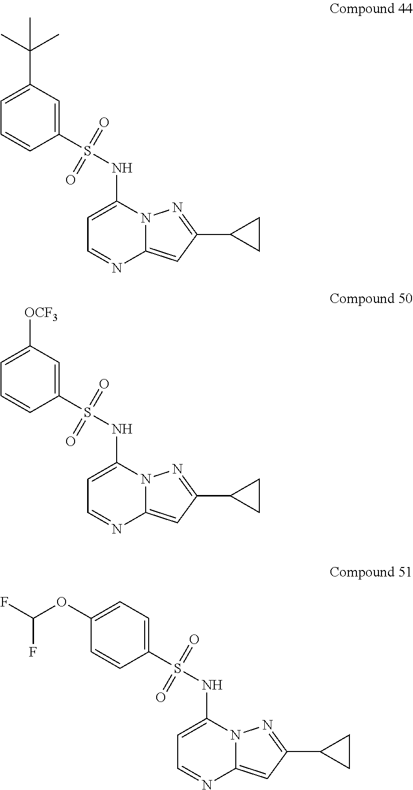 [SMILES: CC(C)(C)c1cccc(S(=O)(=O)Nc2ccnc3cc(C4CC4)nn23)c1 ; O=S(=O)(Nc1ccnc2cc(C3CC3)nn12)c1cccc(OC(F)(F)F)c1 ; O=S(=O)(Nc1ccnc2cc(C3CC3)nn12)c1ccc(OC(F)F)cc1]

Further particular compounds of Formula (I) and Formula (II) include those wherein:
R¹ is halo (such as bromo), tert-butyl, trifluoromethyl, trifluoromethoxy, or difluoromethoxy; and
m is 1; and
R₁ is meta or para to the sulfonamide, preferably para to the sulfonamide; and
n is 0 (so there is no R₂ group present); and
X is CH₂, CH₂CH₂, or C(CH₃)(CH₃); and
R₃ is hydrogen; and
R₄ is hydrogen; and
W is N.
Examples of such compounds are shown below:

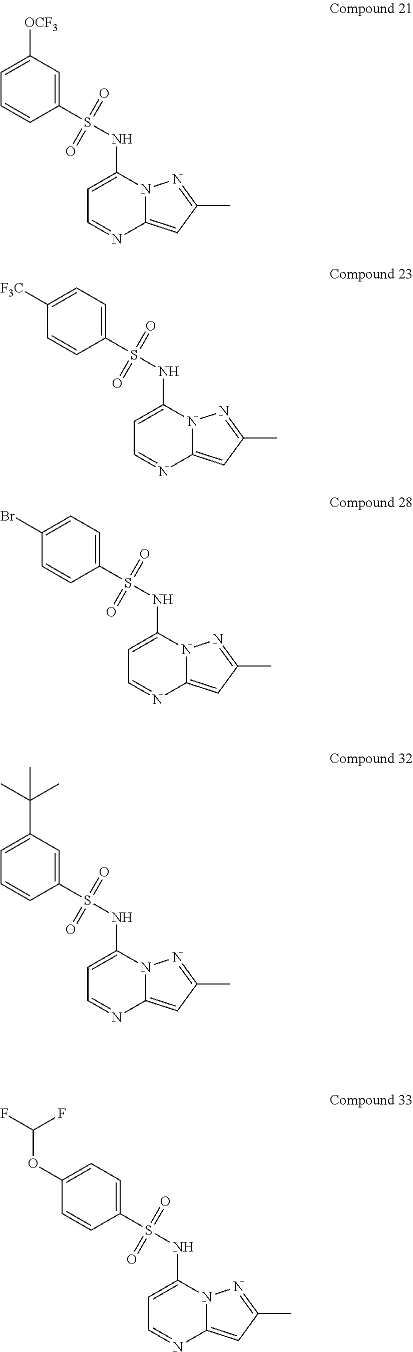 [SMILES: Cc1cc2nccc(NS(=O)(=O)c3cccc(OC(F)(F)F)c3)n2n1 ; Cc1cc2nccc(NS(=O)(=O)c3ccc(C(F)(F)F)cc3)n2n1 ; Cc1cc2nccc(NS(=O)(=O)c3ccc(Br)cc3)n2n1 ; Cc1cc2nccc(NS(=O)(=O)c3cccc(C(C)(C)C)c3)n2n1 ; Cc1cc2nccc(NS(=O)(=O)c3ccc(OC(F)F)cc3)n2n1]

Especially preferred examples of such compounds are shown below:

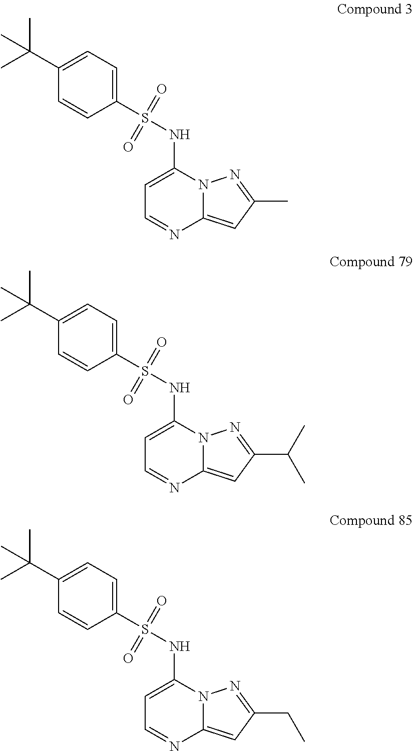

Preferred compounds of Formula (I) are compounds of Formula (II) wherein:
m is 1; and
R₁ is butyl (particularly tert-butyl); and
R₁ is meta or para to the sulfonamide, preferably para to the sulfonamide; and
n is 0 (so there is no R₂ group present); and
XR₃ is selected from methyl, cyclopropyl, optionally substituted pyridyl, optionally substituted thiophenyl, optionally substituted pyrazolyl, optionally substituted pyridazinyl, optionally substituted oxazolyl, and optionally substituted pyrrolyl, including pyridyl, cyano-pyridyl, fluoro-pyridyl, methoxy-pyridyl, pyridine-N oxide, methoxy-pyridine-N oxide, ethoxy-pyridyl, ethoxy-pyridyl N-oxide, methyl-pyridyl and methyl-pyridyl N-oxide, thiophenyl-CO₂H, pyrazolyl, methyl-pyrazolyl, dimethyl-pyrazolyl, pyridazinyl, oxazolyl, and methyl-pyrrolyl; most preferably XR₃ is selected from methyl, optionally substituted pyridyl, optionally substituted thiophenyl, optionally substituted pyrazolyl, and optionally substituted pyrrolyl, including pyridyl, fluoro-pyridyl, methoxy-pyridyl, pyridine-N oxide, methoxy-pyridyl-N oxide, ethoxy-pyridyl, ethoxy-pyridyl N-oxide, methyl-pyridyl, methyl-pyridyl N-oxide, thiophenyl-CO₂H, pyrazolyl, methyl-pyrazolyl, and methyl-pyrrolyl;
R₄ is hydrogen; and
W is C-chloro or C-cyano.
Examples of such a compound are shown below:

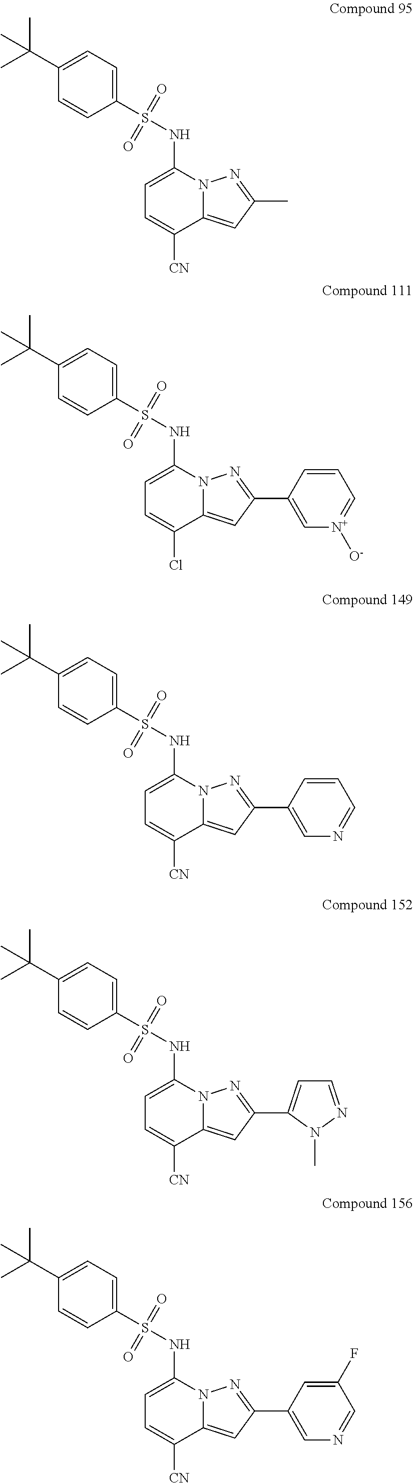

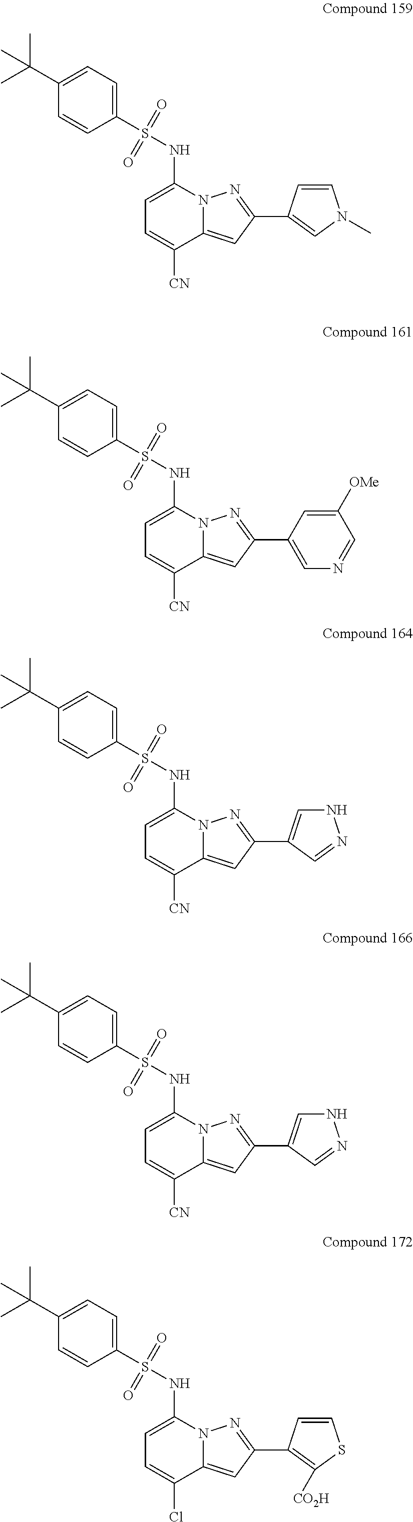

Other preferred compounds of Formula (I) are compounds of Formula (II) wherein:
m is 1; and
R₁ is butyl (particularly tert-butyl); and
R₁ is meta or para to the sulfonamide, preferably para to the sulfonamide; and
n is 1; and
R₂ is halo (such as chloro) or CN, particularly CN; and
the R₂ group is ortho to the sulfonamide; and
XR₃ is selected from methyl, cyclopropyl, optionally substituted pyridyl, optionally substituted thiophenyl, optionally substituted pyrazolyl, optionally substituted pyridazinyl, optionally substituted oxazolyl, and optionally substituted pyrrolyl, including pyridyl, cyano-pyridyl, fluoro-pyridyl, methoxy-pyridyl, pyridine-N oxide, methoxy-pyridine-N oxide, methoxy-pyridine-N oxide, ethoxy-pyridyl, ethoxy-pyridyl N-oxide, methyl-pyridyl and methyl-pyridyl N-oxide, thiophenyl-CO₂H, pyrazolyl, methyl-pyrazolyl, dimethyl-pyrazolyl, pyridazinyl, oxazolyl, and methyl-pyrrolyl; most preferably XR₃ is selected from methyl, optionally substituted pyridyl, optionally substituted thiophenyl, optionally substituted pyrazolyl, and optionally substituted pyrrolyl, including pyridyl, fluoro-pyridyl, methoxy-pyridyl, pyridine-N oxide, methoxy-pyridyl-N oxide, ethoxy-pyridyl, ethoxy-pyridyl N-oxide, methyl-pyridyl, methyl-pyridyl N-oxide, thiophenyl-CO₂H, pyrazolyl, methyl-pyrazolyl, and methyl-pyrrolyl;
R₄ is hydrogen; and
W is CH.
An example of such a compound is shown below:

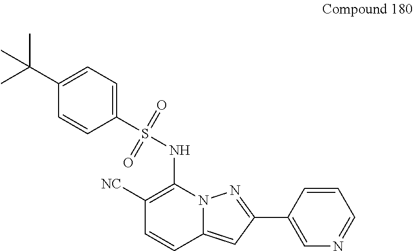 [SMILES: CC(C)(C)c1ccc(S(=O)(=O)Nc2c(C#N)ccc3cc(-c4cccnc4)nn23)cc1]

It will be appreciated that, in the compounds described above:
R₁ is trifluoromethoxy when R₁ is Z_{q1}B, q₁ is 2, the first Z group is O, the second Z group is CR₅R₆, and each of R₅, R₆ and B is fluoro;
R₁ is trifluoromethyl when R₁ is Z_{q1}B, q₁ is 1, Z is CR₅R₆, and each of R₅, R₆ and B is fluoro;
R₁ is tert-butyl when R₁ is Z_{q1}B, q₁ is 2, the first Z group is CR₅R₆ where each of R₅ and R₆ is methyl, the second Z group is CR₅R₆ where each of R₅ and R₆ is hydrogen, and B is hydrogen;
R₁ is isopropyl when R₁ is Z_{q1}B, q₁ is 1, the Z group is CR₅R₆ where each of R₅ and R₆ is methyl, and B is hydrogen; or R₁ is isopropyl when R₁ is Z_{q1}B, q₁ is 2, the first Z group is CR₅R₆ where one of R₅ and R₆ is methyl and the other is H, the second Z group is CR₅R₆ where each of R₅ and R₆ is hydrogen, and B is hydrogen;
R₁ is methyl when R₁ is Z_{q1}B, q₁ is 1, the Z group is CR₅R₆ where each of R₅ and R₆ is hydrogen, and B is hydrogen;
R₁ is difluoromethoxy when R₁ is Z_{q1}B, q₁ is 2, the first Z group is O, the second Z group is CR₅R₆, one of R₅, R₆ and B is hydrogen, and two of R₅, R₆ and B are fluoro;
R₁ is methoxy when R₁ is Z_{q1}B, q₁ is 2, the first Z group is O, the second Z group is CR₅R₆ where each of R₅ and R₆ is hydrogen, and B is hydrogen;
R₁ is carboxy-methyl, (CO)CH₃ when R₁ is Z_{q1}B, q₁ is 2, the first Z group is CO, the second Z group is CR₅R₆ where each of R₅ and R₆ is hydrogen, and B is hydrogen;
R₁ is methyl sulfonyl, SO₂CH₃ when R₁ is Z_{q1}B, q₁ is 2, the first Z group is SO₂, the second Z group is CR₅R₆ where each of R₅ and R₆ is hydrogen, and B is hydrogen;
R₁ is (CH₂)₃OCH₃ when R₁ is Z_{q1}B, q₁ is 5, each of the first three Z groups and the fifth Z group is CR₅R₆ where each of R₅ and R₆ is hydrogen, the fourth Z group is O, and B is hydrogen;
R₁ is C(CH₃)(CH₃)CN when R₁ is Z_{q1}B, q₁ is 1, the Z group is CR₅R₆ where each of R₅ and R₆ is methyl, and B is cyano.

Specific compounds of the invention include the compounds of Formula (I) listed in Table 1, and any salt or solvate thereof, including a solvate of such a salt:

TABLE 1

| Compound number | Structure |
|---|---|
| 1 | 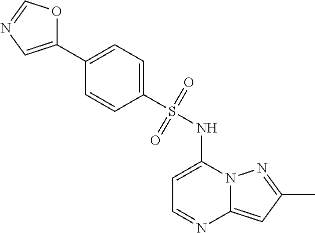 [SMILES: Cc1cc2nccc(NS(=O)(=O)c3ccc(-c4cnco4)cc3)n2n1] |
| 2 | 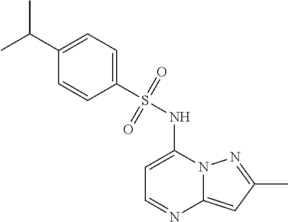 [SMILES: Cc1cc2nccc(NS(=O)(=O)c3ccc(C(C)C)cc3)n2n1] |
| 3 | 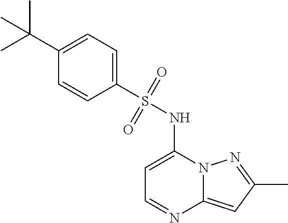 [SMILES: Cc1cc2nccc(NS(=O)(=O)c3ccc(C(C)(C)C)cc3)n2n1] |
| 4 | 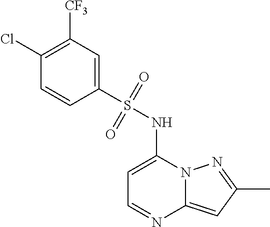 [SMILES: Cc1cc2nccc(NS(=O)(=O)c3ccc(Cl)c(C(F)(F)F)c3)n2n1] |
| 5 | 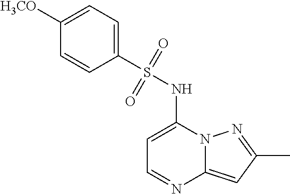 [SMILES: COc1ccc(S(=O)(=O)Nc2ccnc3cc(C)nn23)cc1] |
| 6 | 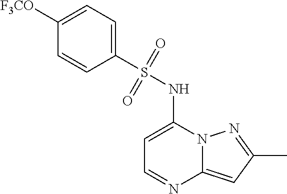 [SMILES: Cc1cc2nccc(NS(=O)(=O)c3ccc(OC(F)(F)F)cc3)n2n1] |
| 7 | 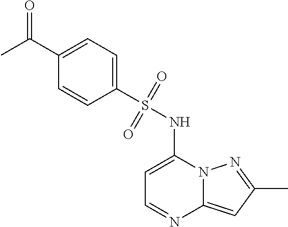 [SMILES: CC(=O)c1ccc(S(=O)(=O)Nc2ccnc3cc(C)nn23)cc1] |
| 8 | 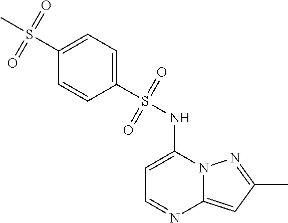 |
| 9 | 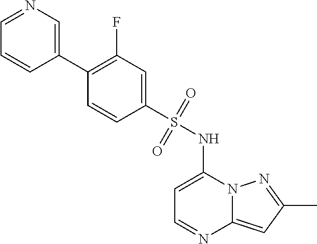 [SMILES: Cc1cc2nccc(NS(=O)(=O)c3ccc(-c4cccnc4)c(F)c3)n2n1] |
| 10 | 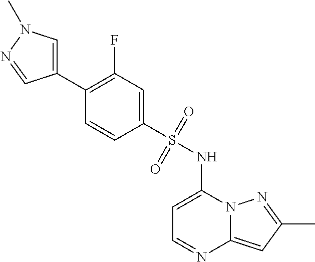 [SMILES: Cc1cc2nccc(NS(=O)(=O)c3ccc(-c4cnn(C)c4)c(F)c3)n2n1] |
| 11 | 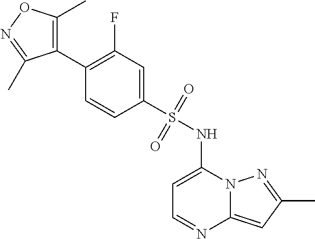 [SMILES: Cc1cc2nccc(NS(=O)(=O)c3ccc(-c4c(C)noc4C)c(F)c3)n2n1] |
| 12 | 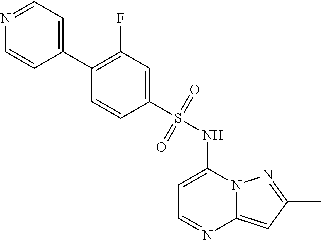 [SMILES: Cc1cc2nccc(NS(=O)(=O)c3ccc(-c4ccncc4)c(F)c3)n2n1] |
| 13 | 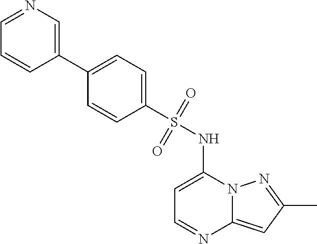 [SMILES: Cc1cc2nccc(NS(=O)(=O)c3ccc(-c4cccnc4)cc3)n2n1] |
| 14 | 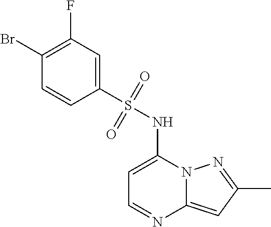 [SMILES: Cc1cc2nccc(NS(=O)(=O)c3ccc(Br)c(F)c3)n2n1] |
| 15 | 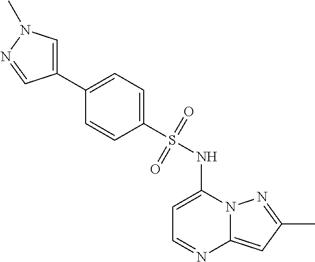 [SMILES: Cc1cc2nccc(NS(=O)(=O)c3ccc(-c4cnn(C)c4)cc3)n2n1] |
| 16 | 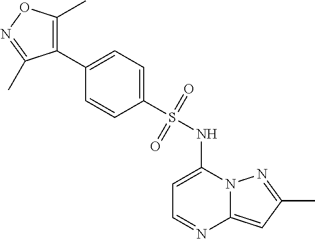 [SMILES: Cc1cc2nccc(NS(=O)(=O)c3ccc(-c4c(C)noc4C)cc3)n2n1] |
| 17 | 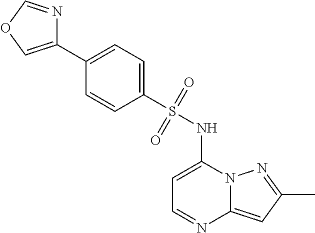 [SMILES: Cc1cc2nccc(NS(=O)(=O)c3ccc(-c4cocn4)cc3)n2n1] |
| 18 | 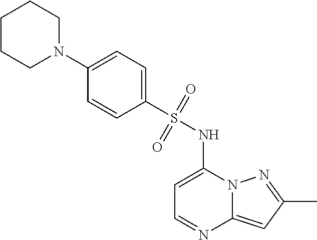 [SMILES: Cc1cc2nccc(NS(=O)(=O)c3ccc(N4CCCCC4)cc3)n2n1] |
| 19 | 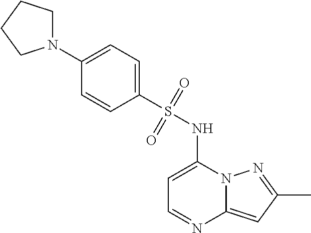 [SMILES: Cc1cc2nccc(NS(=O)(=O)c3ccc(N4CCCC4)cc3)n2n1] |
| 20 | 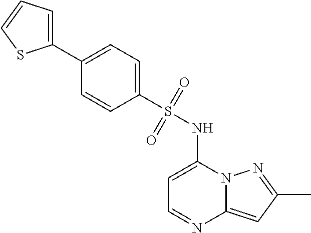 [SMILES: Cc1cc2nccc(NS(=O)(=O)c3ccc(-c4cccs4)cc3)n2n1] |
| 21 | 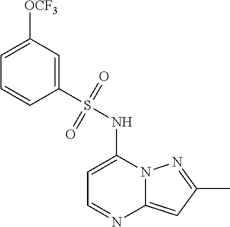 [SMILES: Cc1cc2nccc(NS(=O)(=O)c3cccc(OC(F)(F)F)c3)n2n1] |
| 22 | 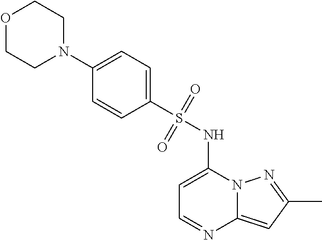 [SMILES: Cc1cc2nccc(NS(=O)(=O)c3ccc(N4CCOCC4)cc3)n2n1] |
| 23 | 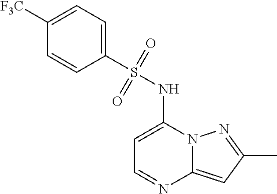 [SMILES: Cc1cc2nccc(NS(=O)(=O)c3ccc(C(F)(F)F)cc3)n2n1] |
| 24 | 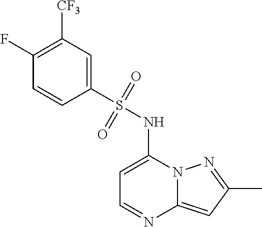 [SMILES: Cc1cc2nccc(NS(=O)(=O)c3ccc(F)c(C(F)(F)F)c3)n2n1] |
| 25 | 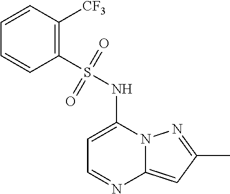 [SMILES: Cc1cc2nccc(NS(=O)(=O)c3ccccc3C(F)(F)F)n2n1] |
| 26 | 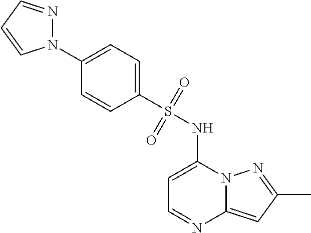 [SMILES: Cc1cc2nccc(NS(=O)(=O)c3ccc(-n4cccn4)cc3)n2n1] |
| 27 | 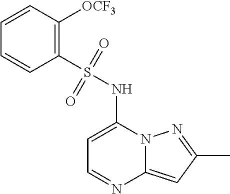 [SMILES: Cc1cc2nccc(NS(=O)(=O)c3ccccc3OC(F)(F)F)n2n1] |
| 28 | 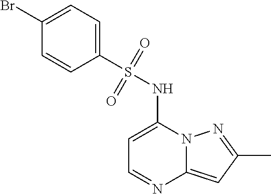 [SMILES: Cc1cc2nccc(NS(=O)(=O)c3ccc(Br)cc3)n2n1] |
| 29 | 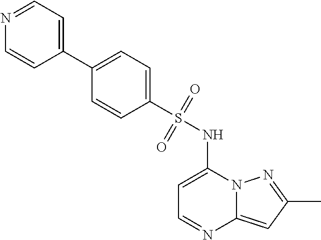 [SMILES: Cc1cc2nccc(NS(=O)(=O)c3ccc(-c4ccncc4)cc3)n2n1] |
| 30 | 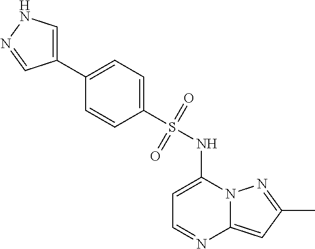 [SMILES: Cc1cc2nccc(NS(=O)(=O)c3ccc(-c4cn[nH]c4)cc3)n2n1] |
| 31 | 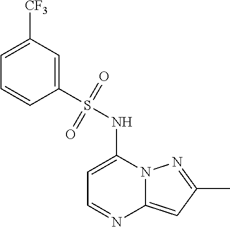 [SMILES: Cc1cc2nccc(NS(=O)(=O)c3cccc(C(F)(F)F)c3)n2n1] |
| 32 | 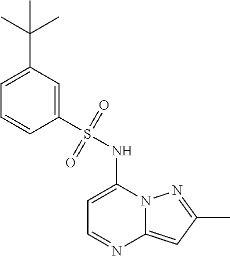 [SMILES: Cc1cc2nccc(NS(=O)(=O)c3cccc(C(C)(C)C)c3)n2n1] |
| 33 | 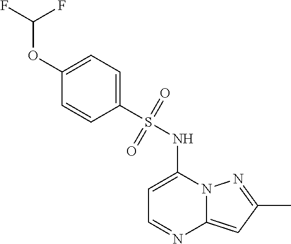 [SMILES: Cc1cc2nccc(NS(=O)(=O)c3ccc(OC(F)F)cc3)n2n1] |
| 34 | 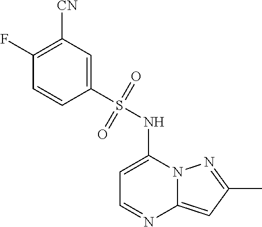 [SMILES: Cc1cc2nccc(NS(=O)(=O)c3ccc(F)c(C#N)c3)n2n1] |
| 35 | 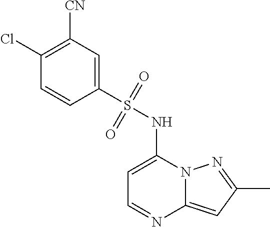 [SMILES: Cc1cc2nccc(NS(=O)(=O)c3ccc(Cl)c(C#N)c3)n2n1] |
| 36 | 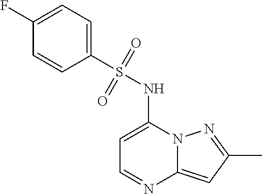 [SMILES: Cc1cc2nccc(NS(=O)(=O)c3ccc(F)cc3)n2n1] |
| 37 | 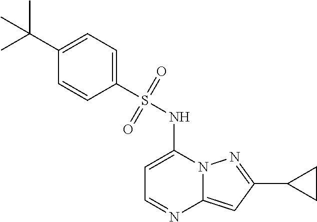 [SMILES: CC(C)(C)c1ccc(S(=O)(=O)Nc2ccnc3cc(C4CC4)nn23)cc1] |
| 38 | 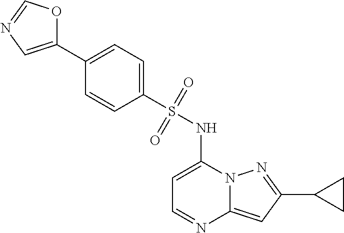 [SMILES: O=S(=O)(Nc1ccnc2cc(C3CC3)nn12)c1ccc(-c2cnco2)cc1] |
| 39 | 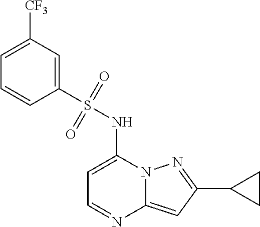 [SMILES: O=S(=O)(Nc1ccnc2cc(C3CC3)nn12)c1cccc(C(F)(F)F)c1] |
| 40 | 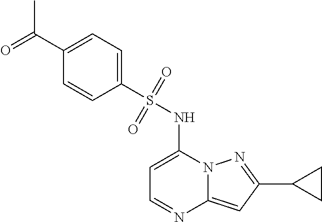 [SMILES: CC(=O)c1ccc(S(=O)(=O)Nc2ccnc3cc(C4CC4)nn23)cc1] |
| 41 | 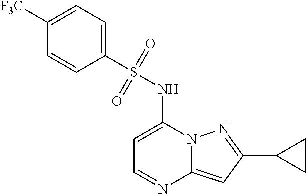 [SMILES: O=S(=O)(Nc1ccnc2cc(C3CC3)nn12)c1ccc(C(F)(F)F)cc1] |
| 42 | 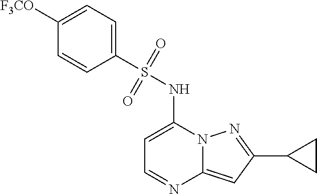 [SMILES: O=S(=O)(Nc1ccnc2cc(C3CC3)nn12)c1ccc(OC(F)(F)F)cc1] |
| 43 | 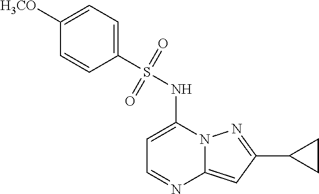 [SMILES: COc1ccc(S(=O)(=O)Nc2ccnc3cc(C4CC4)nn23)cc1] |
| 44 | 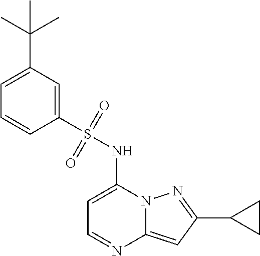 [SMILES: CC(C)(C)c1cccc(S(=O)(=O)Nc2ccnc3cc(C4CC4)nn23)c1] |
| 45 | 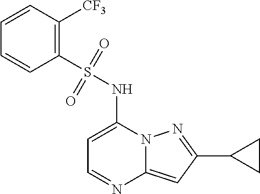 [SMILES: O=S(=O)(Nc1ccnc2cc(C3CC3)nn12)c1ccccc1C(F)(F)F] |
| 46 | 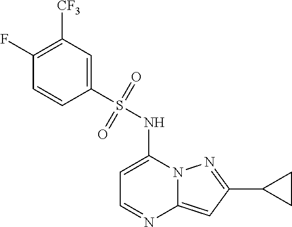 [SMILES: O=S(=O)(Nc1ccnc2cc(C3CC3)nn12)c1ccc(F)c(C(F)(F)F)c1] |
| 47 | 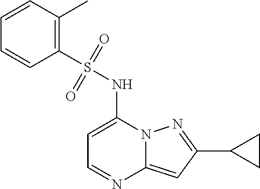 [SMILES: Cc1ccccc1S(=O)(=O)Nc1ccnc2cc(C3CC3)nn12] |
| 48 | 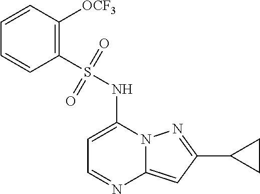 [SMILES: O=S(=O)(Nc1ccnc2cc(C3CC3)nn12)c1ccccc1OC(F)(F)F] |
| 49 | 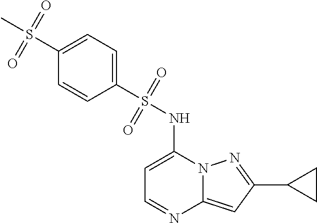 [SMILES: CS(=O)(=O)c1ccc(S(=O)(=O)Nc2ccnc3cc(C4CC4)nn23)cc1] |
| 50 | 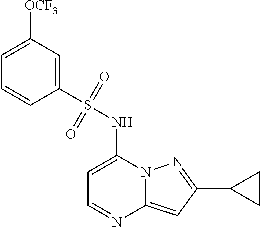 [SMILES: O=S(=O)(Nc1ccnc2cc(C3CC3)nn12)c1cccc(OC(F)(F)F)c1] |
| 51 | 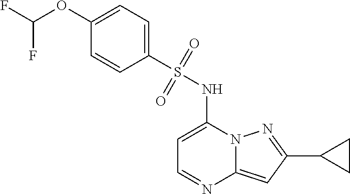 [SMILES: O=S(=O)(Nc1ccnc2cc(C3CC3)nn12)c1ccc(OC(F)F)cc1] |
| 52 | 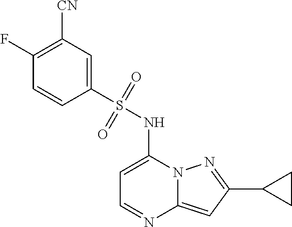 [SMILES: N#Cc1cc(S(=O)(=O)Nc2ccnc3cc(C4CC4)nn23)ccc1F] |
| 53 | 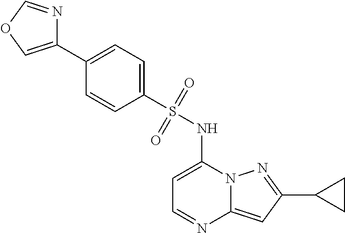 [SMILES: O=S(=O)(Nc1ccnc2cc(C3CC3)nn12)c1ccc(-c2cocn2)cc1] |
| 54 | 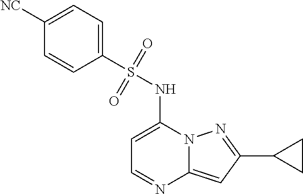 [SMILES: N#Cc1ccc(S(=O)(=O)Nc2ccnc3cc(C4CC4)nn23)cc1] |
| 55 | 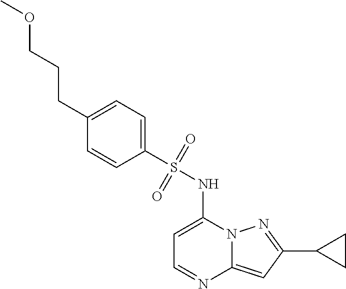 [SMILES: COCCCc1ccc(S(=O)(=O)Nc2ccnc3cc(C4CC4)nn23)cc1] |
| 56 | 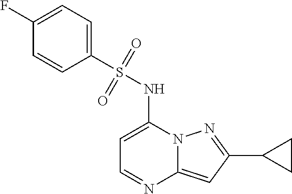 [SMILES: O=S(=O)(Nc1ccnc2cc(C3CC3)nn12)c1ccc(F)cc1] |
| 57 | 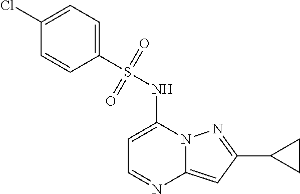 [SMILES: O=S(=O)(Nc1ccnc2cc(C3CC3)nn12)c1ccc(Cl)cc1] |
| 58 | 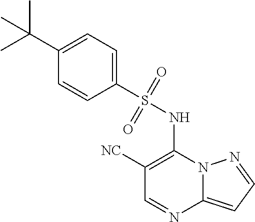 [SMILES: CC(C)(C)c1ccc(S(=O)(=O)Nc2c(C#N)cnc3ccnn23)cc1] |
| 59 | 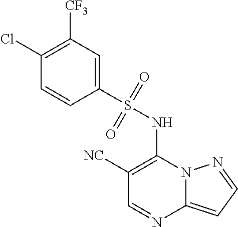 [SMILES: N#Cc1cnc2ccnn2c1NS(=O)(=O)c1ccc(Cl)c(C(F)(F)F)c1] |
| 60 | 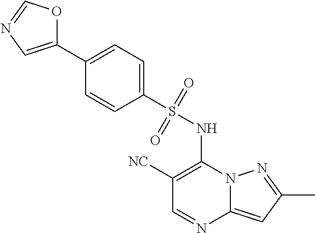 [SMILES: Cc1cc2ncc(C#N)c(NS(=O)(=O)c3ccc(-c4cnco4)cc3)n2n1] |
| 61 | 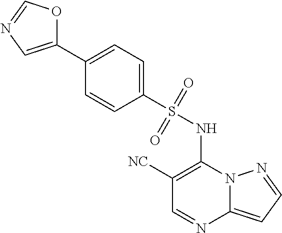 [SMILES: N#Cc1cnc2ccnn2c1NS(=O)(=O)c1ccc(-c2cnco2)cc1] |
| 62 | 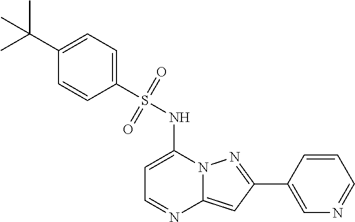 [SMILES: CC(C)(C)c1ccc(S(=O)(=O)Nc2ccnc3cc(-c4cccnc4)nn23)cc1] |
| 63 | 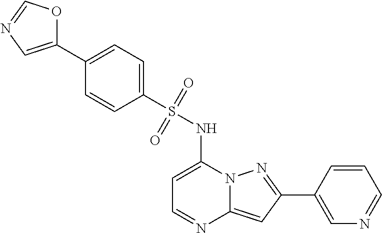 [SMILES: O=S(=O)(Nc1ccnc2cc(-c3cccnc3)nn12)c1ccc(-c2cnco2)cc1] |
| 64 | 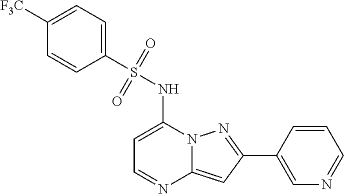 [SMILES: O=S(=O)(Nc1ccnc2cc(-c3cccnc3)nn12)c1ccc(C(F)(F)F)cc1] |
| 65 | 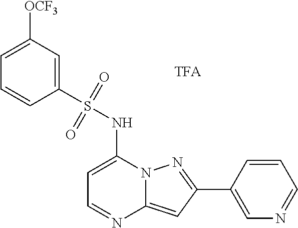 [SMILES: O=S(=O)(Nc1ccnc2cc(-c3cccnc3)nn12)c1cccc(OC(F)(F)F)c1] |
| 66 | 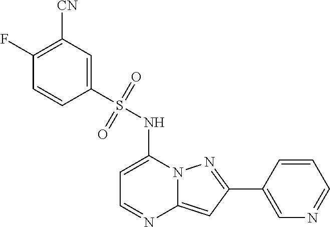 [SMILES: N#Cc1cc(S(=O)(=O)Nc2ccnc3cc(-c4cccnc4)nn23)ccc1F] |
| 67 | 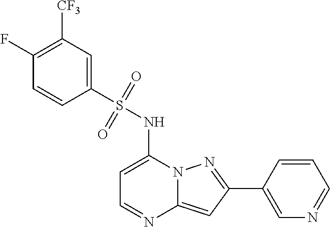 [SMILES: O=S(=O)(Nc1ccnc2cc(-c3cccnc3)nn12)c1ccc(F)c(C(F)(F)F)c1] |
| 68 | 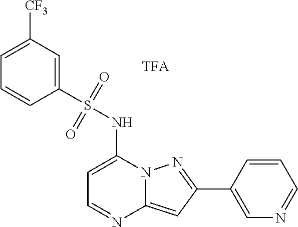 [SMILES: O=S(=O)(Nc1ccnc2cc(-c3cccnc3)nn12)c1cccc(C(F)(F)F)c1] |
| 69 | 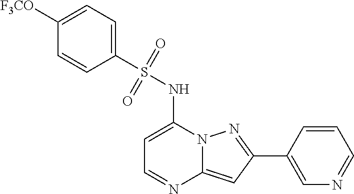 [SMILES: O=S(=O)(Nc1ccnc2cc(-c3cccnc3)nn12)c1ccc(OC(F)(F)F)cc1] |
| 70 | 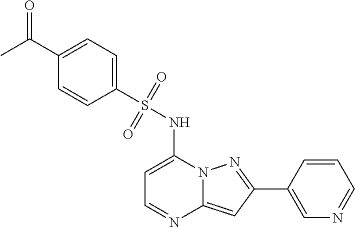 [SMILES: CC(=O)c1ccc(S(=O)(=O)Nc2ccnc3cc(-c4cccnc4)nn23)cc1] |
| 71 | 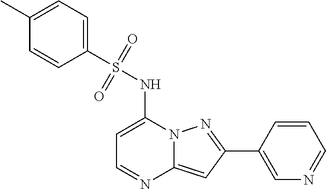 [SMILES: Cc1ccc(S(=O)(=O)Nc2ccnc3cc(-c4cccnc4)nn23)cc1] |
| 72 | 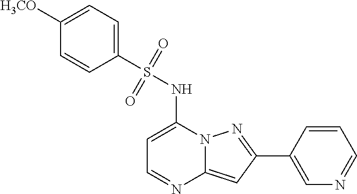 [SMILES: COc1ccc(S(=O)(=O)Nc2ccnc3cc(-c4cccnc4)nn23)cc1] |
| 73 | 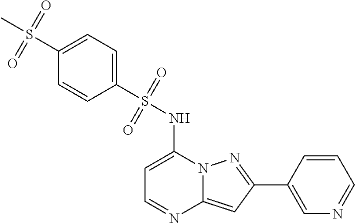 [SMILES: CS(=O)(=O)c1ccc(S(=O)(=O)Nc2ccnc3cc(-c4cccnc4)nn23)cc1] |
| 74 | 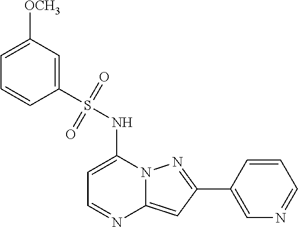 [SMILES: COc1cccc(S(=O)(=O)Nc2ccnc3cc(-c4cccnc4)nn23)c1] |
| 75 | 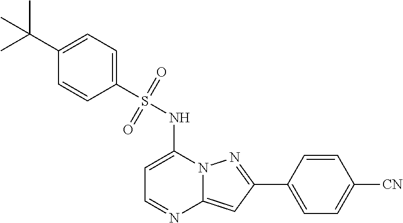 [SMILES: CC(C)(C)c1ccc(S(=O)(=O)Nc2ccnc3cc(-c4ccc(C#N)cc4)nn23)cc1] |
| 76 | 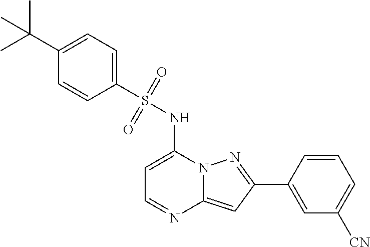 [SMILES: CC(C)(C)c1ccc(S(=O)(=O)Nc2ccnc3cc(-c4cccc(C#N)c4)nn23)cc1] |
| 77 | 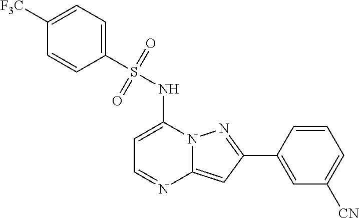 [SMILES: N#Cc1cccc(-c2cc3nccc(NS(=O)(=O)c4ccc(C(F)(F)F)cc4)n3n2)c1] |
| 78 | 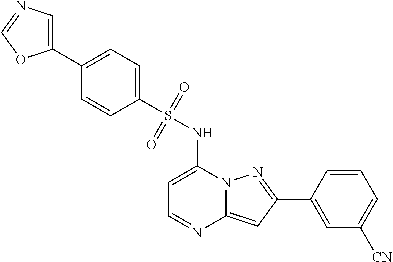 [SMILES: N#Cc1cccc(-c2cc3nccc(NS(=O)(=O)c4ccc(-c5cnco5)cc4)n3n2)c1] |
| 79 | 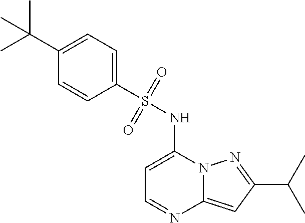 [SMILES: CC(C)c1cc2nccc(NS(=O)(=O)c3ccc(C(C)(C)C)cc3)n2n1] |
| 80 | 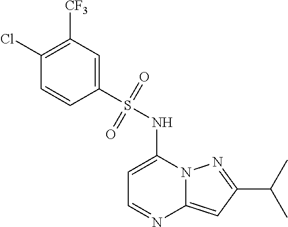 [SMILES: CC(C)c1cc2nccc(NS(=O)(=O)c3ccc(Cl)c(C(F)(F)F)c3)n2n1] |
| 81 | 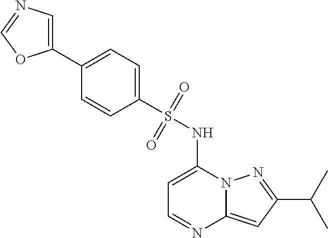 [SMILES: CC(C)c1cc2nccc(NS(=O)(=O)c3ccc(-c4cnco4)cc3)n2n1] |
| 82 | 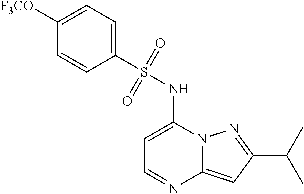 [SMILES: CC(C)c1cc2nccc(NS(=O)(=O)c3ccc(OC(F)(F)F)cc3)n2n1] |
| 83 | 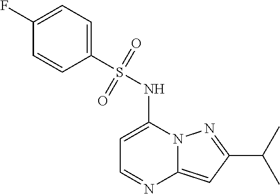 [SMILES: CC(C)c1cc2nccc(NS(=O)(=O)c3ccc(F)cc3)n2n1] |
| 84 | 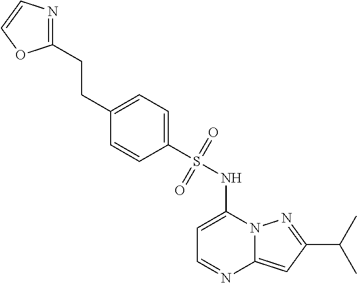 [SMILES: CC(C)c1cc2nccc(NS(=O)(=O)c3ccc(CCc4ncco4)cc3)n2n1] |
| 85 | 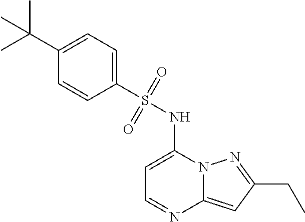 [SMILES: CCc1cc2nccc(NS(=O)(=O)c3ccc(C(C)(C)C)cc3)n2n1] |
| 86 | 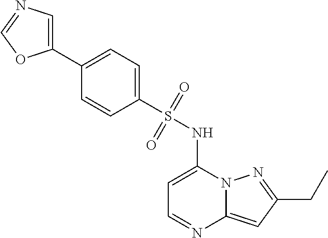 [SMILES: CCc1cc2nccc(NS(=O)(=O)c3ccc(-c4cnco4)cc3)n2n1] |
| 87 | 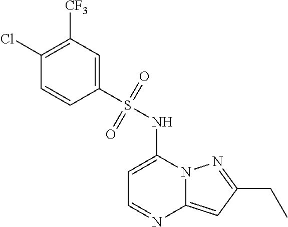 [SMILES: CCc1cc2nccc(NS(=O)(=O)c3ccc(Cl)c(C(F)(F)F)c3)n2n1] |
| 88 | 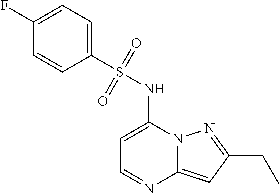 [SMILES: CCc1cc2nccc(NS(=O)(=O)c3ccc(F)cc3)n2n1] |
| 89 | 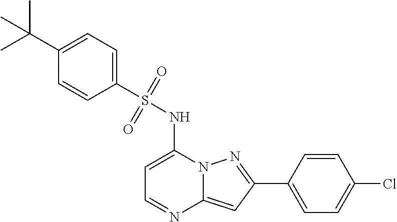 [SMILES: CC(C)(C)c1ccc(S(=O)(=O)Nc2ccnc3cc(-c4ccc(Cl)cc4)nn23)cc1] |
| 90 | 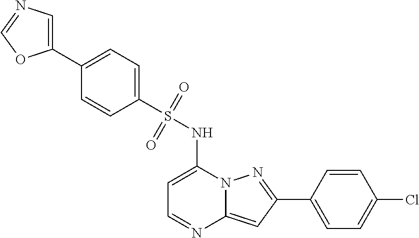 [SMILES: O=S(=O)(Nc1ccnc2cc(-c3ccc(Cl)cc3)nn12)c1ccc(-c2cnco2)cc1] |
| 91 | 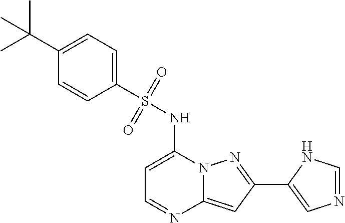 [SMILES: CC(C)(C)c1ccc(S(=O)(=O)Nc2ccnc3cc(-c4cnc[nH]4)nn23)cc1] |
| 92 | 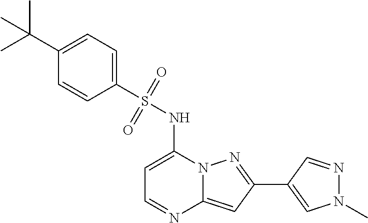 [SMILES: Cn1cc(-c2cc3nccc(NS(=O)(=O)c4ccc(C(C)(C)C)cc4)n3n2)cn1] |
| 93 | 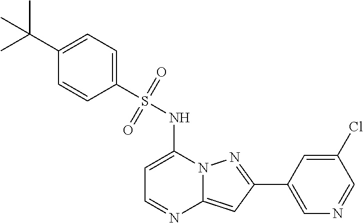 [SMILES: CC(C)(C)c1ccc(S(=O)(=O)Nc2ccnc3cc(-c4cncc(Cl)c4)nn23)cc1] |
| 94 | 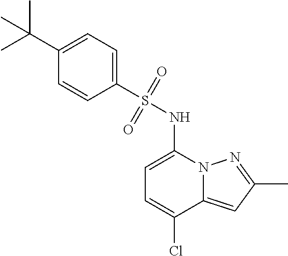 [SMILES: Cc1cc2c(Cl)ccc(NS(=O)(=O)c3ccc(C(C)(C)C)cc3)n2n1] |
| 95 | 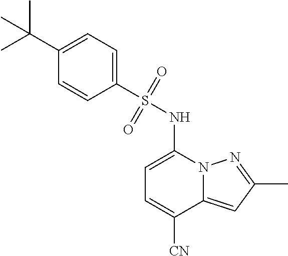 [SMILES: Cc1cc2c(C#N)ccc(NS(=O)(=O)c3ccc(C(C)(C)C)cc3)n2n1] |
| 96 | 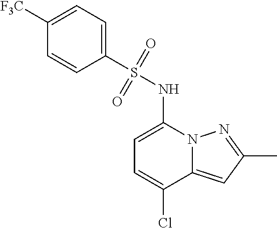 [SMILES: Cc1cc2c(Cl)ccc(NS(=O)(=O)c3ccc(C(F)(F)F)cc3)n2n1] |
| 97 | 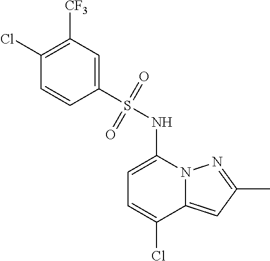 [SMILES: Cc1cc2c(Cl)ccc(NS(=O)(=O)c3ccc(Cl)c(C(F)(F)F)c3)n2n1] |
| 98 | 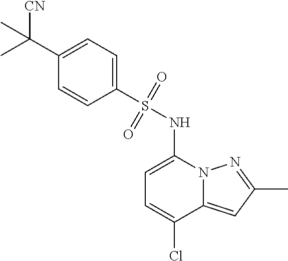 [SMILES: Cc1cc2c(Cl)ccc(NS(=O)(=O)c3ccc(C(C)(C)C#N)cc3)n2n1] |
| 99 | 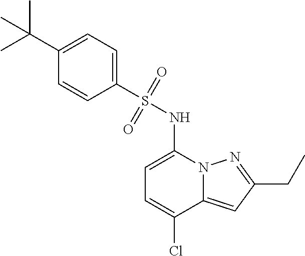 [SMILES: CCc1cc2c(Cl)ccc(NS(=O)(=O)c3ccc(C(C)(C)C)cc3)n2n1] |
| 100 | 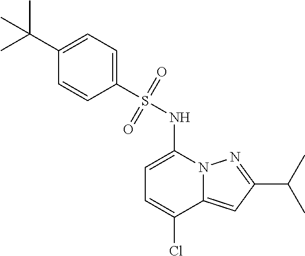 [SMILES: CC(C)c1cc2c(Cl)ccc(NS(=O)(=O)c3ccc(C(C)(C)C)cc3)n2n1] |
| 101 | 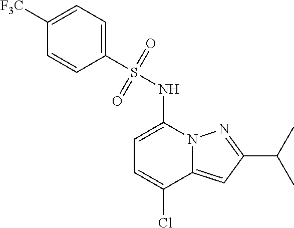 [SMILES: CC(C)c1cc2c(Cl)ccc(NS(=O)(=O)c3ccc(C(F)(F)F)cc3)n2n1] |
| 102 | 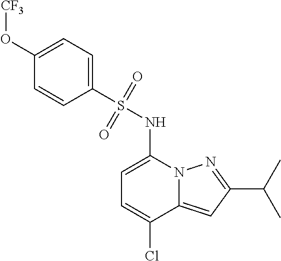 [SMILES: CC(C)c1cc2c(Cl)ccc(NS(=O)(=O)c3ccc(OC(F)(F)F)cc3)n2n1] |
| 103 | 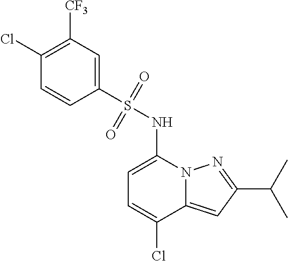 [SMILES: CC(C)c1cc2c(Cl)ccc(NS(=O)(=O)c3ccc(Cl)c(C(F)(F)F)c3)n2n1] |
| 104 | 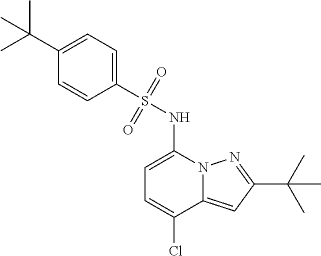 [SMILES: CC(C)(C)c1ccc(S(=O)(=O)Nc2ccc(Cl)c3cc(C(C)(C)C)nn23)cc1] |
| 105 | 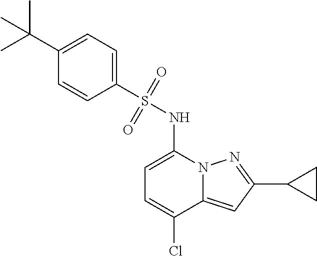 [SMILES: CC(C)(C)c1ccc(S(=O)(=O)Nc2ccc(Cl)c3cc(C4CC4)nn23)cc1] |
| 106 | 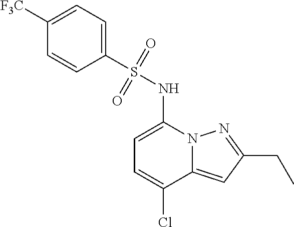 [SMILES: CCc1cc2c(Cl)ccc(NS(=O)(=O)c3ccc(C(F)(F)F)cc3)n2n1] |
| 107 | 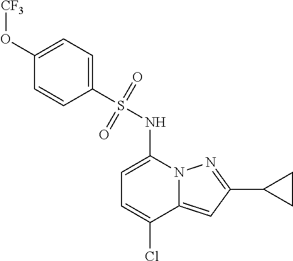 [SMILES: O=S(=O)(Nc1ccc(Cl)c2cc(C3CC3)nn12)c1ccc(OC(F)(F)F)cc1] |
| 108 | 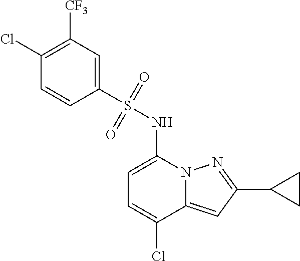 [SMILES: O=S(=O)(Nc1ccc(Cl)c2cc(C3CC3)nn12)c1ccc(Cl)c(C(F)(F)F)c1] |
| 109 | 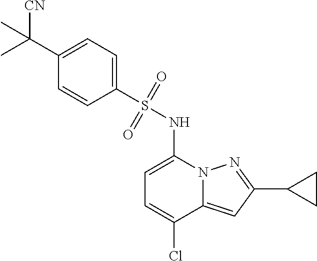 [SMILES: CC(C)(C#N)c1ccc(S(=O)(=O)Nc2ccc(Cl)c3cc(C4CC4)nn23)cc1] |
| 110 | 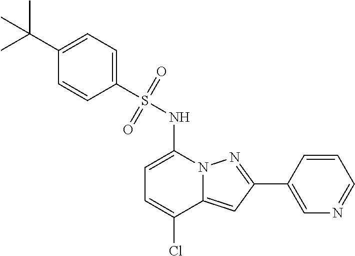 [SMILES: CC(C)(C)c1ccc(S(=O)(=O)Nc2ccc(Cl)c3cc(-c4cccnc4)nn23)cc1] |
| 111 | 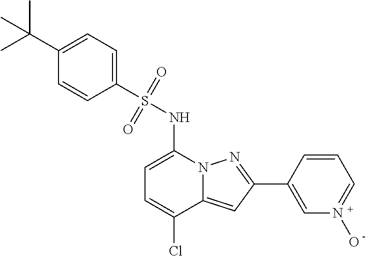 |
| 112 | 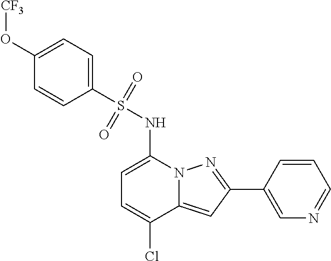 [SMILES: O=S(=O)(Nc1ccc(Cl)c2cc(-c3cccnc3)nn12)c1ccc(OC(F)(F)F)cc1] |
| 113 | 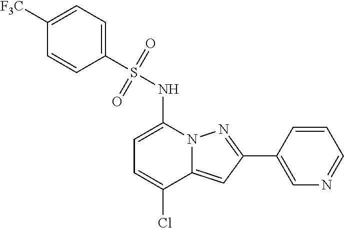 [SMILES: O=S(=O)(Nc1ccc(Cl)c2cc(-c3cccnc3)nn12)c1ccc(C(F)(F)F)cc1] |
| 114 | 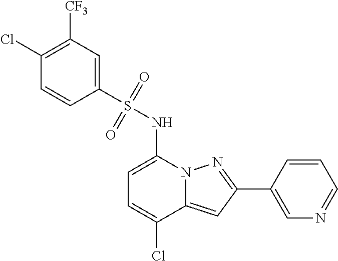 [SMILES: O=S(=O)(Nc1ccc(Cl)c2cc(-c3cccnc3)nn12)c1ccc(Cl)c(C(F)(F)F)c1] |
| 115 | 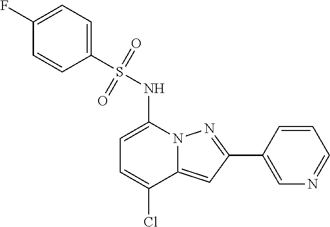 [SMILES: O=S(=O)(Nc1ccc(Cl)c2cc(-c3cccnc3)nn12)c1ccc(F)cc1] |
| 116 | 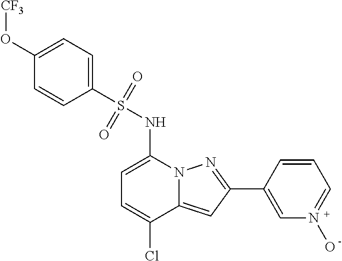 |
| 117 | 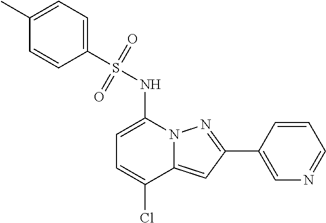 [SMILES: Cc1ccc(S(=O)(=O)Nc2ccc(Cl)c3cc(-c4cccnc4)nn23)cc1] |
| 118 | 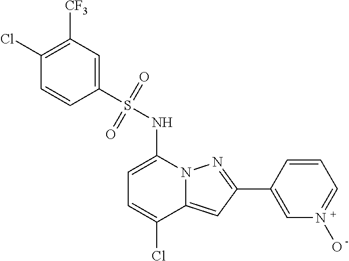 |
| 119 | 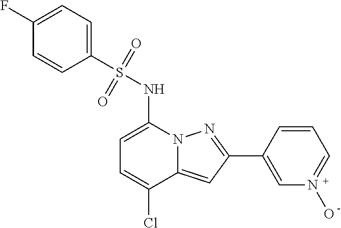 |
| 120 | 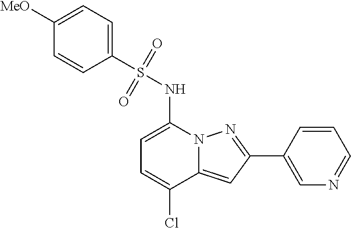 [SMILES: COc1ccc(S(=O)(=O)Nc2ccc(Cl)c3cc(-c4cccnc4)nn23)cc1] |
| 121 | 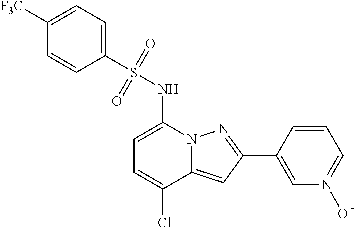 |
| 122 | 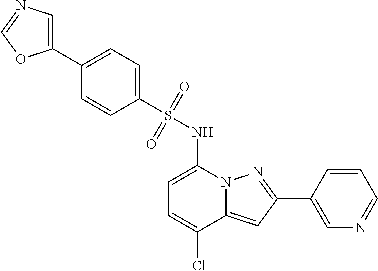 [SMILES: O=S(=O)(Nc1ccc(Cl)c2cc(-c3cccnc3)nn12)c1ccc(-c2cnco2)cc1] |
| 123 | 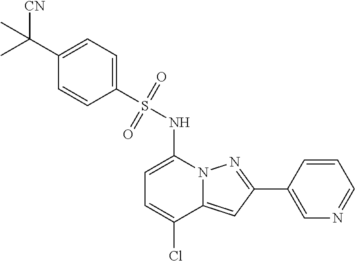 [SMILES: CC(C)(C#N)c1ccc(S(=O)(=O)Nc2ccc(Cl)c3cc(-c4cccnc4)nn23)cc1] |
| 124 | 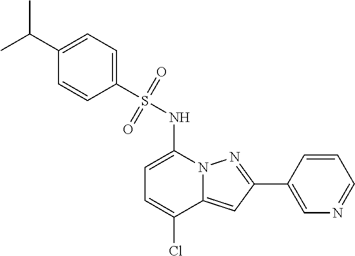 [SMILES: CC(C)c1ccc(S(=O)(=O)Nc2ccc(Cl)c3cc(-c4cccnc4)nn23)cc1] |
| 125 | 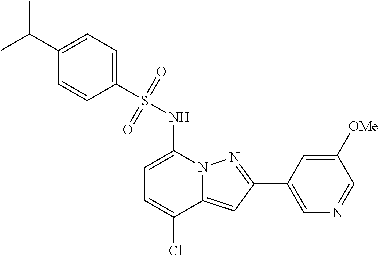 [SMILES: COc1cncc(-c2cc3c(Cl)ccc(NS(=O)(=O)c4ccc(C(C)C)cc4)n3n2)c1] |
| 126 | 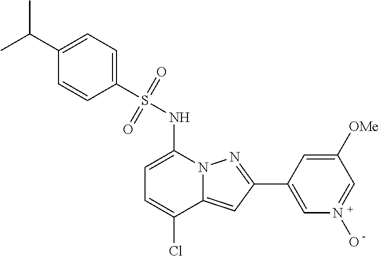 |
| 127 | 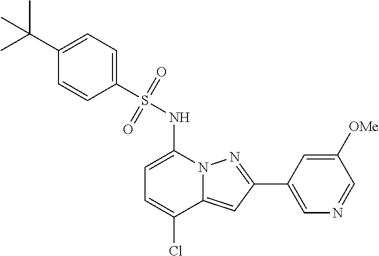 [SMILES: COc1cncc(-c2cc3c(Cl)ccc(NS(=O)(=O)c4ccc(C(C)(C)C)cc4)n3n2)c1] |
| 128 | 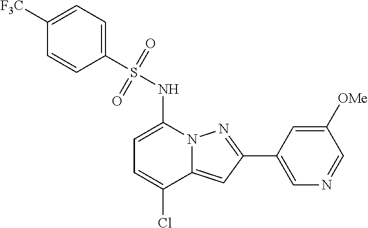 [SMILES: COc1cncc(-c2cc3c(Cl)ccc(NS(=O)(=O)c4ccc(C(F)(F)F)cc4)n3n2)c1] |
| 129 | 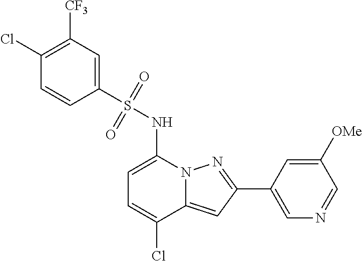 [SMILES: COc1cncc(-c2cc3c(Cl)ccc(NS(=O)(=O)c4ccc(Cl)c(C(F)(F)F)c4)n3n2)c1] |
| 130 | 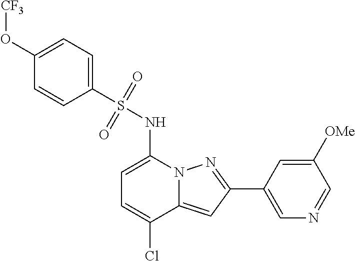 [SMILES: COc1cncc(-c2cc3c(Cl)ccc(NS(=O)(=O)c4ccc(OC(F)(F)F)cc4)n3n2)c1] |
| 131 | 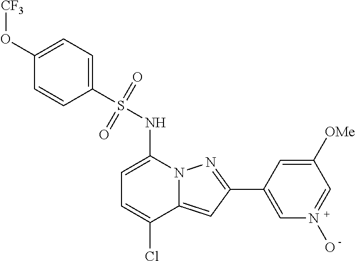 |
| 132 | 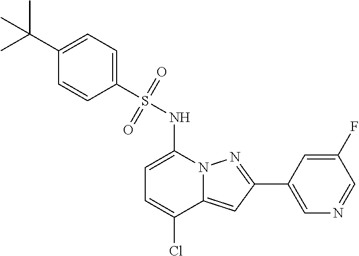 [SMILES: CC(C)(C)c1ccc(S(=O)(=O)Nc2ccc(Cl)c3cc(-c4cncc(F)c4)nn23)cc1] |
| 133 | 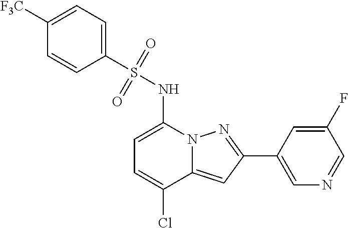 [SMILES: O=S(=O)(Nc1ccc(Cl)c2cc(-c3cncc(F)c3)nn12)c1ccc(C(F)(F)F)cc1] |
| 134 | 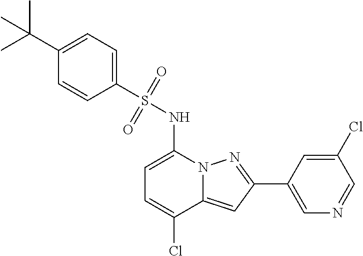 [SMILES: CC(C)(C)c1ccc(S(=O)(=O)Nc2ccc(Cl)c3cc(-c4cncc(Cl)c4)nn23)cc1] |
| 135 | 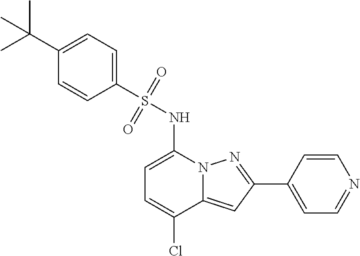 [SMILES: CC(C)(C)c1ccc(S(=O)(=O)Nc2ccc(Cl)c3cc(-c4ccncc4)nn23)cc1] |
| 136 | 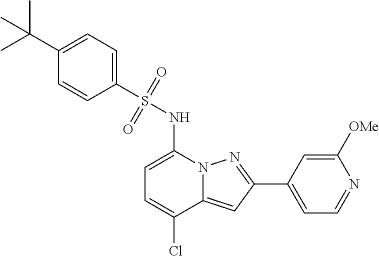 [SMILES: COc1cc(-c2cc3c(Cl)ccc(NS(=O)(=O)c4ccc(C(C)(C)C)cc4)n3n2)ccn1] |
| 137 | 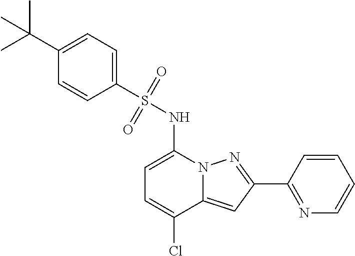 [SMILES: CC(C)(C)c1ccc(S(=O)(=O)Nc2ccc(Cl)c3cc(-c4ccccn4)nn23)cc1] |
| 138 | 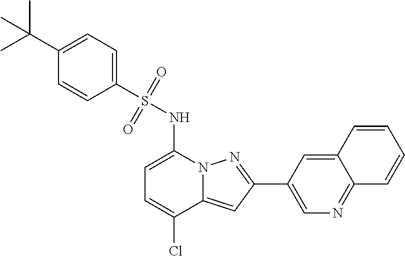 [SMILES: CC(C)(C)c1ccc(S(=O)(=O)Nc2ccc(Cl)c3cc(-c4cnc5ccccc5c4)nn23)cc1] |
| 139 | 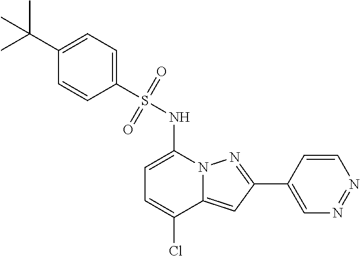 [SMILES: CC(C)(C)c1ccc(S(=O)(=O)Nc2ccc(Cl)c3cc(-c4ccnnc4)nn23)cc1] |
| 140 | 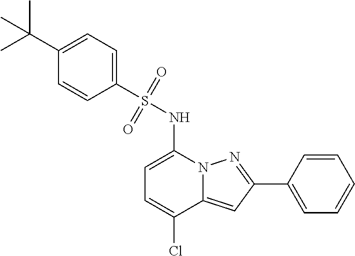 [SMILES: CC(C)(C)c1ccc(S(=O)(=O)Nc2ccc(Cl)c3cc(-c4ccccc4)nn23)cc1] |
| 141 | 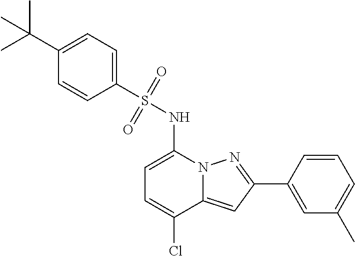 [SMILES: Cc1cccc(-c2cc3c(Cl)ccc(NS(=O)(=O)c4ccc(C(C)(C)C)cc4)n3n2)c1] |
| 142 | 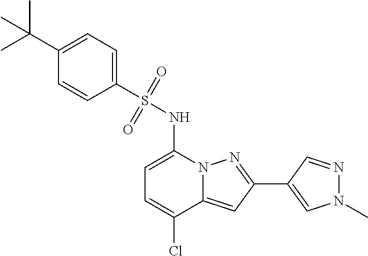 [SMILES: Cn1cc(-c2cc3c(Cl)ccc(NS(=O)(=O)c4ccc(C(C)(C)C)cc4)n3n2)cn1] |
| 143 | 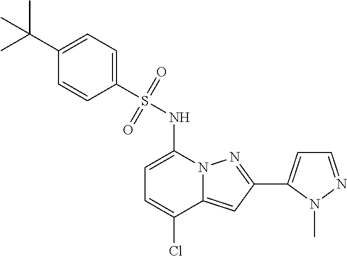 [SMILES: Cn1nccc1-c1cc2c(Cl)ccc(NS(=O)(=O)c3ccc(C(C)(C)C)cc3)n2n1] |
| 144 | 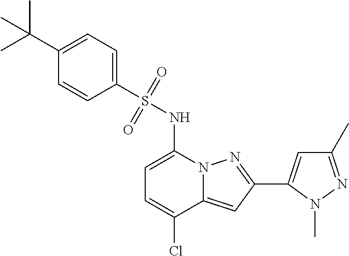 [SMILES: Cc1cc(-c2cc3c(Cl)ccc(NS(=O)(=O)c4ccc(C(C)(C)C)cc4)n3n2)n(C)n1] |
| 145 | 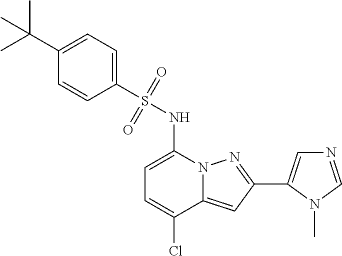 [SMILES: Cn1cncc1-c1cc2c(Cl)ccc(NS(=O)(=O)c3ccc(C(C)(C)C)cc3)n2n1] |
| 146 | 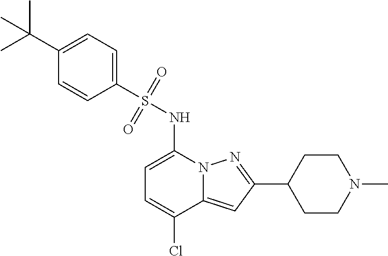 [SMILES: CN1CCC(c2cc3c(Cl)ccc(NS(=O)(=O)c4ccc(C(C)(C)C)cc4)n3n2)CC1] |
| 147 | 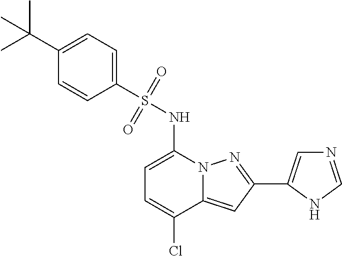 [SMILES: CC(C)(C)c1ccc(S(=O)(=O)Nc2ccc(Cl)c3cc(-c4cnc[nH]4)nn23)cc1] |
| 148 | 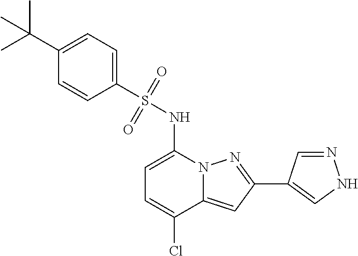 [SMILES: CC(C)(C)c1ccc(S(=O)(=O)Nc2ccc(Cl)c3cc(-c4cn[nH]c4)nn23)cc1] |
| 149 | 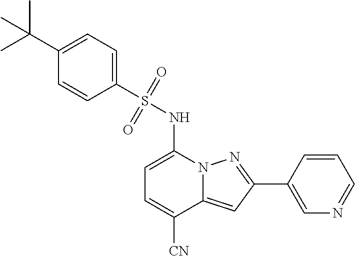 [SMILES: CC(C)(C)c1ccc(S(=O)(=O)Nc2ccc(C#N)c3cc(-c4cccnc4)nn23)cc1] |
| 150 | 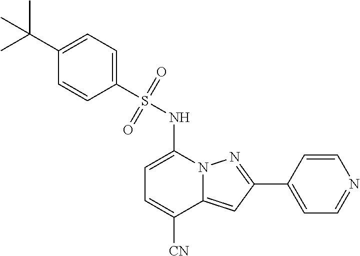 [SMILES: CC(C)(C)c1ccc(S(=O)(=O)Nc2ccc(C#N)c3cc(-c4ccncc4)nn23)cc1] |
| 151 | 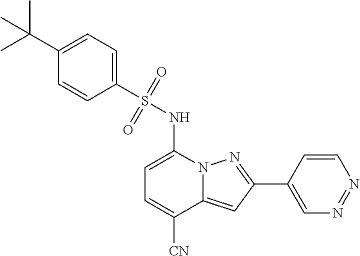 [SMILES: CC(C)(C)c1ccc(S(=O)(=O)Nc2ccc(C#N)c3cc(-c4ccnnc4)nn23)cc1] |
| 152 | 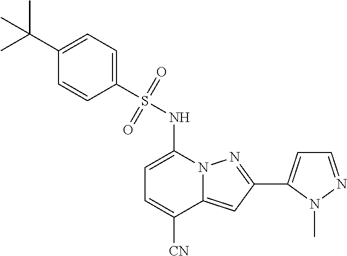 [SMILES: Cn1nccc1-c1cc2c(C#N)ccc(NS(=O)(=O)c3ccc(C(C)(C)C)cc3)n2n1] |
| 153 | 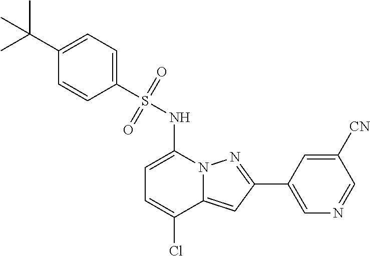 [SMILES: CC(C)(C)c1ccc(S(=O)(=O)Nc2ccc(Cl)c3cc(-c4cncc(C#N)c4)nn23)cc1] |
| 154 | 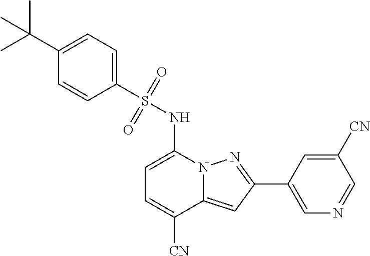 [SMILES: CC(C)(C)c1ccc(S(=O)(=O)Nc2ccc(C#N)c3cc(-c4cncc(C#N)c4)nn23)cc1] |
| 155 | 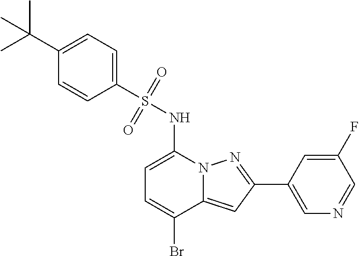 [SMILES: CC(C)(C)c1ccc(S(=O)(=O)Nc2ccc(Br)c3cc(-c4cncc(F)c4)nn23)cc1] |
| 156 | 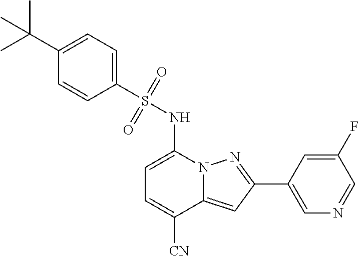 [SMILES: CC(C)(C)c1ccc(S(=O)(=O)Nc2ccc(C#N)c3cc(-c4cncc(F)c4)nn23)cc1] |
| 157 | 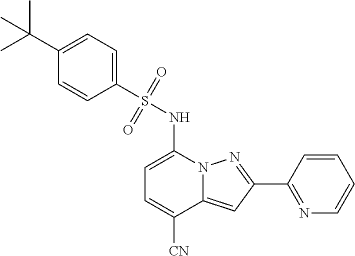 [SMILES: CC(C)(C)c1ccc(S(=O)(=O)Nc2ccc(C#N)c3cc(-c4ccccn4)nn23)cc1] |
| 158 | 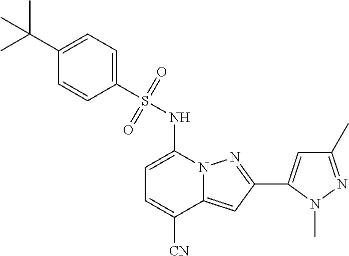 [SMILES: Cc1cc(-c2cc3c(C#N)ccc(NS(=O)(=O)c4ccc(C(C)(C)C)cc4)n3n2)n(C)n1] |
| 159 | 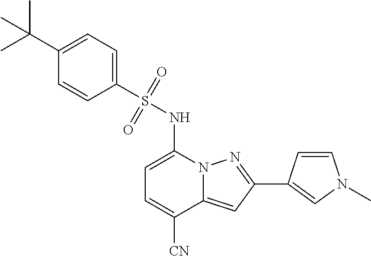 [SMILES: Cn1ccc(-c2cc3c(C#N)ccc(NS(=O)(=O)c4ccc(C(C)(C)C)cc4)n3n2)c1] |
| 160 | 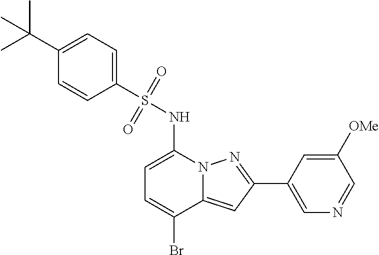 [SMILES: COc1cncc(-c2cc3c(Br)ccc(NS(=O)(=O)c4ccc(C(C)(C)C)cc4)n3n2)c1] |
| 161 | 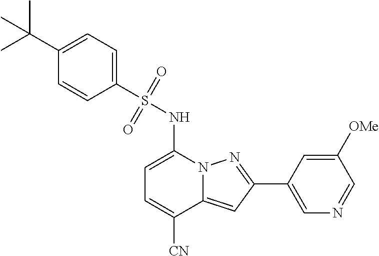 [SMILES: COc1cncc(-c2cc3c(C#N)ccc(NS(=O)(=O)c4ccc(C(C)(C)C)cc4)n3n2)c1] |
| 162 | 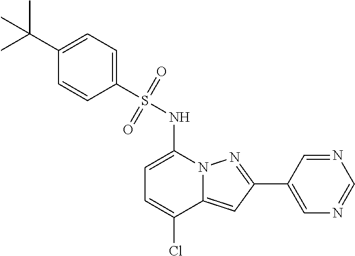 [SMILES: CC(C)(C)c1ccc(S(=O)(=O)Nc2ccc(Cl)c3cc(-c4cncnc4)nn23)cc1] |
| 163 | 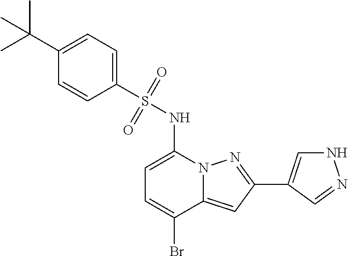 [SMILES: CC(C)(C)c1ccc(S(=O)(=O)Nc2ccc(Br)c3cc(-c4cn[nH]c4)nn23)cc1] |
| 164 | 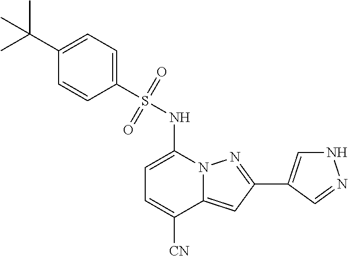 [SMILES: CC(C)(C)c1ccc(S(=O)(=O)Nc2ccc(C#N)c3cc(-c4cn[nH]c4)nn23)cc1] |
| 165 | 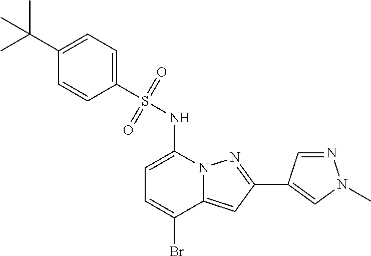 [SMILES: Cn1cc(-c2cc3c(Br)ccc(NS(=O)(=O)c4ccc(C(C)(C)C)cc4)n3n2)cn1] |
| 166 | 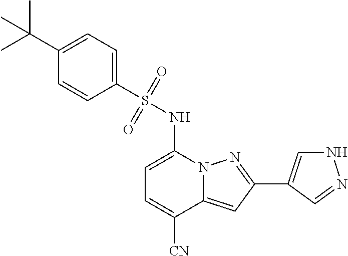 [SMILES: CC(C)(C)c1ccc(S(=O)(=O)Nc2ccc(C#N)c3cc(-c4cn[nH]c4)nn23)cc1] |
| 167 | 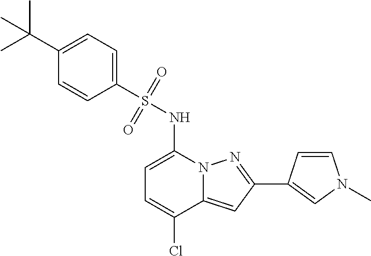 [SMILES: Cn1ccc(-c2cc3c(Cl)ccc(NS(=O)(=O)c4ccc(C(C)(C)C)cc4)n3n2)c1] |
| 168 | 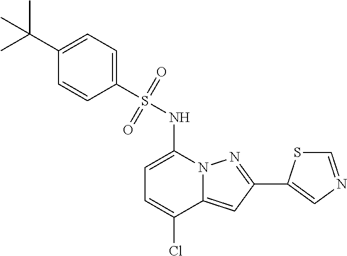 [SMILES: CC(C)(C)c1ccc(S(=O)(=O)Nc2ccc(Cl)c3cc(-c4cncs4)nn23)cc1] |
| 169 | 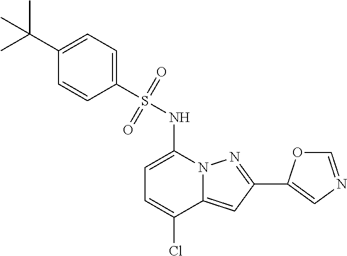 [SMILES: CC(C)(C)c1ccc(S(=O)(=O)Nc2ccc(Cl)c3cc(-c4cnco4)nn23)cc1] |
| 170 | 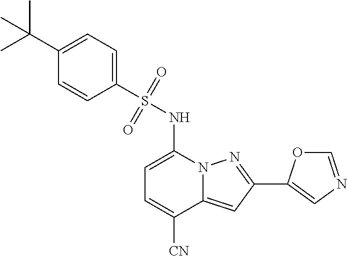 [SMILES: CC(C)(C)c1ccc(S(=O)(=O)Nc2ccc(C#N)c3cc(-c4cnco4)nn23)cc1] |
| 171 | 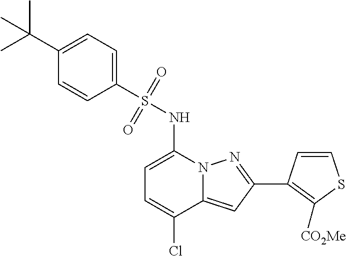 [SMILES: COC(=O)c1sccc1-c1cc2c(Cl)ccc(NS(=O)(=O)c3ccc(C(C)(C)C)cc3)n2n1] |
| 172 | 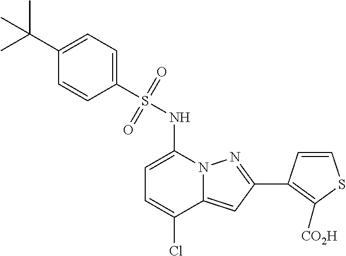 [SMILES: CC(C)(C)c1ccc(S(=O)(=O)Nc2ccc(Cl)c3cc(-c4ccsc4C(=O)O)nn23)cc1] |
| 173 | 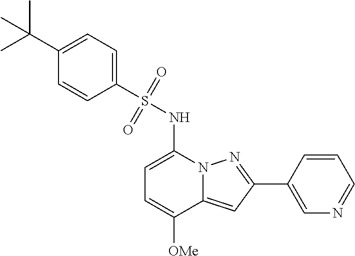 [SMILES: COc1ccc(NS(=O)(=O)c2ccc(C(C)(C)C)cc2)n2nc(-c3cccnc3)cc12] |
| 174 | 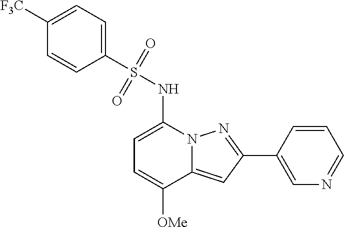 [SMILES: COc1ccc(NS(=O)(=O)c2ccc(C(F)(F)F)cc2)n2nc(-c3cccnc3)cc12] |
| 175 | 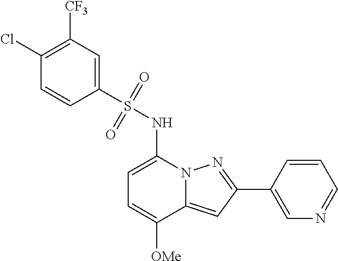 [SMILES: COc1ccc(NS(=O)(=O)c2ccc(Cl)c(C(F)(F)F)c2)n2nc(-c3cccnc3)cc12] |
| 176 | 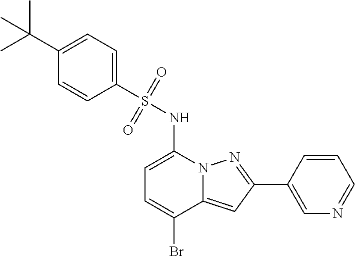 [SMILES: CC(C)(C)c1ccc(S(=O)(=O)Nc2ccc(Br)c3cc(-c4cccnc4)nn23)cc1] |
| 177 | 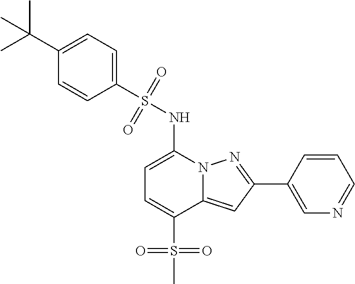 [SMILES: CC(C)(C)c1ccc(S(=O)(=O)Nc2ccc(S(C)(=O)=O)c3cc(-c4cccnc4)nn23)cc1] |
| 178 | 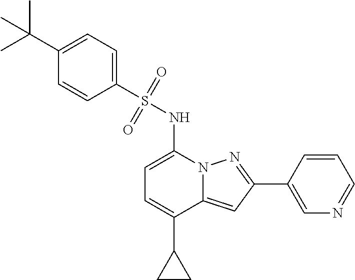 [SMILES: CC(C)(C)c1ccc(S(=O)(=O)Nc2ccc(C3CC3)c3cc(-c4cccnc4)nn23)cc1] |
| 179 | 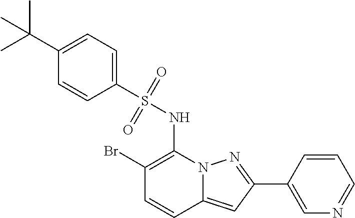 [SMILES: CC(C)(C)c1ccc(S(=O)(=O)Nc2c(Br)ccc3cc(-c4cccnc4)nn23)cc1] |
| 180 | 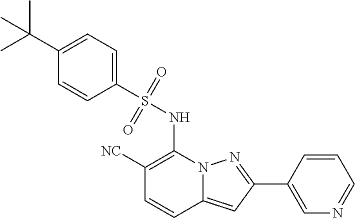 [SMILES: CC(C)(C)c1ccc(S(=O)(=O)Nc2c(C#N)ccc3cc(-c4cccnc4)nn23)cc1] |
| 181 | 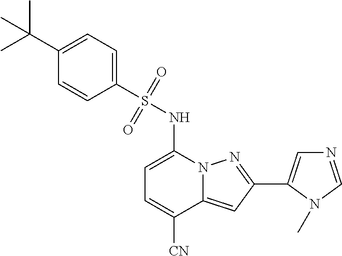 [SMILES: Cn1cncc1-c1cc2c(C#N)ccc(NS(=O)(=O)c3ccc(C(C)(C)C)cc3)n2n1] |
| 182 | 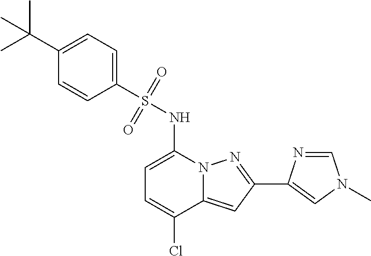 [SMILES: Cn1cnc(-c2cc3c(Cl)ccc(NS(=O)(=O)c4ccc(C(C)(C)C)cc4)n3n2)c1] |
| 183 | 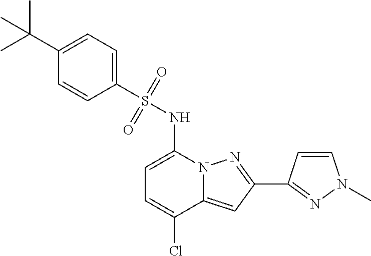 [SMILES: Cn1ccc(-c2cc3c(Cl)ccc(NS(=O)(=O)c4ccc(C(C)(C)C)cc4)n3n2)n1] |
| 184 | 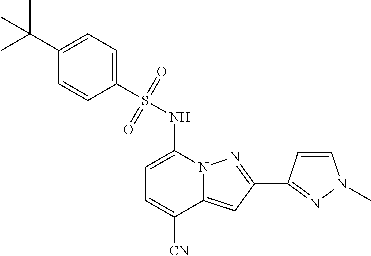 [SMILES: Cn1ccc(-c2cc3c(C#N)ccc(NS(=O)(=O)c4ccc(C(C)(C)C)cc4)n3n2)n1] |
| 185 | 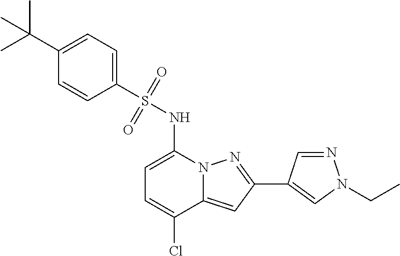 [SMILES: CCn1cc(-c2cc3c(Cl)ccc(NS(=O)(=O)c4ccc(C(C)(C)C)cc4)n3n2)cn1] |
| 186 | 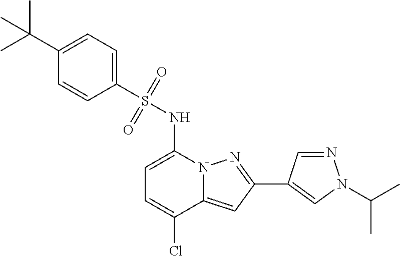 [SMILES: CC(C)n1cc(-c2cc3c(Cl)ccc(NS(=O)(=O)c4ccc(C(C)(C)C)cc4)n3n2)cn1] |
| 187 | 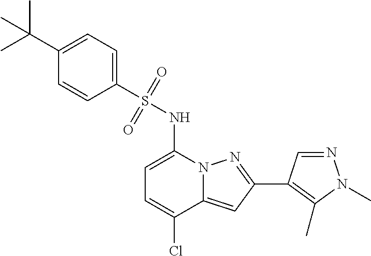 [SMILES: Cc1c(-c2cc3c(Cl)ccc(NS(=O)(=O)c4ccc(C(C)(C)C)cc4)n3n2)cnn1C] |
| 188 | 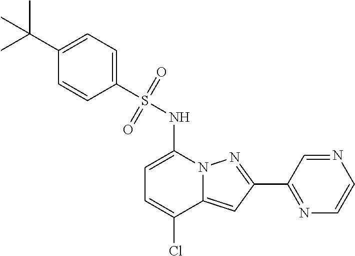 [SMILES: CC(C)(C)c1ccc(S(=O)(=O)Nc2ccc(Cl)c3cc(-c4cnccn4)nn23)cc1] |
| 189 | 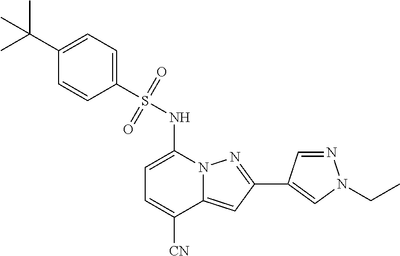 [SMILES: CCn1cc(-c2cc3c(C#N)ccc(NS(=O)(=O)c4ccc(C(C)(C)C)cc4)n3n2)cn1] |
| 190 | 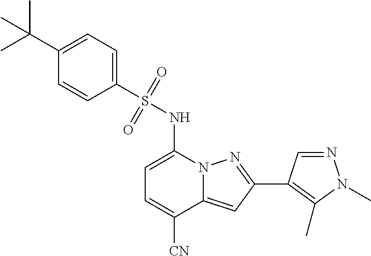 [SMILES: Cc1c(-c2cc3c(C#N)ccc(NS(=O)(=O)c4ccc(C(C)(C)C)cc4)n3n2)cnn1C] |
| 191 | 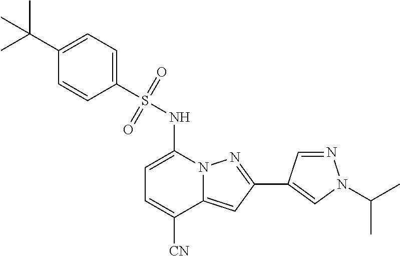 [SMILES: CC(C)n1cc(-c2cc3c(C#N)ccc(NS(=O)(=O)c4ccc(C(C)(C)C)cc4)n3n2)cn1] |
| 192 | 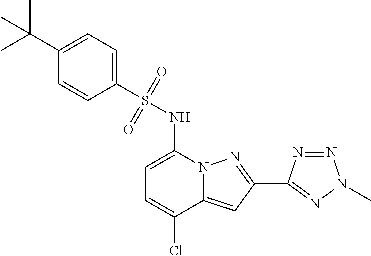 [SMILES: Cn1nnc(-c2cc3c(Cl)ccc(NS(=O)(=O)c4ccc(C(C)(C)C)cc4)n3n2)n1] |
| 193 | 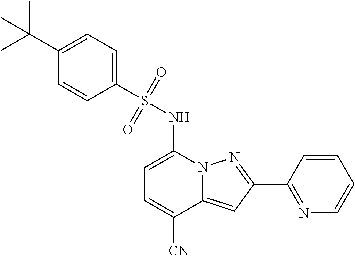 [SMILES: CC(C)(C)c1ccc(S(=O)(=O)Nc2ccc(C#N)c3cc(-c4ccccn4)nn23)cc1] |
| 194 | 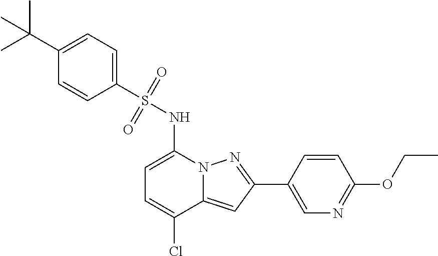 [SMILES: CCOc1ccc(-c2cc3c(Cl)ccc(NS(=O)(=O)c4ccc(C(C)(C)C)cc4)n3n2)cn1] |
| 195 | 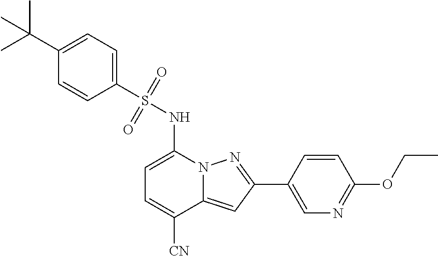 [SMILES: CCOc1ccc(-c2cc3c(C#N)ccc(NS(=O)(=O)c4ccc(C(C)(C)C)cc4)n3n2)cn1] |
| 196 | 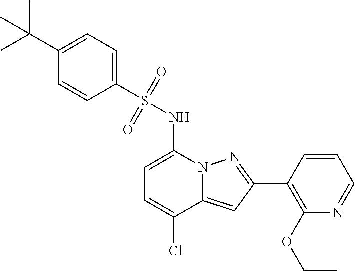 [SMILES: CCOc1ncccc1-c1cc2c(Cl)ccc(NS(=O)(=O)c3ccc(C(C)(C)C)cc3)n2n1] |
| 197 | 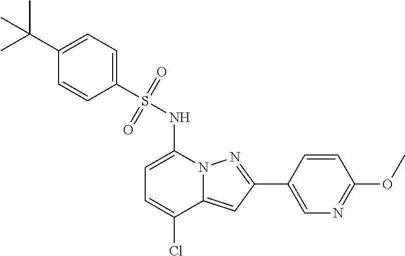 [SMILES: COc1ccc(-c2cc3c(Cl)ccc(NS(=O)(=O)c4ccc(C(C)(C)C)cc4)n3n2)cn1] |
| 198 | 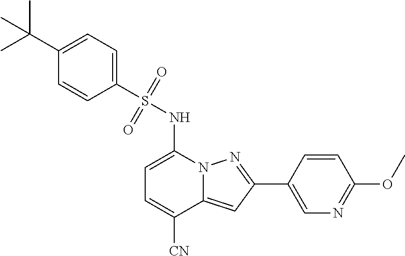 [SMILES: COc1ccc(-c2cc3c(C#N)ccc(NS(=O)(=O)c4ccc(C(C)(C)C)cc4)n3n2)cn1] |
| 199 | 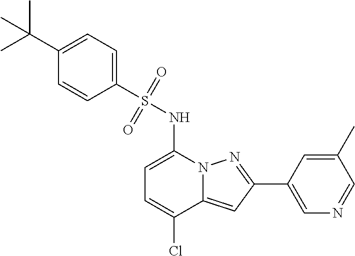 [SMILES: Cc1cncc(-c2cc3c(Cl)ccc(NS(=O)(=O)c4ccc(C(C)(C)C)cc4)n3n2)c1] |
| 200 | 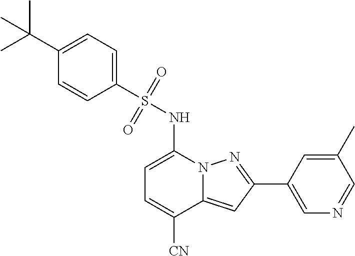 [SMILES: Cc1cncc(-c2cc3c(C#N)ccc(NS(=O)(=O)c4ccc(C(C)(C)C)cc4)n3n2)c1] |
| 201 | 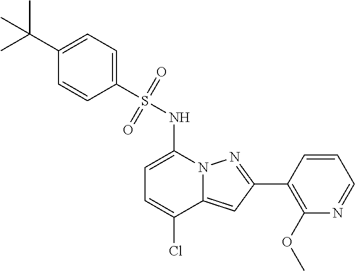 [SMILES: COc1ncccc1-c1cc2c(Cl)ccc(NS(=O)(=O)c3ccc(C(C)(C)C)cc3)n2n1] |
| 202 | 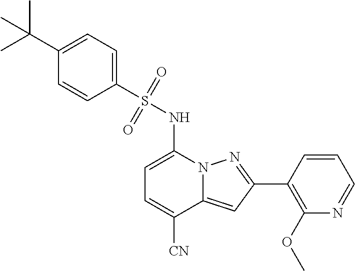 [SMILES: COc1ncccc1-c1cc2c(C#N)ccc(NS(=O)(=O)c3ccc(C(C)(C)C)cc3)n2n1] |
| 203 | 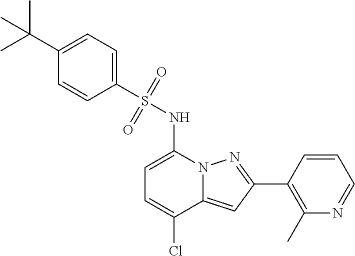 [SMILES: Cc1ncccc1-c1cc2c(Cl)ccc(NS(=O)(=O)c3ccc(C(C)(C)C)cc3)n2n1] |
| 204 | 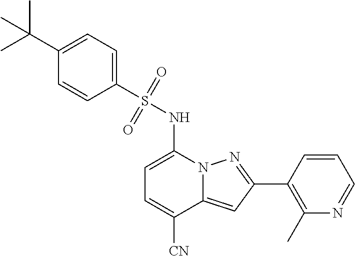 [SMILES: Cc1ncccc1-c1cc2c(C#N)ccc(NS(=O)(=O)c3ccc(C(C)(C)C)cc3)n2n1] |
| 205 | 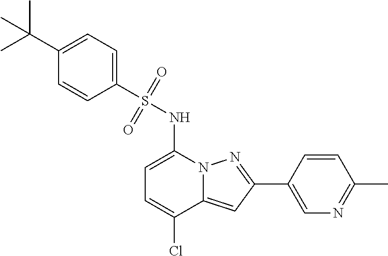 [SMILES: Cc1ccc(-c2cc3c(Cl)ccc(NS(=O)(=O)c4ccc(C(C)(C)C)cc4)n3n2)cn1] |
| 206 | 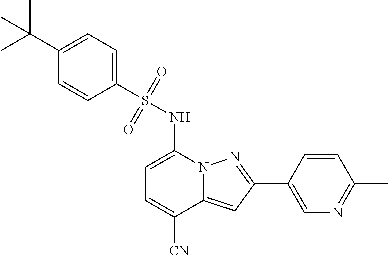 [SMILES: Cc1ccc(-c2cc3c(C#N)ccc(NS(=O)(=O)c4ccc(C(C)(C)C)cc4)n3n2)cn1] |
| 207 | 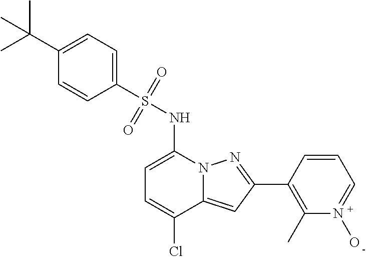 |
| 208 | 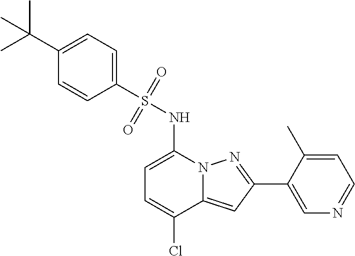 [SMILES: Cc1ccncc1-c1cc2c(Cl)ccc(NS(=O)(=O)c3ccc(C(C)(C)C)cc3)n2n1] |
| 209 | 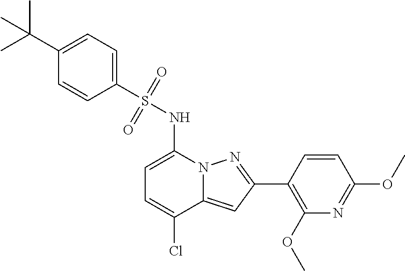 [SMILES: COc1ccc(-c2cc3c(Cl)ccc(NS(=O)(=O)c4ccc(C(C)(C)C)cc4)n3n2)c(OC)n1] |
| 210 | 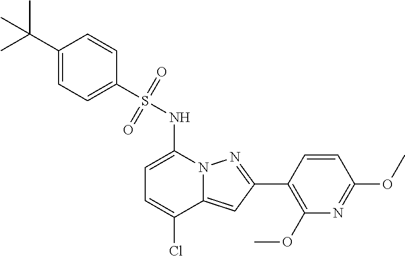 [SMILES: COc1ccc(-c2cc3c(Cl)ccc(NS(=O)(=O)c4ccc(C(C)(C)C)cc4)n3n2)c(OC)n1] |
| 211 | 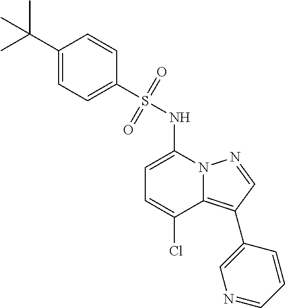 [SMILES: CC(C)(C)c1ccc(S(=O)(=O)Nc2ccc(Cl)c3c(-c4cccnc4)cnn23)cc1] |

The compound of Formula (I) may be used as such, or in the form of a salt or solvate thereof, including a solvate of such a salt. Preferably a salt or solvate is one which is pharmaceutically acceptable.

Suitable salts of the compound of Formula (I) include metal salts, for example alkali metal or alkaline earth metal salts, for example sodium, potassium, calcium and magnesium salts; or salts with ammonia, primary, secondary or tertiary amines, or amino acids, for example mono-, di- or tri-alkylamines, hydroxyalkylamines, and nitrogen-containing heterocyclic compounds, for example isopropylamine, trimethylamine, diethylamine, tri(i-propyl)amine, tri(n-propyl)amine, ethanolamine, 2-dimethylaminoethanol, lysine, histidine, arginine, choline, caffeine, glucamine, procaine, hydrabamine, betaine, ethylenediamine, N-alkylglucamines, theobromine, purines, piperazine, piperidine, morpholine, n-alkyl piperidines, etc; or salts such as trifluoroacetic acid (TFA) salt. For example, pharmaceutically acceptable salts of a compound of Formula (I) include acid addition salts such as hydrochloride, hydrobromide, citrate, tartrate and maleate salts and salts formed with phosphoric and sulphuric acid. In another aspect suitable pharmaceutically acceptable salts are base salts such as an alkali metal salt for example sodium or potassium, an alkaline earth metal salt for example calcium or magnesium, or organic amine salt for example triethylamine.

Many organic compounds can form complexes with solvents in which they are reacted or trom which they are precipitated or crystallized. These complexes are known as solvates. For example, a complex with water is known as a hydrate. Such solvates form part of the invention.

The compound of Formula (I) or its salt or solvate (including a solvate of such a salt) may itself act as a prodrug, or may be converted into a prodrug by known methods. A further aspect of the invention provides a prodrug of the compound of Formula (I) or its salt or solvate (including a solvate of such a salt). Pharmaceutically acceptable prodrugs are described in T. Higuchi and V. Stella (Prodrugs as novel delivery systems, vol 14 of the ACS Symposium Series), and in Edward B. Roche, ed. (Bioreversible carriers in drug design, American Pharm Assoc and Pergamon Press, 1987), both of which are incorporated herein by reference. In one embodiment, a prodrug is a compound having a group that is cleavable from the molecule to generate a biologically active form. Thus the prodrug may be converted within the body into an active form or an active metabolite or residue thereof, due to the presence of particular enzymes or conditions that cleave the prodrug molecule. The cleavable group within the prodrug may be linked by any suitable bond, such as an ester bond or an amide bond (derived from any suitable amine, for example a mono-, di- or tri-alkylamine, or any of the amines mentioned above). For example, the prodrug may be an in vivo hydrolysable ester, such as an ester of a CO₂H group present in the compound of Formula (I) with any suitable alcohol, for example a C₁₋₆alkanol. Alternatively, it may be an ester of any —OH group present in the compound of Formula (I) with any suitable acid, for example any carboxylic or sulfonic acid. Prodrugs that are in vivo hydrolysable esters of a compound of Formula (I) are pharmaceutically acceptable esters that hydrolyse in the human body to produce the parent compound. Such esters can be identified by administering, for example intravenously, to a test animal, the compound under test and subsequently examining the test animal's body fluids. Suitable in vivo hydrolysable esters for carboxy include methoxymethyl and for hydroxy include formyl and acetyl, especially acetyl.

The present invention also provides a process for the preparation of a compound of Formula (I), which comprises a process according to Scheme 1 or Scheme 2 or Scheme 3 or Scheme 4, as described below.

The present invention provides a process for the preparation of a compound of Formula (I) wherein n is 0, which comprises converting cyanoacetic acid (A) to cyanoenamine (B) by treatment with diethylamine, treating the cyanoenamine (B) with a pyrazole amine (D) to produce an amino substituted pyrazolopyrimidine (E), then:
(i) converting the amino substituted pyrazolopyrimidine (E) to a secondary sulfonamide (J) using a sulfonyl chloride (F), and optionally derivatising the secondary sulfonamide (J) to a tertiary sulfonamide (K) using a base and an appropriate alkyl halide; or
(ii) converting the amino substituted pyrazolopyrimidine (E) to a secondary amine (G) using a base and an appropriate alkyl halide, then converting the secondary amine (G) to the tertiary sulfonamide (K) using a sulfonyl chloride (F); or
(iii) condensing the amino substituted pyrazolopyrimidine (E) and a sulfonyl chloride (F) to a di-substituted sulfonamide (H), then converting the di-substituted sulfonamide (H) to the secondary sulfonamide (J), and optionally derivatising the secondary sulfonamide (H) to a tertiary sulfonamide (J) using a base and an appropriate alkyl halide; and
(iv) optionally adding appropriate substituents to an R₁ or R₃ group of the secondary sulfonamide (J) or of the tertiary sulfonamide (K);

as shown in Scheme 1 below, wherein R₁, X, R₃, R₄ and m have the meanings given for the general Formula (I), and Z is a halogen atom (most likely bromine):

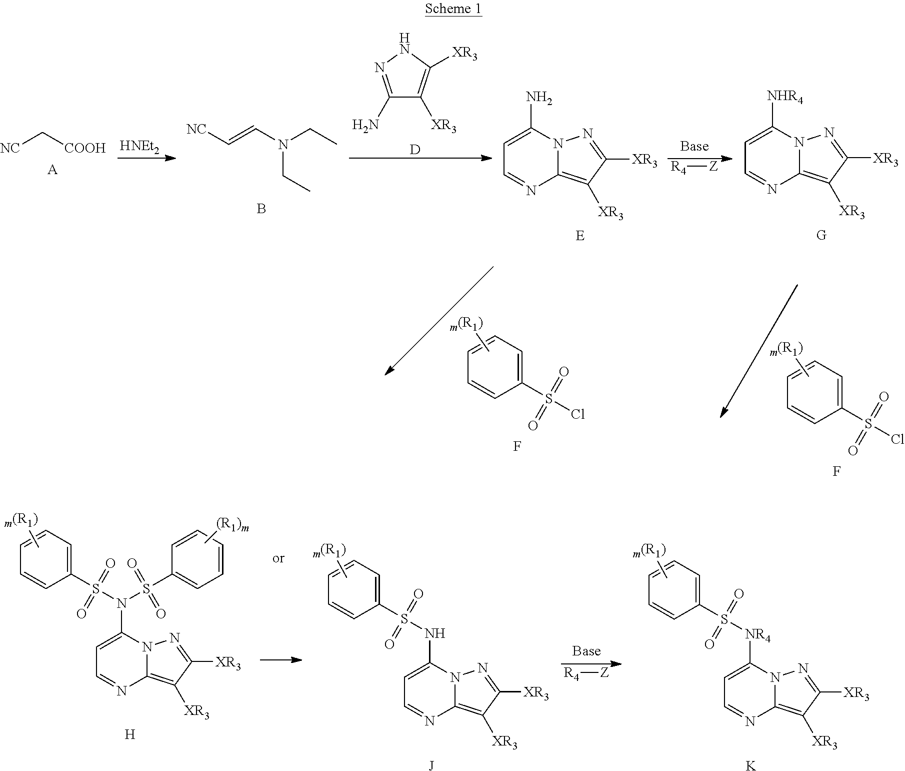

The cyanoacetic acid of formula A may be converted to the cyanoenamine of formula B by treatment with diethylamine in a solvent such as triethyl orthoformate. This may be treated with a pyrazole amine, D, in a suitable base such as pyridine to produce an amino substituted pyrazolopyrimidine E. This may either be converted to the secondary sulfonamide J which may then, if desired, be derivatised to the tertiary sulfonamide K or it may first be converted to the secondary amine G, before conversion to the tertiary sulfonamide K. Conversion of the compounds of formula E or G to the compounds of formula J or K respectively may be achieved by the use of a sulfonyl chloride F. This reagent is either used with a base such as pyridine, triethylamine or diisopropylethylamine in the presence or absence of a catalytic quantity of an agent such as dimethylaminopyridine and using a solvent such as dichloromethane, or by the use of sodium hydride as base in a dipolar aprotic solvent such as DMF prior to addition of the sulfonyl chloride. Conversion of the compounds of formula E or J to the compounds of formula G or K respectively may be achieved by the use of a base such as sodium hydride followed by the appropriate alkyl halide. Condensation of compounds of formula E and F in the presence of base may sometimes proceed to the di-substituted sulfonamide H. In this case, the desired product J may be prepared by use of an agent such as tetrabutyl ammonium fluoride in a solvent such as THF.

The present invention further provides a process for the preparation of a compound of Formula (I) wherein n is 1 or 2, which comprises reacting a pyrazole amine (D) with a dimethyl acetal (M) to produce a pyrazole imidamide (N), treating the pyrazole imidamide (N) with a nitrile to form a pyrazolo pyridine (P), then:
(i) converting the pyrazolo pyridine (P) to a secondary sulfonamide (R) using a sulfonyl chloride (F), and optionally derivatising the secondary sulfonamide (P) to a tertiary sulfonamide (S) using a base and an appropriate alkyl halide; or
(ii) converting the pyrazolo pyridine (P) to a secondary amine (Q) using a base and an appropriate alkyl halide, then converting the secondary amine (Q) to a tertiary sulfonamide (S) using a sulfonyl chloride (F); and
(iii) optionally adding appropriate substituents to an R₁ or R₃ group of the secondary sulfonamide (R) or of the tertiary sulfonamide (S);

as shown in Scheme 2 below, wherein R₁, R₂, X, R₃, R₄ and m have the meanings given for the general Formula (I), and Z is a halogen atom (most likely bromine):

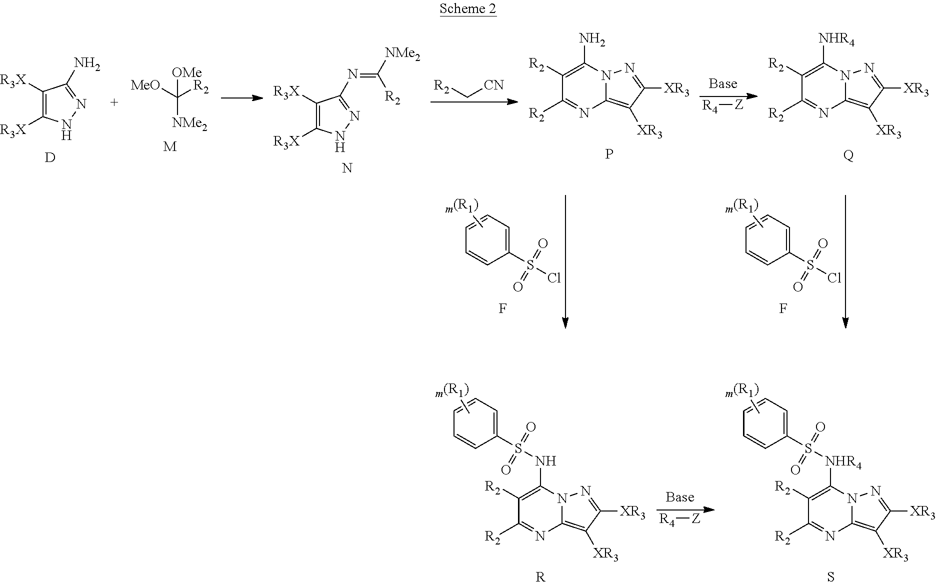

When R₂ is present in a compound of Formula I (that is, when n=1 or 2), the compounds may be prepared as shown in Scheme 2. The pyrazole amine D may be reacted with the dimethyl acetal M in a solvent such as xylene to produce the pyrazole imidamide N. This on treatment with a nitrile in the presence of an organic base such as piperidine and a solvent, for example ethanol, results in the formation of a pyrazolo pyridine P. This may either be converted to the secondary sulfonamide R which may then, if desired, be derivatised to the tertiary sulfonamide S or it may first be converted to the secondary amine Q, before conversion to the tertiary sulfonamide S. Conversion of the compounds of formula P or Q to the compounds of formula R or S respectively may be achieved by the use of a sulfonyl chloride F. This reagent is either used with a base such as pyridine, triethylamine or diisopropylethylamine in the presence or absence of a catalytic quantity of an agent such as dimethylaminopyridine and using a solvent such as dichloromethane, or by the use of sodium hydride as base in a dipolar aprotic solvent such as DMF prior to addition of the sulfonyl chloride. Conversion of the compounds of formula P or R to the compounds of formula Q or S respectively may be achieved by the use of a base such as sodium hydride followed by the appropriate alkyl halide.

The present invention also provides a process for the preparation of a compound of Formula (I) wherein W is CR₁₀, which comprises the steps shown in either Scheme 3 or Scheme 4 below.

The present invention provides a process for the preparation of a compound of Formula (I) wherein W is CR₁₀, which comprises:
(i) converting aminopyridine (T) to sulfonamide (U) by the use of a sulfonyl chloride (F);
(ii) treating the sulfonamide (U) with an acetylene moiety (Y) in the presence of coupling reagents to produce pyridine acetylene (Z);
(iii) converting the pyridine acetylene (Z) by treatment with mesitlyenesulfonylhydroxylamine (AA) to an aminopyridinium salt (AB);
(iv) treating the aminopyridinium salt (AB) with a base to produce pyrazolopyridine (AC);

as shown in Scheme 3 below, wherein R₁, R₂, X, R₃, R₄, R₁₀ and m have the meanings given for the general Formula (I):

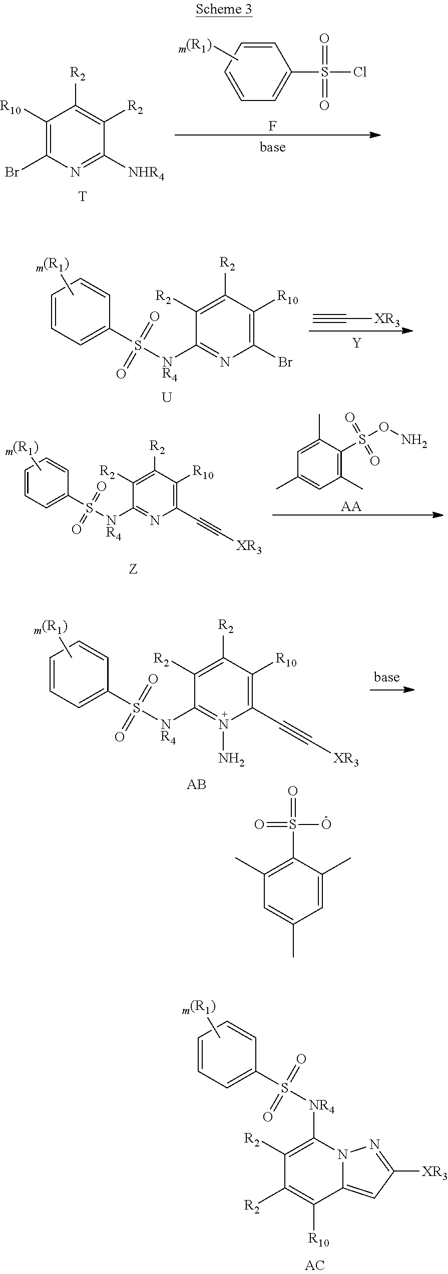

In Scheme 3 the aminopyridine of formula T may be converted to the sulfonamide of formula U by the use of a sulfonyl chloride F. This reagent is either used with a base such as pyridine, triethylamine or diisopropylethylamine in the presence or absence of a catalytic quantity of an agent such as dimethylaminopyridine and using a solvent such as dichloromethane, or by the use of sodium hydride as base in a dipolar aprotic solvent such as DMF prior to addition of the sulfonyl chloride. Treatment of U with an acetylene moiety Y in the presence of coupling reagents such as a mixture of Bis(triphenylphosphine)palladium(II) chloride, copper(I)iodide and triethylamine in a solvent such as DMF will give rise to the pyridine acetylene Z. On treatment with mesitlyenesulfonylhydroxylamine AA, Z may be converted to an aminopyridinium salt of formula AB which on treatment with a base such as potassium carbonate will ring close to produce the pyrazolopyridine AC.

The present invention also provides a process for the preparation of a compound of Formula (I) wherein W is CR₁₀, which comprises:
(i) coupling an aminopyridine (AD or AH) with sulfonyl chloride (F) to produce pyridine sulfonamide (AE or AJ);
(ii) deprotonating the pyridine sulfonamide (AE) with a base in a suitable solvent, and quenching the resulting anion with an ester or activated amide of formula AF, to form ketone (AG); or converting the pyridine sulfonamide (AJ) to ketone (AG) by treatment with suitable reagents and a compound of formula AK;
(iii) converting the ketone (AG) to oxime (AL) using hydroxylamine hydrochloride in a suitable solvent;
(iv) dehydrating oxime (AL) using a dehydrating agent to produce azirine (AM);
(v) rearranging the azirine (AM) using ferrous chloride to produce a compound of formula AN;

as shown in Scheme 4 below, wherein R₁, R₂, X, R₃, R₄, R₁₀ and m have the meanings given for the general Formula (I):

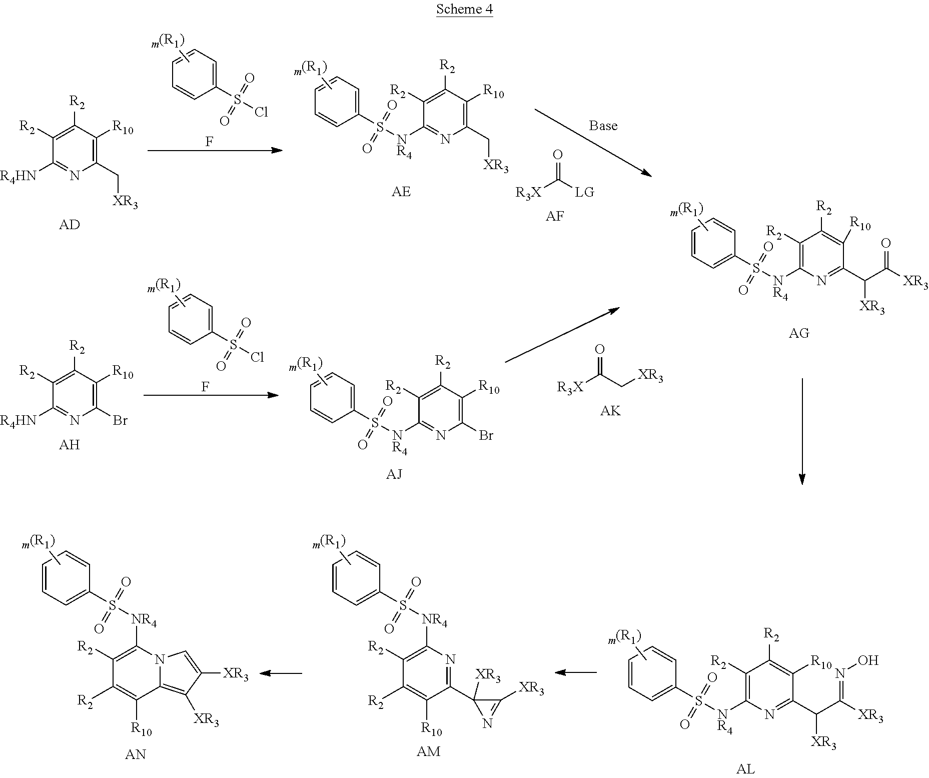

Scheme 4 may be used as an alternative route to Scheme 3. In Scheme 4, the aminopyridine AD or AH may be coupled with the sulfonyl chloride F under conditions as described for the equivalent reaction described in Scheme 3. The resulting pyridine sulfonamide AE may be deprotonated with a base such as sodium bis(trimethylsilyl)amide in a solvent such as THF and the resulting anion quenched with a species of formula AF (wherein LG may for example be an alcohol such that AF is an ester, or it may be a species such as N-methoxy-methylamine so that AF is an activated amide). The pyridine sulfonamide AJ on the other hand is converted to AG by treatment with reagents such as mixtures of 4,5-bis(diphenylphosphino)-9,9-dimethylxanthene, palladium(II) acetate and potassium phosphate in a suitable solvent such as dioxan. The resulting ketone AG can then be converted to the oxime AL using hydroxylamine hydrochloride in a suitable solvent. Dehydration of AL can be carried out using a dehydrating agent such as trifluoroacetic anhydride and triethylamine in DME or similar as solvent to afford the azirine AM which can then be rearranged using iron (II) chloride or similar to the compound of formula AN.

All the above schemes comprehend that interconversion between R groups can be carried out by normal means. For example if R₁₀ is a nitrile, this can be introduced into the species AC and AN by treatment of a molecule where R₁₀ is a halogen with agents such as mixtures of Zn(CN)₂, 1, 1′-Bis (diphenylphosphino)ferrocene, Pd₂dba₃, catalytic quantities of Zn dust in solvents such as DMF and at elevated temperatures. Similarly in the event that XR₃ is pyridyl, this may be converted to the corresponding N-oxide by treatment with metachloroperoxybenzoic acid in a solvent such as dichloromethane as a final step from structures J or K in Scheme 1, structures R or S in Scheme 2 or structures AC or AK in schemes 3 and 4 respectively.

If protecting groups are required to allow certain functional groups to be carried through transformations elsewhere in a molecule, these can be introduced and removed by standard means. Thus in the Schemes above, as well as corresponding to the definitions in Formula 1, R₁, X, R₃, R₄, and R₁₀ can also represent appropriately protected forms of these groups.

It will be appreciated that many of the relevant starting materials are commercially available or may be made by any convenient method as described in the literature or known to the skilled chemist or described in the Examples herein, or can be prepared by methods analogous to such methods. For example, reagents such as C or E may be commercially available or prepared by routes as illustrated in the Examples herein by anyone skilled in the art. Should R₁ or XR₃ contain functionality requiring protection to allow the synthetic scheme to be carried out, appropriate groups can be selected by anyone skilled in the art. The structures of reagents C and E are shown below:

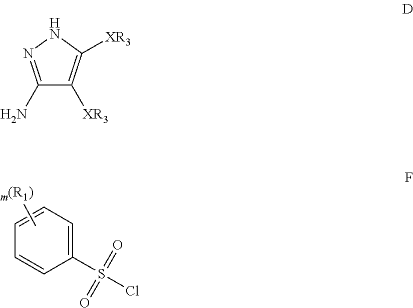

In a further aspect of the invention, there is provided an intermediate compound for use in the synthesis of a compound of Formula (I). There is further provided the use of an intermediate compound to synthesise a compound of Formula (I). Such intermediate compounds include the intermediate compounds I-CXXXI disclosed in the Examples herein and listed in Table 2.

TABLE 2

| Intermediate | |
|---|---|
| compound number | Disclosed in Example number |
| I | 1 |
| II | 1, 2, 4, 5, 6, 7, 8, 9, 10, 11, 12 |
| III | 1 |
| IV | 1 |
| V | 1 |
| VI | 1 |
| VII | 2 |
| VIII | 2 |
| IX | 2 |
| X | 2 |
| XI | 2, 3, 4, 5, 6, 7, 8, 10, 11, 12, 13, |
| | 14, 18, 26, 29, 30, 32 |
| XII | 2 |
| XIII | 3 |
| XIV | 3 |
| XV | 3 |
| XVI | 3 |
| XVII | 4 |
| XVIII | 4 |
| XIX | 4 |
| XX | 4 |
| XXI | 4 |
| XXII | 5 |
| XXIII | 5 |
| XXIV | 5 |
| XXV | 5 |
| XXVI | 5 |
| XXVII | 6 |
| XXVIII | 6 |
| XXIX | 6 |
| XXX | 6 |
| XXXI | 6 |
| XXXII | 7 |
| XXXIII | 7 |
| XXXIV | 7 |
| XXXV | 8 |
| XXXVI | 8 |
| XXXVII | 8 |
| XXXVIII | 9 |
| XXXIX | 9 |
| XL | 9 |
| XLI | 9 |
| XLII | 9 |
| XLIII | 10 |
| XLIV | 10 |
| XLV | 10, 15 |
| XLVI | 10 |
| XLVII | 10 |
| XLVIII | 10 |
| XLIX | 10 |
| L | 11 |
| LI | 11, 30 |
| LII | 11 |
| LIII | 11 |
| LIV | 11 |
| LV | 12 |
| LVI | 12 |
| LVII | 12 |
| LVIII | 12 |
| LVIX | 12 |
| LX | 13 |
| LXI | 13 |
| LXII | 13, 25, 32 |
| LXIII | 13 |
| LXIV | 13 |
| LXV | 13, 20, 25 |
| LXVI | 13 |
| LXVII | 13 |
| LXVIII | 14 |
| LXIX | 14, 15, |
| LXX | 14, 26, 27, 29 |
| LXXI | 14 |
| LXXII | 14 |
| LXXIII | 14 |
| LXXV | 15 |
| LXXVI | 15 |
| LXXVII | 15 |
| LXXVIII | 15 |
| LXXIX | 17, 22, 23, 24 |
| LXXX | 18, 30 |
| LXXXI | 18, 19, 21, 27, 30 |
| LXXXII | 18 |
| LXXXIII | 18 |
| LXXXIV | 18 |
| LXXXV | 18 |
| LXXXVI | 19 |
| LXXXVII | 19 |
| LXXXVIII | 19 |
| LXXXIX | 20 |
| XC | 20 |
| XCI | 20 |
| XCII | 20 |
| XCIII | 21 |
| XCIV | 21 |
| XCV | 21 |
| XCVI | 21 |
| XCVII | 21 |
| XCVIII | 22 |
| XCIX | 22 |
| C | 22 |
| CI | 22 |
| CII | 23 |
| CIII | 23 |
| CIV | 23 |
| CV | 23 |
| CVI | 24 |
| CVII | 24 |
| CVIII | 24 |
| CIX | 24 |
| CX | 25 |
| CXI | 25 |
| CXII | 25 |
| CXIII | 26 |
| CXIV | 26 |
| CXV | 26 |
| CXVI | 26 |
| CXVII | 26 |
| CXVIII | 27 |
| CXIX | 27 |
| CXX | 27 |
| CXXI | 29 |
| CXXII | 29 |
| CXXIII | 29 |
| CXXIV | 29 |
| CXXV | 30 |
| CXXVI | 30 |
| CXXVII | 30 |
| CXXVIII | 32 |
| CXXIX | 32 |
| CXXX | 32 |
| CXXXI | 32 |

A resulting compound of the invention may be converted into any other compound of the invention by methods analogous to known methods. For example: a resulting compound of Formula (I) may be converted into a salt or solvate thereof; the oxidation state of an atom in a heterocyclic ring may be increased or decreased by oxidation or reduction using known methods; an ester may be converted to the corresponding acid by hydrolysis (eg using an aqueous hydroxide such as NaOH) or an acid maybe converted to a corresponding metal salt (eg using an aqueous metal hydroxide, such as NaOH to produce the sodium salt). During synthesis of any compound of the invention, protecting groups may be used and removed as desired.

The amount of the compound of the invention which is required to achieve a therapeutic effect will, of course, depend upon whether the effect is prophylactic or curative, and will vary with the route of administration, the subject under treatment, and the form of disease being treated. It is generally preferable to use the lowest dose that achieves the desired effect. The compound of the invention may generally be administered at a dose of from 0.1 to 1500 mg/kg per day, preferably 0.1 to 500 mg/kg per day, typically from 0.5 to 20 mg/kg/day, for example about 3 mg/kg/day. Unit dose forms may conveniently contain an amount of compound of the invention which is effective at such dosage or as a multiple of the same, for example units containing 5 mg to 500 mg, usually around 10 mg to 200 mg.

For example, a pharmaceutical composition of this invention may be administered to humans so that, for example, a daily dose of 0.5 to 20 mg/kg body weight (and preferably of 0.5 to 3 mg/kg body weight) is received. This daily dose may be given in divided doses as necessary, the precise amount of the compound received and the route of administration depending on the weight, age and sex of the patient being treated and on the particular disease or condition being treated according to principles known in the art. Typically unit dosage forms may contain about 1 mg to 500 mg of a compound of Formula (I). For example, a unit dosage form containing up to 10 mg/kg may be given twice per day, such as 1.5 mg/kg twice per day or 5 mg/kg twice per day or 10 mg/kg twice per day.

The compound of the present invention may be administered one or more times per day, tor example, two or three times per day, or even more often, for example, four or five times per day.

The compounds of this invention may be administered in standard manner for the disease or condition that it is desired to treat. For these purposes the compounds of this invention may be formulated by means known in the art into the required form. While it is possible for the active ingredient to be administered alone, it is preferable for it to be present in a suitable composition formulated as required. Suitable formulations according to the invention include those suitable for oral (including sub-lingual), parenteral (including subcutaneous, intradermal, intramuscular, intravenous, and intraarticular), nasal, inhalation, topical (including dermal, buccal, and sublingual), vaginal and rectal administration. The most suitable route may depend upon, for example, the nature and stage of the condition and disorder of the recipient.

For oral administration, the compounds can be formulated as liquids or solids. Forms suitable for oral administration include for example tablets, capsules, pills, lozenges, granulates, dragees, wafers, aqueous or oily solutions, suspensions, syrups, or emulsions.

Forms suitable for parenteral use include for example sterile aqueous or oily solutions or suspensions or sterile emulsions or infusions.

Forms suitable for nasal administration include for example drops, sprays and aerosols.

Forms suitable for inhalation include for example finely divided powders, aerosols, fine particle dusts or mists which may be generated by means of various types of metered dose pressurized aerosols, nebulizers or insufflators.

Forms suitable for topical administration to the skin include, for example, gels, creams, ointments, emulsions, pastes, foams or adhesive patches. For female patients, the composition may be in a form suitable for intravaginal administration.

Forms suitable for rectal administration include suppositories, rectal capsules and enema solutions.

Forms suitable for transdermal administration generally comprise an adjuvant that enhances the transdermal delivery of the compound of the invention. Suitable adjuvants are known in the art.

A pharmaceutical composition of the present invention may be in unit dosage form. Suitable oral unit dosage forms include those mentioned above. For administration by injection or infusion unit dosage forms include, for example, vials and ampoules. Unit dosage forms for topical administration to the skin include blister packs or sachets, each blister or sachet containing a unit dose of, for example, a gel, cream or ointment, for example, as described above. A metered dosing device may be provided, for example, a pump device, for dosing a predetermined volume of a topical composition, for example, a cream, ointment or gel. A preparation may provide delayed or sustained release, for example a depot preparation or an adhesive patch.

Preferred formulations are those suitable for oral administration, for example in the form of tablets, capsules, pills or the like, or in the form of solutions suitable for injection such as in water for injections BP or aqueous sodium chloride.

To make a composition according to the invention, suitable carriers are well known in the art and include pharmaceutical grade starch, mannitol, lactose, magnesium stearate, sodium saccharin, talcum, cellulose, glucose, sucrose, (or other sugar), magnesium carbonate, gelatin, oil, alcohol, detergents, emulsifiers or water (preferably sterile).

A liquid formulation will generally consist of a suspension or solution of the compound or physiologically acceptable salt in a suitable aqueous or non-aqueous liquid carrier(s), for example water, ethanol, glycerine, polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and microcrystalline cellulose.

A composition in the form of a capsule can be prepared using routine encapsulation procedures. For example, powders, granules or pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Compositions for oral administration may be designed to protect the active ingredient against degradation as it passes through the alimentary tract, for example by an outer coating of the formulation on a tablet or capsule.

Conveniently the composition is in unit dose form such as a tablet or capsule.

In addition to the compounds of the present invention, the pharmaceutical composition of this invention may also contain, or be co-administered (simultaneously or sequentially) with, one or more pharmacological agents of value in treating one or more diseases or conditions referred to hereinabove. For example, pharmaceutical compositions as described above may also comprise one or more further active ingredients in addition to a compound of the invention, for example, a further active ingredient with efficacy in the treatment or prevention of IBD or of conditions associated with IBD.

The compounds of the invention are compounds which modulate at least one function or characteristic of mammalian CCR9, for example, a human CCR9 protein. The ability of a compound to modulate the function of CCR9 can be demonstrated in a binding assay (such as a ligand binding or agonist binding assay), a migration assay, a signaling assay (such as activation of a mammalian G protein, induction of rapid and transient increase in the concentration of cytosolic free calcium) and/or cellular response assay (such as stimulation of chemotaxis, exocytosis or inflammatory mediator release by leukocytes). In particular, compounds of the invention may be evaluated in one or more of the following assays: (1) human CCR9 FLIPR assay using recombinant cell lines expressing human CCR9 or MOLT-4 cells (for example, identifying active compounds as those having Kᵢ≦10 μM, preferred compounds as those having Kᵢ≦1 μM) and most preferred compounds as those having a Kᵢ≦500 nM); (2) chemotaxis assay using MOLT-4 cells (for example, identifying active compounds as those having Kᵢ≦10 μM, preferred compounds as those having Kᵢ≦1 μM and most preferred compounds as those having a Kᵢ≦500 nM); (3) chemotaxis assay using mouse and rat thymocytes (for example, identifying active compounds as those having Kᵢ≦1 μM, and preferred compounds as those having Kᵢ≦500 nM and most preferred compounds as those having a Kᵢ≦500 nM).

As previously outlined the compounds of the invention are CCR9 modulators, in particular they are partial agonists, antagonists or inverse agonists of CCR9. Each of the above indications for the compounds of the Formula (I) represents an independent and particular embodiment of the invention. Whilst we do not wish to be bound by theoretical considerations, some of the preferred compounds of the invention may show selective CCR9 modulation for any one of the above indications relative to modulating activity against any other particular receptor, including any other particular chemokine receptor (for example, CCR1, CCR2, CCR3, CCR4, CCR5, CCR6, CCR7, CCR8, CCR10, CXCR1, CXCR2, CXCR3, CXCR4, CXCR5, CXCR6, CXCR7, CX3CR₁, XCR1, ChemR₂₃ or CMKLR₁); by way of non-limiting example they may show 100-1000 fold selectivity for CCR9 over activity against any other particular chemokine receptor.

The invention will now be illustrated but not limited by the following Examples. Each exemplified compound represents a particular and independent aspect of the invention.

Where optically active centres exist in the compounds of Formula (I), we disclose all individual optically active forms and combinations of these as individual specific embodiments of the invention, as well as their corresponding racemates.

Analytical TLC was performed on Merck silica gel 60 F₂₅₄ aluminium-backed plates. Compounds were visualised by UV light and/or stained either with iodine, potassium permanganate or ninhydrin solution. Flash column chromatography was performed on silica gel (100-200 M) or flash chromatography. ¹H-NMR spectra were recorded on a Bruker Avance-400 MHz spectrometer with a BBO (Broad Band Observe) and BBFO (Broad Band Fluorine Observe) probe. Chemical shifts (δ) are expressed in parts per million (ppm) downfield by reference to tetramethylsilane as the internal standard. Splitting patterns are designated as s (singlet), d (doublet), t (triplet), q (quartet), m (multiplet) and bs (broad singlet). Coupling constants (J) are given in hertz (Hz). LC-MS analyses were performed on either an Acquity BEH C-18 column (2.10×100 mm, 1.70 μm) or on a Acquity HSS-T3 column (2.10×100 mm, 1.80 μm) using the Electrospray Ionisation (ESI) technique. Purity assessment for final compounds was based on the following 2 LCMS methods. Method 1 consisted of the following: Acquity BEH C-18 column 2.10 mm×100 mm, 1.70 μm. Mobile phase; A, 5 mM ammonium acetate in water; B, acetonitrile; gradient, 90% A to 10% A in 8 min with 10 min run time and a flow rate of 0.3 mL/min. Method 2 consisted of the following: Acquity HSS-T3 column 2.10 mm×100 mm, 1.8 μm. Mobile phase; A, 0.1% TFA in water; B, acetonitrile; gradient, 90% A to 10% A in 8 min with 10 min run time and a flow rate of 0.3 mL/min.

### Example 1

### Synthesis of Compound 1 [N-(2-methylpyrazolo[1,5-a]pyrimidin-7-yl)-4-(oxazol-5-yl)benzenesulfonamide] and Compounds 2-36

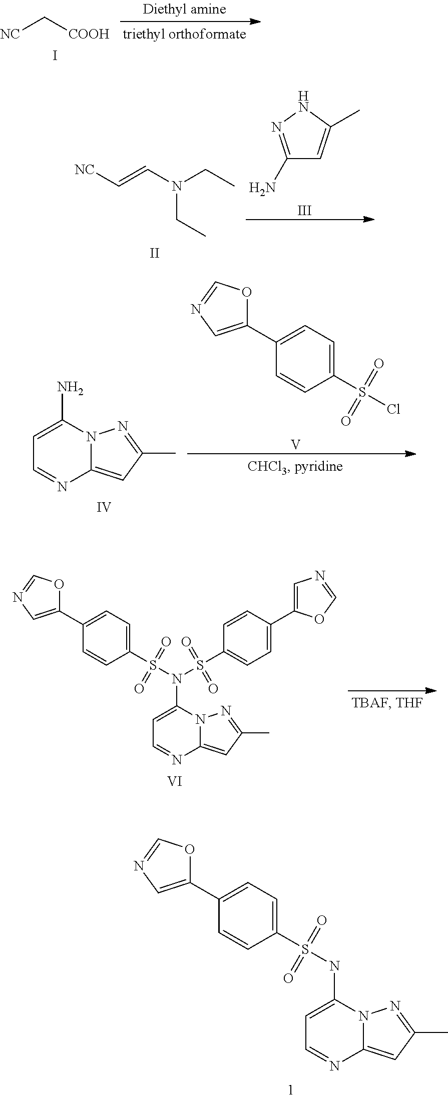

Synthesis of II:

A mixture of cyanoacetic acid (I; 20 g, 235 mmol), triethylorthoformate (34.04 g; 235 mmol) and diethylamine (17.17 g; 235 mmol) was heated at 140° C. for 3 hours. The reaction mixture was concentrated at reduced pressure and then diluted with a saturated solution of sodium bicarbonate. The organic layer was extracted with ethyl acetate, which was washed with water, brine, dried over Na₂SO₄, filtered and concentrated under vacuum to afford crude solid (II; 15 g;), which was used in the next step without further purification.

Synthesis of IV:

To a stirred solution of 5-methyl-1H-pyrazol-3-amine (III; 5 g; 51.5 mmol) in pyridine (60 mL) was added 3-(diethylamino)acrylonitrile (II; 9.6 g; 77 mmol). The reaction mixture was heated at 120° C. for 14 hours and then cooled and concentrated under reduced pressure. The crude mixture was purified by column chromatography using 2% MeOH-DCM to obtain 2-methylpyrazolo[1,5-a]pyrimidin-7-amine as a brown solid (IV; 3 g; 39.3% yield). ¹H NMR (400 MHz, DMSO-d6): δ 7.97-7.96 (d, J=5.2 Hz, 1H), 7.58 (bs, 2H), 6.14 (s, 1H), 5.98-5.97 (d, J=5.2 Hz, 1H), 2.38 (s, 3H). MS (M+1): 149.2.

### Synthesis of Compound 1; N-(2-methylpyrazolo[1,5-a]pyrimidin-7-yl)-4-(oxazol-5-yl)benzenesulfonamide

To a stirred solution of 2-methylpyrazolo[1,5-a]pyrimidin-7-amine, (IV; 100 mg; 0.67 mmol) in chloroform (10 mL) was added pyridine (160 mg; 2.02 mmol) and 4-(oxazol-5-yl)benzene-1-sulfonyl chloride (V; 246 mg; 1.01 mmol) at 0° C. The reaction mixture was heated at 80° C. for 14 hours. The reaction mixture was cooled and concentrated at reduced pressure to afford the di-substituted sulfonamide product the structure of which was confirmed by LCMS (vi; 90% purity). The crude product was dissolved in THF (5 mL) in presence of TBAF (0.5 mL) and stirred at room temperature for 2 hours. The reaction mixture was concentrated at reduced pressure, diluted with water and the aqueous layer was extracted with EtOAc (3×25 mL). The combined organic layers were washed with brine, dried over Na₂SO₄, filtered and concentrated to afford the crude compound, which was purified by column chromatography (4% MeOH-DCM)) to obtain the title compound N-(2-methylpyrazolo[1,5-a]pyrimidin-7-yl)-4-(oxazol-5-yl)benzenesulfonamide (1; 25 mg; 11% yield). ¹HNMR (400 MHz, DMSO-d6): δ 13.46 (bs, 1H), 8.54 (s, 1H), 8.04-8.02 (d, J=7.2 Hz, 1H), 7.97-7.95 (d, J=8.4 Hz, 2H), 7.91-7.88 (d, J=8.4 Hz, 2H), 7.85 (s, 1H), 6.65-6.63 (d, J=7.2 Hz, 1H), 6.62 (s, 1H), 2.33 (s, 3H). MS (M+1): 356.07. (LCMS purity 94.37%, 4.19 min) (2).

The Following Compounds were Prepared in Essentially the Same Manner Using the Appropriate Sulfonyl Chloride in the Final Step:

| | | LCMS | Purity | |
|---|---|---|---|---|
| CPD | Structure | (M + 1) | (LCMS) | ¹H NMR |
| 2 | 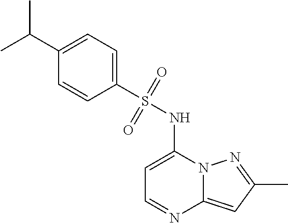 [SMILES: Cc1cc2nccc(NS(=O)(=O)c3ccc(C(C)C)cc3)n2n1] | 331.12 | 96.13%, Rt = 5.26 min (2) | ¹HNMR (400 MHz, DMSO- d6): δ 13.28 (bs, 1H), 8.01- 7.99 (d, J = 7.0 Hz, 1H), 7.79-7.77 (d, J = 8.0 Hz, 2H), 7.42-7.40 (d, J = 8.0 Hz, 2H), 6.65-6.63 (d, J = 7.0 Hz, 1H), 6.19 (s, 1H), 2.98-2.91 (m, 1H), 2.32 (s, 3H), 1.20-1.19 (d, J = 6.8 Hz, 6H). |
| 3 | 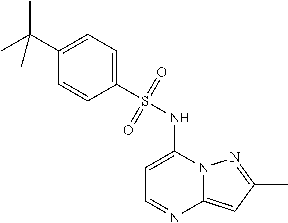 [SMILES: Cc1cc2nccc(NS(=O)(=O)c3ccc(C(C)(C)C)cc3)n2n1] | 345.13 | 98.57%, Rt = 5.45 min (2) | ¹HNMR (400 MHz, DMSO- d6): δ 13.20 (bs, 1H), 7.99- 7.97 (d, J = 6.8 Hz, 1H), 7.79-7.77 (d, J = 8.4 Hz, 2H), 7.57-7.54 (d, J = 8.4 Hz, 2H), 6.63-6.61 (d, J = 6.8 Hz, 1H), 6.17 (s, 1H), 2.32 (s, 3H), 1.28 (s, 9H). |
| 4 | 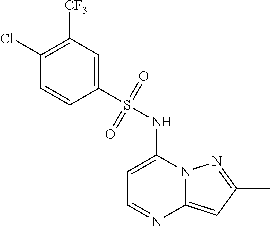 [SMILES: Cc1cc2nccc(NS(=O)(=O)c3ccc(Cl)c(C(F)(F)F)c3)n2n1] | 391.02 | 98.49%, Rt = 5.52 min (2) | ¹HNMR (400 MHz, DMSO- d6): δ 13.52 (bs, 1H), 8.17- 8.15 (m, 2H), 8.07-8.05 (d, J = 6.8 Hz, 1H), 7.94-7.92 (d, J = 5.6 Hz, 1H), 6.62-6.60 (d, J = 7.2 Hz, 1H), 6.23 (s, 1H), 2.33 (s, 3H). |
| 5 | 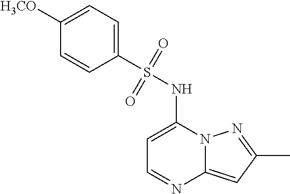 [SMILES: COc1ccc(S(=O)(=O)Nc2ccnc3cc(C)nn23)cc1] | 319.11 | 99.45%, Rt = 4.32 min (2) | ¹HNMR (400 MHz, DMSO- d6): δ 13.14 (bs, 1H), 8.0-7.98 (d, J = 7.2 Hz, 1H), 7.81-7.79 (d, J = 8.4 Hz, 2H), 7.07-7.05 (d, J = 8.4 Hz, 2H), 6.63-6.61 (d, J = 7.2 Hz, 1H), 6.19 (s, 1H), 3.81 (s, 3H), 2.32 (s, 3H). |
| 6 | 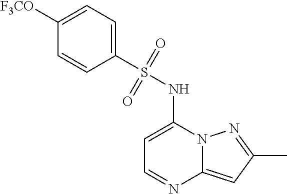 [SMILES: Cc1cc2nccc(NS(=O)(=O)c3ccc(OC(F)(F)F)cc3)n2n1] | 373.06 | 98.08%, Rt = 5.20 min (2) | ¹HNMR (400 MHz, DMSO- d6): δ 13.37 (bs, 1H), 8.07- 8.05 (d, J = 7.2 Hz, 1H), 8.02-8.0 (d, J = 8.8 Hz, 2H), 7.56-7.54 (d, J = 8.8 Hz, 2H), 6.68-6.66 (d, J = 7.2 Hz, 1H), 6.24 (s, 1H), 2.34 (s, 3H). |
| 7 | 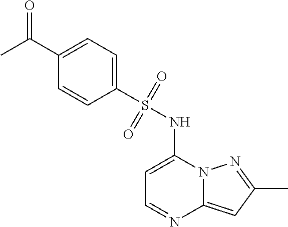 [SMILES: CC(=O)c1ccc(S(=O)(=O)Nc2ccnc3cc(C)nn23)cc1] | 331.14 | 99.52%, Rt = 4.22 min (2) | ¹HNMR (400 MHz, DMSO- d6): δ 13.37 (bs, 1H), 8.11- 8.09 (d, J = 8.4 Hz, 2H), 8.06-8.04 (d, J = 7.2 Hz, 1H), 8.01-7.99 (d, J = 8.4 Hz, 2H), 6.66-6.65 (d, J = 7.2 Hz, 1H), 6.24 (s, 1H), 2.66 (s, 3H), 2.34 (s, 3H). |
| 8 | 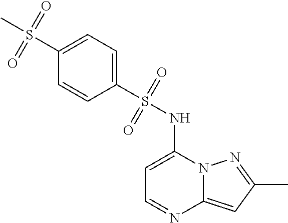 | 367.06 | 98.98%, Rt = 3.93 min (2) | ¹HNMR (400 MHz, DMSO- d6): δ 13.50 (bs, 1H), 8.14- 8.12 (m, 5H), 6.68-6.66 (d, J = 8.0 Hz, 1H), 6.25 (s, 1H), 3.27 (s, 3H), 2.34 (s, 3H). |
| 9 | 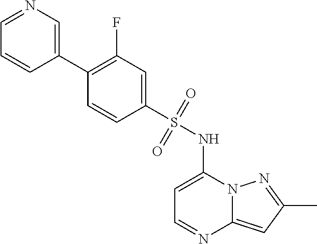 [SMILES: Cc1cc2nccc(NS(=O)(=O)c3ccc(-c4cccnc4)c(F)c3)n2n1] | 384.11 | 98.41%, Rt = 3.69 min (2) | ¹HNMR (400 MHz, DMSO- d6): δ 13.43 (bs, 1H), 8.77 (s, 1H), 8.64-8.63 (d, J = 4.4 Hz, 1H), 8.02-7.98 (m, 2H), 7.80- 7.74 (m. 3H), 7.55-7.51 (m, 1H), 6.59-6.58 (d, J = 6 Hz, 1H), 6.16 (s, 1H), 2.33 (s, 3H). |
| 10 | 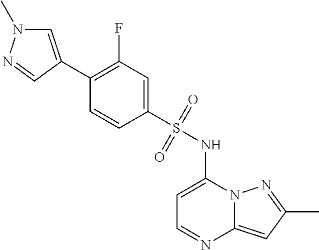 | 387.35 | 95.88%, Rt = 1.44 min (2) | ¹HNMR (400 MHz, DMSO- d6): δ 13.34 (bs, 1H), 8.24- 8.23 (d, J = 2 Hz, 1H), 8.01- 8.0 (d, J = 6.8 Hz, 1H), 7.96 (s, 1H), 7.90-7.86 (m, 1H), 7.68-7.66 (d, J = 9.6 Hz, 2H), 6.61-6.60 (d, J = 6.8 Hz, 1H), 6.20 (s, 1H), 3.89 (s, 3H), 2.33 (s, 3H). |
| 11 | 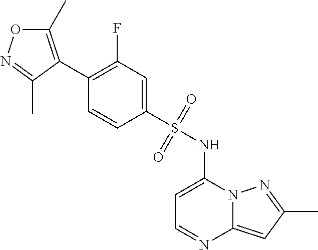 [SMILES: Cc1cc2nccc(NS(=O)(=O)c3ccc(-c4c(C)noc4C)c(F)c3)n2n1] | 402.15 | 98.44%, Rt = 4.98 min (2) | ¹HNMR (400 MHz, DMSO- d6): δ 13.44 (bs, 1H), 8.08- 8.06 (d, J = 6.8 Hz, 1H), 7.81-7.78 (d, J = 8.4 Hz, 1H), 7.67-7.63 (m, 1H), 6.73-6.71 (d, J = 7.2 Hz, 2H), 6.24 (s, 1H), 2.34 (s, 6H), 2.16 (s, 3H). |
| 12 | 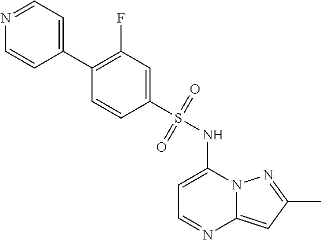 [SMILES: Cc1cc2nccc(NS(=O)(=O)c3ccc(-c4ccncc4)c(F)c3)n2n1] | 384.04 | 99.29%, Rt = 3.57 min (2) | ¹HNMR (400 MHz, DMSO- d6): δ 13.44 (bs, 1H), 8.71- 8.69 (d, J = 5.6 Hz, 2H), 8.07-8.06 (d, J = 7.2 Hz, 1H), 7.83-7.80 (d, J = 12 Hz, 3H), 6.62-6.61 (d, J = 4.4 Hz, 2H), 6.70-6.68 (d, J = 6.4 Hz, 1H), 6.24 (s, 1H), 2.34 (s, 3H). |
| 13 | 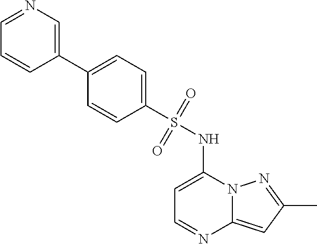 [SMILES: Cc1cc2nccc(NS(=O)(=O)c3ccc(-c4cccnc4)cc3)n2n1] | 366.06 | 97.71%, Rt = 3.40 min (2) | ¹HNMR (400 MHz, DMSO- d6 with D2O): δ 8.87 (s, 1H), 8.58-8.57 (d, J = 4.4 Hz, 1H), 8.11-8.09 (d, J = 7.6 Hz, 1H), 7.97-7.85 (m, 5H), 7.52-7.51 (d, J = 5.2 Hz, 1H), 6.55- 6.54 (d, J = 6.4 Hz, 1H), 6.15 (s, 1H), 2.31 (s, 3H). |
| 14 | 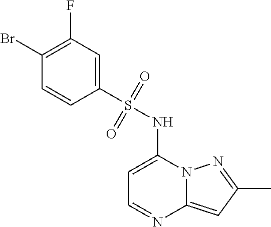 [SMILES: Cc1cc2nccc(NS(=O)(=O)c3ccc(Br)c(F)c3)n2n1] | 386.96 | 99.05%, Rt = 5.07 min (2) | ¹HNMR (400 MHz, DMSO- d6): δ 8.21 (bs, 1H), 7.85-7.83 (m, 2H), 7.76-7.74 (d, J = 8.8 Hz, 1H), 7.64-7.58 (m, 1H), 6.33 (m, 1H), 6.06 (s, 1H), 2.31 (s, 3H). |
| 15 | 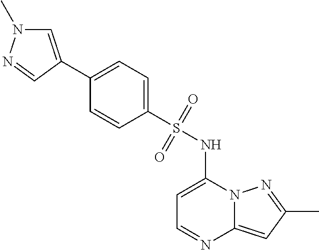 | 369.15 | 99.67%, Rt = 4.19 min (2) | ¹HNMR (400 MHz, DMSO- d6): δ 13.25 (bs, 1H), 8.25 (s, 1H), 8.02-8.0 (d, J = 7.2 Hz, 1H), 7.94 (s, 1H), 7.84-7.81 (d, J = 8.4 Hz, 2H), 7.73-7.71 (d, J = 8.4 Hz, 2H), 6.67-6.65 (d, J = 7.2 Hz, 1H), 6.21 (s, 1H), 3.86 (s, 3H), 2.33 (s, 3H). |
| 16 | 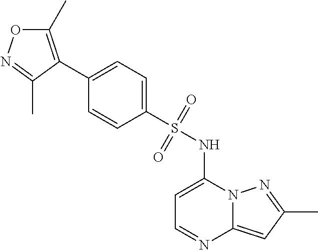 [SMILES: Cc1cc2nccc(NS(=O)(=O)c3ccc(-c4c(C)noc4C)cc3)n2n1] | 384.17 | 99.81%, Rt = 4.75 min (2) | ¹HNMR (400 MHz, DMSO- d6): δ 13.39 (bs, 1H), 8.06- 8.04 (d, J = 7 Hz, 1H), 7.96- 7.94 (d, J = 8.4 Hz, 2H), 7.60-7.58 (d, J = 8.4 Hz, 2H), 6.73-6.72 (d, J = 7 Hz, 1H), 6.23 (s, 1H), 2.43 (s, 3H), 2.33 (s, 3H), 2.25 (s, 3H). |
| 17 | 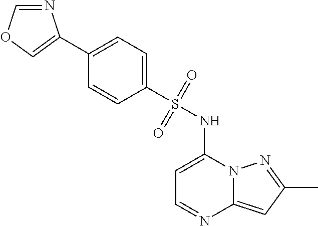 [SMILES: Cc1cc2nccc(NS(=O)(=O)c3ccc(-c4cocn4)cc3)n2n1] | 356.09 | 95.76%, Rt = 3.63 min (2) | ¹HNMR (400 MHz, DMSO- d6): δ 13.33 (bs, 1H), 8.83 (s, 1H), 8.53 (s, 1H), 8.047-8.029 (d, J = 7.2 Hz, 1H), 7.98-7.92 (m, 4H), 6.67-6.65 (d, J = 7.2 Hz, 1H), 6.23 (s, 1H), 2.33 (s, 3H). |
| 18 | 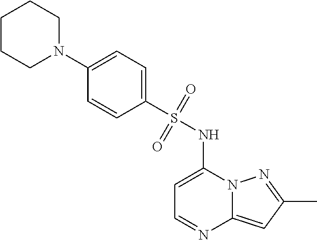 [SMILES: Cc1cc2nccc(NS(=O)(=O)c3ccc(N4CCCCC4)cc3)n2n1] | 372.20 | 96.72%, Rt = 4.42 min (2) | ¹HNMR (400 MHz, DMSO- d6): δ 13.13 (bs, 1H), 7.96- 7.95 (d, J = 6.8 Hz, 1H), 7.63-7.61 (d, J = 8.4 Hz, 2H), 6.98-6.96 (d, J = 8.4 Hz, 2H), 6.62-6.60 (d, J = 6.8 Hz, 1H), 6.17 (s, 1H), 3.24 (m, 4H), 2.32 (s, 3H), 1.56 (m, 6H). |
| 19 | 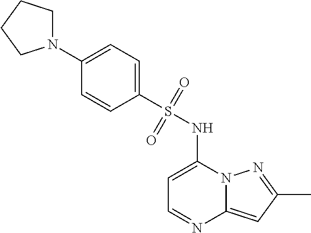 [SMILES: Cc1cc2nccc(NS(=O)(=O)c3ccc(N4CCCC4)cc3)n2n1] | 358.16 | 97.95%, Rt = 5.03 min (2) | ¹HNMR (400 MHz, DMSO- d6): δ 7.89-7.88 (d, J = 6 Hz, 1H), 7.61-7.59 (d, J = 8 Hz, 2H), 6.56-6.52 (m, 3H), 6.11 (s, 1H), 3.25 (bs, 5H), 2.31 (s, 3H), 1.94 (s, 4H). |
| 20 | 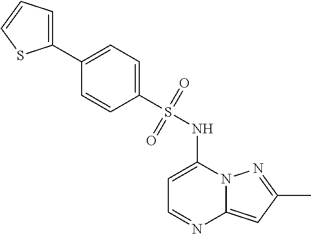 [SMILES: Cc1cc2nccc(NS(=O)(=O)c3ccc(-c4cccs4)cc3)n2n1] | 371.06 | 99.12%, Rt = 4.54 min (2) | ¹HNMR (400 MHz, DMSO- d6): δ 7.83-7.79 (t, J = 6.8 Hz, 3H), 7.75-7.72 (d, J = 8.4 Hz, 2H), 7.61-7.58 (m, 2H), 7.16-7.14 (t, J = 4 Hz, 2H), 6.30-6.29 (d, J = 5.6 Hz, 1H), 6.01 (s, 1H), 2.30 (s, 3H). |
| 21 | 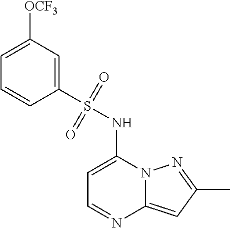 [SMILES: Cc1cc2nccc(NS(=O)(=O)c3cccc(OC(F)(F)F)c3)n2n1] | 373.05 | 98.86%, Rt = 4.47 min (2) | ¹HNMR (400 MHz, DMSO- d6): δ 13.45 (bs, 1H), 8.07- 8.06 (d, J = 6.8 Hz 1H), 7.93- 7.91 (d, J = 8 Hz, 1H), 7.78 (s, 1H), 7.72 (s, 1H), 7.65- 7.63 (d, J = 7.2 Hz, 1H), 6.67-6.5 (d, J = 7.2 Hz, 1H), 6.24 (s, 1H), 2.34 (s, 3H). |
| 22 | 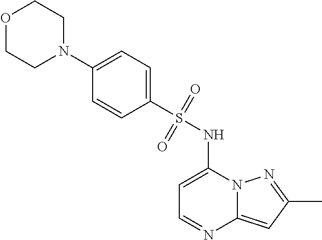 [SMILES: Cc1cc2nccc(NS(=O)(=O)c3ccc(N4CCOCC4)cc3)n2n1] | 374.20 | 95.57%, Rt = 4.23 min (2) | ¹HNMR (400 MHz, DMSO- d6 with d-TFA): δ 7.97-7.95 (d, J = 7.6 Hz, 1H), 7.71-7.69 (d, J = 8 Hz, 2H), 7.02-6.99 (d, J = 8.8 Hz, 2H), 6.68-6.66 (d, J = 6.8 Hz, 1H), 6.17 (s, 1H), 3.71 (s, 4H), 3.21 (s, 4H), 2.31 (s, 3H). |
| 23 | 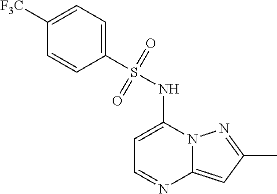 [SMILES: Cc1cc2nccc(NS(=O)(=O)c3ccc(C(F)(F)F)cc3)n2n1] | 357.10 | 99.05%, Rt = 5.12 min (2) | ¹HNMR (400 MHz, DMSO- d6): δ 13.42 (bs, 1H), 8.10- 8.07 (m, 3H), 7.96-7.93 (d, J = 8.4 Hz, 2H), 6.67-6.66 (d, J = 7.2 Hz, 1H), 6.25 (s, 1H), 2.34 (s, 3H). |
| 24 | 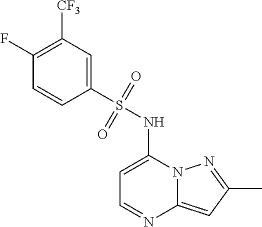 [SMILES: Cc1cc2nccc(NS(=O)(=O)c3ccc(F)c(C(F)(F)F)c3)n2n1] | 375.11 | 99.85%, Rt = 5.24 min (2) | ¹HNMR (400 MHz, DMSO- d6): δ 13.46 (bs, 1H), 8.29- 8.26 (m, 1H), 8.16-8.15 (d, J = 6.4 Hz, 1H), 8.11-8.09 (d, J = 8 Hz, 1H), 7.76-7.72 (t, J = 9.6 Hz, 1H), 6.70-6.68 (d, J = 7.2 Hz, 1H), 6.26 (s, 1H), 2.34 (s, 3H). |
| 25 | 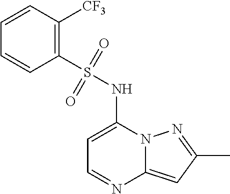 [SMILES: Cc1cc2nccc(NS(=O)(=O)c3ccccc3C(F)(F)F)n2n1] | 357.06 | 97.77% Rt = 4.09 min (2) | ¹HNMR (400 MHz, DMSO- d6): δ 13.40 (bs, 1H), 8.28- 8.27 (d, J = 7.6 Hz, 1H), 8.07-8.05 (d, J = 7.2 Hz, 1H), 7.96-7.94 (d, J = 7.6 Hz, 1H), 7.90-7.83 (m, 2H), 6.64-6.62 (d, J = 7.2 Hz, 1H), 6.25 (s, 1H), 2.32 (s, 3H). |
| 26 | 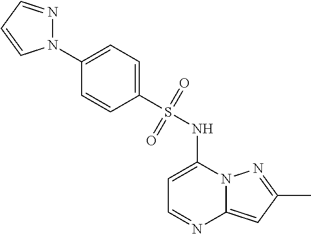 [SMILES: Cc1cc2nccc(NS(=O)(=O)c3ccc(-n4cccn4)cc3)n2n1] | 355.18 | 98.77%, Rt = 4.45 min (2) | ¹HNMR (400 MHz, DMSO- d6): δ 13.34 (bs, 1H), 8.61- 8.60 (d, J = 2 Hz, 1H), 8.04- 8.02 (t, J = 7.6 Hz, 3H), 7.99- 7.97 (d, J = 9.2 Hz, 2H), 7.81 (s, 1H), 6.68-6.66 (d, J = 7.6 Hz, 1H), 6.60 (s, 1H), 6.23 (s, 1H), 2.34 (s, 3H). |
| 27 | 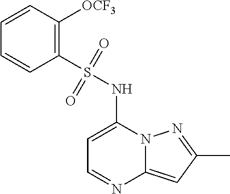 [SMILES: Cc1cc2nccc(NS(=O)(=O)c3ccccc3OC(F)(F)F)n2n1] | 373.07 | 98.10%, Rt = 4.21 min (2) | ¹HNMR (400 MHz, DMSO- d6): δ 13.36 (bs, 1H), 8.08- 8.06 (m, 2H), 7.76-7.72 (m, 1H), 7.58-7.51 (m, 2H), 6.71- 6.69 (d, J = 6.8 Hz, 1H), 6.22 (s, 1H), 2.33 (s, 3H). |
| 28 | 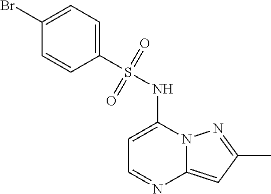 [SMILES: Cc1cc2nccc(NS(=O)(=O)c3ccc(Br)cc3)n2n1] | 369.01 | 98.24%, Rt = 4.13 min (2) | ¹HNMR (400 MHz, DMSO- d6): δ 7.95-7.94 (d, J = 6.6 Hz, 1H), 7.78-7.76 (d, J = 8.8 Hz, 2H), 7.73-7.71 (d, J = 8.8 Hz, 2H), 6.48-6.47 (d, J = 6.6 Hz, 1H), 6.15 (s, 1H), 2.32 (s, 3H). |
| 29 | 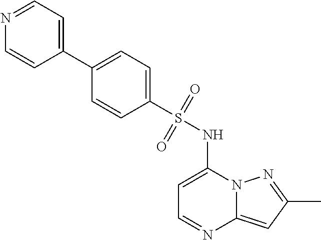 [SMILES: Cc1cc2nccc(NS(=O)(=O)c3ccc(-c4ccncc4)cc3)n2n1] | 364.00 (M − 1) | 99.24%, Rt = 3.61 min (2) | ¹HNMR (400 MHz, DMSO- d6): δ 13.32 (bs, 1 H), 8.71- 8.69 (d, J = 6 Hz, 2H), 8.07- 7.97 (m, 5H), 7.80-7.78 (d, J = 6 Hz, 2H), 6.71-6.70 (d, J = 7.2 Hz, 1H), 6.24 (s, 1H), 2.34 (s, 3H). |
| 30 | 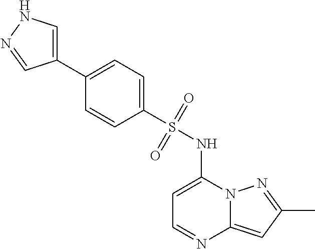 [SMILES: Cc1cc2nccc(NS(=O)(=O)c3ccc(-c4cn[nH]c4)cc3)n2n1] | 355.15 | 98.94%, Rt = 3.85 min (2) | ¹HNMR (400 MHz, DMSO- d6 with TFA): δ 8.22 (s, 2H), 8.01-7.99 (d, J = 7.2 Hz, 1H), 7.86-7.84 (d, J = 8.4 Hz, 2H), 7.78-7.76 (d, J = 8.4 Hz, 2H), 6.69-6.67 (d, J = 7.2 Hz, 1H), 6.20 (s, 1H), 2.32 (s, 3H). |
| 31 | 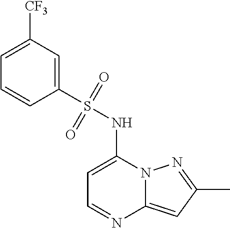 [SMILES: Cc1cc2nccc(NS(=O)(=O)c3cccc(C(F)(F)F)c3)n2n1] | 357.09 | 97.49%, Rt = 4.32 min (2) | ¹HNMR (400 MHz, DMSO- d6): δ 13.50 (bs, 1 H), 8.21- 8.19 (d, J = 7.6 Hz, 1H), 8.11 (s, 1H), 8.10-8.08 (d, J = 7.2 Hz, 1H), 8.03-8.01 (d, J = 7.6 Hz, 1H), 7.859-7.820 (t, J = 7.6 Hz, 1H), 6.711-6.693 (d, J = 7.2 Hz, 1H), 6.26 (s, 1H), 2.34 (s, 3H). |
| 32 | 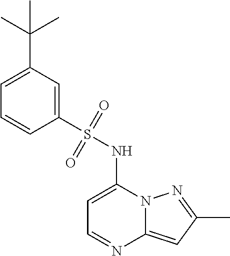 [SMILES: Cc1cc2nccc(NS(=O)(=O)c3cccc(C(C)(C)C)c3)n2n1] | 345.22 | 96.58%, Rt = 4.96 min (2) | ¹HNMR (400 MHz, DMSO- d6): δ 8.01-7.99 (d, J = 6.8 Hz, 1H), 7.86 (s, 1H), 7.69- 7.67 (d, J = 7.6 Hz, 1H), 7.65-7.63 (d, J = 8.0 Hz, 1H), 7.50-7.46 (t, J = 7.8 Hz, 1H), 6.65-6.63 (d, J = 6.8 Hz, 1H), 6.18 (s, 1H), 2.31 (s, 3H), 1.30 (s, 9H). |
| 33 | 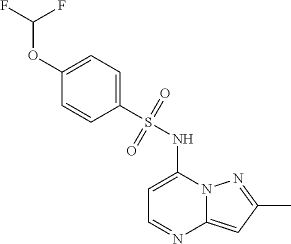 [SMILES: Cc1cc2nccc(NS(=O)(=O)c3ccc(OC(F)F)cc3)n2n1] | 352.97 (M − 1) | 98.12%, Rt = 4.07 min (2) | ¹HNMR (400 MHz, DMSO- d6): δ 13.38 (bs, 1 H), 8.05- 8.03 (d, J = 7.2 Hz, 1H), 7.94-7.92 (d, J = 8.4 Hz, 2H), 7.36 (s, 1H), 7.34-7.32 (d, J = 8.4 Hz, 2H), 6.66-6.64 (d, J = 7.2 Hz, 1H), 6.23 (s, 1H), 2.33 (s, 3H). |
| 34 | 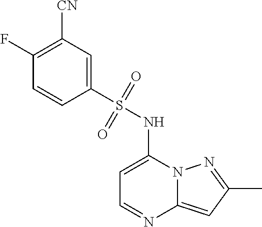 [SMILES: Cc1cc2nccc(NS(=O)(=O)c3ccc(F)c(C#N)c3)n2n1] | 332.07 | 98.88%, Rt = 3.85 min (2) | ¹HNMR (400 MHz, DMSO- d6): δ 8.43-8.41 (m, 1H), 8.26-8.21 (m, 1H), 8.06-8.04 (d, δ 7 Hz, 1H), 7.71-7.67 (m, 1H), 6.64-6.62 (d, J = 7 Hz, 1H), 6.23 (s, 1H), 2.34 (s, 3H). |
| 35 | 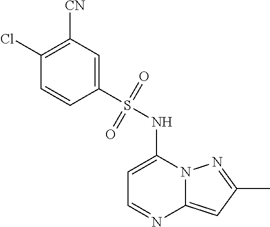 [SMILES: Cc1cc2nccc(NS(=O)(=O)c3ccc(Cl)c(C#N)c3)n2n1] | 348.10 | 99.12%, Rt = 4.84 min (2) | ¹HNMR (400 MHz, DMSO- d6): δ 13.48 (bs, 1 H), 8.41- 8.41 (d, J = 7.2 Hz, 1H), 8.16- 8.14 (d, J = 8.4 Hz, 1H), 8.06- 8.04 (d, J = 7.2 Hz, 1H), 7.93- 7.91 (d, J = 8.4 Hz, 1H), 6.63- 6.61 (d, J = 6.8 Hz, 1H), 6.24 (s, 1H), 2.34 (s, 3H). |
| 36 | 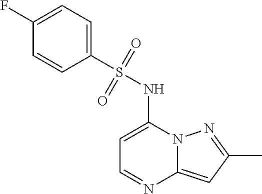 [SMILES: Cc1cc2nccc(NS(=O)(=O)c3ccc(F)cc3)n2n1] | 307.09 | 98.48%, Rt = 4.36 min (2) | ¹HNMR (400 MHz, DMSO- d6): δ 13.31 (bs, 1 H), 8.05- 8.03 (d, J = 7 .2 Hz, 1H), 7.96- 7.92 (m, 2H), 7.41-7.37 (t, J = 8.0 Hz, 2H), 6.66-6.64 (d, J = 7.2 Hz, 1H), 6.23 (s, 1H), 2.33 (s, 3H). |

### Example 2

### Synthesis of Compound 37 [4-(tert-butyl)-N-(2-cyclopropylpyrazolo[1,5-a]pyrimidin-7-yl)benzenesulfonamide] and Compounds 38-57

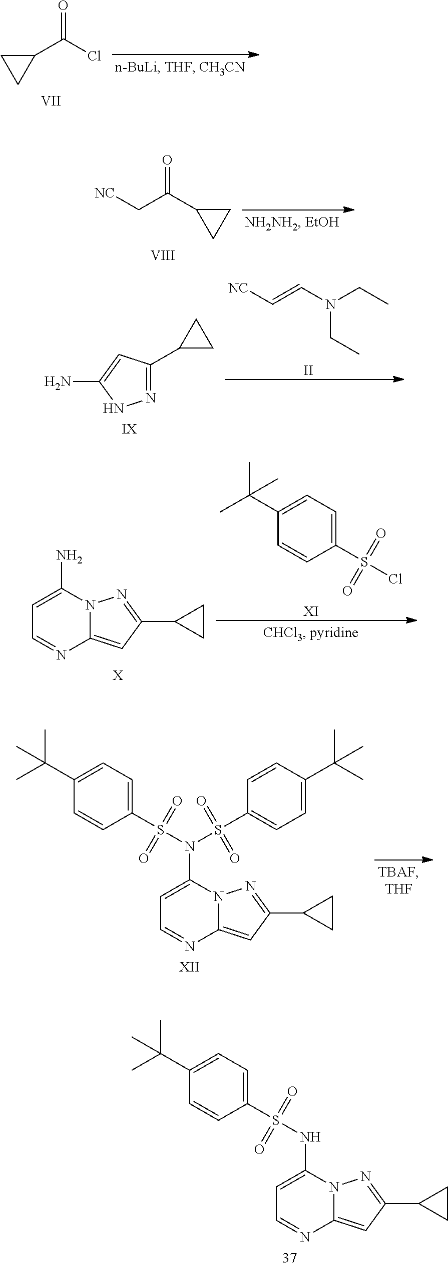

Synthesis of VIII:

To a stirred solution of acetonitrile (2.3 g; 56 mmol) in THF (20 mL) was added n-butyl lithium (35 mL; 56 mmol) dropwise at −78° C. under an argon atmosphere. The reaction mixture was stirred for 30 minutes maintaining the same temperature. A solution of cyclopropanecarbonyl chloride (VII; 3 g; 28 mmol) in THF (10 mL) was added to the reaction mixture and the stirring continued for 1.5 hours at −50° C. The reaction mixture was diluted with IN hydrochloric acid and extracted sequentially with ethyl acetate and dichloromethane (3×25 mL). The combined organic layers were washed with brine, dried over Na₂SO₄, filtered and concentrated under vacuum to afford 3-cyclopropyl-3-oxopropanenitrile as a crude solid (VIII; 3 g). This was used in the next step without further purification.

Synthesis of IX:

A mixture of compound VIII (3 g; 27 mmol) and hydrazine hydrate (2.25 mL; an excess) was dissolved in ethanol (150 mL) and the reaction mixture heated at 80° C. for 20 hours. The reaction mixture was then cooled and concentrated under reduced pressure. The crude product was purified by column chromatography (2% MeOH-DCM) to obtain 3-cyclopropyl-1H-pyrazol-5-amine as a yellow oil (IX; 1.5 g; 44% yield).

Synthesis of X:

To a stirred solution of 3-cyclopropyl-1H-pyrazol-5-amine (IXx; 1.5 g; 12 mmol) in pyridine (20 mL) was added 3-(diethylamino)acrylonitrile (II; 2.4 g; 19 mmol). The reaction mixture was heated at 120° C. for 14 hours, whereupon it was cooled and concentrated under reduced pressure. The crude mixture was purified by column chromatography using 2% MeOH-DCM to obtain 2-cyclopropylpyrazolo[1,5-a]pyrimidin-7-amine as a yellow solid (X; 1 g; 45% yield). ¹H NMR (400 MHz, DMSO-d6): δ 7.95-7.93 (d, J=5.2 Hz, 1H), 7.53 (bs, 2H), 6.02 (s, 1H), 5.97-5.95 (d, J=5.2 Hz, 1H), 2.07-2.0 (m, 1H), 0.98-0.96 (m, 2H), 0.82-0.81 (m, 2H). MS (M+1): 175.03.

### Synthesis of Compound 37: 4-(tert-butyl)-N-(2-cyclopropylpyrazolo[1,5-a]pyrimidin-7-yl)benzenesulfonamide

To a stirred solution of compound 2-cyclopropylpyrazolo[1,5-a]pyrimidin-7-amine (X; 200 mg; 1.14 mmol) in chloroform (10 mL) at 0° C. was added pyridine (270 mg; 3.44 mmol) and 4-tertbutylsulfonyl chloride (XI; 400 mg; 1.72 mmol). The reaction mixture was heated at 80° C. for 14 hours. The reaction mixture was cooled and concentrated at reduced pressure to afford the di-substituted sulfonamide product (XII) which was confirmed by LCMS. The crude product was further dissolved in THF (4 mL) in presence of TBAF (0.2 mL) and stirred at room temperature to 60° C. for 3 hours. The reaction mixture was concentrated, diluted with water and extracted with EtOAc (3×25 mL). The combined organic layers were washed with brine, dried over Na₂SO₄, filtered and concentrated to afford the crude compound, which was purified by column chromatography (4% MeOH-DCM) to afford the title compound 4-(tert-butyl)-N-(2-cyclopropylpyrazolo[1,5-a]pyrimidin-7-yl)benzenesulfonamide (37; 35 mg; 9% yield). ¹H NMR (400 MHz, DMSO-d6): δ 13.23 (bs, 1H), 7.98-7.97 (d, J=7.0 Hz, 1H), 7.80-7.78 (d, J=8.4 Hz, 2H), 7.57-7.55 (d, J=8.4 Hz, 2H), 6.65-6.63 (d, J=7.0 Hz, 1H), 6.06 (s, 1H), 2.07-2.0 (m, 1H), 1.28 (s, 9H), 1.01-0.96 (m, 2H), 0.82-0.78 (m, 2H). MS (M+1): 371.18. (LCMS purity 98.93%, 5.81 min) (2).

The Following Compounds were Prepared in Essentially the Same Manner Using the Appropriate Sulfonyl Chloride in the Final Step:

| | | LCMS | Purity | |
|---|---|---|---|---|
| CPD | Structure | (M + 1) | (LCMS) | ¹H NMR |
| 38 | 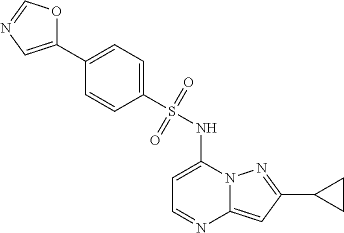 [SMILES: O=S(=O)(Nc1ccnc2cc(C3CC3)nn12)c1ccc(-c2cnco2)cc1] | 382.13 | 92.45%, Rt = 4.58 min (2) | ¹H NMR (400 MHz, DMSO-d6): δ 13.33 (bs, 1H), 8.54 (s, 1H), 8.02-8.06 (d, J = 7.2 Hz, 1H), 7.98-7.96 (dd, J = 8.4 Hz, 2H), 7.91-7.89 (dd, J = 8.4 Hz, 2H), 7.86 (s, 1H), 6.65-6.63 (d, J = 7.2 Hz, 1H), 6.10 (s, 1H), 2.07- 2.04 (m, 1H), 1.07-1.01 (m, 2H), 0.93-0.82 (m, 2H). |
| 39 | 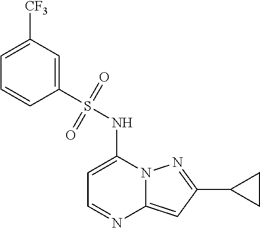 [SMILES: O=S(=O)(Nc1ccnc2cc(C3CC3)nn12)c1cccc(C(F)(F)F)c1] | 383.06 | 99.16%, Rt = 4.52 min (2) | ¹H NMR (400 MHz, DMSO-d6): δ 13.45 (bs, 1H), 8.21-8.19 (d, J = 7.6 Hz, 1H), 8.13 (s, 1H), 8.07-8.05 (d, J = 7.2 Hz, 1H), 8.03-8.01 (d, J = 7.2 Hz, 1H), 7.85-7.81 (t, J = 8.0 Hz, 1H), 6.68-6.66 (d, J = 7.2 Hz, 1H), 6.13 (s, 1H), 2.07-2.04 (m, 1H), 1.02-0.98 (m, 2H), 0.84-0.80 (m, 2H). |
| 40 | 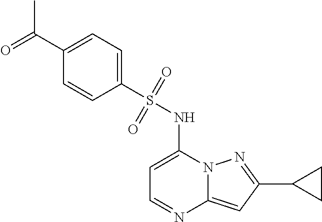 [SMILES: CC(=O)c1ccc(S(=O)(=O)Nc2ccnc3cc(C4CC4)nn23)cc1] | 357.06 | 98.94%, Rt = 3.91 min (2) | ¹H NMR (400 MHz, DMSO-d6): δ 13.38 (bs, 1H), 8.11-8.09 (d, J = 7.2 Hz, 2H), 8.02-7.99 (m, 3H), 6.64-6.62 (d, J = 7.6 Hz, 1H), 6.11 (s, 1H), 2.61 (s, 3H), 2.07-2.04 (m, 1H), 1.01-1.00 (m, 2H), 0.82 (m, 2H). |
| 41 | 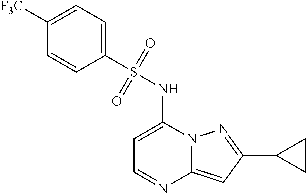 [SMILES: O=S(=O)(Nc1ccnc2cc(C3CC3)nn12)c1ccc(C(F)(F)F)cc1] | 383.14 | 99.32%, Rt = 4.51 min (2) | ¹H NMR (400 MHz, DMSO-d6): δ 8.07-8.05 (d, J = 8.0 Hz, 2H), 7.95-7.89 (m, 3H), 6.50-6.48 (d, J = 6.4 Hz, 1H), 6.05 (s, 1H), 2.04-2.01 (m, 1H), 0.99-0.97 (m, 2H), 0.80-0.79 (m, 2H). |
| 42 | 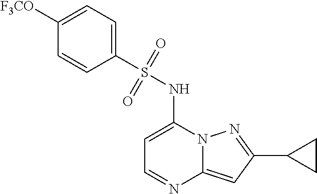 [SMILES: O=S(=O)(Nc1ccnc2cc(C3CC3)nn12)c1ccc(OC(F)(F)F)cc1] | 399.13 | 99.87%, Rt = 4.61 min (2) | ¹H NMR (400 MHz, DMSO-d6 with TFA): δ 8.02-7.99 (d, J = 8.8 Hz, 1H), 7.97-7.95 (d, J = 7.6 Hz, 2H), 7.45-7.43 (d, J = 7.6 Hz, 2H), 6.68-6.66 (d, J = 7.2 Hz, 1H), 6.05 (s, 1H), 2.04- 2.03 (m, 1H), 0.98-0.96 (m, 2H), 0.80 (m, 2H). |
| 43 | 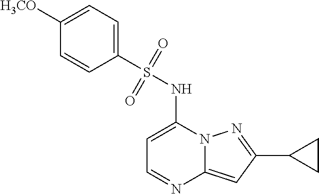 [SMILES: COc1ccc(S(=O)(=O)Nc2ccnc3cc(C4CC4)nn23)cc1] | 345.10 | 98.77%, Rt = 4.03 min (2) | ¹H NMR (400 MHz, DMSO-d6): δ 7.94-7.92 (d, J = 7.2 Hz, 1H), 7.80-7.77 (d, J = 8.8 Hz, 2H), 7.06-7.04 (d, J = 8.8 Hz, 2H), 6.56-6.54 (d, J = 6.8 Hz, 1H), 6.04 (s, 1H), 3.80 (s, 3H), 2.03 (m, 1H), 0.99-0.97 (m, 2H), 0.80-0.75 (m, 2H). |
| 44 | 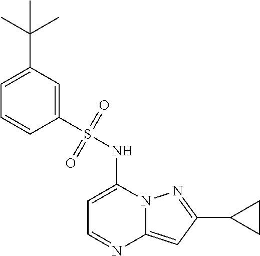 [SMILES: CC(C)(C)c1cccc(S(=O)(=O)Nc2ccnc3cc(C4CC4)nn23)c1] | 371.17 | 98.06%, Rt = 4.90 min (2) | ¹H NMR (400 MHz, DMSO-d6): δ 13.20 (bs, 1H), 7.99-7.97 (d, J = 7.2 Hz, 1H), 7.87 (s, 1H), 7.69-7.67 (d, J = 7.6 Hz, 1H), 7.65-7.63 (d, J = 8.0 Hz, 1H), 7.50-7.46 (t, J = 7.8 Hz, 1H), 6.65-6.63 (d, J = 7.2 Hz, 1H), 6.07 (s, 1H), 2.04-2.01 (m, 1H), 1.30 (s, 9H), 0.99-0.96 (m, 2H), 0.82-0.78 (m, 2H). |
| 45 | 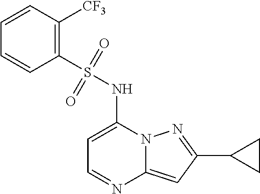 [SMILES: O=S(=O)(Nc1ccnc2cc(C3CC3)nn12)c1ccccc1C(F)(F)F] | 383.09 | 99.61%, Rt = 4.35 min (2) | ¹H NMR (400 MHz, DMSO-d6): δ 13.38 (bs, 1H), 8.28-8.26 (d, J = 7.6 Hz, 1H), 8.03-8.01 (d, J = 7.2 Hz, 1H), 7.96-7.94 (d, J = 7.2 Hz, 1H), 7.89-7.80 (m, 2H), 6.60-6.58 (d, J = 7.2 Hz, 1H), 6.09 (s, 1H), 2.08-2.01 (m, 1H), 1.02-0.97 (m, 2H), 0.82- 0.80 (m, 2H). |
| 46 | 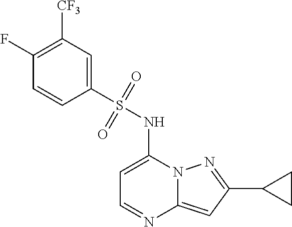 [SMILES: O=S(=O)(Nc1ccnc2cc(C3CC3)nn12)c1ccc(F)c(C(F)(F)F)c1] | 401.10 | 99.79%, Rt = 4.75 min (2) | ¹H NMR (400 MHz, DMSO-d6): δ 13.52 (bs, 1H), 8.27-8.25 (m, 1H), 8.18-8.16 (d, J = 7.2 Hz, 1H), 8.07-8.05 (d, J = 7.2 Hz, 1H), 7.76-7.71 (t, J = 9.8 Hz, 2H), 6.67-6.65 (d, J = 7.2 Hz, 1H), 6.13 (s, 1H), 2.07-2.03 (m, 1H), 1.02-1.00 (m, 2H), 0.83 (m, 2H). |
| 47 | 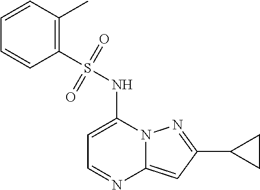 [SMILES: Cc1ccccc1S(=O)(=O)Nc1ccnc2cc(C3CC3)nn12] | 329.13 | 98.66%, Rt = 4.17 min (2) | ¹H NMR (400 MHz, DMSO-d6): δ 13.23 (bs, 1H), 7.98-7.96 (d, J = 7.2 Hz, 2H), 7.52-7.47 (m, 1H), 7.39-7.35 (m, 2H), 6.59- 6.57 (d, J = 7.6 Hz, 1H), 6.07 (s, 1H), 2.60 (s, 3H), 2.08-2.03 (m, 1H), 1.02-0.98 (m, 2H), 0.82- 0.80 (m, 2H). |
| 48 | 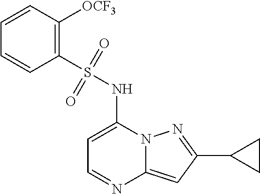 [SMILES: O=S(=O)(Nc1ccnc2cc(C3CC3)nn12)c1ccccc1OC(F)(F)F] | 399.16 | 96.01%, Rt = 4.88 min (2) | ¹H NMR (400 MHz, DMSO-d6): δ 13.36 (bs, 1H), 8.09-8.07 (d, J = 6.0 Hz, 1H), 8.04-8.02 (d, J = 7.2 Hz, 1H), 7.76-7.71 (m, 1H), 7.58-7.50 (m, 2H), 6.69-6.67 (d, J = 7.2 Hz, 1H), 6.12 (s, 1H), 2.05-2.03 (m, 1H), 1.02-0.97 (m, 2H), 0.83-0.79 (m, 2H). |
| 49 | 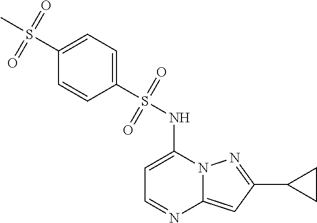 [SMILES: CS(=O)(=O)c1ccc(S(=O)(=O)Nc2ccnc3cc(C4CC4)nn23)cc1] | 393.17 | 98.11%, Rt = 4.05 min (2) | ¹H NMR (400 MHz, DMSO-d6): δ 13.21 (bs, 1H), 8.12-8.07 (m, 4H), 8.00-7.99 (d, J = 6.4 Hz, 1H), 6.59-6.57 (d, J = 6.8 Hz, 1H), 6.08 (s, 1H), 3.20 (s, 3H), 2.04 (m, 1H), 1.00-0.98 (m, 2H), 0.81-0.80 (m, 2H). |
| 50 | 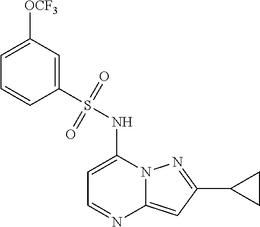 [SMILES: O=S(=O)(Nc1ccnc2cc(C3CC3)nn12)c1cccc(OC(F)(F)F)c1] | 399.15 | 97.60%, Rt = 5.01 min (2) | ¹H NMR (400 MHz, DMSO-d6): δ 13.46 (bs, 1H), 8.06-8.04 (d, J = 7.2 Hz, 1H), 7.93-7.91 (d, J = 8.0 Hz, 1H), 7.79 (s, 1H), 7.74-7.70 (t, J = 8.0 Hz, 1H), 7.65-7.63 (m, 1H) 6.66-6.64 (d, J = 7.2 Hz, 1H), 6.13 (s, 1H), 2.09-2.02 (m, 1H), 1.03-0.98 (m, 2H), 0.84-0.80 (m, 2H). |
| 51 | 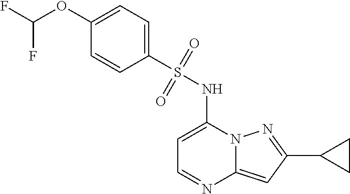 [SMILES: O=S(=O)(Nc1ccnc2cc(C3CC3)nn12)c1ccc(OC(F)F)cc1] | 381.15 | 99.72%, Rt = 4.75 min (2) | ¹H NMR (400 MHz, DMSO-d6): δ 13.31 (bs, 1H), 8.02-8.00 (d, J = 7.2 Hz, 1H), 7.94-7.92 (d, J = 8.8 Hz, 2H), 7.36-7.32 (m, 3H), 6.64-6.63 (d, J = 7.2 Hz, 1H), 6.10 (s, 1H), 2.07-2.01 (m, 1H), 1.01-0.99 (m, 2H), 0.83- 0.81 (m, 2H). |
| 52 | 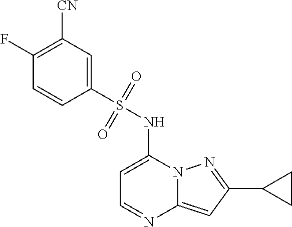 [SMILES: N#Cc1cc(S(=O)(=O)Nc2ccnc3cc(C4CC4)nn23)ccc1F] | 358.07 | 97.71%, Rt = 4.41 min (2) | ¹H NMR (400 MHz, DMSO-d6): δ 13.41 (bs, 1H), 8.44-8.42 (m, 1H), 8.26-8.22 (m, 1H), 8.04- 8.02 (d, J = 7.2 Hz, 1H), 7.72- 7.67 (t, J = 9.0 Hz, 1H), 6.65- 6.63 (d, J = 6.8 Hz, 1H), 6.12 (s, 1H), 2.07-2.02 (m, 1H), 1.03- 0.99 (m, 2H), 0.86-0.82 (m, 2H). |
| 53 | 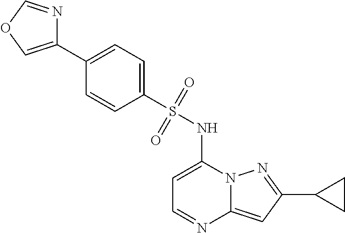 [SMILES: O=S(=O)(Nc1ccnc2cc(C3CC3)nn12)c1ccc(-c2cocn2)cc1] | 382.16 | 98.98%, Rt = 4.63 min (2) | ¹H NMR (400 MHz, DMSO-d6): δ 13.30 (bs, 1H), 8.77 (s, 1H), 8.52 (s, 1H), 8.00-7.92 (m, 5H), 6.65-6.63 (d, J = 7.2 Hz, 1H), 6.09 (s, 1H), 2.08-2.02 (m, 1H), 1.02-0.97 (m, 2H), 0.83-0.81 (m, 2H). |
| 54 | 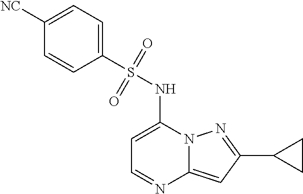 [SMILES: N#Cc1ccc(S(=O)(=O)Nc2ccnc3cc(C4CC4)nn23)cc1] | 340.03 | 93.01%, Rt = 4.35 min (2) | ¹H NMR (400 MHz, DMSO-d6): δ 13.41 (bs, 1H), 8.04 (m, 5H), 6.65-6.63 (d, J = 7.2 Hz, 1H), 6.13 (s, 1H), 2.09-2.02 (m, 1H), 1.03-0.98 (m, 2H), 0.84-0.80 (m, 2H). |
| 55 | 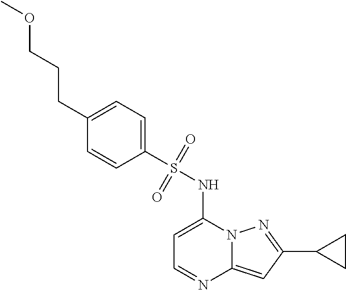 [SMILES: COCCCc1ccc(S(=O)(=O)Nc2ccnc3cc(C4CC4)nn23)cc1] | 387.25 | 98.42%, Rt = 4.98 min (2) | ¹H NMR (400 MHz, DMSO-d6): δ 13.23 (bs, 1H), 7.98-7.96 (d, J = 7.2 Hz, 1H), 7.79-7.77 (d, J = 8.0 Hz, 2H), 7.38-7.36 (d, J = 8.4 Hz, 2H), 6.64-6.62 (d, J = 7.2 Hz, 1H), 6.07 (s, 1H), 3.29 (m, 2H), 3.22 (s, 3H), 2.68- 2.64 (m, 2H), 2.06-2.02 (m, 1H), 1.81-1.77 (m, 2H), 1.02-0.97 (m, 2H), 0.82-0.79 (m, 2H). |
| 56 | 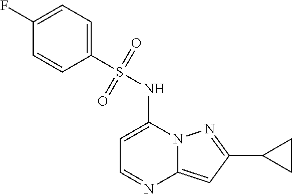 [SMILES: O=S(=O)(Nc1ccnc2cc(C3CC3)nn12)c1ccc(F)cc1] | 333.13 | 99.83%, Rt = 4.07 min (2) | ¹H NMR (400 MHz, DMSO-d6): δ 13.31 (bs, 1H), 8.02-8.0 (d, J = 7.2 Hz, 1H), 7.96-7.92 (m, 2H), 7.41-7.37 (t, J = 8.8 Hz, 2H), 6.64-6.62 (d, J = 7.2 Hz, 1H), 6.10 (s, 1H), 2.05 (m, 1H), 1.01- 0.99 (m, 2H), 0.82-0.79 (m, 2H). |
| 57 | 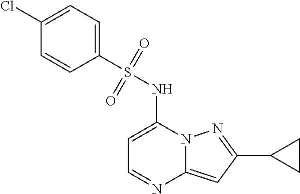 [SMILES: O=S(=O)(Nc1ccnc2cc(C3CC3)nn12)c1ccc(Cl)cc1] | 349.08 | 98.91%, Rt = 4.43 min (2) | ¹H NMR (400 MHz, DMSO-d6): δ 13.35 (bs, 1H), 8.02-8.0 (d, J = 7.2 Hz, 1H), 7.89-7.87 (d, J = 8.8 Hz, 2H), 7.64-7.61 (d, J = 8.4 Hz, 2H), 6.62-6.60 (d, J = 7.2 Hz, 1H), 6.10 (s, 1H), 2.05 (m, 1H), 1.03-0.99 (m, 2H), 0.83-0.81 (m, 2H). |

### Example 3

### Synthesis of Compound 58 [(4-(tert-butyl)-N-(6-cyanopyrazolo[1,5-a]pyrimidin-7-yl)benzenesulfonamide] and Compounds 59-61

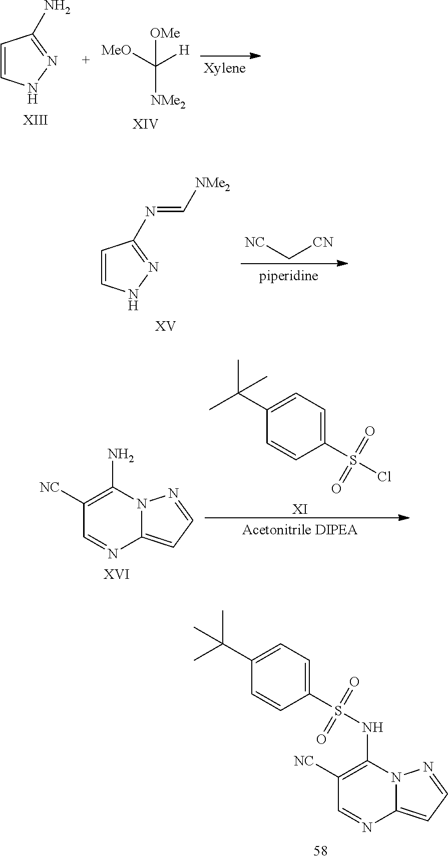

Synthesis of XV:

A mixture of XIII (3 g; 36.14 mmol) and dimethylformamide dimemethyl acetal (XIV; 4.3 g; 36.14 mmol) was heated to a reflux in xylene (40 mL) for 3 hours. The reaction mixture was then allowed to cool to room temperature and the product was collected by filtration and crystallized from toluene to afford N,N-dimethyl-N′-(1H-pyrazol-3-yl) formimidamide as a yellow solid (XV; 3.8 g; 76% yield). ¹H NMR (400 MHz, DMSO-d6): δ 12.03 (bs, 1H), 7.94 (s, 1H), 5.77 (s, 1H), 2.98 (s, 3H), 2.88 (s, 3H).

Synthesis of XVI:

A mixture of XV (2.4 g; 17.39 mmol) and malononitrile (1.14 g; 17.39 mmol) was heated to a reflux in ethanol (20 mL) in the presence of piperidine (2.9 g; 34 mmol) for 12 hours. The reaction mixture was then allowed to cool to room temperature and the solid product formed, was collected and crystallized to afford 7-aminopyrazolo[1,5-a]pyrimidine-6-carbonitrile (XVI; 1.9 g; 68% yield). ¹H NMR (400 MHz, DMSO-d6): δ 8.95 (bs, 2H), 8.32 (s, 1H), 8.24-8.23 (d, J=1.6, 1H), 6.59 (d, J=1.6, 1H). MS (M−1): 158.

Synthesis of Compound 58; (4-(tert-butyl)-N-(6-cyanopyrazolo[1,5-a]pyrimidin-7-yl)benzenesulfonamide):

To a stirred solution 7-aminopyrazolo[1,5-a]pyrimidine-6-carbonitrile (XVI; 0.5 g; 3.14 mmol) in acetonitrile (10 mL) was added DIPEA (1.21 g; 9.43 mmol) and 4-tertbutylphenylsulfonyl chloride (XI; 0.87 g; 3.77 mmol) at 0° C. The reaction mixture was then heated at 90° C. for 12 hours. The reaction mixture was concentrated at reduced pressure, diluted with cold water and extracted with dichloromethane (3×20 mL). The combined organic layers were washed with brine, dried over Na₂SO₄, filtered and concentrated under vacuum. The crude compound was purified through a Combiflash® column using 3% MeOH-DCM as an eluent to afford the title compound, 4-(tert-butyl)-N-(6-cyanopyrazolo[1,5-a]pyrimidin-7-yl)benzenesulfonamide, as a white solid (58; 0.050 g, 4% yield). ¹H NMR (400 MHz, DMSO-d6): δ 8.58 (s, 1H), 8.09 (d, J=2.0 Hz, 1H), 7.87-7.84 (d, J=8.4 Hz, 2H), 7.58-7.56 (d, J=8.4 Hz, 2H), 6.48 (d, J=2.0 Hz, 1H), 1.29 (s, 9H). MS (M+1): 356.09. (LCMS purity 97.26%, 4.87 min) (1).

The Following Compounds were Prepared in Essentially the Same Manner Using the Appropriate Sulfonyl Chloride in the Final Step:

| | | LCMS | Purity | |
|---|---|---|---|---|
| CPD | Structure | (M + 1) | (LCMS) | ¹H NMR |
| 59 | 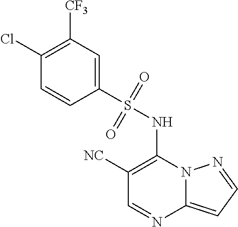 [SMILES: N#Cc1cnc2ccnn2c1NS(=O)(=O)c1ccc(Cl)c(C(F)(F)F)c1] | 401.99 | 95.07%, Rt = 4.98 min (1) | ¹H NMR (400 MHz, DMSO-d6): δ 8.50 (s, 1H), 8.36 (m, 1H), 8.20-8.18 (d, J = 8.8 Hz, 1H), 8.10 (s, 1H), 7.93-7.90 (d, J = 8.4. Hz, 1H), 6.5 (s, 1H). |
| 60 | 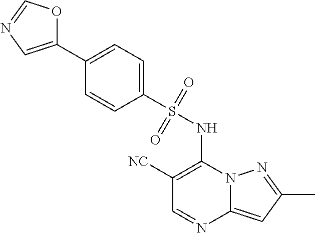 [SMILES: Cc1cc2ncc(C#N)c(NS(=O)(=O)c3ccc(-c4cnco4)cc3)n2n1] | 379.06 (M − 1) | 98.28%, Rt = 3.77 min (1) | ¹H NMR (400 MHz, DMSO-d6): δ 8.50 (s, 1H), 8.05 (s, 1H), 7.99-7.57 (m, 2H), 7.85-7.81 (m, 3H), 6.19 (s, 1H), 2.26 (s, 3H). |
| 61 | 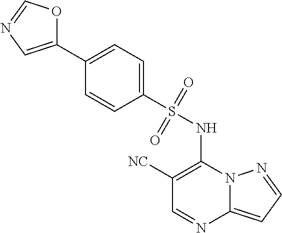 [SMILES: N#Cc1cnc2ccnn2c1NS(=O)(=O)c1ccc(-c2cnco2)cc1] | 365.06 (M − 1) | 98.95%, Rt = 3.55 min (1) | ¹H NMR (400 MHz, DMSO-d6): δ 8.51 (s, 1H), 8.13 (s, 1H), 8.02-8.00 (m, 2H), 7.96-7.95 (d, J = 4 Hz, 1H), 7.86-7.84 (m, 2H), 7.82 (s, 1H) 6.40-6.4 (d, J = 2Hz, 1H). |

### Example 4

### Synthesis of Compound 62 [4-(tert-butyl)-N-(2-(pyridin-3-yl)pyrazolo[1,5-a]pyrimidin-7-yl)benzenesulfonamide] and Compounds 63-74

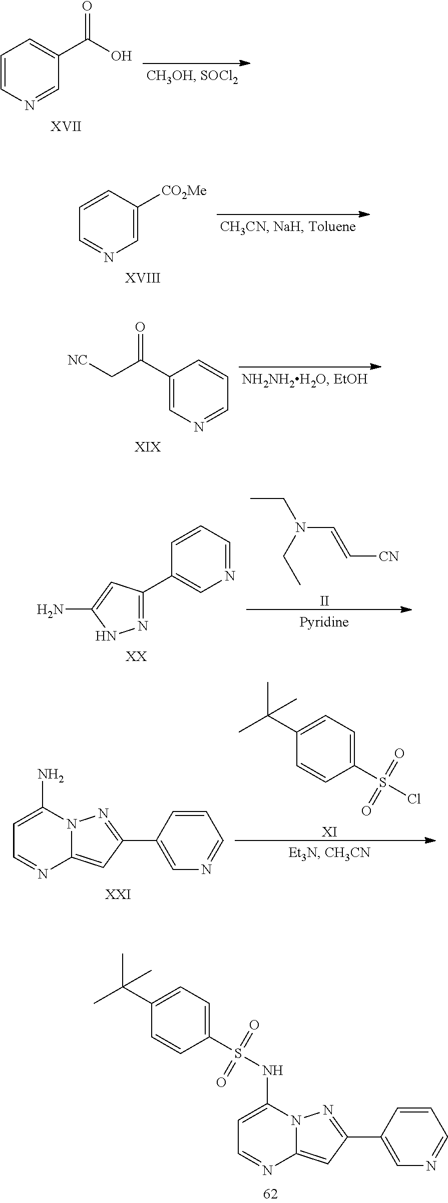

Synthesis of XVIII:

To a stirred solution of nicotinic acid (XVII; 10 g; 81 mmol) in methanol (90 mL) was added thionyl chloride (14.48 g; 122 mmol) drop wise at 0° C. The reaction mixture was heated to a reflux for 12 hours. The reaction mixture was cooled, concentrated and diluted with water. The aqueous layer was extracted with ethyl acetate (3×50 mL). The combined organic layers were washed with sodium bicarbonate, brine, dried over Na₂SO₄, filtered and concentrated under vacuum to afford methyl nicotinate as white solid (XVIII; 8 g, 75% yield). ¹H NMR (400 MHz, DMSO-d6): δ 9.08 (s, 1H), 8.23-8.80 (dd, J=1.2 Hz, 4.8 Hz, 1H), 8.30-8.827 (m, 1H), 7.58-7.55 (dd, J=5.0 Hz, 8 Hz, 1H), 3.88 (s, 3H). MS (M+1): 138.19.

Synthesis of XIX:

To a stirred solution of methyl nicotinate (XVIII; 8 g; 58 mmol) in toluene (110 mL) was added sodium hydride (2.8 g; 110 mmol) at 0° C. The reaction mixture was stirred at 0° C. for 30 minutes and then acetonitrile (12 g; 91 mmol) was added. The reaction mixture was heated to a reflux for 72 hours. The reaction mixture was cooled, concentrated at reduced pressure and diluted with ice cold water. The reaction mixture was acidified using glacial acetic acid. The aqueous layer was extracted with ethyl acetate (3×100 mL). The combined organic layers were washed with brine, dried over Na₂SO₄, filtered and concentrated under vacuum to afford 3-oxo-3-(pyridin-3-yl)propanenitrile as a yellow solid (XIX; 6 g, 70% yield). MS (M−1): 145.01.

Synthesis of XX:

To a stirred solution of 3-oxo-3-(pyridin-3-yl)propanenitrile (XIX; 5.8 g; 40 mmol) in ethanol (190 mL) was added hydrazine hydrate (3.97 g; 80 mmol). The reaction mixture was heated to a reflux for 12 hours. The reaction mixture was cooled and concentrated to afford 3-(pyridin-3-yl)-1H-pyrazol-5-amine as a crude yellow solid (XX; 4 g, 63% yield). MS (M+1): 160.9.

Synthesis of XXI:

To a stirred solution of 3-(pyridin-3-yl)-1H-pyrazol-5-amine (XX; 8 g; 50 mmol) in pyridine (80 mL) was added 3-(diethylamino)acrylonitrile (II; 9.3 g; 67 mmol). The reaction mixture was heated at 100° C. for 14 hours. The reaction mixture was cooled and concentrated under reduced pressure. The crude mixture was purified by column chromatography using 5% MeOH-DCM to obtain 2-(pyridin-3-yl)pyrazolo[1,5-a]pyrimidin-7-amine as a yellow solid (XXI; 1.8 g; 17% yield). ¹H NMR (400 MHz, DMSO-d6): δ 9.26 (s, 1H), 8.60-8.59 (d, J=4.8 Hz, 1H), 8.39-8.37 (d, J=8 Hz, 1H), 8.08-8.07 (d, J=5.2 Hz, 1H), 7.78 (bs, 2H), 7.53-7.50 (m, 1H), 6.97 (s, 1H), 6.13-6.11 (d, J=5.2 Hz, 1H). MS (M+1): 212.2.

### Synthesis of Compound 62; 4-(tert-butyl)-N-(2-(pyridin-3-yl)pyrazolo[1,5-a]pyrimidin-7-yl)benzenesulfonamide

To a stirred solution of 2-(pyridin-3-yl)pyrazolo[1,5-a]pyrimidin-7-amine (XXI; 0.4 g; 1.88 mmol) in acetonitrile (25 mL) was added triethylamine (0.62 g; 5.68 mmol) and 4-tertbutylphenylsulfonyl chloride (XI, 0.65 g; 2.84 mmol) at 0° C. The reaction mixture was heated at 70° C. for 12 hours. The reaction mixture was concentrated at reduced pressure and diluted with cold water and extracted with dichloromethane (3×20 mL). The combined organic layers were washed with brine, dried over Na₂SO₄, filtered and concentrated under vacuum. The crude compound was purified using preparative HPLC to afford the title compound as a white solid (62; 0.025 g, 4% yield). ¹H NMR (400 MHz, DMSO-d6): δ 13.61 (bs, 1H), 9.20 (s, 1H), 8.64-8.63 (d, J=4.0 Hz, 1H), 8.40-8.38 (d, J=7.2 Hz, 1H), 8.12-8.10 (d, J=8.4 Hz, 1H), 7.86-7.84 (m, 2H), 7.60-7.58 (m, 2H), 7.54-7.51 (m, 1H), 7.01 (s, 1H), 6.79-6.78 (m, 1H), 1.28 (s, 9H). MS (M+1): 408.14. (LCMS purity 96.89%, 4.78 min) (1).

The Following Compounds were Prepared in Essentially the Same Manner Using the Appropriate Sulfonyl Chloride in the Final Step:

| | | LCMS | Purity | |
|---|---|---|---|---|
| CPD | Structure | (M + 1) | (LCMS) | ¹H NMR |
| 63 | 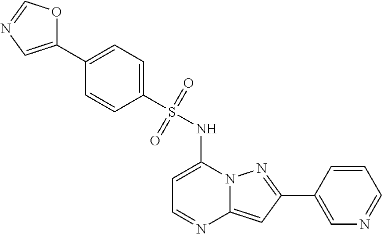 [SMILES: O=S(=O)(Nc1ccnc2cc(-c3cccnc3)nn12)c1ccc(-c2cnco2)cc1] | 419.14 | 97.58%, Rt = 3.92 min (1) | ¹H NMR (400 MHz, DMSO- d6): δ 9.14 (bs, 1H), 8.58 (d, J = 3.2 Hz, 1H), 8.44 (s, 1H), 8.37-8.35 (d, J = 8 Hz, 1H), 8.0-7.97 (m, 3H), 7.88-7.86 (m, 2H), 7.77 (s, 1H), 7.54- 7.50 (m, 1H), 6.9 (s, 1H), 6.6-6.59 (d, J = 4, 1H). |
| 64 | 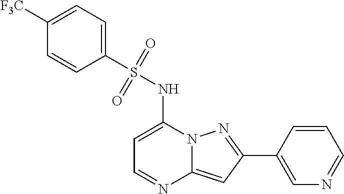 [SMILES: O=S(=O)(Nc1ccnc2cc(-c3cccnc3)nn12)c1ccc(C(F)(F)F)cc1] | 420.07 | 98.34%, Rt = 4.93 min (1) | ¹H NMR (400 MHz, DMSO- d6): δ 9.19 (s, 1H), 8.62-8.61 (d, J = 4 Hz, 1H), 8.39-8.37 (d, J = 8 Hz, 1H), 8.13-8.11 (d, J = 8 Hz, 2H), 8.08-8.06 (d, J = 8 Hz, 1H), 7.93-7.91 (d, J = 8 Hz, 2H), 7.53-7.49 (m, 1H), 6.98 (s, 1H), 6.63- 6.61 (d, J = 8 Hz, 1H). |
| 65 | 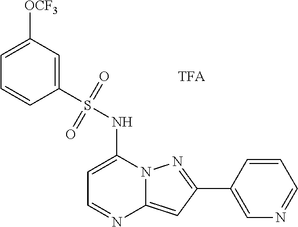 [SMILES: O=S(=O)(Nc1ccnc2cc(-c3cccnc3)nn12)c1cccc(OC(F)(F)F)c1 ; "TFA"] | 436.12 | 97.53%, Rt = 4.68 min (1) | ¹H NMR (400 MHz, DMSO- d6 with D2O): δ 9.23 (s, 1H), 8.70-8.69 (d, J = 4 Hz, 1H), 8.61-8.59 (d, J = 8 Hz, 1H), 8.13-8.11 (d, J = 7.2 Hz, 1H), 7.97-7.95 (d, J = 7.6 Hz, 1H), 7.84 (s, 1H), 7.75- 7.70 (m, 2H), 7.63-7.61 (m, 1H), 7.05 (s, 1H) 6.80-6.79 (d, J = 6.8 Hz, 1H). |
| 66 | 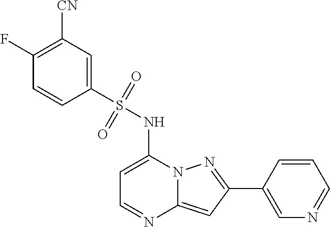 [SMILES: N#Cc1cc(S(=O)(=O)Nc2ccnc3cc(-c4cccnc4)nn23)ccc1F] | 395.13 | 97.44%, Rt = 4.05 min (1) | ¹H NMR (400 MHz, DMSO- d6): δ 9.21 (s, 1H), 8.65-8.64 (d, J = 3.6 Hz, 1H), 8.50-8.48 (m, 1H), 8.41-8.39 (d, J = 8.4 Hz, 1H), 8.32-8.28 (m, 1H), 8.15-8.13 (m, 1H), 7.73-7.69 (m, 1H), 7.55-7.52 (m, 1H), 7.05 (s, 1H) 6.75-6.74 (d, J = 7.2, 1H). |
| 67 | 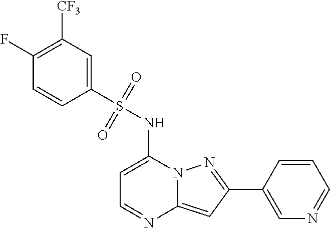 [SMILES: O=S(=O)(Nc1ccnc2cc(-c3cccnc3)nn12)c1ccc(F)c(C(F)(F)F)c1] | 438.13 | 99.57%, Rt = 4.72 min (1) | ¹H NMR (400 MHz, DMSO- d6): δ 11.94 (bs, 1H), 9.17 (s, 1H), 8.58-8.57 (d, J = 3.6 Hz, 1H), 8.36-8.34 (d, J = 7.6 Hz, 1H), 8.24-8.22 (m, 1H), 8.16-8.15 (d, J = 6.4 Hz, 1H), 7.98-7.97 (d, J = 5.6 Hz, 1H), 7.67-7.62 (t, J = 9.6 Hz, 1H), 7.50-7.47 (m, 1H), 6.89 (s, 1H) 6.44-6.43 (d, J = 5.6 Hz, 1H). |
| 68 | 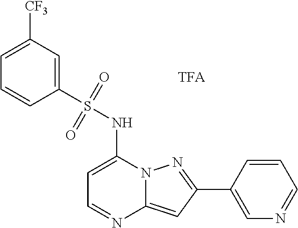 [SMILES: O=S(=O)(Nc1ccnc2cc(-c3cccnc3)nn12)c1cccc(C(F)(F)F)c1 ; "TFA"] | 420.04 | 98.38%, Rt = 4.43 min (1) | ¹H NMR (400 MHz, DMSO- d6): δ 9.21 (s, 1H), 8.65-8.63 (m, 1H), 8.41-8.38 (m, 1H), 8.26-8.24 (d, J = 7.6 Hz, 1H), 8.19-8.15 (m, 2H), 8.03- 8.01 (d, J = 8.4 Hz, 1H), 7.86-7.82 (t, J = 8 Hz, 1H), 7.55-7.51 (m, 1H), 7.04 (s, 1H) 6.77-6.76 (d, J = 6.8 Hz, 1H). |
| 69 | 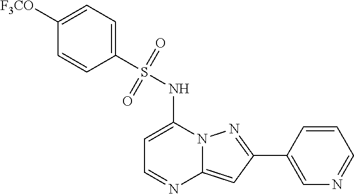 [SMILES: O=S(=O)(Nc1ccnc2cc(-c3cccnc3)nn12)c1ccc(OC(F)(F)F)cc1] | 436.1 | 99.89%, Rt = 1.06 min (1) | ¹H NMR (400 MHz, DMSO- d6): δ 9.13 (s, 1H), 8.54-8.53 (d, J = 4.8 Hz, , 1H), 8.33- 8.31 (d, J = 8 Hz, 1H), 7.95- 7.93 (d, J = 8.8 Hz, 2H), 8.87-8.85 (d, J = 8 Hz, 1H), 7.47-7.42 (m, 3H), 6.79 (s, 1H), 6.33-6.31 (d, J = 5.6 Hz, 1H). |
| 70 | 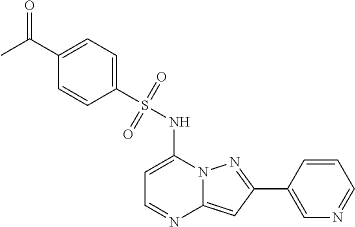 [SMILES: CC(=O)c1ccc(S(=O)(=O)Nc2ccnc3cc(-c4cccnc4)nn23)cc1] | 394.1 | 98.71%, Rt = 1.09 min (1) | ¹H NMR (400 MHz, DMSO- d6): δ 9.19 (s, 1H), 8.63-8.61 (d, J = 4.8 Hz, 1H), 8.39- 8.37 (d, J = 8 Hz, 1H), 8.1- 8.0 (m, 5H), 7.52-7.49 (m, 1H), 7.01 (s, 1H), 6.73-6.71 (d, J = 7.2 Hz, 1H). 2.65 (s, 3H). |
| 71 | 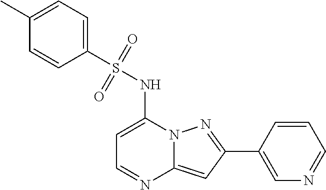 [SMILES: Cc1ccc(S(=O)(=O)Nc2ccnc3cc(-c4cccnc4)nn23)cc1] | 366.06 | 97.02%, Rt = 4.14 min (1) | ¹H NMR (400 MHz, DMSO- d6): δ 11.65 (bs, 1H), 9.26 (s, 1H), 8.71-8.70 (d, J = 4.4 Hz, 1H), 8.55-8.53 (d, J = 8 Hz, 1H), 8.12-8.10 (d, J = 7.6 Hz, 1H), 7.82-7.80 (d, J = 8.4 Hz, 2H), 7.67-7.64 (m, 1H), 7.38-7.36 (d, J = 8 Hz, 2H), 7.05 (s, 1H), 6.76-6.74 (d, J = 7.2 Hz, 1H), 2.36 (s, 3H). |
| 72 | 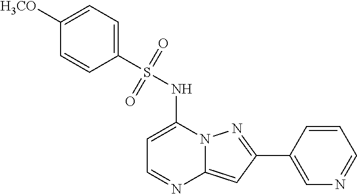 [SMILES: COc1ccc(S(=O)(=O)Nc2ccnc3cc(-c4cccnc4)nn23)cc1] | 382.07 | 97.13%, Rt = 3.89 min (1) | ¹H NMR (400 MHz, DMSO- d6): δ 13.59 (bs, 1H), 9.24 (s, 1H), 8.68 (m, 1H), 8.49-8.47 (d, J = 8 Hz, 1H), 8.11-8.09 (d, J = 7.2 Hz, 1H), 7.87- 7.85 (d, J = 8.8 Hz, 2H), 7.62-7.58 (m, 1H), 7.10-7.08 (d, J = 8 Hz, 2H), 7.03 (s, 1H), 6.76-6.74 (d, J = 7.2 Hz, 1H), 3.81 (s, 3H). |
| 73 | 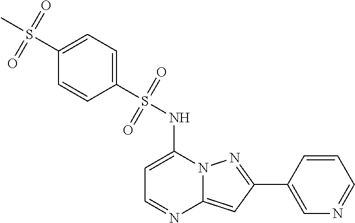 [SMILES: CS(=O)(=O)c1ccc(S(=O)(=O)Nc2ccnc3cc(-c4cccnc4)nn23)cc1] | 430.08 | 97.91%, Rt = 3.64 min (1) | ¹H NMR (400 MHz, DMSO- d6): δ 9.23 (s, 1H), 8.66-8.64 (m, 1H), 8.41-8.39 (m, 1H), 8.18-8.09 (m, 5H), 7.54 (s, 1H), 7.02 (s, 1H), 6.72-6.70 (d, J = 6.8 Hz, 1H), 3.27 (s, 3H). |
| 74 | 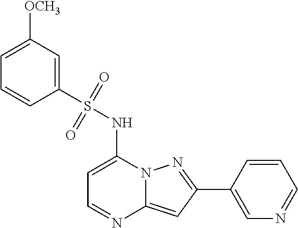 [SMILES: COc1cccc(S(=O)(=O)Nc2ccnc3cc(-c4cccnc4)nn23)c1] | 382.13 | 98.45%, Rt = 4.07 min (1) | ¹H NMR (400 MHz, DMSO- d6): δ 13.56 (bs, 1H), 9.27 (s, 1H), 8.70 (s, 1H), 8.55-8.53 (d, J = 8 Hz, 1H), 8.14-8.12 (d, J = 7.2 Hz, 1H), 7.66- 7.63 (m, 1H), 7.50-7.47 (m, 2H), 7.41 (s, 1H), 7.20-7.17 (m, 1H), 7.06 (s, 1H), 6.79- 6.77 (d, J = 7.2 Hz, 1H), 3.82 (s, 3H). |

### Example 5

### Synthesis of Compound 75 [4-(tert-butyl)-N-(2-(4-cyanophenyl)pyrazolo[1,5-a]pyrimidin-7-yl)benzenesulfonamide]

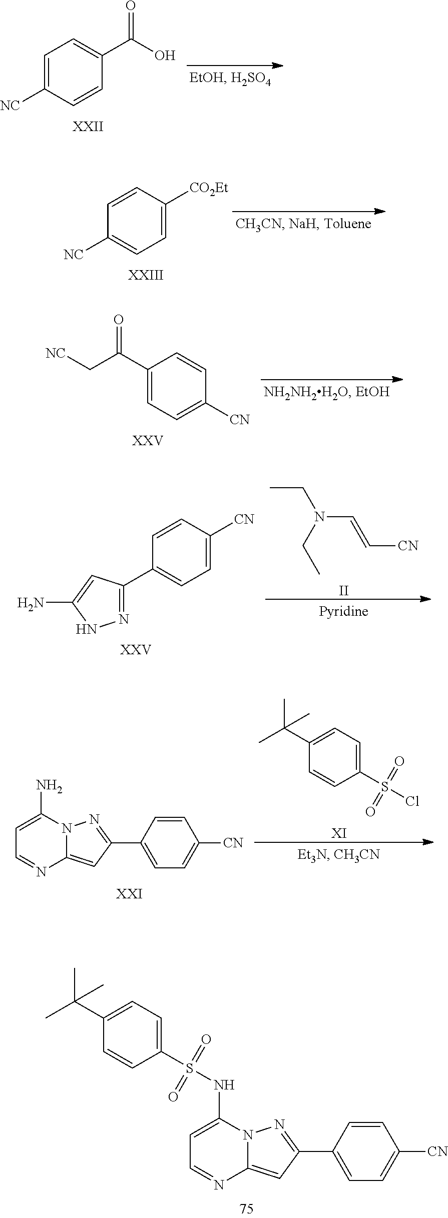

Synthesis of XXIII:

To a stirred solution of 4-cyanobenzoic acid (XXII; 10 g; 68 mmol) in ethanol (150 mL) was added a catalytic quantity of sulfuric acid (1 mL). The reaction mixture was heated to a reflux for 12 hours. The reaction mixture was cooled, concentrated at reduced pressure and diluted with water. The aqueous layer was extracted with ethyl acetate (3×50 mL). The combined organic layers were washed sequentially with sodium bicarbonate and brine, then dried over Na₂SO₄, filtered and concentrated under vacuum to afford ethyl 4-cyanobenzoate as a white solid (XXIII; 10 g, 84% yield). ¹H NMR (400 MHz, DMSO-d6) δ 8.10-8.08 (d, J=8.4 Hz, 2H), 8.01-7.99 (d, J=8.4 Hz, 2H), 4.37-4.32 (q, J=7.2 Hz, 2H), 1.35-1.31 (t, J=7.2 Hz, 3H).

Synthesis of XXIV:

To a stirred solution of acetonitrile (1.4 g; 30 mmol) in THF (30 mL) was added sodium hydride (2.28 g; 50 mmol) at 0° C. The stirring was continued for 30 minutes and then a solution of ethyl 4-cyanobenzoate (XXIII; 5 g; 28 mmol) in THF (20 mL) was added. The reaction mixture was stirred at 80° C. for 12 hours. The reaction mixture was cooled, concentrated at reduced pressure and diluted with ice cold water. The reaction mixture was acidified using glacial acetic acid. The aqueous layer was extracted with ethyl acetate (3×25 mL). The combined organic layers were washed with brine, dried over Na₂SO₄, filtered and concentrated under vacuum to afford 4-(2-cyanoacetyl)benzonitrile as a brown solid (XXIV; 1.5 g, 31% yield). ¹H NMR (400 MHz, DMSO-d6) δ 8.04-8.02 (d, J=8.4 Hz, 2H), 7.86-7.83 (d, J=8.4 Hz, 2H), 4.11 (s, 2H).

Synthesis of XXV:

To a stirred solution of 4-(2-cyanoacetyl)benzonitrile (XXIV; 1.5 g; 8.8 mmol) in ethanol (30 mL) was added hydrazine hydrate (1.32 g; 26 mmol). The reaction mixture was heated to a reflux for 12 hours. The reaction mixture was cooled and concentrated to afford 4-(5-amino-1H-pyrazol-3-yl)benzonitrile as a crude off white solid (XXV; 0.6 g, 29% yield).

Synthesis of XXVI:

To a stirred solution of 4-(5-amino-1H-pyrazol-3-yl)benzonitrile (XXV; 1.7 g; 9.2 mmol) in pyridine (15 mL) was added 3-(diethylamino)acrylonitrile (II; 1.7 g; 13.8 mmol). The reaction mixture was heated at 100° C. for 18 hours. The reaction mixture was cooled and concentrated under reduced pressure. The crude mixture was purified by column chromatography using 2% MeOH-DCM to obtain 4-(7-aminopyrazolo[1,5-a]pyrimidin-2-yl)benzonitrile as an off white solid (XXVI; 0.6 g; 27% yield). MS (M+1): 236.05.

### Synthesis of Compound 75; 4-(tert-butyl)-N-(2-(4-cyanophenyl)pyrazolo[1,5-a]pyrimidin-7-yl)benzenesulfonamide

To a stirred solution of 4-(7-aminopyrazolo[1,5-a]pyrimidin-2-yl)benzonitrile (XXVI; 0.2 g; 0.85 mmol) in pyridine (5 mL) was added 4-tertbutylphenylsulfonyl chloride (XI, 0.24 g; 1.02 mmol) and catalytic DMAP at 0° C. The reaction mixture was heated to a reflux for 12 hours. The reaction mixture was concentrated under reduced pressure and the purity improved using Combiflash® column chromatography and further purified using preparative HPLC to afford the title compound 4-(tert-butyl)-N-(2-(4-cyanophenyl)pyrazolo[1,5-a]pyrimidin-7-yl)benzenesulfonamide (75; 0.015 g, 4% yield) as white solid. ¹H NMR (400 MHz, DMSO-d6): δ 8.22-8.20 (d, J=8.4 Hz, 2H), 8.09-8.07 (d, J=6.8 Hz, 1H), 7.95-7.93 (d, J=8.0 Hz, 2H), 7.85-7.83 (d, J=8.8 Hz, 2H), 7.59-7.56 (d, J=8.8 Hz, 2H), 7.01 (s, 1H), 6.75-6.73 (d, J=6.8 Hz, 1H), 1.28 (s, 9H). MS (M+1): 432.20. (LCMS purity 98.46%, 6.13 min) (2).

### Example 6

### Synthesis of Compound 76 [4-(tert-butyl)-N-(2-(3-cyanophenyl)pyrazolo[1,5-a]pyrimidin-7-yl)benzenesulfonamide] and Compounds 77-78

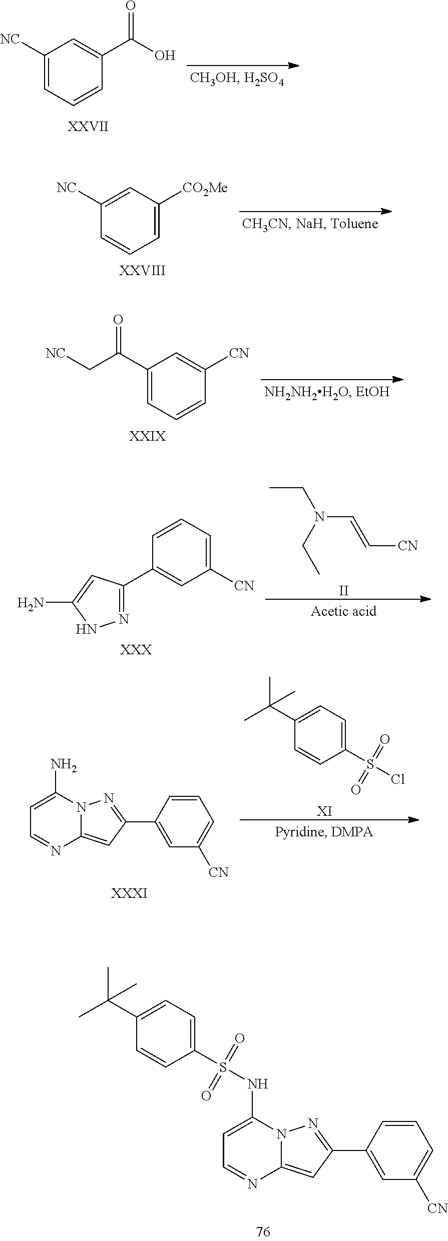

Synthesis of XXVIII:

To a stirred solution of 3-cyanobenzoic acid (XXVII; 6 g; 41 mmol) in methanol (80 mL) was added catalytic sulfuric acid (5 mL). The reaction mixture was heated to a reflux for 12 hours. The reaction mixture was cooled, concentrated under reduced pressure and diluted with water. The aqueous layer was extracted with ethyl acetate (3×50 mL). The combined organic layers were washed with sodium bicarbonate, brine, dried over Na₂SO₄, filtered and concentrated under vacuum to afford methyl 3-cyanobenzoate as a white solid (XXVIII; 3 g, 62% yield). ¹H NMR (400 MHz, CDCl₃): δ 8.36 (s, 1H), 8.26-8.24 (d, J=8.0 Hz, 1H), 7.84-7.82 (d, J=8.0 Hz, 1H), 7.63-7.58 (m 1H), 3.95 (s, 3H).

Synthesis of XXIX:

To a stirred solution of acetonitrile (3.8 g; 93 mmol) in toluene (60 mL) was added sodium hydride (1.48 g; 38 mmol) at 0° C. The stirring was continued for 30 minutes and then methyl 3-cyanobenzoate (XXVIII; 3 g; 18 mmol) was added. The reaction mixture was stirred at 100° C. for 12 hours. The reaction mixture was cooled and concentrated and diluted with ice cold water. The reaction mixture was acidified using IN HCl. The aqueous layer was extracted with ethyl acetate (3×25 mL). The combined organic layers were washed with brine, dried over Na₂SO₄, filtered and concentrated under vacuum to afford 3-(2-cyanoacetyl)benzonitrile as a yellow solid (XXIX; 2.7 g, 18% yield). MS (M−1): 169.10.

Synthesis of XXX:

To a stirred solution of 3-(2-cyanoacetyl)benzonitrile (XXIX; 6 g; 35 mmol) in ethanol (80 mL) was added hydrazine hydrate (15 mL). The reaction mixture was heated to a reflux for 12 hours. The reaction mixture was cooled, concentrated at reduced pressure and triturated with hexane to afford the title compound 3-(5-amino-1H-pyrazol-3-yl)benzonitrile as a crude sticky green solid (XXX; 4 g, 71% yield). MS (M+1): 185.1.

Synthesis of XXXI:

To a stirred solution of 3-(5-amino-1H-pyrazol-3-yl)benzonitrile (XXX; 3 g; 17 mmol) in pyridine (80 mL) was added 3-(diethylamino)acrylonitrile (II; 3.5 g; 29 mmol). The reaction mixture was heated at 100° C. for 18 hours. The reaction mixture was cooled and concentrated under reduced pressure. The crude mixture was purified by column chromatography using 4% MeOH-DCM to afford 3-(7-aminopyrazolo[1,5-a]pyrimidin-2-yl)benzonitrile as a light brown solid (XXXI; 2.1 g; 58% yield). MS (M+1): 236.0.

### Synthesis of Compound 76; 4-(tert-butyl)-N-(2-(3-cyanophenyl)pyrazolo[1,5-a]pyrimidin-7-yl)benzenesulfonamide

To a stirred solution of 3-(7-aminopyrazolo[1,5-a]pyrimidin-2-yl)benzonitrile (XXXI; 0.1 g; 0.43 mmol) in pyridine (3 mL) was added 4-tertbutylphenylsulfonyl chloride (XI; 0.22 g; 94 mmol) and catalytic DMAP at 0° C. The reaction mixture was heated to a reflux for 36 hours. The reaction mixture was concentrated at reduced pressure and purified through Combiflash® column chromatography using 10% MeOH-DCM as an eluent to afford 4-(tert-butyl)-N-(2-(3-cyanophenyl)pyrazolo[1,5-a]pyrimidin-7-yl)benzenesulfonamide as a white solid (76; 0.024 g, 12% yield). ¹H NMR (400 MHz, DMSO-d6): δ 13.64 (bs, 1H), 8.46 (s, 1H), 8.38-8.36 (d, J=8 Hz, 1H), 8.12-8.10 (d, J=7.2 Hz, 1H), 7.91-7.84 (m, 3H), 7.72-7.68 (t, J=7.6 Hz, 1H), 7.60-7.58 (d, J=8 Hz, 2H), 7.05 (s, 1H), 6.70-6.79 (d, J=6.8 Hz, 1H), 1.29 (s, 9H). MS (M+1): 432.26. (LCMS purity 97.29%, 6.09 min) (2).

The Following Compounds were Prepared in Essentially the Same Manner Using the Appropriate Sulfonyl Chloride in the Final Step:

| | | LCMS | Purity | |
|---|---|---|---|---|
| CPD | Structure | (M + 1) | (LCMS) | ¹H NMR |
| 77 | 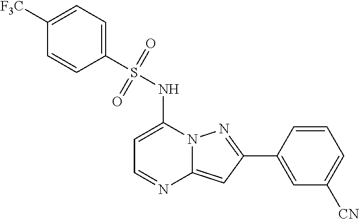 [SMILES: N#Cc1cccc(-c2cc3nccc(NS(=O)(=O)c4ccc(C(F)(F)F)cc4)n3n2)c1] | 444.17 | 95.76%, Rt = 5.81 min (2) | ¹H NMR (400 MHz, DMSO- d6): δ 8.47 (s, 1H), 8.39-8.37 (d, J = 8.4 Hz, 1H), 8.15-8.13 (m, 3H), 7.97-7.90 (m, 3H), 7.72-7.68 (m, 1H), 7.08 (s, 1H), 6.75-6.73 (d, J = 7.2 Hz, 1H). |
| 78 | 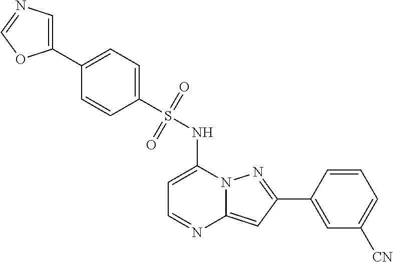 [SMILES: N#Cc1cccc(-c2cc3nccc(NS(=O)(=O)c4ccc(-c5cnco5)cc4)n3n2)c1] | 443.27 | 97.85%, Rt = 5.0 min (2) | ¹H NMR (400 MHz, DMSO- d6): δ 8.50-8.48 (s, 1H), 8.41 (s, 1H) 8.37-8.35 (d, J = 8.0 Hz, 1H), 7.95-7.91 (m, 3H), 7.88- 7.79 (m, 5H), 7.69-7.65 (t, J = 7.6 Hz, 1H), 6.90 (s, 1H), 6.42-6.40 (d, J = 5.2 Hz, 1H). |

### Example 7

### Synthesis of Compound 79 [4-(tert-butyl)-N-(2-isopropylpyrazolo[1,5-a]pyrimidin-7-yl)benzenesulfonamide] and Compounds 80-84

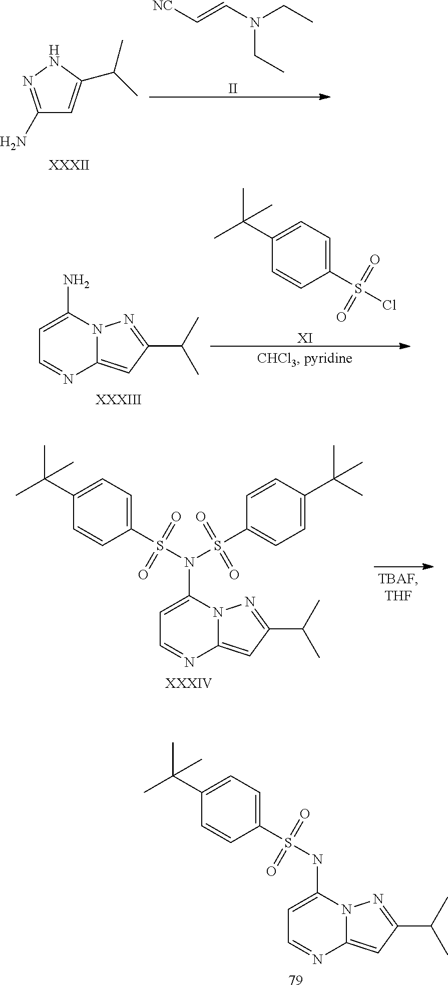

Synthesis of XXXIII:

To a stirred solution of 5-isopropyl-1H-pyrazol-3-amine (XXXII; 1.5 g; 12 mmol) and 3-(diethylamino)acrylonitrile (II; 2.3 g; 18 mmol) in toluene (50 mL) was added acetic acid (16.5 mL). The reaction mixture was heated at 140° C. in a microwave for 10 minutes. The reaction mixture was cooled and concentrated under reduced pressure. The crude mixture was purified by column chromatography using 5% MeOH-DCM to obtain 2-isopropylpyrazolo[1,5-a]pyrimidin-7-amine as a light brown solid (XXXIII; 1.2 g; 56% yield). ¹H NMR (400 MHz, DMSO-d6): δ 7.97-7.96 (d, J=5.2 Hz, 1H), 7.52 (bs, 2H), 6.18 (s, 1H), 5.98-5.97 (d, J=5.2 Hz, 1H), 3.09-3.02 (m, 1H), 1.30-1.28 (d, J=6.8 Hz, 6H). MS (M+1): 177.0.

### Synthesis of Compound 79; 4-(tert-butyl)-N-(2-isopropylpyrazolo[1,5-a]pyrimidin-7-yl)benzenesulfonamide

To a stirred solution of 2-isopropylpyrazolo[1,5-a]pyrimidin-7-amine (xxxiii; 0.25 g; 1.42 mmol) in chloroform (10 mL) was added pyridine (0.35 mL; 4.2 mmol) and 4-tertbutylbenzenesulfonyl chloride (xi; 0.65 g; 2.1 mmol) at 0° C. The reaction mixture was heated at 80° C. for 6 hours. The reaction mixture was cooled and concentrated at reduced pressure to afford the di-substituted sulfonamide product which was confirmed by LCMS (XXXIV). The crude product was further dissolved in THF in presence of TBAF and stirred at room temperature for 3 hours. The reaction mixture was concentrated and diluted with water. The aqueous layer was extracted with EtOAc (3×25 mL). The combined organic layers were washed with brine, dried over Na₂SO₄, filtered and concentrated to afford the crude compound, which was purified by column chromatography (5% MeOH-DCM)) to obtain the title compound (79; 180 mg; 38% yield). ¹HNMR (400 MHz, DMSO-d6): δ 13.30 (bs, 1H), 8.03-8.01 (d, J=7.6 Hz, 1H), 7.81-7.79 (d, J=8.4 Hz, 2H), 7.58-7.56 (d, J=8.4 Hz, 2H), 6.69-6.67 (d, J=7.2 Hz, 1H), 6.24 (s, 1H), 3.06-3.0 (m, 1H), 1.28 (s, 9H), 1.25-1.23 (d, J=6.8 Hz, 6H). MS (M+1): 373.24 LCMS purity 97.39%, 4.95 min (1).

The following compounds were prepared in essentially the same manner using the appropriate sulfonyl chloride in the final step.

| | | LCMS | Purity | |
|---|---|---|---|---|
| CPD | Structure | (M + 1) | (LCMS) | ¹H NMR |
| 80 | 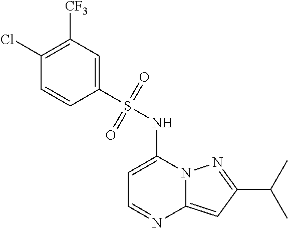 [SMILES: CC(C)c1cc2nccc(NS(=O)(=O)c3ccc(Cl)c(C(F)(F)F)c3)n2n1] | 419.12 | 95.91%, Rt = 5.98 min (1) | ¹HNMR (400 MHz, DMSO- d6): δ 13.59 (bs, 1H), 8.22- 8.18 (m, 2H), 8.11-8.09 (d, J = 6.8 Hz, 1H), 7.96-7.94 (d, J = 8.4 Hz, 1H), 6.68-6.66 (d, J = 7.2 Hz, 1H), 6.30 (s, 1H), 3.07-3.03 (m, 1H), 1.26-1.25 (d, J = 6.8 Hz, 6H). |
| 81 | 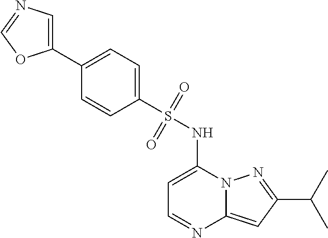 [SMILES: CC(C)c1cc2nccc(NS(=O)(=O)c3ccc(-c4cnco4)cc3)n2n1] | 382.01 (M − 1) | 98.47%, Rt = 4.03 min (1) | ¹HNMR (400 MHz, DMSO- d6): δ 13.43 (bs, 1H), 8.53 (s, 1H), 8.05-8.03 (d, J = 7.2 Hz, 1H), 7.99-7.97 (d, J = 8.4 Hz, 2H), 7.91-7.89 (d, J = 8.4 Hz, 2H), 7.86 (s, 1H), 6.67-6.66 (d, J = 7.2 Hz, 1H), 6.27 (s, 1H), 3.06-3.02 (m, 1H), 1.26- 1.24 (d, J = 6.8 Hz, 6H). |
| 82 | 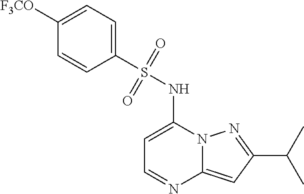 [SMILES: CC(C)c1cc2nccc(NS(=O)(=O)c3ccc(OC(F)(F)F)cc3)n2n1] | 401.15 | 99.61%, Rt = 4.83 min (1) | ¹HNMR (400 MHz, DMSO- d6): δ 13.42 (bs, 1H), 8.07- 8.01 (m, 3H), 7.56-7.54 (d, J = 8.0 Hz, 2H), 6.67-6.65 (d, J = 7.2 Hz, 1H), 6.28 (s, 1H), 3.06-3.02 (m, 1H), 1.26-1.24 (d, J = 6.8 Hz, 6H). |
| 83 | 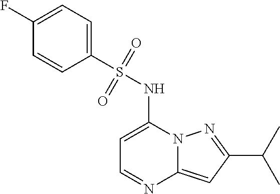 [SMILES: CC(C)c1cc2nccc(NS(=O)(=O)c3ccc(F)cc3)n2n1] | 335.11 | 99.12%, Rt = 4.24 min (1) | ¹HNMR (400 MHz, DMSO- d6): δ 13.35 (bs, 1H), 8.04- 7.94 (m, 3H), 7.39 (m, 2H), 6.64 (m, 1H), 6.25 (s, 1H), 3.07-3.03 (m, 1H), 1.25-1.24 (s, 6H). |
| 84 | 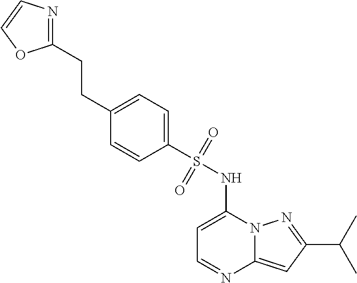 [SMILES: CC(C)c1cc2nccc(NS(=O)(=O)c3ccc(CCc4ncco4)cc3)n2n1] | 412.35 | 97.29% Rt = 4.87 min (2) | ¹H NMR (400 MHz, DMSO- d6): δ 13.28 (bs, 1H), 8.01- 7.92 (m, 2H), 7.79-7.77 (d, J = 8 Hz, 2H), 7.45-7.42 (d, J = 13.6 Hz, 2H), 7.08 (s, 1H), 6.64-6.63 (d, J = 7.2 Hz, 1H), 6.23 (s, 1H), 3.07 (s, 5H), 1.25-1.24 (d, J = 6.8 Hz, 6H). |

### Example 8

### Synthesis of Compound 85 [4-(tert-butyl)-N-(2-ethylpyrazolo[1,5-a]pyrimidin-7-yl)benzenesulfonamide] and Compounds 86-88

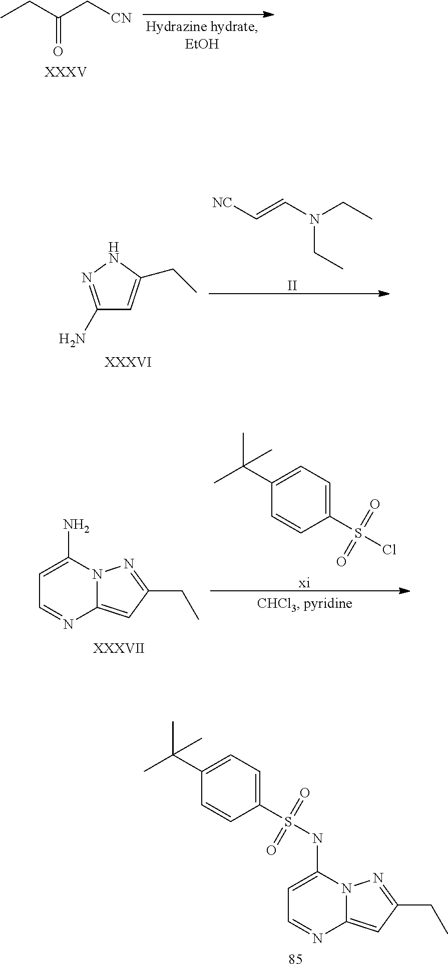

Synthesis of XXXVI:

To a stirred solution of 3-oxopentanenitrile (XXXV; 2 g; 21 mmol) in ethanol (60 mL) was added hydrazine hydrate (1.3 mL; 41 mmol). The reaction mixture was heated to a reflux for 12 hours. The reaction mixture was cooled and concentrated under reduced pressure. The crude mixture was purified by column chromatography using 8% MeOH-DCM to obtain 5-ethyl-1H-pyrazol-3-amine as a brown sticky solid (XXXVI; 1.8 g; 78% yield). ¹H NMR (400 MHz, DMSO-d6): δ 11.0 (bs, 1H), 5.17 (s, 1H), 4.5 (bs, 2H), 2.4 (m, 2H), 1.1 (m, 3H). MS (M+1): 111.93.

Synthesis of XXXVII:

To a stirred solution of 5-ethyl-1H-pyrazol-3-amine (XXXVI; 1.65 g; 15 mmol) and 3-(diethylamino)acrylonitrile (II; 2.76 g; 22 mmol) in toluene (22 mL) was added acetic acid (27 mL). The reaction mixture was heated at 140° C. in a microwave for 40 minutes. The reaction mixture was cooled and concentrated under reduced pressure. The crude mixture was purified by column chromatography using 6% MeOH-DCM to obtain 2-ethylpyrazolo[1,5-a]pyrimidin-7-amine as a sticky brown solid (XXXVII; 1.7 g; 70% yield). ¹H NMR (400 MHz, DMSU-d6): δ 7.97-7.96 (d, J=5.2 Hz, 1H), 7.57 (bs, 2H), 6.18 (s, 1H), 5.99-5.98 (d, J=5.2 Hz, 1H), 2.77-2.71 (q, J=7.6 Hz, 2H), 1.28-1.24 (t, J=7.6 Hz, 3H). MS (M+1): 162.96.

### Synthesis of Compound 85; 4-(tert-butyl)-N-(2-ethylpyrazolo[1,5-a]pyrimidin-7-yl)benzenesulfonamide

To a stirred solution of compound 2-ethylpyrazolo[1,5-a]pyrimidin-7-amine (XXXVII; 0.20 g; 1.23 mmol) in chloroform (10 mL) was added pyridine (0.3 mL; 3.7 mmol) and 4-tertbutylbenzenesulfonyl chloride (XI; 0.57 g; 2.4 mmol) at 0° C. The reaction mixture was heated at 80° C. for 10 hours, whereupon it was allowed to cool and was concentrated at reduced pressure. The reaction mixture was diluted with water and the aqueous layer was extracted with EtOAc (3×25 mL). The combined organic layers were washed with brine, dried over Na₂SO₄, filtered and concentrated to afford the crude compound, which was purified by column chromatography (5% MeOH-DCM) to obtain the title compound 4-(tert-butyl)-N-(2-ethylpyrazolo[1,5-a]pyrimidin-7-yl)benzenesulfonamide (85; 60 mg; 14% yield). ¹HNMR (400 MHz, DMSO-d6): δ 13.32 (bs, 1H), 8.03-8.01 (d, J=7.2 Hz, 1H), 7.81-7.79 (d, J=8.4 Hz, 2H), 7.58-7.56 (d, J=8.4 Hz, 2H), 6.70-6.68 (d, J=7.2 Hz, 1H), 6.24 (s, 1H), 2.72-2.66 (q, J=7.6 Hz, 2H), 1.28 (s, 9H), 1.23-1.19 (t, J=7.6 Hz, 3H). MS (M+1): 359.22. (LCMS purity 99.46%, 5.24 min) (2).

The Following Compounds were Prepared in Essentially the Same Manner Using the Appropriate Sulfonyl Chloride in the Final Step:

| | | LCMS | Purity | |
|---|---|---|---|---|
| CPD | Structure | (M + 1) | (LCMS) | ¹H NMR |
| 86 | 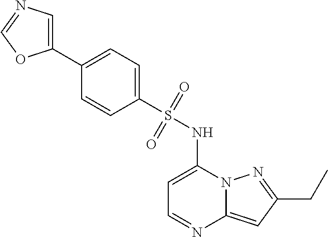 [SMILES: CCc1cc2nccc(NS(=O)(=O)c3ccc(-c4cnco4)cc3)n2n1] | 370.09 | 98.62%, Rt = 3.75 min (2) | ¹HNMR (400 MHz, DMSO-d6): δ 13.41 (bs, 1H), 8.53 (s, 1H), 8.03-8.01 (d, J = 6.8 Hz, 1H), 7.97-7.95 (d, J = 8.4 Hz, 2H), 7.90-7.88 (m, 2H), 7.85 (s, 1H), 6.63-6.61 (d, J = 6.8, 1H), 6.24 (s, 1H), 2.73-2.67 (q, J = 7.6 Hz, 2H), 1.24-1.20 (t, J = 7.6 Hz, 3H). |
| 87 | 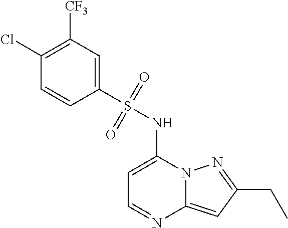 [SMILES: CCc1cc2nccc(NS(=O)(=O)c3ccc(Cl)c(C(F)(F)F)c3)n2n1] | 405.14 | 99.53%, Rt = 5.74 min (2) | ¹HNMR (400 MHz, DMSO-d6): δ 13.62 (bs, 1H), 8.19-8.17 (m, 2H), 8.12-8.10 (d, J = 7.2 Hz, 1H), 7.96-7.94 (d, J = 8 Hz, 1H), 6.69-6.67 (d, J = 7.2 Hz, 1H), 6.30 (s, 1H), 2.74-2.68 (q, J = 7.4 Hz, 2H), 1.24-1.21 (t, J = 7.4 Hz, 3H). |
| 88 | 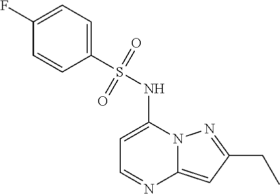 [SMILES: CCc1cc2nccc(NS(=O)(=O)c3ccc(F)cc3)n2n1] | 321.08 | 99.32%, Rt = 3.98 min (2) | ¹HNMR (400 MHz, DMSO-d6): δ 13.34 (bs, 1H), 8.05-8.04 (d, J = 7.2 Hz, 1H), 7.96-7.93 (m, 2H), 7.41-7.37 (m, 2H), 6.67- 6.65 (d, J = 7.2 Hz, 1H), 6.26 (s, 1H), 2.73-2.67 (q, J = 7.6 Hz, 2H), 1.24-1.21 (t, J = 7.6 Hz, 3H). |

### Example 9

### Synthesis of Compound 89 [4-(tert-butyl)-N-(2-(4-chlorophenyl)pyrazolo[1,5-a]pyrimidin-7-yl)benzenesulfonamide] and Compound 90

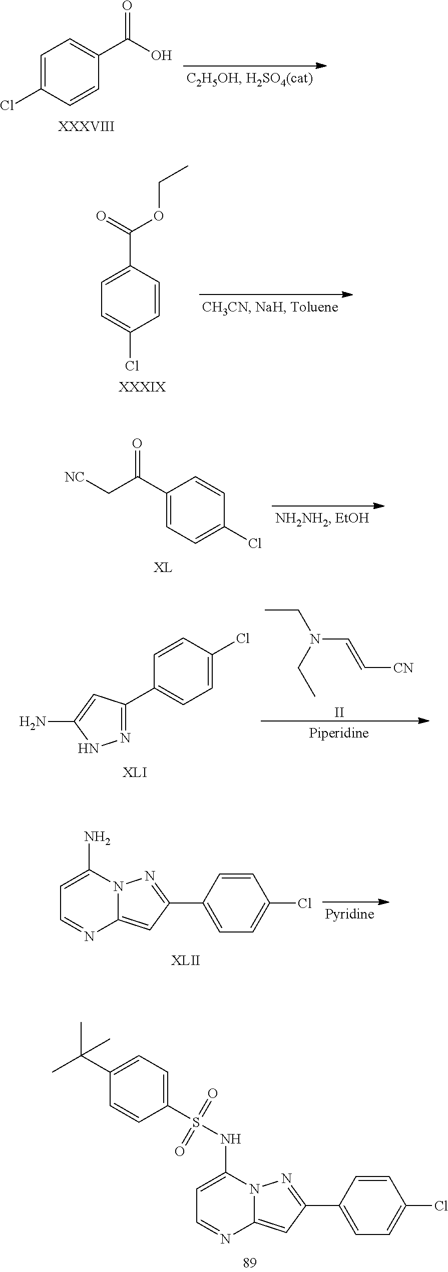

Synthesis of XXXIX:

To a stirred solution of 4-chlorobenzoic acid (XXXVIII; 15 g; 9.61 mmol) in ethanol (150 mL) was added a catalytic quantity of sulfuric acid (3 mL). The reaction mixture was heated to a reflux for 12 hours, whereupon it was cooled, concentrated under reduced pressure and diluted with water. The aqueous layer was extracted with ethyl acetate (3×60 mL). The combined organic layers were washed with successively with sodium bicarbonate and brine, followed by drying over Na₂SO₄, filtration and concentration under vacuum to afford ethyl 4-chlorobenzoate as a white solid (XXXIX; 12 g, 71% yield). ¹H NMR (400 MHz, CDCl₃): δ 7.96-7.94 (d, J=8.4 Hz, 2H), 7.60-7.58 (d, J=8.4 Hz, 2H), 4.33-4.28 (q, J=7.2 Hz, 2H), 1.33-1.29 (t, J=7.2 Hz, 3H).

Synthesis of XL:

To a stirred solution of acetonitrile (10 mL) in toluene (100 mL) was added sodium hydride (3.26 g; 81 mmol) at 0° C. The stirring was continued for 30 minutes and then ethyl 4-chlorobenzoate (XXXIX; 5 g; 27 mmol) was added. The reaction mixture was stirred at 100° C. for 12 hours. The reaction mixture was cooled, concentrated at reduced pressure and diluted with ice cold water. The reaction mixture was acidified using IN hydrochloric acid. The aqueous layer was extracted with ethyl acetate (3×25 mL). The combined organic layers were washed with brine, dried over Na₂SO₄, filtered and concentrated under vacuum to afford the title compound 3-(4-chlorophenyl)-3-oxopropanenitrile as a crude light yellow solid (XL; 3 g, 61% yield).

Synthesis of XLI:

To a stirred solution of 3-(4-chlorophenyl)-3-oxopropanenitrile (XL; 1.5 g; 8.3 mmol) in ethanol (75 mL) was added hydrazine hydrate (0.8 g; 16.7 mmol). The reaction mixture was heated to a reflux for 12 hours. The reaction mixture was cooled and concentrated under reduced pressure and then diluted with water. The aqueous layer was extracted using ethyl acetate (3×20 mL) and triturated with hexane to afford 3-(4-chlorophenyl)-1H-pyrazol-5-amine as a yellow solid (XLI; 1 g, 60% yield). ¹H NMR (400 MHz, CDCl₃): δ 11.81 (bs, 1H), 7.67-7.65 (d, J=8.4 Hz, 2H), 7.42-7.40 (d, J=8.4 Hz, 2H), 5.74 (s, 1H), 4.85 (bs, 2H). MS (M−1): 192.27.

Synthesis of XLII:

To a stirred solution of 3-(4-chlorophenyl)-1H-pyrazol-5-amine (XLI; 0.6 g; 3.1 mmol) in piperidine (0.53 g; 4.6 mmol) was added 3-(diethylamino)acrylonitrile (II; 0.58 g; 4.6 mmol). The reaction mixture was heated at 100° C. for 18 hours. On cooling, solvent was removed under reduced pressure. The crude mixture was triturated with hexane to afford 2-(4-chlorophenyl)pyrazolo[1,5-a]pyrimidin-7-amine as an off-white solid (XLII; 0.5 g; 65% yield). ¹H NMR (400 MHz, DMSO-d6): δ 8.08-8.05 (m, 3H), 7.75 (bs, 2H), 7.56-7.51 (d, J=8.4 Hz, 2H), 6.89 (s, 1H), 6.1-6.09 (m, 1H).

### Synthesis of Compound 89; 4-(tert-butyl)-N-(2-(4-chlorophenyl)pyrazolo[1,5-a]pyrimidin-7-yl)benzenesulfonamide

To a stirred solution of 2-(4-chlorophenyl)pyrazolo[1,5-a]pyrimidin-7-amine (XLII 0.1 g; 0.41 mmol) in pyridine (5 mL) was added 4-tertbutylphenylsulfonylchloride (XI 0.12 g; 0.49 mmol) and catalytic DMAP at 0° C. The reaction mixture was heated to a retlux tor 24 hours. On cooling, the reaction mixture was concentrated and purified using Combiflash® column chromatography and 3% MeOH-DCM as an eluent to afford the title compound 4-(tert-butyl)-N-(2-(4-chlorophenyl)pyrazolo[1,5-a]pyrimidin-7-yl)benzenesulfonamide as a white solid (89; 0.024 g, 12% yield). ¹H NMR (400 MHz, DMSO-d6): δ 13.53 (bs, 1H), 8.09-8.03 (m, 3H), 7.85-7.83 (d, J=8 Hz, 2H), 7.59-7.52 (m, 4H), 6.91 (s, 1H), 6.78-6.77 (m, 1H), 1.29 (s, 9H). MS (M+1): 441.10. (LCMS purity 96.19%, 5.23 min) (1).

The Following Compound was Prepared in Essentially the Same Manner Using the Appropriate Sulfonyl Chloride in the Final Step:

| | | LCMS | Purity | |
|---|---|---|---|---|
| CPD | Structure | (M + 1) | (LCMS) | ¹H NMR |
| 90 | 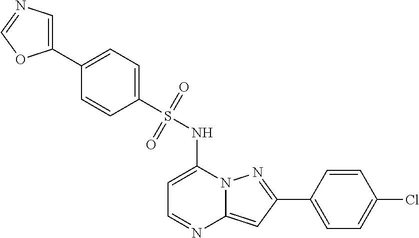 [SMILES: O=S(=O)(Nc1ccnc2cc(-c3ccc(Cl)cc3)nn12)c1ccc(-c2cnco2)cc1] | 452.05 | 95.05%, Rt = 5.51 min (2) | ¹H NMR (400 MHz, DMSO- d6): δ 13.62 (bs, 1H), 8.53 (s, 1H), 8.10-8.0 (m, 5H), 7.92- 7.90 (d, J = 8.4 Hz, 2H), 7.86 (s, 1H), 7.55-7.53 (d, J = 8.4 Hz, 2H), 6.92 (s, 1H), 6.73-6.71 (d, J = 7.2 Hz, 1H). |

### Example 10

### Synthesis of Compound 91 [N-(2-(1H-imidazol-4-yl)pyrazolo[1,5-a]pyrimidin-7-yl)-4-(tert-butyl)benzenesulfonamide]

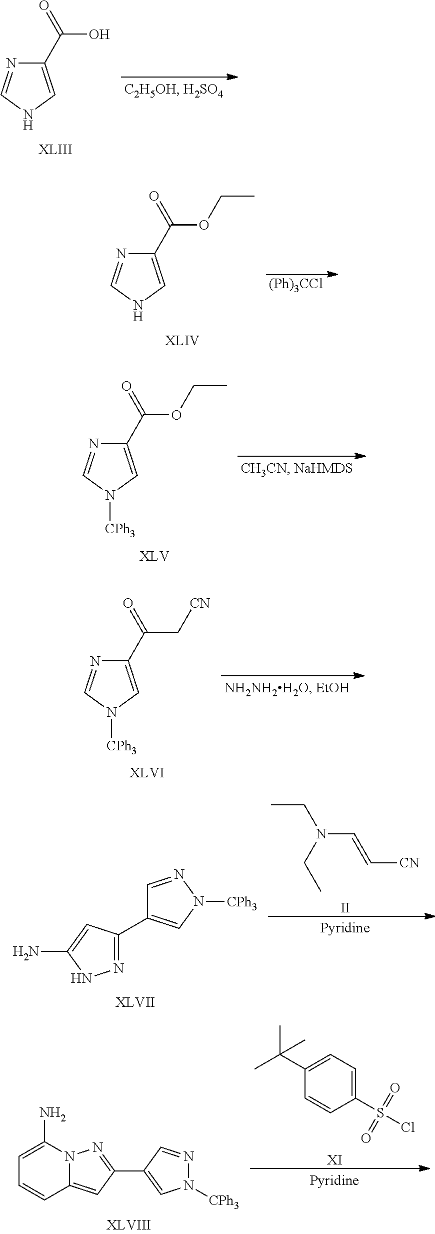

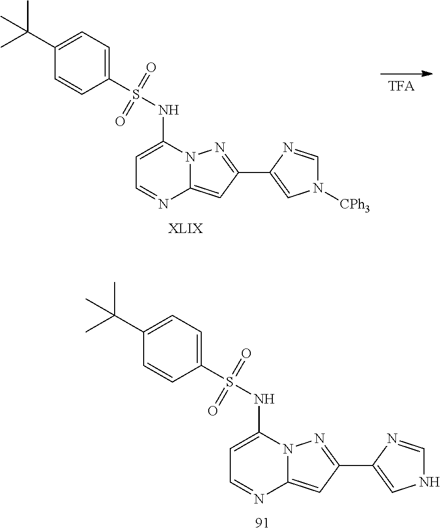

Synthesis of XLIV:

To a stirred solution of 1H-imidazole-4-carboxylic acid (XLIII; 5 g; 44.64 mmol) in ethanol (100 ml) was added sulfuric acid (3 ml). The reaction mixture was heated at 80° C. for 12 h. The reaction mixture was cooled, concentrated at reduced pressure and diluted with water. The aqueous layer was extracted with ethyl acetate. The combined organic layers were washed with sodium bicarbonate and brine solution, dried over Na₂SO₄, filtered and concentrated under vacuum to afford ethyl imidazole-4-carboxylate as a white solid (XLIV, 4.75 g, 76% yield). ¹H NMR (400 MHz, DMSO-d6) δ 12.75 (bs, 1H), 7.77 (s, 2H), 4.24-4.19 (q, J=7.2 Hz, 2H), 1.28-1.24 (t, J=6.8 Hz, 3H). MS (M+1) 141.12.

Synthesis of XLV:

To a stirred solution of XLIV, (2 g; 14 mmol) in dimethylformide (50 ml) was added trityl chloride (3.98 g; 14 mmol) and triethylamine (1.73 g, 17 mmol) at 0° C. The resulting solution was stirred for 12 h at room temperature. The reaction mixture was cooled, concentrated at reduced pressure and diluted with water. The aqueous layer was extracted with ethyl acetate. The combined organic layers were washed with brine, dried over Na₂SO₄, filtered and concentrated under vacuum to afford ethyl 1-trityl-1H-imidazole-4-carboxylate as a brown solid (XLV; 3 g, 55% yield). MS (M+1) 383.34.

Synthesis of XLVI:

To a stirred solution of acetonitrile (0.32 g; 7.80 mmol) in tetrahydrofuran (20 ml) was added sodium bis(trimethylsilyl)amide (15.7 ml, 1.0M in THF, 15.69 mmol at 0° C. The stirring was continued for 30 minutes and then a solution of ethyl 1-trityl-1H-imidazole-4-carboxylate (XLV; 2 g; 5.23 mmol) in THF (20 ml) was added. The reaction mixture was stirred at 80° C. for 12 h. The reaction mixture was cooled, concentrated at reduced pressure and diluted with ice cold water. The aqueous layer was extracted with ethyl acetate, and the resulting organic layer washed with brine, dried over Na₂SO₄, filtered and concentrated under vacuum to afford 3-oxo-3-(1-trityl-1H-imidazol-4-yl)propanenitrile as a brown solid (XLVI; 1 g, 50% yield). MS (M+1) 378.34.

Synthesis of XLVII:

To a stirred solution of compound XLVI (1 g; 2.65 mmol) in ethanol (10 ml) was added hydrazine hydrate (10 ml). The reaction mixture was heated at 90° C. for 12 h and then cooled and concentrated to afford 3-(1-trityl-1H-imidazol-4-yl)-1H-pyrazol-5-amine as a crude yellowish solid (XLVII; 0.6 g, 57% yield). MS (M+1) 392.12. The crude material was carried forward to the next step without purification.

Synthesis of XLVIII:

To a stirred solution of compound XLVII (1 g; 2.55 mmol) in pyridine (30 ml) was added 3-(diethylamino)acrylonitrile II (0.47 g; 3.82 mmol). The reaction mixture was heated at 100° C. for 18 h. The reaction mixture was cooled and concentrated under reduced pressure. The crude mixture was purified by column chromatography using 2% methanol in dichloromethane to obtain 2-(1-trityl-1H-imidazol-4-yl)pyrazolo[1,5-a]pyrimidin-7-amine as a yellowish solid (XLVIII; 0.6 g; 54% yield). MS (M+1): 443.12.

Synthesis of XLIX:

To a stirred solution of compound XLVIII (0.5 g; 1.12 mmol) in pyridine (10 ml) was added 4-tert-butylphenylsulfonyl chloride (XI; 0.47 g; 2.03 mmol) and catalytic DMAP. The reaction mixture was heated at 100° C. for 12 h and then concentrated under reduced pressure and purified by column chromatography using 25% ethyl acetate in hexane to afford XLIX (0.3 g, 41% yield) as a yellowish solid. MS (M−1): 637.20.

### Synthesis of Compound 91: N-(2-(1H-imidazol-4-yl)pyrazolo[1,5-a]pyrimidin-7-yl)-4-(tert-butyl)benzenesulfonamide

To a stirred solution of XLIX (0.3 g; 0.47 mmol) in water (4 ml) at 0° C. was added trifluoroacetic acid (6 ml). The reaction mixture was stirred at room temperature for 12 h whereupon it was concentrated under reduced pressure and purified by column chromatography using 5% methanol in dichloromethane to afford the title compound (91; 0.022 g, 12% yield) as a white solid. ¹H NMR (400 MHz, DMSO-d6): δ 13.06 (bs, 1H), 8.03 (s, 1H), 7.95-7.93 (d, J=6.4 Hz, 1H), 7.79-7.74 (m, 3H), 7.53-7.51 (d, J=8.4 Hz, 2H), 6.56 (s, 1H), 6.54-6.53 (d, J=6.8 Hz, 1H), 1.27 (s, 9H). MS (M+1): 397.21. (LCMS purity 99.38%, Rt=4.78 min) (2).

### Example 11

### Synthesis of Compound 92 [4-(tert-butyl)-N-(2-(1-methyl-1H-pyrazol-4-yl)pyrazolo[1,5-a]pyrimidin-7-yl)benzenesulfonamide]

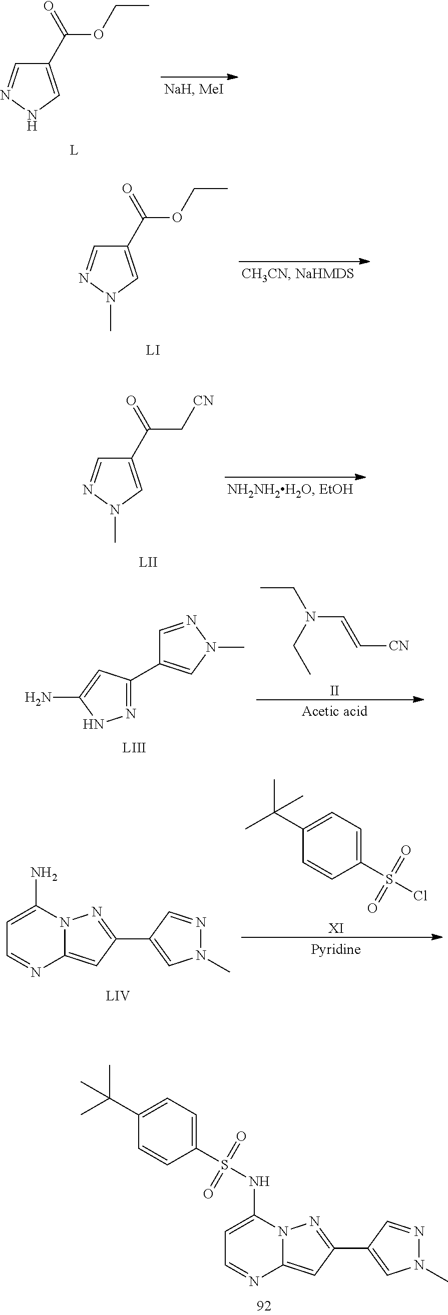

Synthesis of LI:

To a stirred solution of ethyl 1H-pyrazole-4-carboxylate (L, 4 g; 28.5 mmol) in tetrahydrofuran (50 ml) was added sodium hydride (1.36 g, 28.5 mmol) at 0° C. The reaction mixture was stirred for 1 h. Methyl iodide (6.07 g, 42.8 mmol) was added and the reaction was stirred for 18 h at room temperature. The reaction mixture was concentrated under reduced pressure and diluted with water. The resulting aqueous layer was extracted with ethyl acetate. The combined organic layers were washed with sodium bicarbonate, brine, dried over Na₂SO₄, filtered and concentrated under vacuum to afford ethyl 1-methyl-1H-pyrazole-4-carboxylate as a yellow liquid (LI; 3.5 g, 77% yield). ¹H NMR (400 MHz, CDCl₃): δ 7.88 (s, 1H), 7.85 (s, 1H), 4.31-4.25 (q, J=7.2 Hz, 2H), 3.9 (s, 3H), 1.35-1.31 (t, J=7.2 Hz, 3H). MS (M+1) 155.12.

Synthesis of LII:

To a stirred solution of acetonitrile (1.3 g; 34 mmol) and ethyl 1-methyl-1H-pyrazole-4-carboxylate (LI; 3.5 g, 22.72 mmol) in THF (20 ml) was added sodium bis(trimethylsilyl)amide (68.18 ml, 1.0 M in THF, 68.18 mmol) at −78° C. The stirring was continued for 2 h at the same temperature whereupon the reaction mixture was allowed to warm to room temperature, concentrated at reduced pressure and diluted with water. The aqueous layer was extracted with ethyl acetate, which was subsequently washed with brine, dried over Na₂SO₄, filtered and concentrated under vacuum to afford 3-(1-methyl-1H-pyrazol-4-yl)-3-oxopropanenitrile as a yellowish solid (LII; 2.5 g, 69% yield). ¹H NMR (400 MHz, CDCl₃): δ 8.01 (s, 1H), 7.94 (s, 1H), 3.97 (s, 3H), 3.79 (s, 2H). MS (M+1) 150.12.

Synthesis of LIII:

To a stirred solution of 3-(1-methyl-1H-pyrazol-4-yl)-3-oxopropanenitrile (LII; 2.5 g; 16.7 mmol) in ethanol (100 ml) was added hydrazine hydrate (1.67 g, 33.5 mmol). The reaction mixture was heated at 90° C. for 24 h, cooled, concentrated at reduced pressure and triturated with hexane to afford LIII as an off white solid (1.6 g, 59% yield). ¹H NMR (400 MHz, DMSO d6): δ 11.49 (bs, 1H), 7.86 (s, 1H), 7.62 (s, 1H), 5.48 (s, 1H), 4.6 (bs, 2H), 3.82 (s, 3H). MS (M+1): 164.1.

Synthesis of LIV:

To a stirred solution of LIII (0.25 g; 1.53 mmol) in acetic acid (6 ml) was added 3-(diethylamino)acrylonitrile (II; 0.28 g; 2.3 mmol). The reaction mixture was heated at 80° C. for 20 minutes in a microwave reactor. The reaction mixture was cooled and concentrated under reduced pressure. The crude mixture was triturated with dichloromethane to afford LIV (0.1 g; 76% yield). ¹H NMR (400 MHz, DMSO d6): δ 8.46 (bs, 2H), 8.22 (s, 1H), 8.11-8.09 (d, J=5.6 Hz, 1H), 7.93 (s, 1H), 6.62 (s, 1H), 6.15-6.14 (d, J=5.6 Hz, 1H), 3.91 (s, 3H). MS (M+1): 215.0.

### Synthesis of Compound 92; 4-(tert-butyl)-N-(2-(1-methyl-1H-pyrazol-4-yl)pyrazolo[1,5-a]pyrimidin-7-yl)benzenesulfonamide

To a stirred solution of LIV (0.2 g; 0.93 mmol) in chloroform (30 ml) at 0° C. was added pyridine (0.3 ml) and 4-tert-butylbenzenesulfonyl chloride (XI; 0.26 g; 1.11 mmol). The reaction mixture was heated to 100° C. for 42 h, then cooled and concentrated at reduced pressure. The crude mixture was purified by column chromatography using 2% methanol in dichloromethane to afford the title compound as an off white solid (92; 0.025 g, 7% yield). ¹H NMR (400 MHz, DMSO-d6): δ 13.36 (bs, 1H), 8.32 (s, 1H), 8.03-8.01 (d, J=7.6 Hz, 1H), 7.94 (s, 1H), 7.83-7.81 (d, J=8.4 Hz, 2H), 7.58-7.56 (d, J=8.0 Hz, 2H), 6.71-6.70 (d, J=7.8 Hz, 1H), 6.58 (s, 1H), 3.87 (s, 3H), 1.28 (s, 9H). MS (M+1): 411.19. (LCMS purity 98.22%, Rt=5.32 min) (2).

### Example 12

### Synthesis of Compound 93 [4-(tert-butyl)-N-(2-(5-chloropyridin-3-yl)pyrazolo[1,5-a]pyrimidin-7-yl)benzenesulfonamide]

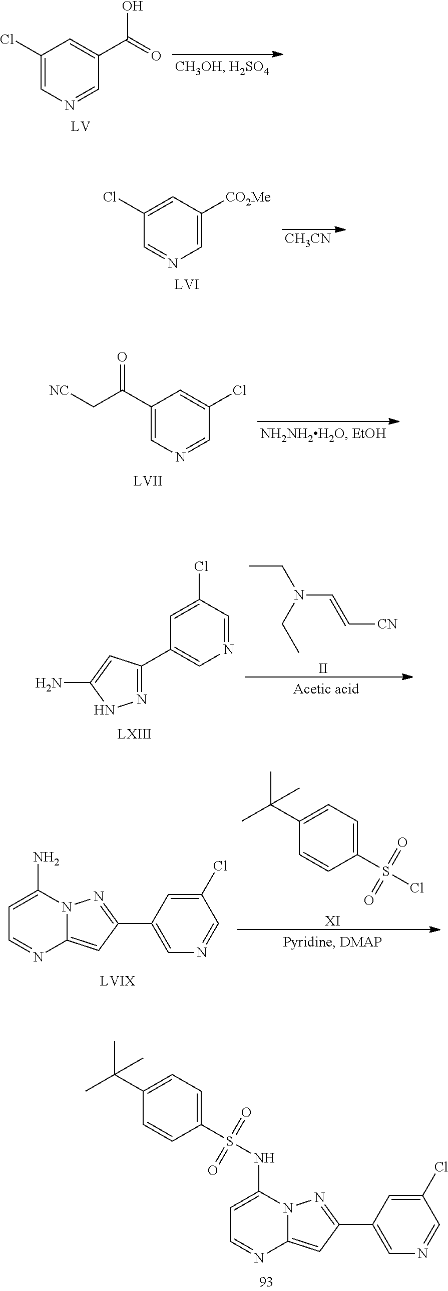

Synthesis of LVI:

To a stirred solution of 5-chloronicotinic acid (LV, 5 g; 31.8 mmol) in methanol (40 ml) was added sulfuric acid (4 ml). The reaction mixture was heated at 75° C. for 12 h. The reaction mixture was cooled, concentrated under reduced pressure and diluted with water. The aqueous layer was extracted with ethyl acetate. The combined organic layers were washed with sodium bicarbonate, brine, dried over Na₂SO₄, filtered and concentrated under vacuum to afford methyl 5-chloronicotinate as a white solid (LVI; 4.4 g, 80% yield). ¹H NMR (400 MHz, CDCl₃): δ 9.08 (d, J=1.6 Hz, 1H), 8.73 (d, J=2.4 Hz, 1H), 8.28-8.27 (t, J=2.0 Hz, 1H), 3.96 (s, 3H). MS (M+1): 172.12

Synthesis of LVII:

To a stirred solution of acetonitrile (0.95 g; 23 mmol) in tetrahydrofuran (30 ml) was added potassium tert butoxide (0.19 g; 23 mmol) at 0° C. The stirring was continued for 30 minutes and then 5-chloronicotinate (LVI; 2.58 g; 19.23 mmol) was added. The reaction mixture was stirred at room temperature for 3 h. The reaction mixture was concentrated and diluted with ice cold water. The aqueous layer was extracted with ethyl acetate. The combined organic layers were washed with brine, dried over Na₂SO₄, filtered and concentrated under vacuum to afford 3-(5-chloropyridin-3-yl)-3-oxopropanenitrile as a red sticky solid (LVII; 2.7 g). MS (M+1): 181.12.

Synthesis of LVIII:

To a stirred solution of 3-(5-chloropyridin-3-yl)-3-oxopropanenitrile (LVII; 2.7 g; 14.9 mmol) in ethanol (15 ml) was added hydrazine hydrate (0.82 g, 16.39 mmol). The reaction mixture was heated at 100° C. for 12 h. The reaction mixture was cooled, concentrated at reduced pressure and triturated with hexane to afford the title compound 3-(5-chloropyridin-3-yl)-1H-pyrazol-5-amine as an off white solid (LVIII; 1.4 g) MS (M+1): 195.1.

Synthesis of LIX:

To a stirred solution of 3-(5-chloropyridin-3-yl)-1H-pyrazol-5-amine (LVIII; 1.45 g; 7.43 mmol) in acetic acid (60 ml) was added 3-(diethylamino)acrylonitrile (II; 1.10 g; 8.9 mmol). The reaction mixture was heated at 100° C. for 18 h. The reaction mixture was cooled and concentrated under reduced pressure. The crude mixture was purified by column chromatography using 4% methanol in dichloromethane to afford 2-(5-chloropyridin-3-yl)pyrazolo[1,5-a]pyrimidin-7-amine as a light brown solid (LIX; 0.9 g; 49% yield). MS (M+1): 246.0.

### Synthesis of Compound 93; 4-(tert-butyl)-N-(2-(5-chloropyridin-3-yl)pyrazolo[1,5-a]pyrimidin-7-yl)benzenesulfonamide

To a stirred solution of 2-(5-chloropyridin-3-yl)pyrazolo[1,5-a]pyrimidin-7-amine (LVIX; 0.2 g; 0.81 mmol) in pyridine (3 ml), 4-(tert-butyl)benzenesulfonyl chloride (XI; 0.22 g; 0.97 mmol) and a catalytic quaniy of DMAP were added. The reaction mixture was heated to 90° C. for 12 h. The reaction mixture was cooled and concentrated at reduced pressure. The crude mixture was purified by column chromatography using 3% methanol in dichloromethane to afford the title product, 4-(tert-butyl)-N-(2-(5-chloropyridin-3-yl)pyrazolo[1,5-a]pyrimidin-7-yl)benzenesulfonamide as an off white solid (93; 0.05 g, 16% yield). ¹H NMR (400 MHz, DMSO-d6): δ13.6 (bs, 1H), 9.17 (d, J=2.0 Hz, 1H), 8.70-8.69 (d, J=2.4 Hz, 1H), 8.49-8.48 (d, J=2.0 Hz, 1H), 8.13-8.11 (d, J=7.2 Hz, 1H), 7.86-7.84 (d, J=8.4 Hz, 2H), 7.60-7.58 (d, J=8.4 Hz, 2H), 7.10 (s, 1H), 6.82-6.80 (d, J=7.8 Hz, 1H), 1.29 (s, 9H). MS (M+1): 442.30. (LCMS purity 95.12%, Rt=6.43 min) (2).

### Example 13

### Synthesis of Compound 94 [4-(tert-butyl)-N-(4-chloro-2-methylpyrazolo[1,5-a]pyridin-7-yl)benzenesulfonamide]; Compound 95 [4-(tert-butyl)-N-(4-cyano-2-methylpyrazolo[1,5-a]pyridin-7-yl)benzene sulfonamide]; and Compounds 96-109

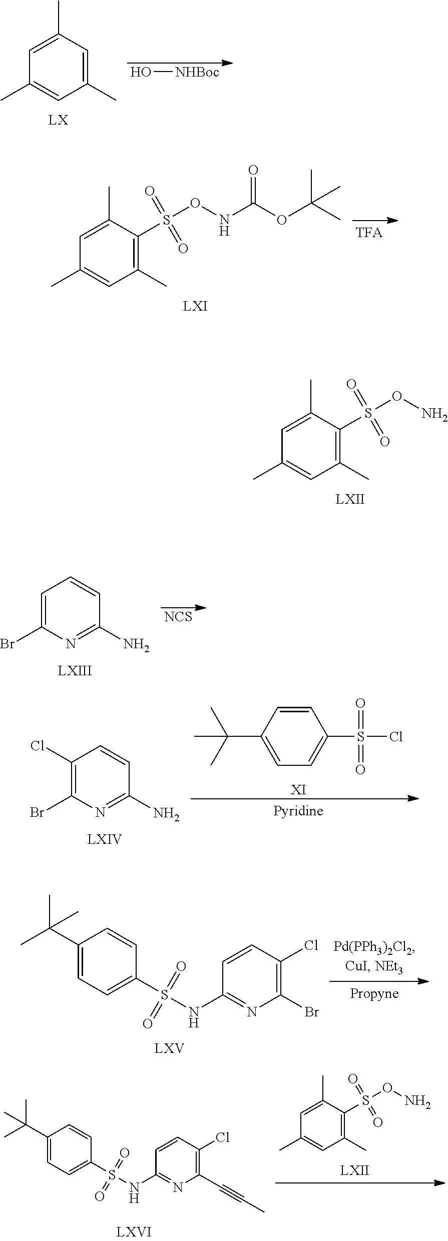

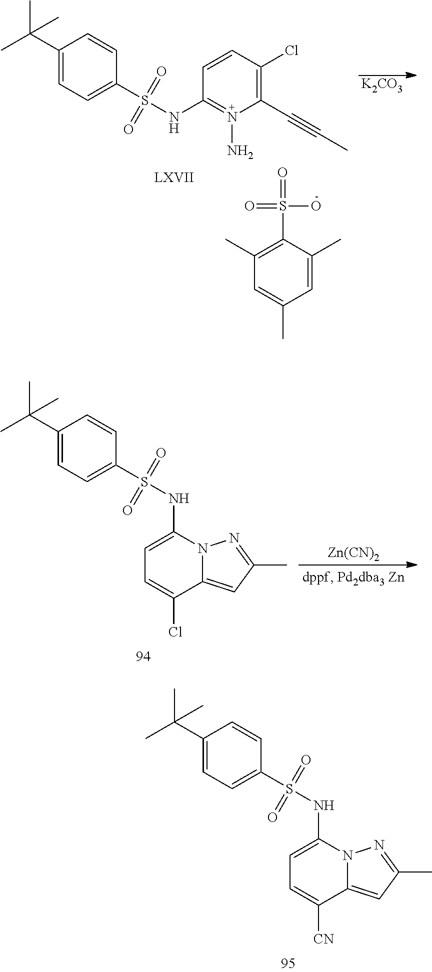

Synthesis of LXI:

To a stirred solution of 2-Mesitylenesulfonyl chloride (LX; 20 g, 91.45 mmol) in methyl tert-butyl ether (200 ml) was added tert-butyl N-hydroxycabamate (12.17 g, 91.45 mmol). The reaction mixture was purged with nitrogen and cooled to 0° C. Triethylamine (8.43 g, 93.27 mmol) was added dropwise with stirring at 0° C. The resultant mixture was stirred for a further 2 h. The reaction mixture was filtered to remove triethylamine hydrochloride and washed with methyl tert-butyl ether. The liquid phase was concentrated at 20° C. to a minimum volume and triturated with n-hexane. The solid so obtained was filtered and dried to afford, tert-butyl ((mesitylsulfonyl)oxy)carbamate as a white solid. (LXI; 23 g, 79% yield). ¹H NMR (400 MHz, DMSO-d6): δ 11.15 (bs, 1H), 7.13 (s, 2H), 2.56 (s, 6H), 2.29 (s, 3H), 1.24 (s, 9H). MS (M+1): 316.15.

Synthesis of LXII:

To a stirred solution of trifluroacetic acid (30 ml) at 0° C. was added LXI (10 g, 31.70 mmol) in portionwise fashion. The reaction mixture was stirred at 0° C. for 2 h, whereupon it was diluted with crushed ice with cold water. A white solid precipitated, which was isolated by filtration, washed with ice cold water until the washings reached a neutral pH. The solid, compound LXII was dried and stored in plastic bottles at −20° C. ¹H NMR (400 MHz, DMSO-d6): δ 6.75 (s, 2H), 2.49 (s, 6H), 2.16 (s, 3H). MS (M+1): 216.15.

Synthesis of LXIV:

To a stirred solution of compound LXIII (20 g; 115.54 mmol) in acetonitrile (360 ml) was added N-chlorosuccinimide (17 g, 127.0 mmol) portionwise at 0° C. The resultant solution was stirred at 90° C. for 18 h. The reaction mixture was diluted with water and extracted with ethyl acetate. The organic layer was washed with brine solution and dried over anhydrous Na₂SO₄, filtered and evaporated under reduced pressure to obtain the crude compound, which was purified by column chromatography using 18% ethyl acetate in hexane to afford 6-bromo-5-chloropyridin-2-amine as off white solid (LXIV; 18 g; 75% yield). ¹H NMR (400 MHz, CDCl₃) δ 7.42-7.40 (d, J=8.8 Hz, 1H), 6.38-6.36 (d, J=8.8 Hz, 1H), 4.63 (bs, 2H). MS (M+1): 206.92 (LCMS Purity 96%).

Synthesis of LXV:

To a stirred solution of compound LXIV (5 g, 24.10 mmol) in chloroform (25 ml) was added pyridine (100 ml) at 0° C. followed by addition of 4-tert-butylbenzenesulfonyl chloride (XI, 6.71 g, 28.41 mmol). The reaction mixture was heated at 90° C. for 12 h, cooled to room temperature and concentrated under reduced pressure. The crude mass was diluted with saturated ammonium chloride solution and extracted with ethyl acetate. The organic layer was washed with brine solution and dried over anhydrous Na₂SO₄, filtered and evaporated under reduced pressure to afford N-(6-bromo-5-chloropyridin-2-yl)-4-(tert-butyl)benzenesulfonamide (LXV; 7.7 g, 79% yield). ¹H NMR (400 MHz, CDCl₃) δ 7.82-7.80 (d, J=8.4 Hz, 2H), 7.64-7.62 (d, J=8.4 Hz, 1H), 7.51-7.49 (d, J=8.4 Hz, 2H), 7.34-7.32 (d, J=8.4 Hz, 1H), 1.37 (s, 9H). MS (M+1): 404.89 (LCMS Purity 95%).

Synthesis of LXVI:

To a stirred solution of LXV (3 g, 7.43 mmol) in dimethylformide (120 ml) in sealable tube was purged with argon for 20 min. Then Bis(triphenylphosphine)palladium(II) chloride (0.15 g, 0.22 mmol), copper(I)iodide (0.035 g, 0.18 mmol), triethylamine (2.25 g, 22.29 mmol) were added. The reaction mixture was cooled and the vessel charged with excess propyne gas for 10 min. The reaction vessel was sealed and heated at 100° C. for 24 h. The reaction mixture was cooled and filtered through a celite bed which was washed with ethyl acetate. All the filtrate was collected and concentrated, diluted with water and extracted with ethyl acetate. The organic layer was washed with brine solution and dried over anhydrous Na₂SO₄, filtered and evaporated under reduced pressure to obtain crude compound, which was purified by column chromatography using 12% ethyl acetate in hexane to afford 4-(tert-butyl)-N-(5-chloro-6-(prop-1-yn-1-yl)pyridin-2-yl)benzenesulfonamide (LXVI; 1.2 g, 44% yield). ¹H NMR (400 MHz, DMSO-d6): δ 11.36 (s, 1H), 8.85-7.83 (m, 3H), 7.61-7.59 (d, J=8 Hz, 2H), 7.10-7.08 (d, J=8 Hz, 1H), 2.12 (s, 3H), 1.27 (s, 9H). MS (M+1): 363.16. (LCMS Purity 96%).

Synthesis of LXVII:

To a stirred solution of LXVI (1.2 g, 3.30 mmol) in dichloromethane (30 ml) was added O-(mesitylsulfonyl) hydroxylamine (LXII; 2.84 g, 13.2 mmol). The reaction mixture was stirred for 12 h at room temperature and then diluted with water and extracted with dichloromethane.

The organic layer was washed with a saturated aqueous solution of sodium bicarbonate and brine solution before being dried over anhydrous Na₂SO₄, filtered and evaporated under reduced pressure to obtain crude compound LXVII, MS (M+1): 378.16. The crude material was carried forward to the next step without purification.

### Synthesis of Compound 94: 4-(tert-butyl)-N-(4-chloro-2-methylpyrazolo[1,5-a]pyridin-7-yl)benzene sulfonamide

To a stirred solution of LXVII (1.5 g, crude) in dimethylformide (20 ml) was added potassium carbonate (1.6 g, 11.85 mmol). The reaction mixture was stirred at 60° C. for 1 h and then concentrated in vacuo. The residue was diluted with water and extracted with ethyl acetate. The organic layer was washed with brine solution and dried over anhydrous Na₂SO₄, filtered and evaporated under reduced pressure to obtain the crude compound, which was purified by column chromatography using 12% ethyl acetate in hexane to afford, the title compound (94; 0.28 g, 20% yield). ¹H NMR (400 MHz, DMSO-d6): δ 11.09 (bs, 1H), 7.85-7.84 (d, J=7.2 Hz, 2H), 7.58-7.56 (d, J=7.6 Hz, 2H), 7.31-7.29 (d, J=7.6 Hz, 1H), 6.63-6.61 (d, J=7.2 Hz, 1H), 6.49 (s, 1H), 2.37 (s, 3H), 1.25 (s, 9H). MS (M+1): 378.15. (LCMS Purity 97.56%, Rt=3.69 min) (2).

### Synthesis of Compound 95: 4-(tert-butyl)-N-(4-cyano-2-methylpyrazolo[1,5-a]pyridin-7-yl)benzene sulfonamide

To a stirred solution of 94 (0.25 g, 0.66 mmol) in dimethylacetamide (10 ml) was added Zn(CN)₂ (0.38 g, 3.3 mmol). The reaction mixture was purged with argon for 20 min, whereupon 1, 1′-Bis (diphenylphosphino)ferrocene (0.01 g, 0.019 mmol), Pd2dba3 (0.009 g, 0.01 mmol) and a catalytic amount of Zn dust were added. The reaction mixture was heated at 120° C. for 2 h in microwave reactor. The reaction mixture was cooled and concentrated, diluted with water and extracted with dichloromethane. The organic layer was washed with brine solution and dried over anhydrous Na₂SO₄, filtered and evaporated under reduced pressure to obtain the crude compound, which was purified by column chromatography using 2% methanol in 2% ammoniated dichloromethane to afford the title compound (95; 0.03 g, 12% yield). ¹H NMR (400 MHz, DMSO-d6): δ 7.92-7.90 (d, J=8.4 Hz, 2H), 7.75-7.73 (d, J=7.6 Hz, 1H), 7.60-7.57 (d, J=8.8 Hz, 2H), 6.70-6.68 (d, J=8.0 Hz, 1H), 6.55 (s, 1H), 2.44 (s, 3H), 1.26 (s, 9H).MS (M+1): 369.22. (LCMS Purity 98.40%, Rt=6.91 min) (2).

The following compounds were prepared in a similar manner using the appropriate sulfonyl chloride and alkyne. The final conversion to the nitrile was not undertaken.

| | | LCMS | Purity | |
|---|---|---|---|---|
| CPD | Structure | (M + 1) | (LCMS) | ¹H NMR |
| 96 | 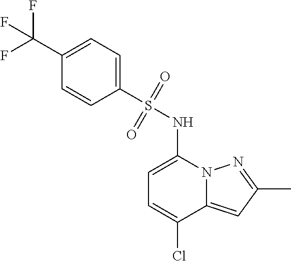 [SMILES: Cc1cc2c(Cl)ccc(NS(=O)(=O)c3ccc(C(F)(F)F)cc3)n2n1] | 390.04 | 99.09%, Rt = 4.61 min (2) | ¹H NMR (400 MHz, DMSO- d6): δ 8.07-8.05 (d, J = 8.0 Hz, 2H), 7.94-7.92 (d, J = 8.4 Hz, 2H), 7.31-7.30 (d, J = 7.6 Hz, 1H), 6.65-6.63 (d, J = 8.0 Hz, 1H), 6.49 (s, 1H), 2.30 (s, 3H). |
| 97 | 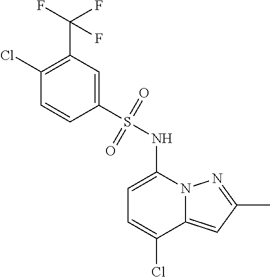 [SMILES: Cc1cc2c(Cl)ccc(NS(=O)(=O)c3ccc(Cl)c(C(F)(F)F)c3)n2n1] | 424.01 | 96.79%, Rt = 4.80 min (2) | ¹H NMR (400 MHz, DMSO- d6): δ 11.90 (bs, 1H), 8.25 (s, 1H), 8.05-8.03 (m, 1H), 7.88- 7.86 (d, J = 8.4 Hz, 1H), 7.35-7.33 (d, J = 8.0 Hz, 1H), 6.73-6.71 (d, J = 8.0 Hz, 1H), 6.50 (s, 1H), 2.28 (s, 3H). |
| 98 | 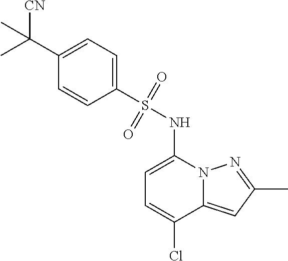 [SMILES: Cc1cc2c(Cl)ccc(NS(=O)(=O)c3ccc(C(C)(C)C#N)cc3)n2n1] | 389.37 | 98.91%, Rt = 5.08 min (1) | ¹H NMR (400 MHz, DMSO- d6): δ 7.97-7.95 (d, J = 8.4 Hz, 2H), 7.72-7.70 (d, J = 8.4 Hz, 2H), 7.32-7.30 (d, J = 8.0 Hz, 1H), 6.65-6.63 (d, J = 8.0 Hz, 1H), 6.50 (s, 1H), 2.36 (s, 3H), 1.67 (s, 6H). |
| 99 | 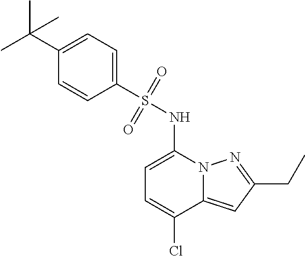 [SMILES: CCc1cc2c(Cl)ccc(NS(=O)(=O)c3ccc(C(C)(C)C)cc3)n2n1] | 392.12. | 95.97% Rt = 5.47 min (2) | ¹H NMR (400 MHz, DMSO- d6): δ 7.85-7.83 (d, J = 8.8 Hz, 2H), 7.57-7.55 (d, J = 8.4 Hz, 2H), 7.32-7.30 (d, J = 8 Hz, 1H), 6.65-6.63 (d, J = 8 Hz, 1H), 6.52 (s, 1H), 2.76- 2.70 (q, J = 7.6 Hz, 2H), 1.25 (s, 9H), 1.2 (t, J = 7.6 Hz, 3H). |
| 100 | 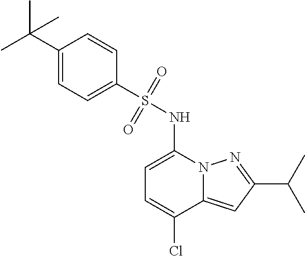 [SMILES: CC(C)c1cc2c(Cl)ccc(NS(=O)(=O)c3ccc(C(C)(C)C)cc3)n2n1] | 406.19 | 95.40% Rt = 5.93 min (2) | ¹H NMR (400 MHz, DMSO- d6): δ 7.84-7.82 (d, J = 8.8 Hz, 2H), 7.56-7.54 (d, J = 8.4 Hz, 2H), 7.32-7.30 (d, J = 8 Hz, 1H), 6.66-6.64 (d, J = 8 Hz, 1H), 6.52 (s, 1H), 3.08- 3.01 (m, 1H), 1.24 (s, 9H), 1.23 (m, 6H). |
| 101 | 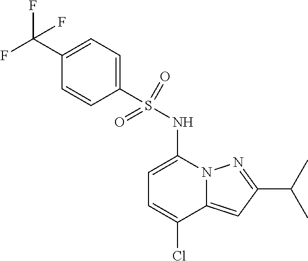 [SMILES: CC(C)c1cc2c(Cl)ccc(NS(=O)(=O)c3ccc(C(F)(F)F)cc3)n2n1] | 418.11 | 99.28%, Rt = 5.03 min (2) | ¹H NMR (400 MHz, DMSO- d6): δ 11.5 (bs, 1H), 8.02- 8.00 (d, J = 8 Hz, 2H), 7.91- 7.89 (d, J = 8.4 Hz, 2H), 7.34-7.32 (d, J = 8 Hz, 1H), 6.71-6.69 (d, J = 8 Hz, 1H), 6.51 (s, 1H), 2.97-2.88 (m, 1H), 1.16-1.14 (m, 6H). |
| 102 | 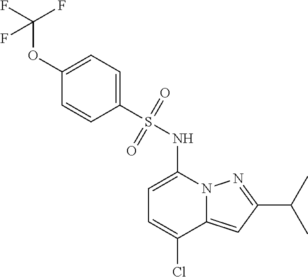 [SMILES: CC(C)c1cc2c(Cl)ccc(NS(=O)(=O)c3ccc(OC(F)(F)F)cc3)n2n1] | 434.09 | 98.06%, Rt = 5.14 min (2) | ¹H NMR (400 MHz, DMSO- d6): δ 12.33 (bs, 1H) 7.98- 7.96 (d, J = 8.4 Hz, 2H), 7.52-7.50 (d, J = 8.4 Hz, 2H), 7.33-7.31 (d, J = 8 Hz, 1H), 6.69-6.67 (d, J = 8 Hz, 1H), 6.52 (s, 1H), 3.02-2.97 (m, 1H), 1.21-1.19 (d, J = 6.8 Hz, 6H). |
| 103 | 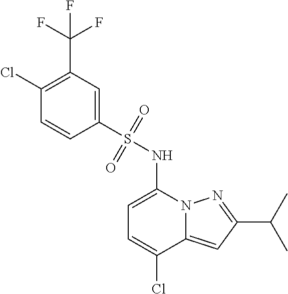 [SMILES: CC(C)c1cc2c(Cl)ccc(NS(=O)(=O)c3ccc(Cl)c(C(F)(F)F)c3)n2n1] | 452.04 | 96.24%, Rt = 5.33 min (2) | ¹H NMR (400 MHz, DMSO- d6): δ 11.39 (bs, 1H), 8.16 (s, 1H), 8.00-7.98 (d, J = 8.0 Hz, 1H), 7.87-7.85 (d, J = 8.4 Hz, 1H), 7.35-7.34 (d, J = 7.6 Hz, 1H), 6.76-6.74 (d, J = 8.4 Hz, 1H), 6.52 (s, 1H), 2.95 (m, 1H), 1.16-1.15 (d, J = 6.8 Hz, 6H). |
| 104 | 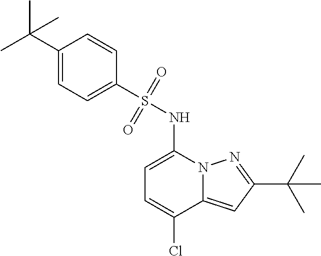 [SMILES: CC(C)(C)c1ccc(S(=O)(=O)Nc2ccc(Cl)c3cc(C(C)(C)C)nn23)cc1] | 420.23 | 98.77%, Rt = 4.21 min (1) | ¹H NMR (400 MHz, DMSO- d6): δ 7.83-7.81 (d, J = 8.0 Hz, 2H), 7.54-7.52 (d, J = 8.4 Hz, 2H), 7.31-7.29 (m, 1H), 6.68-6.66 (m, 1H), 6.52 (s, 1H), 1.28 (s, 9H), 1.24 (s, 9H). |
| 105 | 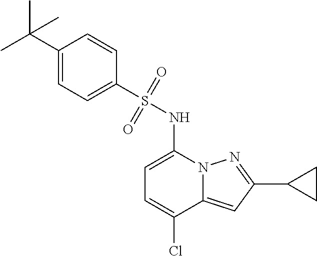 [SMILES: CC(C)(C)c1ccc(S(=O)(=O)Nc2ccc(Cl)c3cc(C4CC4)nn23)cc1] | 404.17 | 97.59% Rt = 5.40 min (2) | ¹H NMR (400 MHz, DMSO- d6): δ 7.84-7.82 (d, J = 8.8 Hz 2H), 7.58-7.55 (d, J = 8.8 Hz, 2H), 7.29-7.27 (d, J = 8.4 Hz, 1H), 6.63-6.61 (d, J = 8 Hz, 1H), 6.40 (s, 1H), 2.06- 2.00 (m, 1H), 1.25 (s, 9H), 0.99-0.95 (m, 2H), 0.82-0.78 (m, 2H). |
| 106 | 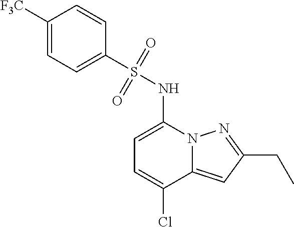 [SMILES: CCc1cc2c(Cl)ccc(NS(=O)(=O)c3ccc(C(F)(F)F)cc3)n2n1] | 404.10 | 96.86% Rt = 4.86 min (2) | ¹H NMR (400 MHz, DMSO- d6): δ 8.03 (m, 2H), 7.92 (m, 2H), 7.30 (m, 1H), 6.64 (m, 1H), 6.50 (s, 1H), 2.65-2.64 (q, J = 7.6 Hz, 2H), 1.2 (t, J = 7.6 Hz, 3H). |
| 107 | 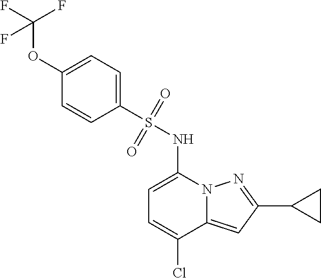 [SMILES: O=S(=O)(Nc1ccc(Cl)c2cc(C3CC3)nn12)c1ccc(OC(F)(F)F)cc1] | 432.04 | 96.36%, Rt = 4.88 min (2) | ¹H NMR (400 MHz, DMSO- d6): δ 7.99-7.98 (d, J = 6 Hz, 2H), 7.54 (m, 2H), 7.30-7.28 (d, J = 5.6 Hz, 1H), 6.64 (m, 1H), 6.39 (s, 1H), 1.99-1.97 (d, J = 8 Hz, 1H), 0.95 (s, 2H), 0.75 (s, 2H). |
| 108 | 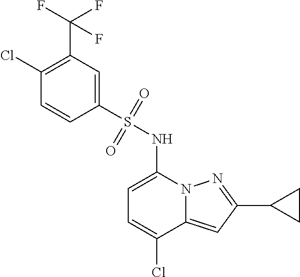 [SMILES: O=S(=O)(Nc1ccc(Cl)c2cc(C3CC3)nn12)c1ccc(Cl)c(C(F)(F)F)c1] | 450.02 | 95.15%, Rt = 5.06 min (2) | ¹H NMR (400 MHz, DMSO- d6): δ 8.19 (s, 1H), 8.03-8.01 (d, J = 8.4 Hz, 1H), 7.89-7.87 (d, J = 8.4 Hz, 1H), 7.32-7.30 (d, J = 8.0 Hz, 1H), 6.72-6.70 (d, J = 8.0 Hz, 1H), 6.38 (s, 1H), 1.94-1.90 (m, 1H), 0.96- 0.92 (m, 2 H), 0.68-0.66 (t, J = 5.2 Hz, 2H). |
| 109 | 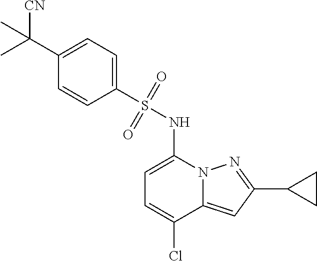 [SMILES: CC(C)(C#N)c1ccc(S(=O)(=O)Nc2ccc(Cl)c3cc(C4CC4)nn23)cc1] | 415.12 | 95.94%, Rt = 4.58 min (2) | ¹H NMR (400 MHz, DMSO- d6): δ 11.52 (bs, 1H), 7.91 (s, 1H), 7.85-7.83 (d, J = 7.6 Hz, 1H), 7.79-7.77 (d, J = 7.6 Hz, 1H), 7.62-7.58 (t, J = 8.0 Hz, 1H), 7.30-7.28 (d, J = 8.0 Hz, 1H), 6.68-6.66 (d, J = 7.6 Hz, 1H), 6.37 (s, 1H), 2.01-1.99 (m, 1H), 1.67 (s, 6H), 0.96- 0.95 (m, 2H), 0.75-0.74 (m, 2H). |

### Example 14

### Synthesis of Compound 110 [4-(tert-butyl)-N-(4-chloro-2-(pyridin-3-yl)pyrazolo[1,5-a]pyridin-7-yl)benzenesulfonamide]; Compound 111 [3-(7-((4-(tert-butyl)phenyl)sulfonamido)-4-chloropyrazolo[1,5-a]pyridin-2-yl)pyridine-1-oxide]; and Compounds 112 to 146

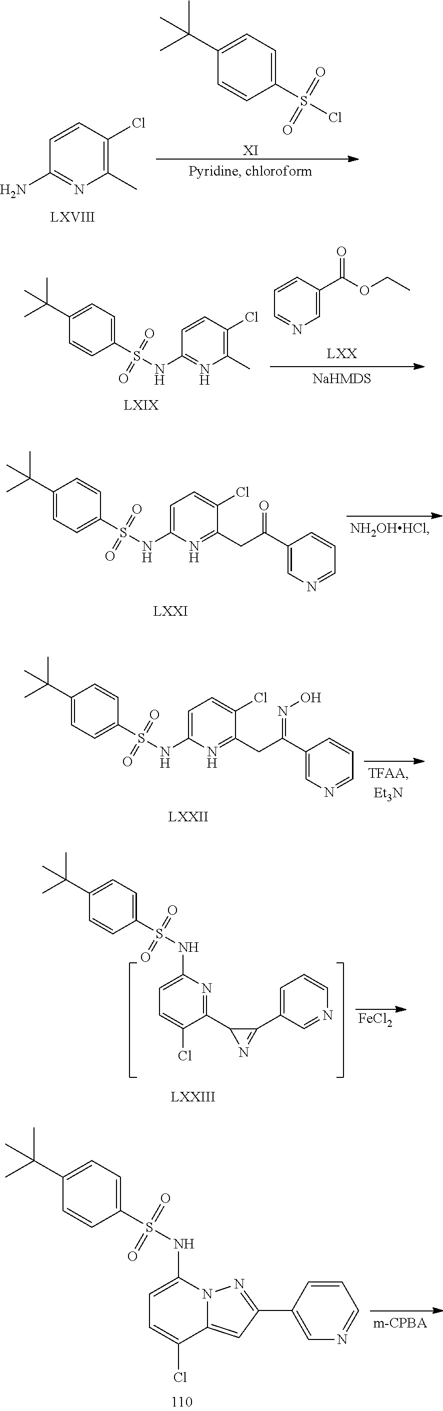

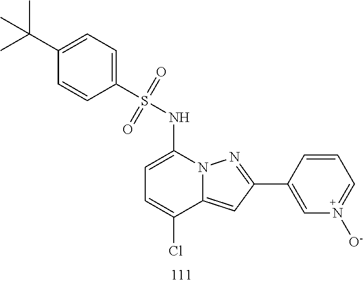

Synthesis of LXIX:

To a stirred solution of compound LXVIII (3 g, 21.12 mmol) in chloroform (60 ml) was added pyridine (15 ml) at 0° C. followed by addition of 4-tert-butylbenzenesulfonyl chloride (XI, 5.89 g, 25.34 mmol). The reaction mixture was heated at 100° C. for 12 h, cooled to room temperature and concentrated under reduced pressure. The crude material was diluted with a saturated ammonium chloride solution and extracted with ethyl acetate. The organic layer was washed with brine solution, dried over anhydrous Na₂SO₄ and evaporated under reduced pressure to afford 4-(tert-butyl)-N-(5-chloro-6-methylpyridin-2-yl)benzenesulfonamide (LXIX; 6 g, 84% yield). ¹H NMR (400 MHz, DMSO-d6) δ 11.11 (bs, 1H), 7.86-7.84 (d, J=8.4 Hz, 2H), 7.72-7.70 (d, J=8.8 Hz, 1H), 7.60-7.58 (d, J=8.4 Hz, 2H), 6.94-6.93 (d, J=7.6 Hz, 1H), 2.36 (s, 3H), 1.27 (s, 9H). MS (M+1): 339.2.

Synthesis of LXXI:

To a stirred solution of compound LXIX (3 g; 8.87 mmol) and ethyl nicotinate (LXX; 1.47 g; 9.75 mmol) in THF (30 ml) was added sodium bis(trimethylsilyl)amide (26.6 ml, 1.0M in THF, 26.61 mmol) dropwise at 0° C. Upon complete addition, the resultant solution was stirred at ambient temperature for 5 h. The reaction mixture was diluted with a saturated solution of ammonium chloride and extracted with ethyl acetate. The organic layer was washed with brine solution and dried over anhydrous Na₂SO₄, filtered and evaporated under reduced pressure to obtain LXXI, as a keto-enol tautomeric mixture. MS (M+1): 444.2. The crude material was carried forward to next step without purification.

Synthesis of LXII:

To a stirred solution of compound LXXI (3 g; 6.75 mmol) in methanol was added hydroxylamine hydrochloride (42.3 g; 33.85 mmol) followed by a 10% aqueous solution of sodium hydroxide (22 ml). The resultant suspension was heated at 100° C. for 5 h and then cooled to room temperature. The reaction mixture was concentrated in vacuo and the residue was diluted with water and extracted with ethyl acetate. The organic layer was washed with brine solution and dried over anhydrous Na₂SO₄, filtered and evaporated under reduced pressure to obtain the crude compound, which was purified by column chromatography using 50% ethyl acetate in hexane to afford desired product (LXXII; 2.8 g; 90% yield). ¹H NMR (400 MHz, DMSO-d6) δ 11.64 (s, 1H), 11.03 (bs, 1H), 8.68 (s, 1H), 8.47 (m, 1H), 7.81-7.75 (m, 3H), 7.69-7.67 (d, J=8.4 Hz, 1H), 7.50-7.47 (d, J=8.4 Hz, 2H), 7.29-7.26 (m, 1H), 6.80-6.78 (d, J=8.8 Hz, 1H), 4.23 (s, 2H), 1.25 (s, 9H). MS (M+1): 459.1.

### Synthesis of Compound 110; 4-(tert-butyl)-N-(4-chloro-2-(pyridin-3-yl)pyrazolo[1,5-a]pyridin-7-yl)benzenesulfonamide

To a stirred solution of LXXII (0.15 g, 0.32 mmol) in 1,2-dimethoxyethane (7 ml) at 0° C. was added trifluroacetic anhydride (0.13 g, 0.64 mmol). The reaction mixture was allowed to stir at 0° C. for 20 minutes, followed by dropwise addition of triethylamine (0.162 g, 1.6 mmol) in 1,2-dimethoxyethane (2 ml). The reaction mixture was stirred at room temperature for 5 h to generate the azirine compound LXIII in situ. To the reaction mixture was further added iron (II) chloride (0.008 g, 0.06 mmol) and the resultant was heated at 90° C. for 2 h. The reaction mixture was cooled and concentrated, diluted with water and extracted with ethyl acetate. The organic layer was washed with brine solution and dried over anhydrous Na₂SO₄, filtered and evaporated under reduced pressure to obtain the crude compound, which was purified by column chromatography using 70% ethyl acetate in hexane to afford the title compound as an off-white solid (110; 0.08 g; 57% yield). ¹H NMR (400 MHz, DMSO-d6): δ 9.19 (s, 1H), 8.60-8.59 (d, J=3.6 Hz, 1H), 8.36-8.35 (d, J=6.8 Hz, 1H), 7.82-7.80 (d, J=8.4 Hz, 2H), 7.52-7.50 (m, 3H), 7.43-7.41 (d, J=8.0 Hz, 1H), 7.31 (s, 1H), 6.85-6.83 (d, J=8.4 Hz, 1H), 1.14 (s, 9H). MS (M+1): 441.10. (LCMS Purity 99.03%, Rt=6.09 min) (2).

### Synthesis of Compound 111; 3-(7-((4-(tert-butyl)phenyl)sulfonamido)-4-chloropyrazolo[1,5-a]pyridin-2-yl)pyridine 1-oxide

To a stirred solution of 110 (0.1 g, 0.22 mmol) in dichloromethane (5 ml) was added meta-chloroperbenzoic acid (0.078 g, 0.44 mmol). The reaction mixture was stirred at room temperature for 12 h and diluted with water. The aqueous layer was extracted with dichloromethane and the combined organic layers were washed with a saturated solution of sodium bicarbonate, brine and dried over anhydrous Na₂SO₄, filtered and evaporated under reduced pressure to obtain the crude compound, which was purified by column chromatography using 7% methanol in dichloromethane to afford the title compound as a pink solid. (111; 0.015 g, 15% yield). ¹H NMR (400 MHz, DMSO-d6): δ 11.30 (bs, 1H), 8.93 (s, 1H), 8.25-8.23 (d, J=6 Hz, 1H), 7.95-7.93 (d, J=8 Hz, 1H), 7.82-7.79 (d, J=8.8 Hz, 2H), 7.51 (m, 3H), 7.28 (m, 2H), 6.72 (m, 1H), 1.19 (s, 9H). MS (M+1): 457.11. (LCMS Purity 95.68%, Rt=6.41 min) (2).

The following compounds were prepared in a similar manner using the appropriate sulfonyl chloride in the first step and the appropriate ester instead of ethyl nicotinate LXX in step 2.

Only pyridine N-oxide final compounds were subject to the final step involving use of mCPBA.

| | | LCMS | Purity | |
|---|---|---|---|---|
| CPD | Structure | (M + 1) | (LCMS) | ¹H NMR |
| 112 | 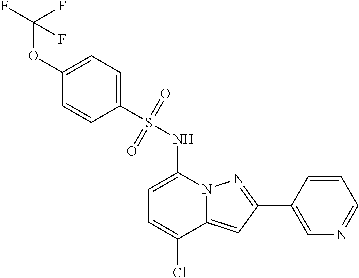 [SMILES: O=S(=O)(Nc1ccc(Cl)c2cc(-c3cccnc3)nn12)c1ccc(OC(F)(F)F)cc1] | 469.02 | 97.52%, Rt = 5.79 min (2) | ¹H NMR (400 MHz, DMSO- d6): δ 9.18-9.18 (s, 1H), 8.62-8.60 (d, J = 1.6 Hz, 1H), 8.34-8.32 (d, J = 8.0 Hz, 1H), 8.02-8.00 (d, J = 8.8 Hz, 2H), 7.54-7.48 (m, 3H), 7.43-7.41 (d, J = 8.0 Hz, 1H), 7.27 (s, 1H), 6.84- 6.82 (d, J = 8.0 Hz, 1H) |
| 113 | 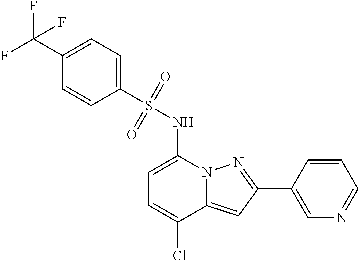 [SMILES: O=S(=O)(Nc1ccc(Cl)c2cc(-c3cccnc3)nn12)c1ccc(C(F)(F)F)cc1] | 453.03 | 98.63%, Rt = 5.73 min (2) | ¹H NMR (400 MHz, DMSO- d6): δ 9.13 (s, 1H) 8.61 (bs, 1H), 8.25 (m, 1H), 8.13 (m, 2H), 7.89 (m, 2H), 7.52 (m, 2H), 7.37-7.33 (s, 1H), 6.83 (m, 1H) |
| 114 | 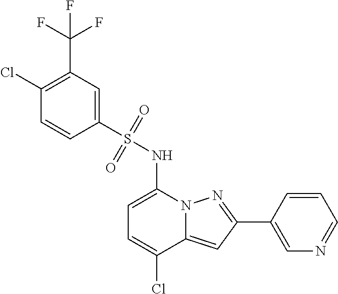 [SMILES: O=S(=O)(Nc1ccc(Cl)c2cc(-c3cccnc3)nn12)c1ccc(Cl)c(C(F)(F)F)c1] | 487.09 | 96.32%, Rt = 5.89 min (2) | ¹H NMR (400 MHz, DMSO- d6): δ 9.15 (s, 1H) 8.63-8.62 (d, J = 4.8 Hz, 1H) 8.28-8.26 (d, J = 8 Hz, 1H), 8.19 (s, 1H), 8.09-8.07 (d, J = 8.0 Hz, 1H), 7.85-7.83 (d, J = 8.0 Hz, 1H), 7.54-7.51 (m, 1H), 7.42-7.39 (m, 1H), 7.31 (s, 1H), 6.81-6.79 (d, J = 7.6 Hz, 1H) |
| 115 | 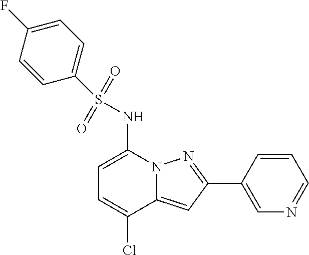 [SMILES: O=S(=O)(Nc1ccc(Cl)c2cc(-c3cccnc3)nn12)c1ccc(F)cc1] | 403.01 | 95.78%, Rt = 5.05 min (1) | ¹H NMR (400 MHz, DMSO- d6): δ 9.22 (s, 1H) 8.63-8.62 (d, J = 3.6 Hz, 1H) 8.39-8.37 (d, J = 8.4 Hz 1H), 8.04-7.97 (m, 2H), 7.56-7.53 (m, 1H), 7.48-7.35 (m, 4H), 6.82-6.80 (d, J = 8.0 Hz, 1H). |
| 116 | 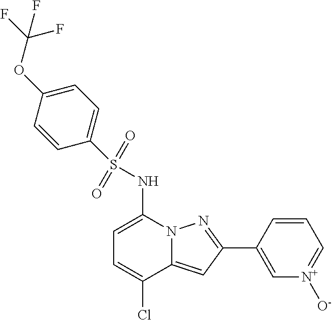 [SMILES: O=S(=O)(Nc1ccc(Cl)c2cc(-c3ccc[n+]([O-])c3)nn12)c1ccc(OC(F)(F)F)cc1] | 485.14 | 98.93%, Rt = 6.06 min (2) | ¹H NMR (400 MHz, DMSO- d6): δ 8.84 (s, 1H) 8.22-8.21 (d, J = 6.0 Hz, 1H) 7.95-7.93 (m, 3H), 7.51-7.48 (t, J = 7.2 Hz, 1H), 7.43-7.41 (d, J = 8.4 Hz, 2H), 7.15 (m, 2H), 6.46 (m, 1H). |
| 117 | 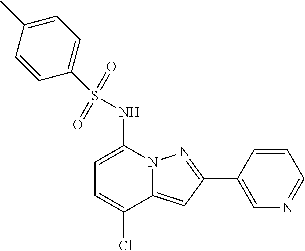 [SMILES: Cc1ccc(S(=O)(=O)Nc2ccc(Cl)c3cc(-c4cccnc4)nn23)cc1] | 399.17 | 95.01%, Rt = 5.06 min (1) | ¹H NMR (400 MHz, DMSO- d6): δ 11.35 (bs, 1H) 9.22 (s, 1H), 8.60 (m, 1H), 8.38-8.36 (d, J = 6.4 Hz, 1H), 7.81- 7.76 (m, 2H), 7.51 (m, 1H), 7.35-7.26 (m, 4H), 6.69 (m, 1H), 2.28 (s, 3H). |
| 118 | 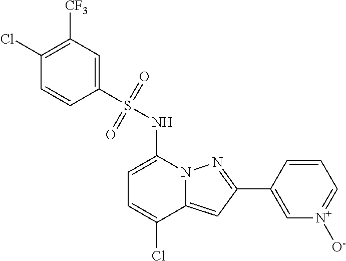 [SMILES: O=S(=O)(Nc1ccc(Cl)c2cc(-c3ccc[n+]([O-])c3)nn12)c1ccc(Cl)c(C(F)(F)F)c1] | 502.87 | 96.08%, Rt = 6.19 min (2) | ¹H NMR (400 MHz, DMSO- d6): δ 8.82 (s, 1H), 8.22-8.21 (d, J = 5.6 Hz, 1H), 8.17 (s, 1H), 8.07-8.04 (d, J = 8.4 Hz, 1H), 7.96-7.94 (d, J = 8.0 Hz, 1H), 7.80-7.78 (d J = 8.4 Hz, 1H), 7.52-7.48 (t, J = 7.2 Hz, 1H), 7.12 (s, 2H), 6.34 (bs, 1H). |
| 119 | 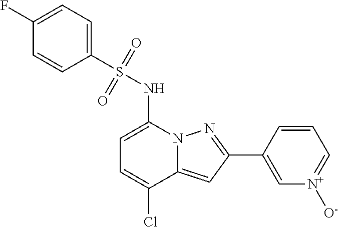 [SMILES: O=S(=O)(Nc1ccc(Cl)c2cc(-c3ccc[n+]([O-])c3)nn12)c1ccc(F)cc1] | 419.22 | 96.07%, Rt = 4.65 min (1) | ¹H NMR (400 MHz, DMSO- d6): δ 8.86 (s, 1H), 8.23-8.21 (d, J = 4.8 Hz, 1H), 7.97- 7.89 (m, 3H), 7.55-7.49 (m, 1H), 7.29-7.27 (d, J = 7.6 Hz, 2H), 7.08 (m, 2H), 6.49- 6.37 (m, 1H). |
| 120 | 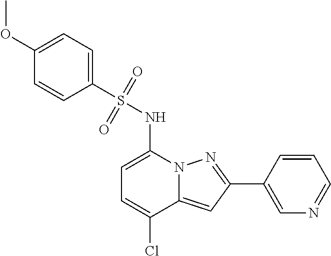 [SMILES: COc1ccc(S(=O)(=O)Nc2ccc(Cl)c3cc(-c4cccnc4)nn23)cc1] | 413.17 (M − 1) | 98.46%, Rt = 4.95 min (1) | ¹H NMR (400 MHz, DMSO- d6): δ 11.36 (bs, 1H), 9.25 (s, 1H), 8.61-8.60 (d, J = 3.2 Hz, 1H), 8.41-8.39 (d, J = 6.4 Hz, 1H), 7.87-7.85 (d, J = 8.0 Hz, 2H), 7.52 (m, 1H), 7.39-7.37 (d, J = 8 Hz, 1H), 7.30 (s, 1H), 7.04-7.02 (d, J = 8.8 Hz, 2H), 6.76-6.74 (d, J = 6.0 Hz, 1H), 3.74 (s, 3H). |
| 121 | 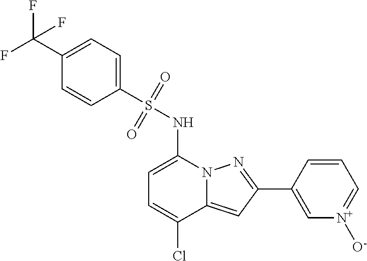 [SMILES: O=S(=O)(Nc1ccc(Cl)c2cc(-c3ccc[n+]([O-])c3)nn12)c1ccc(C(F)(F)F)cc1] | 469.04 | 95.69%, Rt = 4.95 min (1) | ¹H NMR (400 MHz, DMSO- d6): δ 8.83 (s, 1H), 8.23-8.21 (d, J = 6 Hz, 1H), 8.03-8.01 (d, J = 8 Hz, 2H), 7.90-7.89 (d, J = 7.2 Hz, 1H), 7.84- 7.82 (d, J = 7.6 Hz, 2H), 7.51-7.47 (t, J = 7.2 Hz, 1H), 7.21 (m, 2H), 6.56 (s, 1H). |
| 122 | 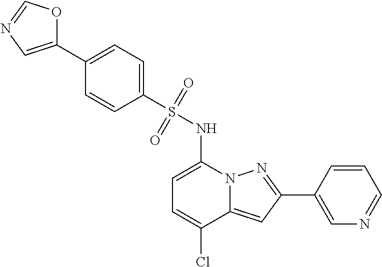 [SMILES: O=S(=O)(Nc1ccc(Cl)c2cc(-c3cccnc3)nn12)c1ccc(-c2cnco2)cc1] | 450.22 (M − 1) | 95.94%, Rt = 4.70 min (1) | ¹H NMR (400 MHz, DMSO- d6): δ 11.65 (bs, 1H), 9.19 (s, 1H), 8.56-8.53 (d, J = 8 Hz, 1H), 8.49 (s, 1H), 8.31- 8.30 (d, J = 5.6 Hz, 1H), 7.99-7.97 (d, J = 8 Hz, 2H), 7.86-7.84 (d, J = 7.2 Hz, 2H), 7.79 (s, 1H), 7.46 (m, 1H), 7.42-7.40 (d, J = 7.6 Hz, 1H), 7.32 (s, 1H), 6.83- 6.81 (d, J = 7.6 Hz, 1H). |
| 123 | 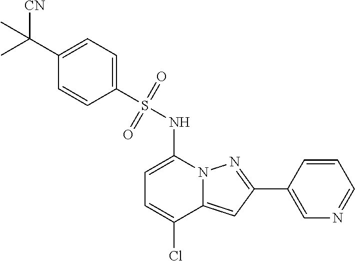 [SMILES: CC(C)(C#N)c1ccc(S(=O)(=O)Nc2ccc(Cl)c3cc(-c4cccnc4)nn23)cc1] | 452.08 | 98.75%, Rt = 5.63 min (2) | ¹H NMR (400 MHz, DMSO- d6): δ 11.52 (bs, 1H), 9.15 (s, 1H), 8.60 (m, 1H), 8.32 (m, 1H), 7.92 (m, 2H), 7.65 (m, 2H), 7.50 (m, 1H), 7.39 (m, 1H), 7.27 (s, 1H), 6.80 (m, 1H), 1.55 (s, 6H). |
| 124 | 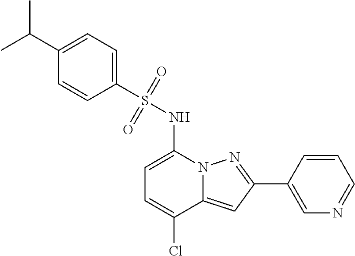 [SMILES: CC(C)c1ccc(S(=O)(=O)Nc2ccc(Cl)c3cc(-c4cccnc4)nn23)cc1] | 427.12 | 98.23%, Rt = 6.21 min (2) | ¹H NMR (400 MHz, DMSO- d6): δ 11.23 (bs, 1H), 9.20 (s, 1H), 8.60 (d, J = 3.6 Hz, 1H), 8.37-8.35 (d, J = 7.6 Hz, 1H), 7.82-7.80 (d, J = 8.0 Hz, 2H), 7.53-7.50 (m, 1H), 7.43-7.37 (m, 3H), 7.32 (s, 1H), 6.84-6.82 (d, J = 8.0 Hz, 1H), 2.88-2.81 (m, 1H), 1.07-1.05 (d, J = 6.8 Hz, 6H). |
| 125 | 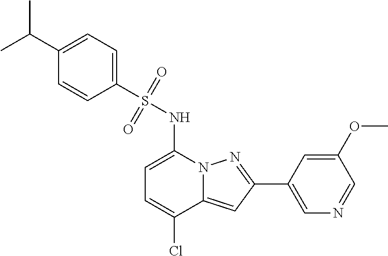 [SMILES: COc1cncc(-c2cc3c(Cl)ccc(NS(=O)(=O)c4ccc(C(C)C)cc4)n3n2)c1] | 455.10 (M − 1) | 96.43% Rt = 5.64 min (1) | ¹H NMR (400 MHz, DMSO- d6): δ 11.27 (bs, 1H), 8.81 (s, 1H), 8.32-8.32 (d, J = 2.8 Hz, 1H), 7.95 (s, 1H), 7.82- 7.80 (d, J = 8.4 Hz, 2H), 7.43-7.36 (m, 4H), 6.84-6.82 (d, J = 8.0 Hz, 1H), 3.93 (s, 3H), 2.88-2.81 (m, 1H), 1.07-1.05 (d, J = 6.8 Hz, 6H) |
| 126 | 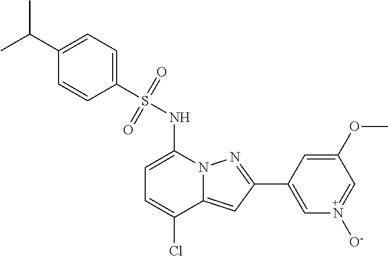 | 473.36 | 95.05% Rt = 5.18 min (1) | ¹H NMR (400 MHz, DMSO- d6): δ 11.44 (bs, 1H), 8.65 (s, 1H), 8.08 (s, 1H), 7.83- 7.81 (d, J = 8.4 Hz, 2H), 7.66 (s, 1H), 7.45-7.44 (d, J = 4.8 Hz, 1H), 7.42 (s, 1H), 7.40-7.38 (d, J = 8.0 Hz, 2H), 6.89-6.87 (d, J = 8.0 Hz, 1H), 3.91 (s, 3H), 2.93- 2.82 (m, 1H), 1.10-1.08 (d, J = 6.8 Hz, 6H). |
| 127 | 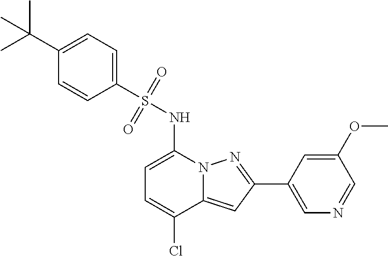 [SMILES: COc1cncc(-c2cc3c(Cl)ccc(NS(=O)(=O)c4ccc(C(C)(C)C)cc4)n3n2)c1] | 471.12 | 98.06%, Rt = 6.79 min (2) | ¹H NMR (400 MHz, DMSO- d6): δ 11.32 (bs, 1H), 8.80 (s, 1H), 8.32-8.31 (d, J = 2.8 Hz, 1H), 7.94 (s, 1H), 7.81-7.79 (d, J = 8.4 Hz, 2H), 7.52-7.49 (d, J = 8.4 Hz, 2H), 7.43-7.41 (d, J = 8.0 Hz, 1H), 7.37 (s, 1H), 6.85-6.83 (d, J = 8.0 Hz, 1H), 3.93 (s, 3H), 1.14 (s, 9H). |
| 128 | 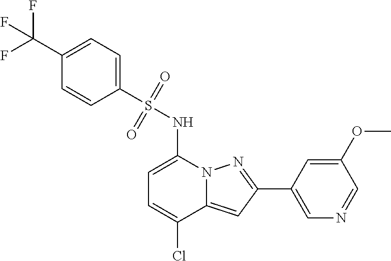 [SMILES: COc1cncc(-c2cc3c(Cl)ccc(NS(=O)(=O)c4ccc(C(F)(F)F)cc4)n3n2)c1] | 483.34 | 98.57%, Rt = 5.54 min (1) | ¹H NMR (400 MHz, DMSO- d6): δ 8.72-8.72 (d, J = 1.6 Hz, 1H), 8.32-8.31 (d, J = 2.8 Hz, 1H), 8.07-8.05 (d, J = 8.0 Hz, 2H), 7.88-7.84 (m, 3H), 7.42-7.40 (d, J = 8.0 Hz, 1H), 7.37 (s, 1H), 6.82-6.80 (d, J = 7.6 Hz, 1H), 3.92 (s, 3H). |
| 129 | 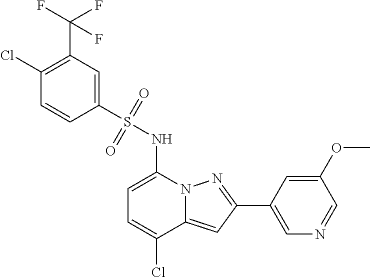 [SMILES: COc1cncc(-c2cc3c(Cl)ccc(NS(=O)(=O)c4ccc(Cl)c(C(F)(F)F)c4)n3n2)c1] | 517.28 | 96.22%, Rt = 5.80 min (1) | ¹H NMR (400 MHz, DMSO- d6): δ 8.72 (s, 1H), 8.34-8.33 (d, J = 2.4 Hz, 1H), 8.17 (s, 1H), 8.11-8.08 (dd, J = 1.6, 2.4 Hz, 1H), 7.88 (s, 1H), 7.85-7.83 (d, J = 8.4 Hz, 1H), 7.42-7.40 (d, J = 8.0 Hz, 1H), 7.38 (s, 1H), 6.85-6.83 (d, J = 8.0 Hz, 1H), 3.93 (s, 3H). |
| 130 | 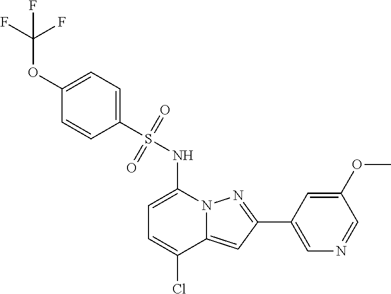 [SMILES: COc1cncc(-c2cc3c(Cl)ccc(NS(=O)(=O)c4ccc(OC(F)(F)F)cc4)n3n2)c1] | 499.32 | 94.22%, Rt = 5.65 min (1) | ¹H NMR (400 MHz, DMSO- d6): δ 8.78 (s, 1H), 8.32 (s, 1H), 8.01-7.99 (d, J = 8.0 Hz, 2H), 7.89 (s, 1H), 7.49- 7.47 (d, J = 8.4 Hz, 2H), 7.42-7.40 (d, J = 7.6 Hz, 1H), 7.37 (s, 1H), 6.83-6.81 (d, J = 8.0 Hz, 1H), 3.93 (s, 3H). |
| 131 | 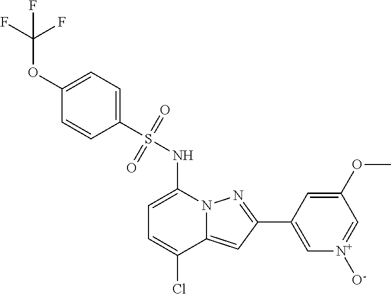 [SMILES: COc1cc(-c2cc3c(Cl)ccc(NS(=O)(=O)c4ccc(OC(F)(F)F)cc4)n3n2)c[n+]([O-])c1] | 515.29 | 95.01%, Rt = 5.26 min (1) | ¹H NMR (400 MHz, DMSO- d6): δ 8.61 (s, 1H), 8.09 (s, 1H), 8.02-8.00 (d, J = 8.8 Hz, 2H), 7.61 (s, 1H), 7.51- 7.49 (d, J = 8.0 Hz, 2H), 7.43 (m, 2H), 6.84 (m, 1H), 3.91 (s, 3H). |
| 132 | 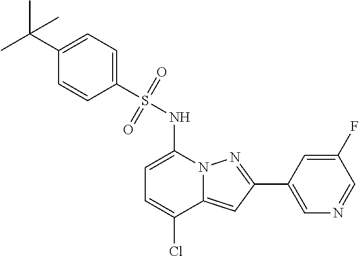 [SMILES: CC(C)(C)c1ccc(S(=O)(=O)Nc2ccc(Cl)c3cc(-c4cncc(F)c4)nn23)cc1] | 459.13 | 97.87% Rt = 5.09 min (2) | ¹H NMR (400 MHz, DMSO- d6): δ 11.25 (bs, 1H), 9.09 (s, 1H), 8.62-8.61 (d, J = 2.8 Hz, 1H), 8.32-8.29 (d, J = 10.4 Hz, 1H), 7.82-7.80 (d, J = 8.4 Hz, 2H), 7.53-7.51 (d, J = 8.4 Hz, 2H), 7.45-7.43 (d, J = 8.0 Hz, 1H), 7.40 (s, 1H), 6.89-6.87 (d, J = 8.0 Hz, 1H), 1.15 (s, 9H). |
| 133 | 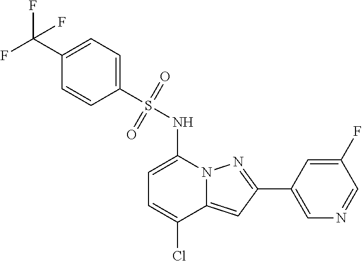 [SMILES: O=S(=O)(Nc1ccc(Cl)c2cc(-c3cncc(F)c3)nn12)c1ccc(C(F)(F)F)cc1] | 471.31 | 99.85%, Rt = 5.61 min (1) | ¹H NMR (400 MHz, DMSO- d6): δ 9.00 (s, 1H), 8.61-8.60 (d, J = 2.4 Hz, 1H), 8.12-8.10 (d, J = 10.0 Hz, 1H), 8.07- 8.05 (d, J = 8.4 Hz, 2H), 7.89- 7.87 (d, J = 8.4 Hz, 2H), 7.45- 7.40 (d, J = 8.0 Hz, 1H), 7.40 (s, 1H), 6.88-6.86 (d, J = 7.8 Hz, 1H). |
| 134 | 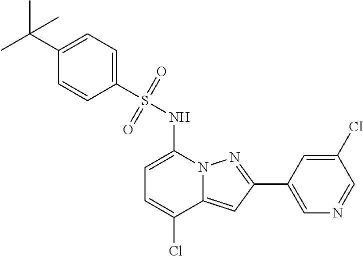 [SMILES: CC(C)(C)c1ccc(S(=O)(=O)Nc2ccc(Cl)c3cc(-c4cncc(Cl)c4)nn23)cc1] | 475.34 | 99.54% Rt = 6.21 min (1) | ¹H NMR (400 MHz, DMSO- d6): δ 9.16 (s, 1H), 8.65-8.64 (d, J = 2.4 Hz, 1H), 8.52 (s, 1H), 7.79-7.77 (d, J = 8.4 Hz, 2H), 7.50-7.48 (d, J = 8.4 Hz, 2H), 7.35 (m, 2H), 6.78 (m, 1H), 1.16 (s, 9H). |
| 135 | 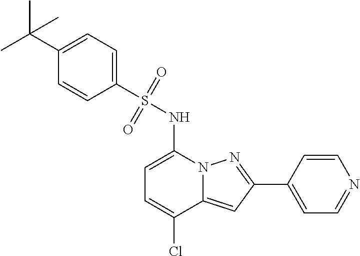 [SMILES: CC(C)(C)c1ccc(S(=O)(=O)Nc2ccc(Cl)c3cc(-c4ccncc4)nn23)cc1] | 441.40 | 96.77% Rt = 5.64 min (1) | ¹H NMR (400 MHz, DMSO- d6): δ 11.58 (bs, 1H), 8.69- 8.68 (d, J = 5.2 Hz, 2H), 8.00- 7.99 (d, J = 5.6 Hz, 2H), 7.81- 7.79 (d, J = 8.4 Hz, 2H), 7.52- 7.50 (d, J = 8.4 Hz, 2H), 7.44- 7.39 (m, 2H), 6.86-6.84 (d, J = 8.0 Hz, 1H), 1.14 (s, 9H). |
| 136 | 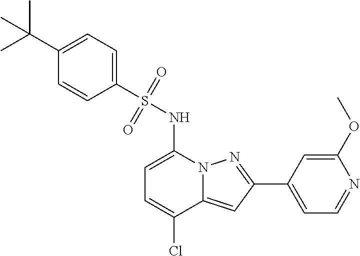 [SMILES: COc1cc(-c2cc3c(Cl)ccc(NS(=O)(=O)c4ccc(C(C)(C)C)cc4)n3n2)ccn1] | 471.14 | 98.38% Rt = 5.20 min (2) | ¹H NMR (400 MHz, DMSO- d6): δ 11.20 (bs, 1H), 8.25- 8.24 (d, J = 5.2 Hz, 1H), 7.81- 7.79 (d, J = 8.0 Hz, 2H), 7.58-7.56 (d, J = 5.2 Hz, 1H), 7.52-7.50 (d, J = 8.4 Hz, 2H), 7.43-7.41 (d, J = 9.6 Hz, 2H), 7.36 (s, 1H), 6.87-6.85 (d, J = 8 Hz, 1H), 3.90 (s, 3H), 1.15 (s, 9H) |
| 137 | 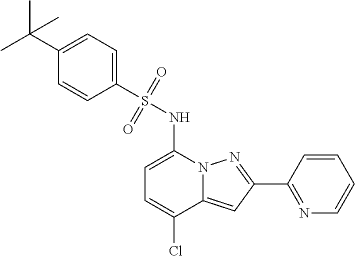 [SMILES: CC(C)(C)c1ccc(S(=O)(=O)Nc2ccc(Cl)c3cc(-c4ccccn4)nn23)cc1] | 441.36 | 99.52% Rt = 5.75 min (1) | ¹H NMR (400 MHz, DMSO- d6): δ 8.65-8.64 (d, J = 4.4 Hz, 1H), 8.12-8.10 (d, J = 7.6 Hz, 1H), 7.96-7.92 (t, J = 7.6 Hz, 1H), 7.82-7.80 (d, J = 8.4 Hz, 2H), 7.52-7.50 (d, J = 8.4 Hz, 2H), 7.43-7.39 (m, 2H), 7.13 (s, 1H), 6.82-6.81 (d, J = 7.6 Hz, 1H), 1.14 (s, 9H). |
| 138 | 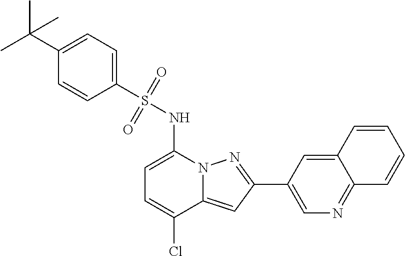 [SMILES: CC(C)(C)c1ccc(S(=O)(=O)Nc2ccc(Cl)c3cc(-c4cnc5ccccc5c4)nn23)cc1] | 491.20 | 99.63% Rt = 6.23 min (1) | ¹H NMR (400 MHz, DMSO- d6): δ 11.37 (bs, 1H), 9.54- 9.53 (d, J = 2.0 Hz, 1H), 8.94 (s, 1H), 8.08-8.06 (d, J = 8.4 Hz, 2H), 7.86-7.79 (m, 3H), 7.70-7.66 (t, J = 6.8 Hz, 1H), 7.53-7.51 (d, J = 8.4 Hz, 2H), 7.46-7.44 (d, J = 6.0 Hz, 2H), 6.87-6.85 (d, J = 8.0 Hz, 1H), 1.09 (s, 9H). |
| 139 | 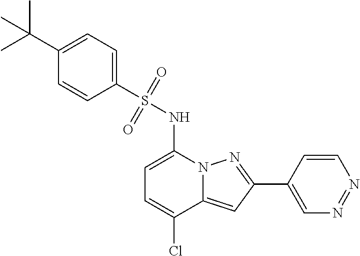 [SMILES: CC(C)(C)c1ccc(S(=O)(=O)Nc2ccc(Cl)c3cc(-c4ccnnc4)nn23)cc1] | 442.37 | 99.53% Rt = 5.43 min (1) | ¹H NMR (400 MHz, DMSO- d6): δ 9.80 (s, 1H), 9.34-9.33 (d, J = 5.2 Hz, 1H), 8.20 (s, 1H), 7.79-7.77 (d, J = 8.4 Hz, 2H), 7.50-7.48 (m, 3H), 7.43 (s, 1H), 6.83-6.80 (d, J = 14.0 Hz, 1H), 1.14 (s, 9H). |
| 140 | 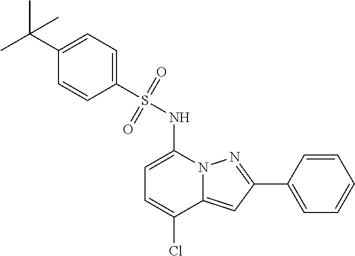 [SMILES: CC(C)(C)c1ccc(S(=O)(=O)Nc2ccc(Cl)c3cc(-c4ccccc4)nn23)cc1] | 440.11 | 97.97% Rt = 5.84 min (2) | ¹H NMR (400 MHz, DMSO- d6): δ 8.02-8.00 (d, J = 7.2 Hz, 2H), 7.84-7.82 (d, J = 8.4 Hz, 2H), 7.54-7.52 (d, J = 8.8 Hz, 2H), 7.48-7.45 (m, 2H), 7.42-7.37 (m, 2H), 7.16 (s, 1H), 6.79-6.77 (d, J = 8.0 Hz, 1H), 1.16 (s, 9H) |
| 141 | 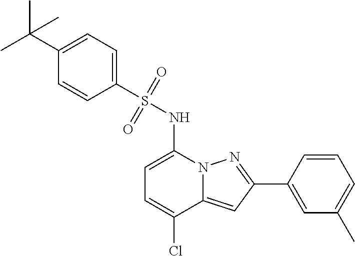 [SMILES: Cc1cccc(-c2cc3c(Cl)ccc(NS(=O)(=O)c4ccc(C(C)(C)C)cc4)n3n2)c1] | 454.41 | 98.56% Rt = 6.64 (1) | ¹H NMR (400 MHz, DMSO- d6): δ 11.35 (bs, 1H), 7.87- 7.83 (m, 3H), 7.80-7.79 (d, J = 6.8 Hz, 1H), 7.54-7.52 (d, J = 8.4 Hz, 2H), 7.39-7.33 (m, 2H), 7.23-7.21 (d, J = 6.8 Hz, 1H), 7.15 (s, 1H), 6.78-6.76 (d, J = 8.0 Hz, 1H), 2.34 (s, 3H), 1.16 (s, 9H). |
| 142 | 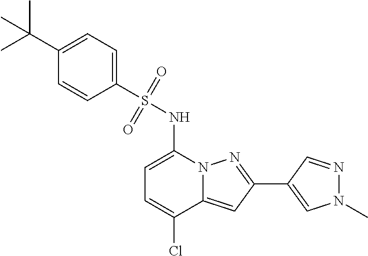 [SMILES: Cn1cc(-c2cc3c(Cl)ccc(NS(=O)(=O)c4ccc(C(C)(C)C)cc4)n3n2)cn1] | 444.13 | 98.64% Rt = 4.66 min (1) | ¹H NMR (400 MHz, DMSO- d6): δ 11.30 (bs, 1H), 8.16 (s, 1H), 7.85-7.83 (m, 3H), 7.56- 7.54 (d, J = 7.2 Hz, 2H), 7.34- 7.32 (d, J = 7.2 Hz, 1H), 6.85 (s, 1H), 6.68-6.66 (d, J = 7.2 Hz, 1H), 3.89 (s, 3H), 1.20 (s, 9H). |
| 143 | 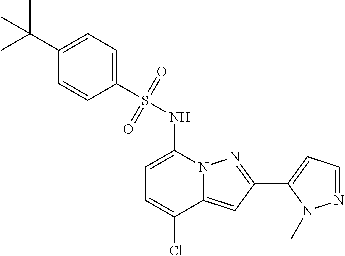 [SMILES: Cn1nccc1-c1cc2c(Cl)ccc(NS(=O)(=O)c3ccc(C(C)(C)C)cc3)n2n1] | 444.17 | 98.83% Rt = 4.86 min (1) | ¹H NMR (400 MHz, DMSO- d6): δ 11.28 (bs, 1H), 7.75- 7.73 (d, J = 8.8 Hz, 2H), 7.52- 7.50 (d, J = 8.4 Hz, 2H), 7.46- 7.44 (d, J = 8.4 Hz, 2H), 7.06 (s, 1H), 6.86-6.84 (d, J = 8 Hz, 1H), 6.80-6.79 (d, J = 2 Hz, 1H), 4.09 (s, 3H), 1.17 (s, 9H). |
| 144 | 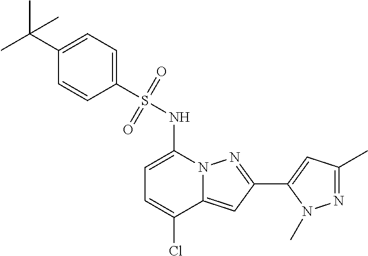 [SMILES: Cc1cc(-c2cc3c(Cl)ccc(NS(=O)(=O)c4ccc(C(C)(C)C)cc4)n3n2)n(C)n1] | 458.40 | 99.64% Rt = 5.91 min (1) | ¹H NMR (400 MHz, DMSO- d6): δ 11.29 (bs, 1H), 7.76- 7.74 (d, J = 8.4 Hz, 2H), 7.52- 7.50 (d, J = 8.8 Hz, 2H), 7.45- 7.43 (d, J = 7.6 Hz, 1H), 6.99 (s, 1H), 6.84-6.82 (d, J = 8.0 Hz, 1H), 6.55 (s, 1H), 4.00 (s, 3H), 2.16 (s, 3H), 1.18 (s, 9H). |
| 145 | 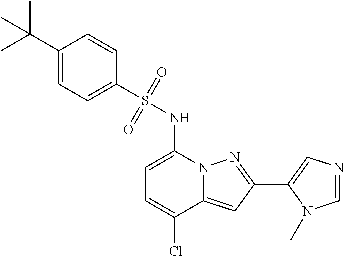 [SMILES: Cn1cncc1-c1cc2c(Cl)ccc(NS(=O)(=O)c3ccc(C(C)(C)C)cc3)n2n1] | 444.46 | 95.53% Rt = 5.32 min (1) | ¹H NMR (400 MHz, DMSO- d6): δ 8.18 (s, 1H), 7.75-7.69 (m, 3H), 7.49-7.47 (d, J = 5.6 Hz, 2H), 7.31-7.30 (d, J = 6.4 Hz, 1H), 6.93 (s, 1H), 6.65- 6.63 (d, J = 8.8 Hz, 1H), 3.96 (s, 3H), 1.20 (s, 9H). |
| 146 | 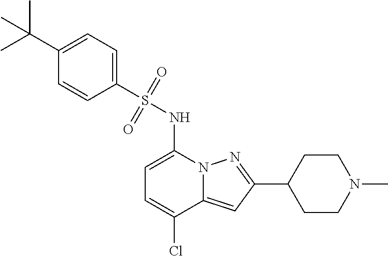 [SMILES: CN1CCC(c2cc3c(Cl)ccc(NS(=O)(=O)c4ccc(C(C)(C)C)cc4)n3n2)CC1] | 461.27 | 99.66% Rt = 6.0 min (1) | ¹H NMR (400 MHz, DMSO- d6): δ 9.41 (bs, 1H), 7.86-7.80 (m, 2H), 7.57-7.55 (d, J = 8.4 Hz, 2H), 7.37-7.31 (m, 1H), 6.75-6.55 (m, 2H), 3.55 (m, 1H), 3.39-2.99 (m, 4H), 2.83 (s, 3H), 2.24-2.21 (m, 2H), 2.07-1.80 (m, 2H), 1.25 (s, 9H). |

### Example 15

### Synthesis of Compound 147 [4-(tert-butyl)-N-(4-chloro-2-(1H-imidazol-4-yl)pyrazolo[1,5-a]pyridin-7-yl)benzenesulfonamide] and Compound 148

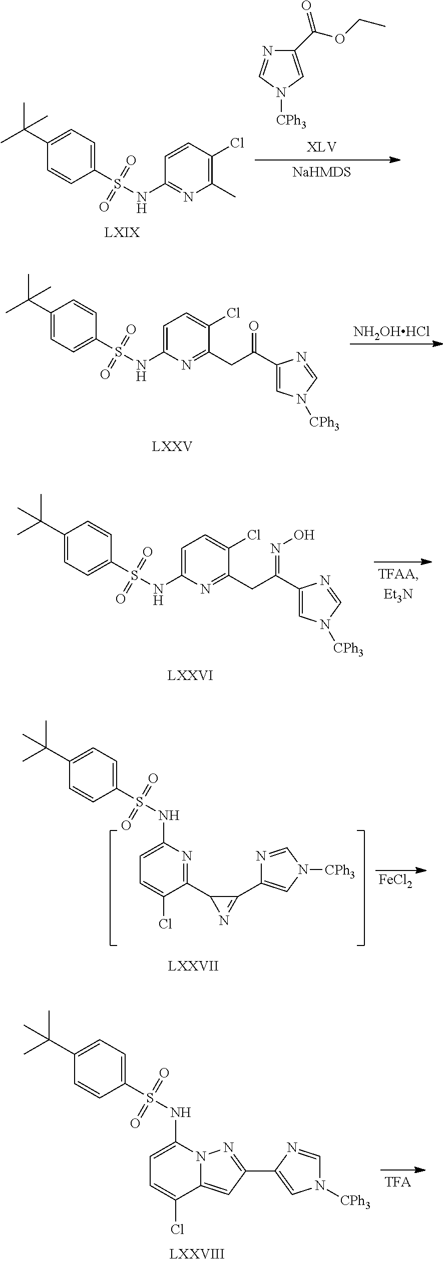

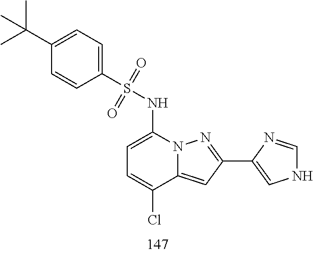 [SMILES: CC(C)(C)c1ccc(S(=O)(=O)Nc2ccc(Cl)c3cc(-c4c[nH]cn4)nn23)cc1]

Synthesis of LXXV:

To a stirred solution of compound LXIX (3 g; 8.87 mmol) and ethyl 1-trityl-1H-imidazole-4-carboxylate (XLV; 10 g; 26.62 mmol) in THF (50 ml) was added sodium bis(trimethylsilyl)amide (44 ml, 1.0 M in THF, 44.35 mmol) dropwise at 0° C. Upon complete addition, the resultant solution was stirred at ambient temperature for 2 h. The reaction mixture was diluted with a saturated solution of ammonium chloride and extracted with ethyl acetate. The organic layer was washed with brine solution and dried over anhydrous Na₂SO₄, filtered and evaporated under reduced pressure to obtain 4-(tert-butyl)-N-(5-chloro-6-(2-oxo-2-(1-trityl-1H-imidazol-4-yl)ethyl)pyridin-2-yl)benzenesulfonamide LXXV, as a keto-enol tautomeric mixture. MS (M+1): 675.12. The crude material was carried forward to next step without purification.

Synthesis of LXXVI:

To a stirred solution of compound LXXV (6 g, tautomeric mixture) in methanol (60 ml) was added hydroxylamine hydrochloride (1.9 g; 26.7 mmol) followed by a 10% aqueous solution of sodium hydroxide (36 ml). The resultant suspension was heated at 100° C. for 12 h and then cooled to room temperature. The reaction mixture was concentrated in vacuo and the residue was diluted with water and extracted with ethyl acetate. The organic layer was washed with brine solution and dried over anhydrous Na₂SO₄, filtered and evaporated under reduced pressure to obtain the crude compound which was purified by column chromatography using 100% ethyl acetate to afford the desired product 4-(tert-butyl)-N-(5-chloro-6-(2-(hydroxyimino)-2-(1-trityl-1H-imidazol-4-yl)ethyl)pyridin-2-yl)benzenesulfonamide as a white solid (LXXVI; 4 g; 67% yield). ¹H NMR (400 MHz, DMSO-d6): δ 10.93 (bs, 1H), 10.79 (bs, 1H), 7.86-7.84 (d, J=8 Hz, 2H), 7.65-7.63 (d, J=8.8 Hz, 1H), 7.48-7.46 (m, 2H), 7.34 (m, 10H), 7.05 (m, 6H), 6.92 (s, 1H), 6.76-6.74 (m, 1H), 4.16 (s, 2H), 1.20 (s, 9H). MS (M+1): 690.11.

Synthesis of LXXVIII:

To a stirred solution of compound LXXVI (1 g, 1.45 mmol) in 1,2-dimethoxyethane (20 ml) at 0° C. was added trifluroacetic anhydride (0.9 g, 4.35 mmol). The reaction mixture was allowed to stir at 0° C. for 20 minutes, followed by dropwise addition of triethylamine (2.93 g, 29 mmol) in 1,2-dimethoxyethane (10 ml). The reaction mixture was stirred at room temperature for 1 h to leave LXXVII prepared in situ. To the reaction mixture was further added iron (II) chloride (0.07 g, 0.58 mmol) and this was heated at 100° C. for 3 h. The reaction mixture was cooled and concentrated, diluted with water and extracted with dichloromethane. The organic layer was washed with brine solution and dried over anhydrous Na₂SO₄, filtered and evaporated under reduced pressure to obtain crude compound, which was purified by column chromatography using 2% methanol in dichloromethane to afford the title compound (LXXVIII; 0.6 g; 67% yield). MS (M−1): 670.11.

### Synthesis of Compound 147: 4-(tert-butyl)-N-(4-chloro-2-(1H-imidazol-4-yl)pyrazolo[1,5-a]pyridin-7-yl)benzenesulfonamide

To a stirred solution of compound LXXVIII, (0.25 g, 0.37 mmol) in water (5 ml) at 0° C. was added trifluoroacetic acid (5 ml). The resultant solution was allowed to stir at 0° C. for 15 min. The reaction mixture was concentrated under reduced pressure to obtain the crude compound, which was purified by preparative HPLC to afford the title compound 147, ¹H NMR (400 MHz, DMSO-d6): δ 8.54 (s, 1H), 7.92 (s, 1H), 7.77-7.75 (d, J=8 Hz, 2H), 7.48-7.46 (d, J=8.4 Hz, 2H), 7.18-7.16 (d, J=8.4 Hz, 1H), 6.86 (s, 1H), 6.45-6.43 (d, J=8.8 Hz, 1H), 1.22 (s, 9H). MS (M+1): 430.15. (LCMS purity 97.87%, Rt=5.75 min) (2).

| | | LCMS | Purity | |
|---|---|---|---|---|
| CPD | Structure | (M + 1) | (LCMS) | ¹H NMR |
| 148 | 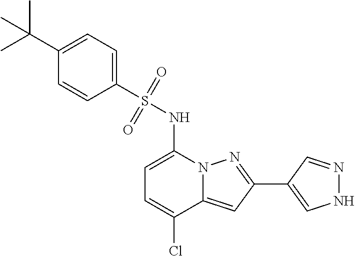 [SMILES: CC(C)(C)c1ccc(S(=O)(=O)Nc2ccc(Cl)c3cc(-c4cn[nH]c4)nn23)cc1] | 430.13 | 98.89% Rt = 6.78 min (1) | ¹H NMR (400 MHz, DMSO- d6): δ 13.06 (bs, 1H), 8.06 (m, 2H), 7.85-7.83 (d, J = 8.4 Hz, 2H), 7.55-7.53 (d, J = 8.4 Hz, 2H), 7.34-7.32 (d, J = 8.0 Hz, 1H), 6.89 (s, 1H), 6.68-6.66 (d, J = 8.0 Hz, 1H), 1.19 (s, 9H). |

### Example 16

### Synthesis of Compound 149 [4-(tert-butyl)-N-(4-cyano-2-(pyridin-3-yl)pyrazolo[1,5-a]pyridin-7-yl)benzenesulfonamide] and Compounds 150 to 152

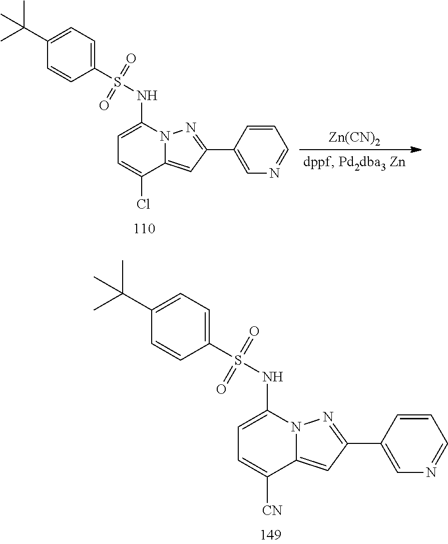

To a stirred solution of 110 (0.15 g, 0.34 mmol) in dimethylacetamide (5 ml) was added Zn(CN)₂ (0.079 g, 0.68 mmol). The reaction mixture was purged with argon for 20 minutes. To the reaction mixture was then added 1, 1′-Bis (diphenylphosphino)ferrocene (0.038 g, 0.068 mmol), Pd₂dba₃ (0.047 g, 0.051 mmol) and a catalytic amount of Zn dust. The reaction mixture was heated at 120° C. for 2 h in a microwave reactor. The reaction mixture was cooled and concentrated, diluted with water and extracted with dichloromethane. The organic layer was washed with brine solution and dried over anhydrous Na₂SO₄, filtered and evaporated under reduced pressure to obtain material which was purified by column chromatography using 2% methanol in 2% ammoniated dichloromethane. This afforded the title compound (149; 0.015 g, 10% yield). ¹H NMR (400 MHz, DMSO-d6): δ 9.32 (s, 1H), 8.66-8.65 (m, 1H), 8.60-8.58 (d, J=8.0 Hz, 1H), 7.84-7.82 (d, J=8.4 Hz, 2H), 7.65-7.62 (m, 2H), 7.54-7.51 (d, J=8.4 Hz, 2H), 7.23 (s, 1H), 6.62-6.60 (d, J=8.4 Hz, 1H), 1.25 (s, 9H). MS (M+1): 432.44. (LCMS Purity 96.63%, Rt=5.36 min) (1).

The following compounds were prepared from the analogous chloro compounds prepared in the examples above.

| | | LCMS | Purity | |
|---|---|---|---|---|
| CPD | Structure | (M + 1) | (LCMS) | ¹H NMR |
| 150 | 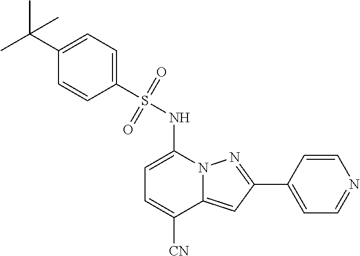 [SMILES: CC(C)(C)c1ccc(S(=O)(=O)Nc2ccc(C#N)c3cc(-c4ccncc4)nn23)cc1] | 432.22 | 99.28% Rt = 5.69 min (2) | ¹H NMR (400 MHz, DMSO- d6): δ 8.69-8.68 (d, J = 5.6 Hz, 2H), 8.11-8.10 (d, J = 4.8 Hz, 2H), 7.77-7.75 (d, J = 8.4 Hz, 2H), 7.53 (d, J = 8.4 Hz, 1H), 7.49-7.47 (d, J = 8.4 Hz, 2H), 7.15 (s, 1H), 6.46-6.44 (d, J = 8.8 Hz, 1H), 1.23 (s, 9H). |
| 151 | 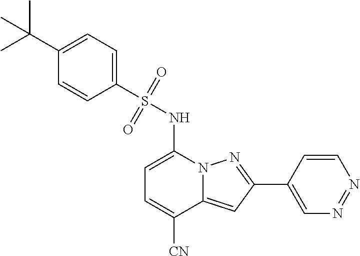 [SMILES: CC(C)(C)c1ccc(S(=O)(=O)Nc2ccc(C#N)c3cc(-c4ccnnc4)nn23)cc1] | 433.12 | 98.49% Rt = 5.0 min (1) | ¹H NMR (400 MHz, DMSO- d6): δ 9.84 (s, 1H), 9.29-9.28 (d, J = 5.6 Hz, 1H), 8.24-8.23 (m, 1H), 7.77-7.75 (d, J = 8 Hz, 2H), 7.54-7.52 (d, J = 8.4 Hz, 1H), 7.49-7.47 (d, J = 8.4 Hz, 2H), 7.27 (s, 1H), 6.47-6.45 (d, J = 8.4 Hz, 1H), 1.25 (s, 9H). |
| 152 | 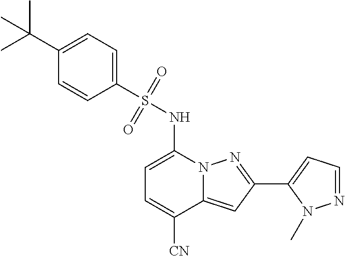 [SMILES: Cn1nccc1-c1cc2c(C#N)ccc(NS(=O)(=O)c3ccc(C(C)(C)C)cc3)n2n1] | 435.14 | 99.83% Rt = 5.19 min (1) | ¹H NMR (400 MHz, DMSO- d6): δ 7.83-7.80 (d, J = 8.4 Hz, 2H), 7.67-7.65 (d, J = 7.6 Hz, 1H), 7.53-7.48 (m, 3H), 6.92 (s, 1H), 6.81 (m, 1H), 6.62-6.60 (d, J = 8.8 Hz, 1H), 4.19 (s, 3H), 1.24 (s, 9H). |

### Example 17

### Synthesis of Compound 153 [4-(tert-butyl)-N-(4-chloro-2-(5-cyanopyridin-3-yl)pyrazolo[1,5-a]pyridin-7-yl)benzenesulfonamide]

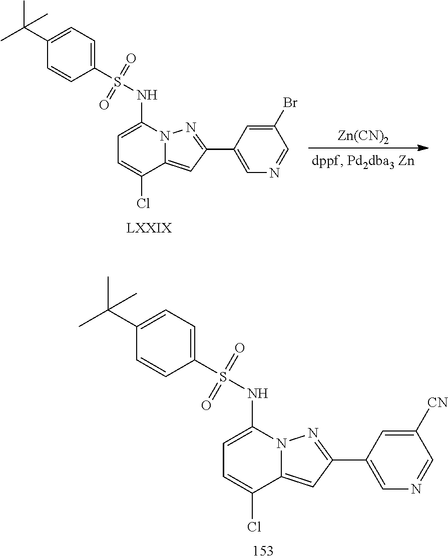

Compound LXXIX was synthesized from LXIX and ethyl 5-bromonicotinate essentially as described in Example 14.

To a stirred solution of compound LXXIX, (0.2 g, 0.39 mmol) in dimethylacetamide (5 ml) was added Zn(CN)₂ (0.09 g, 0.78 mmol). The reaction mixture was purged with argon for 20 minutes followed by addition of 1, 1′-Bis (diphenylphosphino)ferrocene (0.044 g, 0.078 mmol), Pd2dba3 (0.036 g, 0.039 mmol) and a catalytic amount of Zn dust. The reaction mixture was heated at 120° C. for 2 h in a microwave reactor. The reaction mixture was cooled and filtered through a celite bed. The filtrate was concentrated, diluted with water and extracted with dichloromethane. The organic layer was washed with brine solution and dried over anhydrous Na₂SO₄, filtered and evaporated under reduced pressure to obtain the crude compound, which was purified by column chromatography using 2% methanol in 10% ammoniated dichloromethane to afford the title compound (153; 0.015 g, 9% yield). ¹H NMR (400 MHz, DMSO-d6): δ 11.36 (bs, 1H), 9.47-9.46 (d, J=2 Hz, 1H), 9.06-9.05 (d, J=2 Hz, 1H), 8.93-8.92 (m, 1H), 7.84-7.82 (d, J=8.4 Hz, 2H), 7.54-7.52 (d, J=8.4 Hz, 2H), 7.47-7.45 (d, J=8.0 Hz, 2H) 6.92-6.90 (d, J=8.0 Hz, 1H), 1.16 (s, 9H). MS (M+1): 466.42. (LCMS Purity 99.57%, Rt=5.84 min) (1).

### Example 18

### Synthesis of Compound 154 [4-(tert-butyl)-N-(4-cyano-2-(5-cyanopyridin-3-yl)pyrazolo[1,5-a]pyridin-7-yl)benzenesulfonamide]

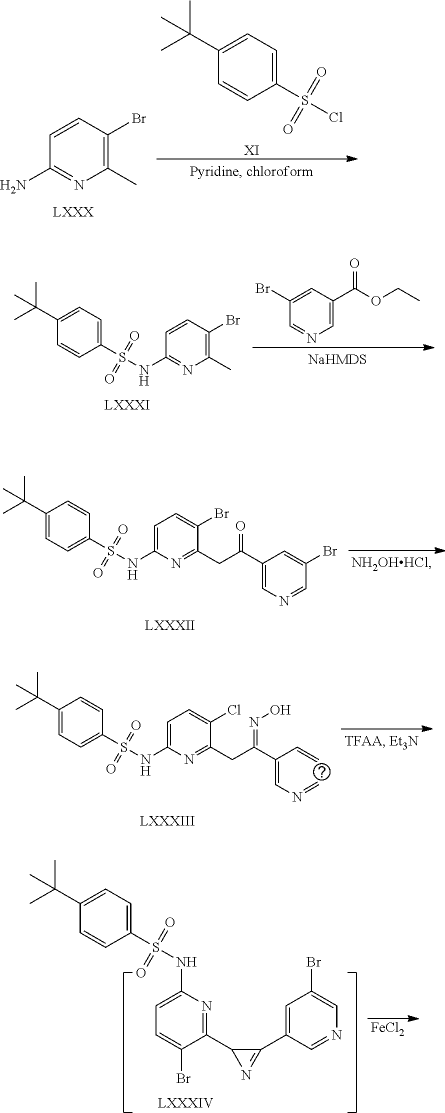

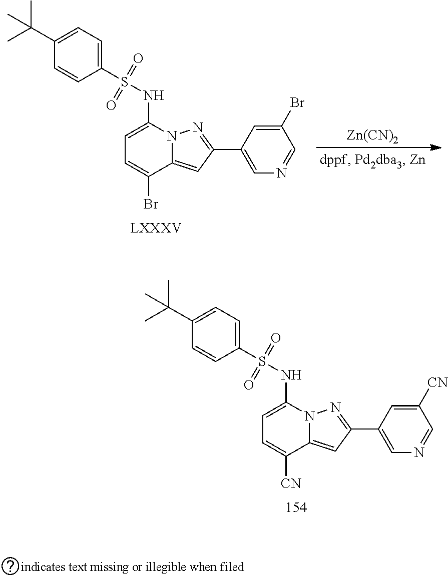

Synthesis of LXXXI:

To a stirred solution of compound LXXX (12 g, 64.15 mmol) in chloroform (120 ml) was added pyridine (25 ml) at 0° C. followed by addition of 4-tert-butylbenzenesulfonyl chloride (XI, 17.92 g, 76.98 mmol). The reaction mixture was heated at 80° C. for 4 h, cooled to room temperature and concentrated under reduced pressure. The crude mass was diluted with a saturated ammonium chloride solution and extracted with ethyl acetate. The organic layer was washed with brine solution, dried over anhydrous Na₂SO₄ and evaporated under reduced pressure to afford N-(5-bromo-6-methylpyridin-2-yl)-4-(tert-butyl)benzenesulfonamide (LXXXI, 22 g, 89% yield). ¹H NMR (400 MHz, DMSO-d6) δ 11.14 (bs, 1H), 7.86-7.82 (m, 3H), 7.60-7.58 (d, J=8.4 Hz, 2H), 6.87-6.85 (d, J=10.4 Hz, 1H), 2.39 (s, 3H), 1.27 (s, 9H). MS (M+1): 383.2.

Synthesis of LXXXII:

To a stirred solution of compound LXXXI (2 g; 5.21 mmol) and ethyl 5-bromonicotinate (2.39 g; 10.42 mmol) in THF (50 ml) was added sodium bis(trimethylsilyl)amide (13.02 ml, 1.0 M in THF, 13.02 mmol) dropwise at 0° C. Upon complete addition, the resultant solution was stirred at ambient temperature for 4 h. The reaction mixture was diluted with a saturated solution of ammonium chloride and extracted with ethyl acetate. The organic layer was washed with brine solution and dried over anhydrous Na₂SO₄, filtered and evaporated under reduced pressure to obtain LXXXII, N-(5-bromo-6-(2-(5-bromopyridin-3-yl)-2-oxoethyl)pyridin-2-yl)-4-(tert-butyl) benzene sulfonamide, as a keto-enol tautomeric mixture. MS (M+1): 566.2. The crude material was carried forward to next step without purification.

Synthesis of LXXXIII:

To a stirred solution of compound LXXXII (2.3 g, tautomeric mixture) in methanol (100 ml) was added hydroxylamine hydrochloride (2.7 g; 40.29 mmol) followed by a 10% aqueous solution of sodium hydroxide (25 ml). The resultant suspension was heated at 80° C. for 12 h and then cooled to room temperature. The reaction mixture was concentrated in vacuo and the residue was diluted with water and extracted with dichloromethane. The organic layer was washed with brine solution and dried over anhydrous Na₂SO₄, filtered and was evaporated under reduced pressure to obtain crude compound, which was purified by column chromatography using 35% ethyl acetate in hexane to afford desired product N-(6-(2-(5-bromopyridin-3-yl)-2-(hydroxyimino)ethyl)-5-chloropyridin-2-yl)-4-(tert-butyl)benzenesulfonamide as white solid (LXXXIII; 1.5 g; 69% yield). ¹H NMR (400 MHz, DMSO-d6) δ 11.79 (s, 1H), 11.01 (bs, 1H), 8.67 (s, 1H), 8.64-8.63 (d, J=2 Hz, 1H), 8.07 (d, J=2 Hz, 1H), 7.85-7.83 (d, J=8.8 Hz, 1H), 7.69-7.67 (m, 2H), 7.47-7.45 (m, 2H), 6.73-6.71 (d, J=8.4 Hz, 1H), 4.23 (s, 2H), 1.25 (s, 9H). MS (M+1): 537.1 (LCMS Purity 95%).

Synthesis of LXXXV:

To a stirred solution of LXXXIII (1 g, 1.71 mmol) in 1,2-dimethoxyethane (30 ml) at 0° C. was added trifluroacetic anhydride (0.72 g, 3.42 mmol). The reaction mixture was allowed to stir for 20 minutes, followed by dropwise addition of triethylamine (1.73 g, 17.1 mmol) in 1,2-dimethoxyethane (5 ml). The reaction mixture was stirred at room temperature for 1 h to generate LXXXIV in situ. To the reaction mixture was added iron (II) chloride (0.043 g, 0.34 mmol) and heated at 100° C. for 3 h. The reaction mixture was cooled and concentrated, diluted with water and extracted with dichloromethane. The organic layer was washed with brine solution and dried over anhydrous Na₂SO₄, filtered and evaporated under reduced pressure to obtain crude compound, which was purified by column chromatography using 2% methanol in dichloromethane to afford N-(4-bromo-2-(5-bromopyridin-3-yl)pyrazolo[1,5-a]pyridin-7-yl)-4-(tert-butyl)benzenesulfonamide as off white solid (LXXXV; 0.5 g; 50% yield).

### Synthesis of Compound 154; 4-(tert-butyl)-N-(4-cyano-2-(5-cyanopyridin-3-yl)pyrazolo[1,5-a]pyridin-7-yl)benzenesulfonamide

To a stirred solution of LXXXV (0.3 g, 0.53 mmol) in dimethylacetamide (5 ml) was added Zn(CN)₂ (0.31 g, 2.65 mmol). The reaction mixture was purged with argon for 20 min and 1, 1′-Bis (diphenylphosphino)ferrocene (0.08 g, 0.16 mmol), Pd2dba3 (0.09 g, 0.1 mmol) and catalytic amount of Zn dust were added. The reaction mixture was heated at 120° C. for 2 h in a microwave reactor. The reaction mixture was cooled and concentrated, diluted with water and extracted with dichloromethane. The organic layer was washed with brine solution and dried over anhydrous Na₂SO₄, filtered and evaporated under reduced pressure to obtain crude compound, which was purified by column chromatography using 1% methanol in dichloromethane to afford the title compound as an off white solid (154; 0.08 g, 33% yield).

¹H NMR (400 MHz, DMSO-d6): δ 9.54 (s, 1H), 9.04 (s, 1H), 8.94 (s, 1H), 7.85-7.83 (d, J=8.4 Hz, 2H), 7.69-7.67 (d, J=8.0 Hz, 1H), 7.55-7.53 (d, J=8.0 Hz, 2H), 7.36 (s, 1H), 6.67-6.65 (d, J=8.0 Hz, 1H), 1.23 (s, 9H). MS (M+1): 457.44. (LCMS Purity 96.01%, Rt=5.63 min) (1).

### Example 19

### Synthesis of Compound 155 [N-(4-bromo-2-(5-fluoropyridin-3-yl)pyrazolo[1,5-a]pyridin-7-yl)-4-(tert-butyl)benzenesulfonamide]; Compound 156 [4-(tert-butyl)-N-(4-cyano-2-(5-fluoropyridin-3-yl)pyrazolo[1,5-a]pyridin-7-yl)benzenesulfonamide] and Compounds 157 to 161

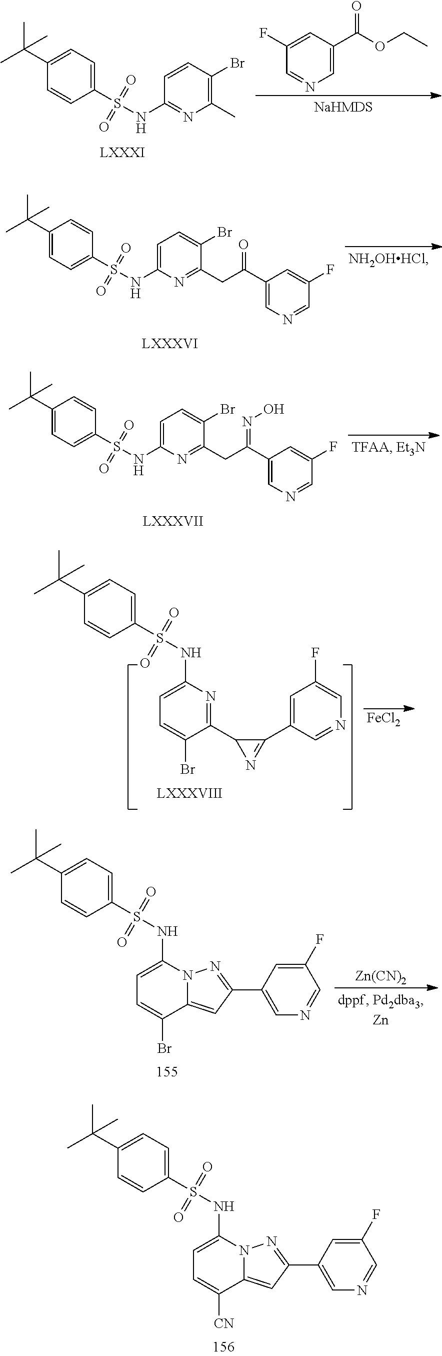

Synthesis of LXXXVI:

To a stirred solution of compound LXXXI (2.5 g; 6.54 mmol) and ethyl 5-fluoronicotinate (2.2 g; 13.08 mmol) in THF (30 ml) was added sodium bis(trimethylsilyl)amide (19.7 ml, 1.0M in THF, 19.62 mmol) dropwise at 0° C. Upon complete addition, the resultant solution was stirred at ambient temperature for 4 h. The reaction mixture was diluted with a saturated solution of ammonium chloride and extracted with ethyl acetate. The separated organic layer was washed with brine solution and dried over anhydrous Na₂SO₄, filtered and evaporated under reduced pressure to obtain LXXXVI, N-(5-bromo-6-(2-(5-fluoropyridin-3-yl)-2-oxoethyl)pyridin-2-yl)-4-(tert-butyl)benzenesulfonamide as a keto-enol tautomeric mixture. MS (M+1): 506.10. The crude material was carried forward to the next step without purification.

Synthesis of LXXXVII:

To a stirred solution of compound LXXXVI (2.4 g, tautomeric mixture) in methanol (100 ml) was added hydroxylamine hydrochloride (1.64 g; 23.71 mmol) followed by a 10% aqueous solution of sodium hydroxide (20 ml). The resultant suspension was heated at 100° C. for 12 h and then cooled to room temperature. The reaction mixture was concentrated in vacuo and the residue was diluted with water and extracted with ethyl acetate. The organic layer was washed with brine solution and dried over anhydrous Na₂SO₄, filtered and was evaporated under reduced pressure to obtain the crude compound, which was purified by column chromatography using 15% ethyl acetate in hexane to afford desired product N-(5-bromo-6-(2-(5-fluoropyridin-3-yl)-2-(hydroxyimino)ethyl)pyridin-2-yl)-4-(tert-butyl)benzenesulfonamide as off white solid (LXXXVII; 1.2 g; 48% yield). MS (M+1): 521.1 (LCMS Purity 96%).

### Synthesis of Compound 155; N-(4-bromo-2-(5-fluoropyridin-3-yl)pyrazolo[1,5-a]pyridin-7-yl)-4-(tert-butyl)benzenesulfonamide

To a stirred solution of LXXXVII (1.2 g, 2.30 mmol) in 1,2-dimethoxyethane (22 ml) at 0° C. was added trifluroacetic anhydride (0.96 g, 4.60 mmol). The reaction mixture was allowed to stir at 0° C. for 20 minutes, followed by the dropwise addition of triethylamine (2.32 g, 23.0 mmol) in 1,2-dimethoxyethane (10 ml). The reaction mixture was stirred at room temperature for 1.5 h forming LXXXVIII in situ. To the reaction mixture was added iron (II) chloride (0.11 g, 0.92 mmol) and this was heated at 100° C. for 3 h. The reaction mixture was cooled and concentrated, diluted with water and extracted with ethyl acetate. The organic layer was washed with brine solution and dried over anhydrous Na₂SO₄, filtered and evaporated under reduced pressure to obtain crude compound, which was purified by column chromatography using 12% ethyl acetate in hexane to afford the title compound as an off white solid. (155; 0.4 g; 35% yield). ¹H NMR (400 MHz, DMSO-d6): δ 11.22 (bs, 1H), 9.10 (s, 1H), 8.61-8.61 (d, J=2.8 Hz, 1H), 8.34-8.32 (d, J=10.0 Hz, 1H), 7.83-7.81 (d, J=8.4 Hz, 2H), 7.58-7.52 (m, 3H), 7.35 (s, 1H), 6.85-6.83 (d, J=8.0 Hz, 1H), 1.16 (s, 9H). MS (M−1): 501.28. (LCMS Purity 98.28%, Rt=5.91 min) (1).

### Synthesis of Compound 156; 4-(tert-butyl)-N-(4-cyano-2-(5-fluoropyridin-3-yl)pyrazolo[1,5-a]pyridin-7-yl)benzenesulfonamide

To a stirred solution of 114 (0.2 g, 0.39 mmol) in dimethylacetamide (10 ml) was added Zn(CN)₂ (0.09 g, 0.78 mmol). The reaction mixture was purged with argon for 20 minute. Then, 1, 1′-Bis (diphenylphosphino)ferrocene (0.043 g, 0.078 mmol), Pd₂dba₃ (0.054 g, 0.058 mmol) and a catalytic amount of Zn dust were added. The reaction mixture was heated at 120° C. for 2 h in a microwave reactor. The reaction mixture was cooled and concentrated, diluted with water and extracted with dichloromethane. The organic layer was washed with brine solution and dried over anhydrous Na₂SO₄, filtered and evaporated under reduced pressure to obtain crude compound, which was purified by column chromatography using 5% methanol in dichloromethane and 10% ammonia hydroxide to afford the title compound (156; 0.06 g, 33% yield). ¹H NMR (400 MHz, DMSO-d6): δ 9.16 (s, 1H), 8.61-8.60 (d, J=2.4 Hz, 1H), 8.37-8.34 (d, J=10.0 Hz, 1H), 7.83-7.81 (d, J=8.0 Hz, 2H), 7.65-7.63 (d, J=8.4 Hz, 1H), 7.54-7.51 (d, J=8.4 Hz, 2H), 7.27 (s, 1H), 6.62-6.60 (d, J=8.0 Hz, 1H), 1.24 (s, 9H). MS (M+1): 450.44. (LCMS Purity 98.83%, Rt=5.60 min) (1).

The following compounds were made in essentially the same manner using the appropriate ethyl ester in the first step.

| | | LCMS | Purity | |
|---|---|---|---|---|
| CPD. | Structure | (M + 1) | (LCMS) | ¹H NMR |
| 157 | 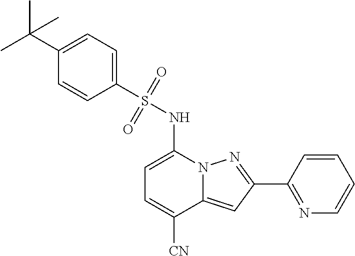 [SMILES: CC(C)(C)c1ccc(S(=O)(=O)Nc2ccc(C#N)c3cc(-c4ccccn4)nn23)cc1] | 432.11 | 99.35% Rt = 5.36 min (1) | ¹H NMR (400 MHz, DMSO- d6): δ 8.65-8.64 (d, J = 8 Hz, 1H), 8.19-8.17 (d, J = 7.6 Hz, 1H), 7.91-7.88 (t, J = 6.8 Hz, 1H), 7.77-7.74 (d, J = 8.4 Hz, 2H), 7.48-7.46 (m, 3H) 7.40- 7.37 (m, 1H), 6.89 (s, 1H), 6.40-6.38 (d, J = 8.4 Hz, 1H), 1.23 (s, 9H). |
| 158 | 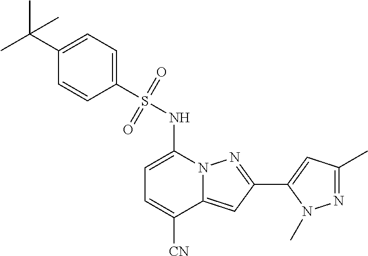 [SMILES: Cc1cc(-c2cc3c(C#N)ccc(NS(=O)(=O)c4ccc(C(C)(C)C)cc4)n3n2)n(C)n1] | 449.28 | 99.00% Rt = 6.87 min (2) | ¹H NMR (400 MHz, DMSO- d6): δ 7.87-7.85 (d, J = 8.4 Hz, 2H), 7.77-7.75 (d, J = 8.4 Hz, 1H), 7.57-7.55 (d, J = 8.4 Hz, 2H), 6.97 (s, 1H), 6.74- 6.72 (d, J = 7.6 Hz, 1H), 6.61 (s, 1H), 4.10 (s, 3H), 2.18 (s, 3H), 1.24 (s, 9H). |
| 159 | 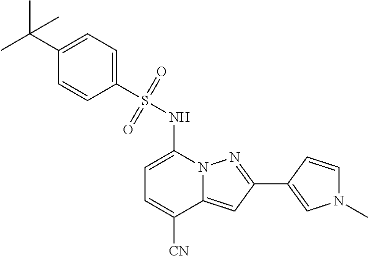 [SMILES: Cn1ccc(-c2cc3c(C#N)ccc(NS(=O)(=O)c4ccc(C(C)(C)C)cc4)n3n2)c1] | 434.27 | 94.42% Rt = 7.04 min (2) | ¹H NMR (400 MHz, DMSO- d6): δ 7.91-7.90 (d, J = 7.2 Hz, 2H), 7.68-7.64 (m, 1H), 7.59-7.57 (d, J = 8.0 Hz, 2H), 7.42-7.41 (d, J = 4.4 Hz, , 1H), 6.81 (m, 2H), 6.59-6.58 (m, 2H), 3.67 (s, 3H), 1.25 (s, 9H). |
| 160 | 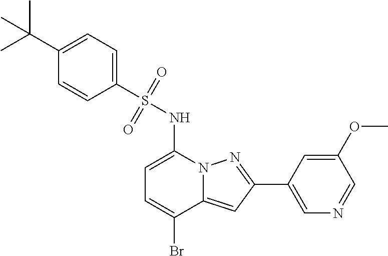 [SMILES: COc1cncc(-c2cc3c(Br)ccc(NS(=O)(=O)c4ccc(C(C)(C)C)cc4)n3n2)c1] | 517.32 | 98.16% Rt = 5.87 min (1) | ¹H NMR (400 MHz, DMSO- d6): δ 11.36 (bs, 1H), 8.81 (d, J = 1.2 Hz, 1H), 8.32-8.31 (J = 2.8 Hz, 1H), 7.95 (s, 1H), 7.82-7.80 (d, J = 8.4 Hz, 2H), 7.56-7.50 (m, 3H), 7.31 (s, 1H), 6.80-6.78 (d, J = 8.0 Hz, 1H), 3.93 (s, 3H), 1.15 (s, 9H). |
| 161 | 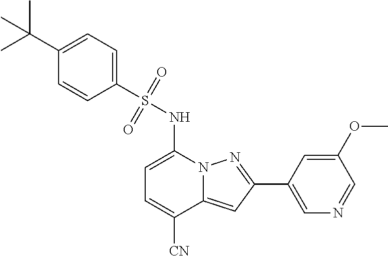 [SMILES: COc1cncc(-c2cc3c(C#N)ccc(NS(=O)(=O)c4ccc(C(C)(C)C)cc4)n3n2)c1] | 462.17 | 99.84% Rt = 6.22 min (2) | ¹H NMR (400 MHz, DMSO- d6): δ 8.81 (s, 1H), 8.28 (s, 1H), 7.91 (s, 1H), 7.76-7.74 (d, J = 8.4 Hz, 2H), 7.48-7.46 (m, 3H), 7.05 (s, 1H), 6.39- 6.37 (d, J = 8.4 Hz, 1H), 3.94 (s, 3H), 1.25 (s, 9H). |

### Example 20

### Synthesis of Compound 162 [4-(tert-butyl)-N-(4-chloro-2-(pyrimidin-5-yl)pyrazolo[1,5-a]pyridin-7-yl)benzenesulfonamide]

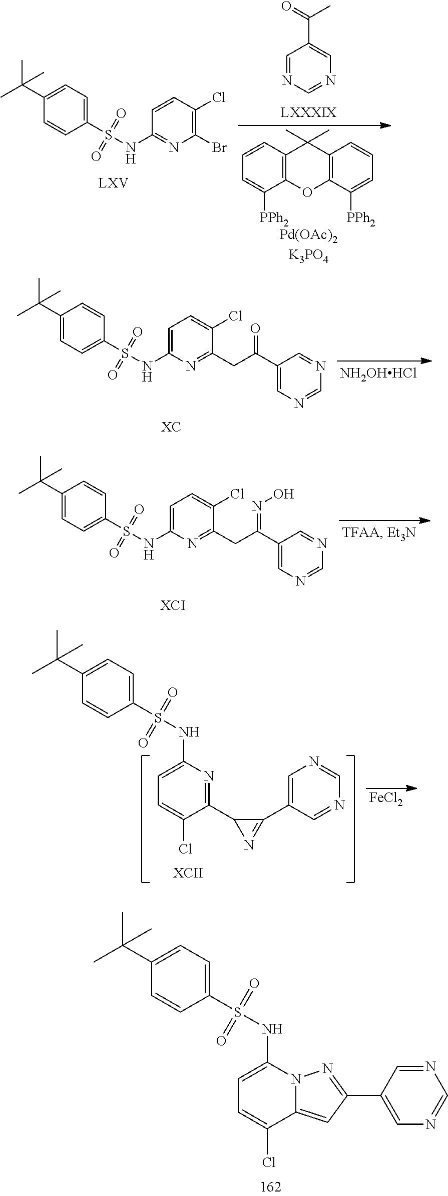

Synthesis of XC:

A stirred solution of compound LXV (0.5 g; 1.24 mmol) and 1-(pyrimidin-5-yl)ethan-1-one (LXXXIX; 0.6 g; 2.48 mmol) in 1,4-dioxane (40 ml) was purged with argon gas for 20 minutes. To the reaction mixture was added 4,5-bis(diphenylphosphino)-9,9-dimethylxanthene (0.57 g, 0.992 mmol), palladium(II)acetate (0.11 g, 0.5 mmol) and potassium phosphate (0.73 g, 3.47 mmol). The resultant solution was stirred at 100° C. for 15 h. The reaction mixture was cooled, concentrated, and filtered through a celite bed. The crude reaction mass was diluted with water and extracted with ethyl acetate. The organic layer was washed with brine solution and dried over anhydrous Na₂SO₄, filtered and evaporated under reduced pressure followed by trituration with hexane to obtain crude 4-(tert-butyl)-N-(5-chloro-6-(2-oxo-2-(pyrimidin-5-yl)ethyl)pyridin-2-yl)benzene sulfonamide XC, as a keto-enol tautomeric mixture. MS (M+1): 445.2.

Synthesis of XCI:

To a stirred solution of compound XC (1.5 g, tautomeric mixture) in methanol (60 ml) was added hydroxylamine hydrochloride (0.93 g; 13.51 mmol) followed by a 10% aqueous solution of sodium hydroxide (193 ml). The resultant suspension was heated at 95° C. for 12 h and then cooled to room temperature. The reaction mixture was concentrated in vacuo and the residue was diluted with water and extracted with ethyl acetate. The organic layer was washed with brine solution and dried over anhydrous Na₂SO₄, filtered and was evaporated under reduced pressure to obtain crude compound, which was purified by column chromatography using 18% ethyl acetate in hexane to afford the desired product (4-(tert-butyl)-N-(5-chloro-6-(2-(hydroxyimino)-2-(pyrimidin-5-yl)ethyl)pyridin-2-yl)benzenesulfonamide as an off white solid (XCI; 0.35 g; 23% yield). ¹H NMR (400 MHz, DMSO-d6) δ 11.90 (s, 1H), 11.03 (bs, 1H), 9.11 (s, 1H), 8.95-8.87 (m, 2H), 7.73-7.69 (m, 3H), 7.53-7.48 (m, 2H), 6.82-6.80 (d, J=8.4 Hz, 1H), 4.26 (s, 2H), 1.26 (s, 9H), MS (M+1): 460.1

### Synthesis of Compound 162: 4-(tert-butyl)-N-(4-chloro-2-(pyrimidin-5-yl)pyrazolo[1,5-a]pyridin-7-yl)benzenesulfonamide

To a stirred solution of compound XCI (0.3 g, 0.65 mmol) in 1,2-dimethoxyethane (10 ml) at 0° C. was added trifluoroacetic anhydride (0.27 g, 1.3 mmol). The reaction mixture was allowed to stir at 0° C. for 20 minutes, followed by dropwise addition of triethylamine (0.66 g, 6.5 mmol) in 1,2-dimethoxyethane (2 ml). The reaction mixture was stirred at room temperature for 3 h resulting in the generation of XCII in situ. To the reaction mixture was further added iron (II) chloride (0.033 g, 0.26 mmol) and this was then heated at 100° C. for 2 h. The reaction mixture was cooled and concentrated, diluted with water and extracted with ethyl acetate. The organic layer was washed with brine solution and dried over anhydrous Na₂SO₄, filtered and evaporated under reduced pressure to obtain crude compound, which was purified by column chromatography using 30% ethyl acetate in hexane to afford the title compound as an off white solid (162; 0.06 g; 20% yield). ¹H NMR (400 MHz, DMSO-d6): δ 11.36 (bs, 1H), 9.34 (s, 2H), 9.21 (s, 1H), 7.80-7.78 (d, J=8.4 Hz, 2H), 7.52-7.50 (d, J=8.4 Hz, 2H), 7.47-7.45 (d, J=8.0 Hz, 1H), 7.43 (s, 1H), 6.91-6.89 (d, J=8.4 Hz, 1H), 1.14 (s, 9H). MS (M+1): 442.37 (LCMS Purity 98.93%, Rt=6.18 min) (1).

### Example 21

### Synthesis of Compound 163 [N-(4-bromo-2-(1H-pyrazol-4-yl)pyrazolo[1,5-a]pyridin-7-yl)-4-(tert-butyl)benzenesulfonamide]; Compound 164 [4-(tert-butyl)-N-(4-cyano-2-(1H-pyrazol-4-yl)pyrazolo[1,5-a]pyridin-7-yl)benzenesulfonamide] and Compounds 165 to 166

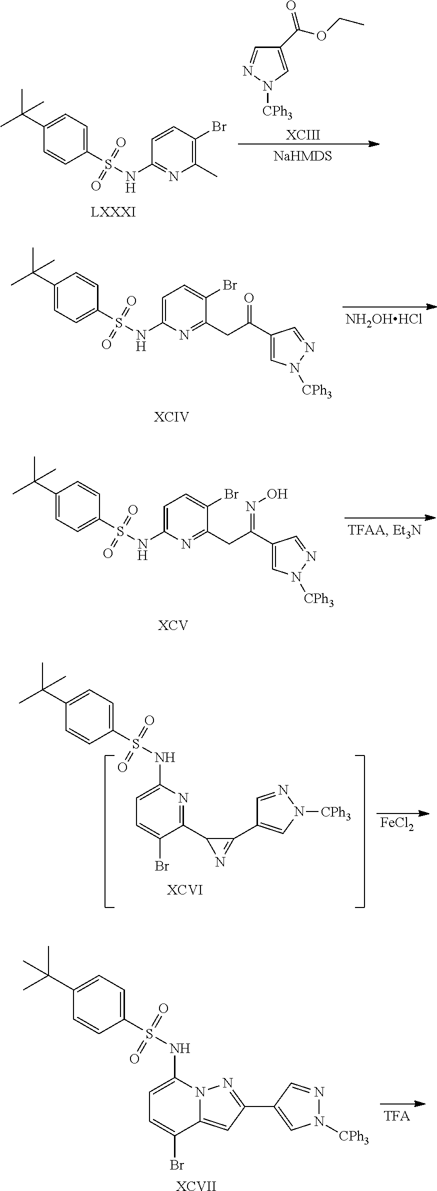

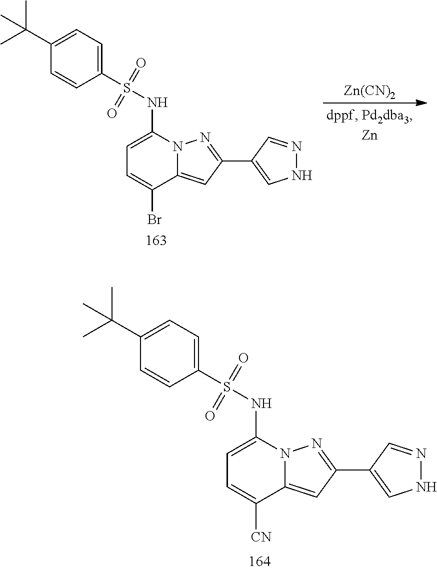

Synthesis of XCIV:

To a stirred solution of compound LXXXI (2 g; 5.23 mmol) and ethyl 1-trityl-1H-pyrazole-4-carboxylate (XCIII; 2.59 g; 6.8 mmol) in THF (50 ml) was added sodium bis(trimethylsilyl)amide (15.7 ml, 1.0 M in THF, 15.7 mmol) drop wise at 0° C. Upon complete addition, the resultant solution was stirred at ambient temperature for 3 h. The reaction mixture was diluted with a saturated solution of ammonium chloride and extracted with ethyl acetate. The organic layer was washed with brine solution and dried over anhydrous Na₂SO₄, filtered and evaporated under reduced pressure to obtain N-(5-bromo-6-(2-oxo-2-(1-trityl-1H-pyrazol-4-yl)ethyl)pyridin-2-yl)-4-(tert-butyl)benzenesulfonamide XCIV, as a keto-enol tautomeric mixture. MS (M+1): 719.12. The crude was carried forward to next step without purification.

Synthesis of XCV:

To a stirred solution of compound XCIV (6.5 g, tautomeric mixture) in methanol (300 ml) was added hydroxylamine hydrochloride (3.13 g; 45.15 mmol) followed by a 10% aqueous solution of sodium hydroxide (40 ml). The resultant suspension was heated at 100° C. for 12 h and then cooled to room temperature. The reaction mixture was concentrated in vacuo and the residue was diluted with water and extracted with ethyl acetate. The organic layer was washed with brine solution and dried over anhydrous Na₂SO₄, filtered and was evaporated under reduced pressure to obtain the desired product (N-(5-bromo-6-(2-(hydroxyimino)-2-(1-trityl-1H-pyrazol-4-yl)ethyl)pyridin-2-yl)-4-(tert-butyl)benzenesulfonamide as an off white solid (XCV; 3 g; 45% yield). MS (M+1): 734.11 (LCMS Purity 93.21%).

Synthesis of XCVII:

To a stirred solution of compound XCV (1 g, 1.36 mmol) in 1,2-dimethoxyethane (20 ml) at 0° C. was added trifluoroacetic anhydride (0.57 g, 2.72 mmol). The reaction mixture was allowed to stir at 0° C. for 20 minutes, followed by dropwise addition of triethylamine (1.37 g, 13.6 mmol) in 1,2-dimethoxyethane (5 ml). The reaction mixture was stirred at room temperature for 1 h to leave XCVI in situ. To the reaction mixture was further added iron (II) chloride (0.068 g, 0.54 mmol) and heated at 100° C. for 3 h. The reaction mixture was cooled and concentrated, diluted with water and extracted with ethyl acetate. The organic layer was washed with brine solution and dried over anhydrous Na₂SO₄, filtered and evaporated under reduced pressure to obtain crude compound, which was purified by column chromatography using 5% ethyl acetate in hexane to afford N-(4-bromo-2-(1-trityl-1H-pyrazol-4-yl)pyrazolo[1,5-a]pyridin-7-yl)-4-(tert-butyl)benzene sulfonamide as off white solid (XCVII; 0.5 g; 51% yield). MS (M+1): 716.1.

### Synthesis of Compound 163; N-(4-bromo-2-(1H-pyrazol-4-yl)pyrazolo[1,5-a]pyridin-7-yl)-4-(tert-butyl)benzenesulfonamide

To a stirred solution of compound XCVII, (0.5 g, 0.69 mmol) in water (5 ml) was added trifluoroacetic acid (2 ml) at 0° C. and stirred for 5 h. The reaction mixture was diluted with water and extracted with dichloromethane. The organic layer was washed with brine solution, saturated aqueous sodium bicarbonate and dried over anhydrous Na₂SO₄, filtered and evaporated under reduced pressure to obtain crude compound, which was triturated with diethyl ether to afford the title compound (163; 0.25 g, 75% yield). ¹H NMR (400 MHz, DMSO-d6): δ 13.07 (bs, 1H), 8.22-8.12 (m, 2H), 7.86-7.83 (d, J=8.8 Hz, 2H), 7.56-7.54 (d, J=8.4 Hz, 2H), 7.46-7.44 (d, J=7.6 Hz, 1H), 6.84 (s, 1H), 6.63-6.61 (d, J=7.6 Hz, 1H), 1.20 (s, 9H). MS (M+1): 476.14. (LCMS Purity 97.60%, Rt=4.48 min) (2).

### Synthesis of Compound 164; 4-(tert-butyl)-N-(4-cyano-2-(1H-pyrazol-4-yl)pyrazolo[1,5-a]pyridin-7-yl)benzenesulfonamide

To a stirred solution of compound 163, (0.2 g, 0.42 mmol) in dimethylacetamide (5 ml) was added Zn(CN)₂ (0.15 g, 0.84 mmol). The reaction vessel and mixture was purged with argon for 20 minutes. To the reaction mixture further added 1, 1′-Bis (diphenylphosphino)ferrocene (0.047 g, 0.084 mmol), Pd₂dba₃ (0.058 g, 0.063 mmol) and a catalytic amount of Zn dust. The reaction mixture was heated at 120° C. for 3 h in a microwave reactor. The reaction mixture was cooled, filtered through a celite bed. The collected filtrate was concentrated, diluted with water and extracted with dichloromethane. The organic layer was washed with brine solution and dried over anhydrous Na₂SO₄, filtered and evaporated under reduced pressure to obtain crude compound, which was purified by column chromatography using 2% methanol in 1% ammoniated dichloromethane to afford the title compound (164; 0.05 g, 28% yield). ¹H NMR (400 MHz, DMSO-d6): δ 13.08 (bs, 1H), 8.13 (m, 2H), 7.75-7.73 (d, J=8.0 Hz, 2H), 7.48-7.46 (d, J=7.6 Hz, 2H), 7.42-7.40 (d, J=8.0 Hz, 1H), 6.65 (s, 1H), 6.35-6.33 (d, J=8.8 Hz, 1H), 1.25 (s, 9H). MS (M+1): 421.24. (LCMS Purity 98.04%, Rt=6.39) (2).

The following compounds were prepared in an essentially similar manner using ethyl 1-methyl-1H-pyrazole-4-carboxylate instead of ethyl 1-trityl-1H-pyrazole-4-carboxylate in the first step. No deprotection chemistry is necessary.

| | | LCMS | Purity | |
|---|---|---|---|---|
| CPD. | Structure | (M + 1) | (LCMS) | ¹H NMR |
| 165 | 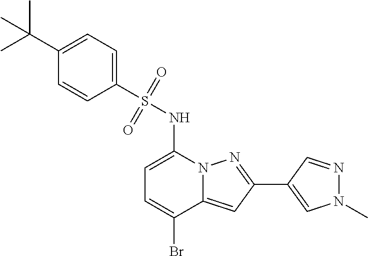 [SMILES: Cn1cc(-c2cc3c(Br)ccc(NS(=O)(=O)c4ccc(C(C)(C)C)cc4)n3n2)cn1] | 490.11 | 96.47% Rt = 4.73 min (2) | ¹H NMR (400 MHz, DMSO- d6): δ 8.17 (s, 1H), 7.87-7.83 (m, 3H), 7.57-7.55 (d, J = 8.0 Hz, 2H), 7.46-7.44 (d, J = 8.0 Hz, 1H), 6.80 (s, 1H), 6.63- 6.61 (d, J = 8.4 Hz, 1H), 3.89 (s, 3H), 1.20 (s, 9H). |
| 166 | 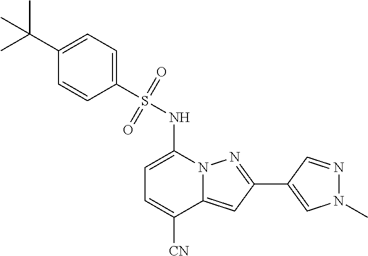 [SMILES: Cn1cc(-c2cc3c(C#N)ccc(NS(=O)(=O)c4ccc(C(C)(C)C)cc4)n3n2)cn1] | 435.48 | 98.90% Rt = 5.29 min (1) | ¹H NMR (400 MHz, DMSO- d6): δ 8.26 (s, 1H), 7.91 (s, 1H), 7.73-7.71 (d, J = 8.0 Hz, 2H), 7.46-7.44 (d, J = 8.0 Hz, 2H), 7.39-7.37 (d, J = 8.4 Hz, 1H), 6.57 (s, 1H), 6.32-6.30 (d, J = 8.4 Hz, 1H), 3.87 (s, 3H), 1.25 (s, 9H). |

### Example 22

### Synthesis of Compound 167 [4-(tert-butyl)-N-(4-chloro-2-(1-methyl-1H-pyrrol-3-yl)pyrazolo[1,5-a]pyri dine-7-yl)benzenesulfonamide]

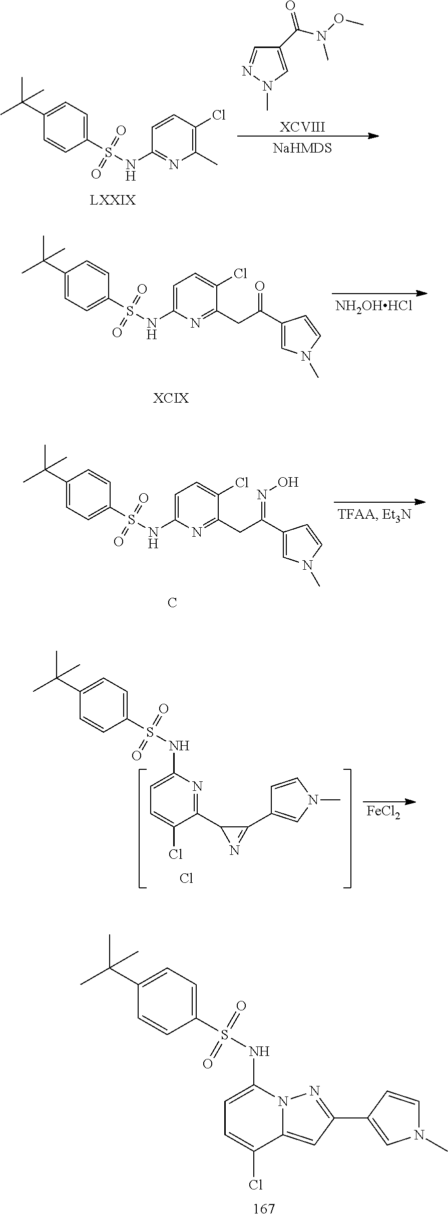

Synthesis of XCIX:

To a stirred solution of compound LXXIX (2 g; 5.90 mmol) and N-methoxy-N, 1-dimethyl-1H-pyrrole-3-carboxamide (XCVIII; 1.48 g; 8.85 mmol) in THF (50 ml) was added sodium bis(trimethylsilyl)amide (47 ml, 1.0 M in THF, 47 mmol) dropwise at 0° C. Upon complete addition, the resultant solution was stirred at ambient temperature for 2 h. The reaction mixture was diluted with a saturated solution of ammonium chloride and extracted with ethyl acetate. The organic layer was washed with brine solution and dried over anhydrous Na₂SO₄, filtered and evaporated under reduced pressure to obtain 4-(tert-butyl)-N-(5-chloro-6-(2-(1-methyl-1H-pyrrol-3-yl)-2-oxoethyl)pyridin-2-yl)benzenesulfonamide XCIX, as a keto-enol tautomeric mixture. MS (M+1): 446.12. The crude material was carried forward to next step without purification.

Synthesis of C:

To a stirred solution of compound XCIX (3 g, tautomeric mixture) in methanol (80 ml) was added hydroxylamine hydrochloride (2.3 g; 33.62 mmol) followed by a 10% aqueous solution of sodium hydroxide (10 ml). The resultant suspension was heated at 100° C. for 12 h and then cooled to room temperature. The reaction mixture was concentrated in vacuo and the residue was diluted with water and extracted with ethyl acetate. The organic layer was washed with brine solution and dried over anhydrous Na₂SO₄, filtered and evaporated under reduced pressure to obtain crude compound, which was purified by column chromatography using 25% ethyl acetate in hexane to afford the desired product 4-(tert-butyl)-N-(5-chloro-6-(2-(hydroxyimino)-2-(1-methyl-1H-pyrrol-3-yl)ethyl)pyridin-2-yl)benzenesulfonamide as an off white solid (C; 0.7 g; 23% yield). MS (M+1): 461.1 (LCMS Purity 99%).

### Synthesis of Compound 167; 4-(tert-butyl)-N-(4-chloro-2-(1-methyl-1H-pyrrol-3-yl)pyrazolo[1,5-a]pyridine-7-yl)benzenesulfonamide

To a stirred solution of C (0.6 g, 1.30 mmol) in 1,2-dimethoxyethane (12 ml) at 0° C. was added trifluoroacetic anhydride (0.54 g, 2.60 mmol). The reaction mixture was allowed to stir at 0° C. for 20 minutes, followed by dropwise addition of triethylamine (1.31 g, 13.0 mmol) in 1,2-dimethoxyethane (2 ml). The reaction mixture was stirred at room temperature for 3 h to form CI in situ. To the reaction mixture was further added iron (II) chloride (0.065 g, 0.52 mmol) and the mixture heated at 100° C. for 2 h. The reaction mixture was cooled and concentrated, diluted with water and extracted with ethyl acetate. The organic layer was washed with brine solution and dried over anhydrous Na₂SO₄, filtered and evaporated under reduced pressure to obtain crude compound, which was purified by column chromatography using 20% ethyl acetate in hexane to afford the title compound as an off white solid (167; 0.02 g). ¹H NMR (400 MHz, DMSO-d6): δ 8.88-8.86 (d, J=8.0 Hz, 2H), 7.57-7.55 (d, J=8.0 Hz, 2H), 7.28-7.26 (d. J=8.0 Hz, 2H), 6.75-6.72 (d, J=12.4 Hz, 2H), 6.61-6.59 (d, J=8 Hz, 1H), 6.47 (s, 1H), 3.66 (s, 3H), 1.21 (s, 9H). MS (M+1): 443.19 (LCMS Purity 94.12%, Rt=5.12 min) (2).

### Example 23

### Synthesis of Compound 168 [4-(tert-butyl)-N-(4-chloro-2-(thiazol-5-yl)pyrazolo[1,5-a]pyridin-7-yl)benzenesulfonamide]

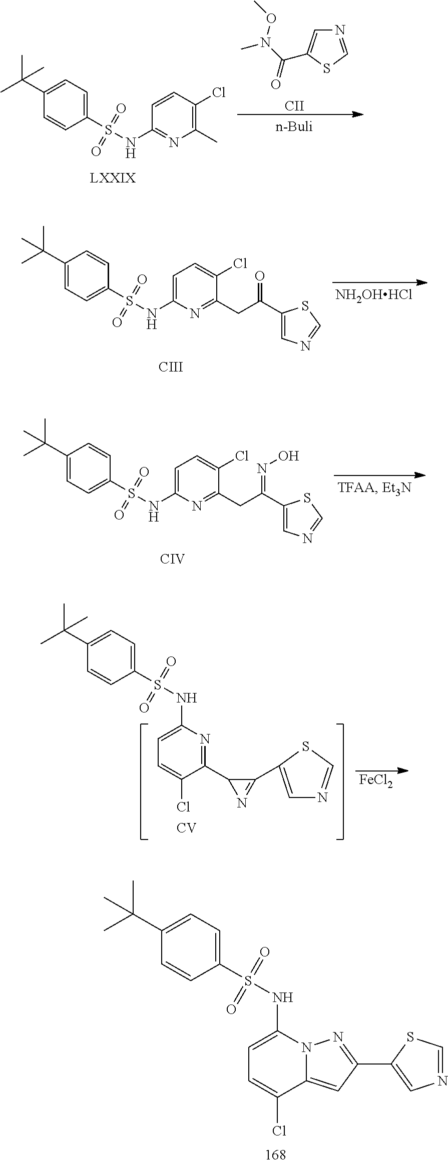

Synthesis of CIII:

To a stirred solution of compound LXXIX (1.5 g; 4.43 mmol) in THF (20 ml) was added n-butyl lithium (14 ml, 1.6M in hexane, 22.15 mmol) dropwise at −78° C. After stirring for 15 min, N-methoxy-N-methylthiazole-5-carboxamide (CII, 1.28 g; 13.3 mmol) in THF was added. The resultant solution was stirred at ambient temperature for 15 min. The reaction mixture was diluted with a saturated solution of ammonium chloride and extracted with ethyl acetate. The organic layer was washed with brine solution and dried over anhydrous Na₂SO₄, filtered and evaporated under reduced pressure to obtain 4-(tert-butyl)-N-(5-chloro-6-(2-oxo-2-(thiazol-5-yl)ethyl)pyridin-2-yl)benzenesulfonamide CIII, as a keto-enol tautomeric mixture. MS (M+1): 450.2. The crude was carried forward to next step without purification.

Synthesis of CIV:

To a stirred solution of compound CIII (2.2 g, tautomeric mixture) in methanol (20 ml) was added hydroxylamine hydrate (1.05 g; 14.69 mmol) followed by a 10% aqueous solution of sodium hydroxide (15 ml). The resultant suspension was heated at 100° C. for 12 h and then cooled to room temperature. The reaction mixture was concentrated in vacuo and the residue was diluted with water and extracted with ethyl acetate. The organic layer was washed with brine solution and dried over anhydrous Na₂SO₄, filtered and evaporated under reduced pressure to obtain crude compound, which was purified by column chromatography using 30% ethyl acetate in hexane to afford desired product 4-(tert-butyl)-N-(5-chloro-6-(2-(hydroxyimino)-2-(thiazol-5-yl)ethyl)pyridin-2-yl)benzenesulfonamide as an off white solid (CIV; 1.2 g; 53% yield). MS (M+1): 465.12.

### Synthesis of Compound 168; 4-(tert-butyl)-N-(4-chloro-2-(thiazol-5-yl)pyrazolo[1,5-a]pyridin-7-yl)benzenesulfonamide

To a stirred solution of compound CIV (0.5 g, 1.07 mmol) in 1,2-dimethoxyethane (12 ml) at 0° C. was added trifluoroacetic anhydride (0.18 g, 0.86 mmol). The reaction mixture was allowed to stir at 0° C. for 20 minutes and triethylamine (0.54 g, 5.35 mmol) in 1,2-dimethoxyethane (2 ml) was added in dropwise fashion. The reaction mixture was stirred at room temperature for 3 h leading to the preparation of Compound CV in situ. To the reaction mixture was further added iron (II) chloride (0.054 g, 0.42 mmol) and the resulting suspension was heated at 100° C. for 2 h. The reaction mixture was cooled and concentrated, diluted with water and extracted with ethyl acetate. The organic layer was washed with brine solution and dried over anhydrous Na₂SO₄, filtered and evaporated under reduced pressure to obtain crude compound, which was purified by column chromatography using 35% ethyl acetate in hexane to afford the title compound as an off white solid (168; 0.04 g). ¹H NMR (400 MHz, DMSO-d6): δ 11.32 (bs, 1H), 9.14 (s, 1H), 8.48 (s, 1H), 7.78-7.76 (d, J=8.4 Hz, 2H), 7.50-7.48 (d, J=7.2 Hz, 2H), 7.35 (m, 1H), 7.13 (s, 1H), 6.66 (m, 1H), 1.18 (s, 9H). MS (M+1): 447.37 (LCMS Purity 96.76%, Rt=5.75 min) (1).

### Example 24

### Synthesis of Compound 169 [4-(tert-butyl)-N-(4-chloro-2-(oxazol-5-yl)pyrazolo[1,5-a]pyridin-7-yl)benzenesulfonamide]; and Compound 170 [4-(tert-butyl)-N-(4-cyano-2-(oxazol-5-yl)pyrazolo[1,5-a]pyridin-7-yl)benzenesulfonamide]

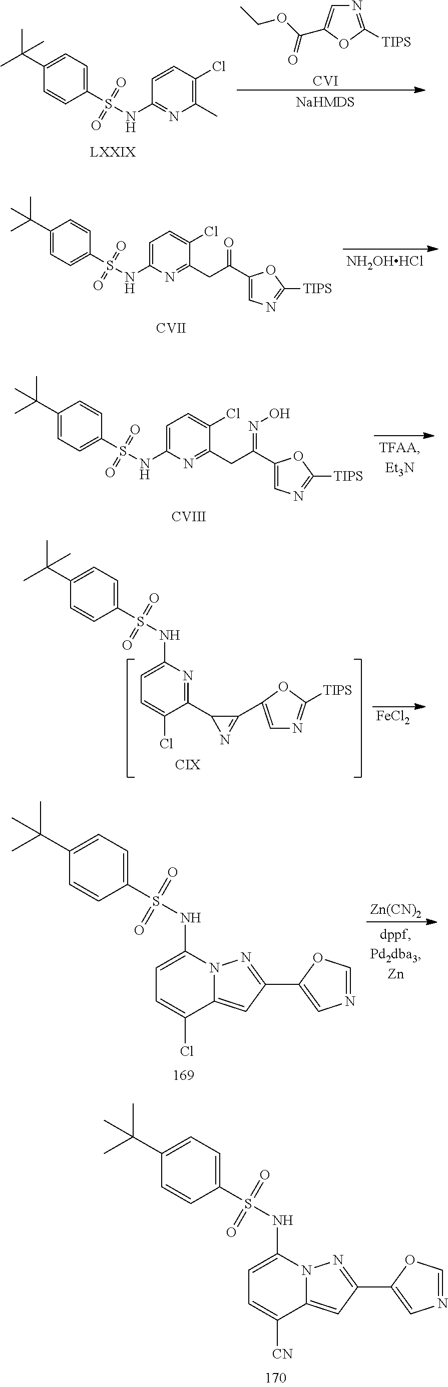

Synthesis of CVII:

To a stirred solution of compound LXXIX (1.7 g; 5.02 mmol) and ethyl 2-(triisopropylsilyl)oxazole-5-carboxylate (CVI; 5.9 g; 20.11 mmol) in THF (25 ml) was added sodium bis(trimethylsilyl)amide (50 m, 1.0 M in THF, 50 mmol) dropwise at 0° C. The resultant solution was stirred at ambient temperature for 3 h. The reaction mixture was diluted with a saturated solution of ammonium chloride and extracted with ethyl acetate. The organic layer was washed with brine solution and dried over anhydrous Na₂SO₄, filtered and evaporated under reduced pressure to obtain 4-(tert-butyl)-N-(5-chloro-6-(2-oxo-2-(2-(triisopropylsilyl)oxazol-5-yl)ethyl)pyridin-2-yl)benzenesulfon amide CVII, as a keto-enol tautomeric mixture. MS (M+1): 590.2. The crude material was carried forward to next step without purification.

Synthesis of CVIII:

To a stirred solution of compound CVII (3.2 g, tautomeric mixture) in methanol (26 ml) was added hydroxylamine hydrate (0.53 g; 16.29 mmol) followed by a 10% aqueous solution of sodium hydroxide (30 ml). The resultant suspension was heated at 100° C. for 12 h and then cooled to room temperature. The reaction mixture was concentrated in vacuo and the residue was diluted with water and extracted with ethyl acetate. The organic layer was washed with brine solution and dried over anhydrous Na₂SO₄, filtered and was evaporated under reduced pressure to obtain crude compound, which was purified by column chromatography using 30% ethyl acetate in hexane to afford the desired product 4-(tert-butyl)-N-(5-chloro-6-(2-(hydroxyimino)-2-(2-(triisopropylsilyl) oxazol-5-yl)ethyl)pyridin-2-yl)benzenesulfonamide as an off white solid (CVIII; 0.3 g; 9% yield. MS (M+1): 605.12.

### Synthesis of Compound 169; 4-(tert-butyl)-N-(4-chloro-2-(oxazol-5-yl)pyrazolo[1,5-a]pyridin-7-yl)benzene sulfonamide

To a stirred solution of CVIII (0.3 g, 0.49 mmol) in 1,2-dimethoxyethane (12 ml) at 0° C. was added trifluoroacetic anhydride (0.082 g, 0.39 mmol). The reaction mixture was allowed to stir at 0° C. for 20 minutes, followed by dropwise addition of triethylamine (0.24 g, 2.45 mmol) in 1,2-dimethoxyethane (2 ml). The reaction mixture was stirred at room temperature for 3 h to prepare CIX in situ. To the reaction mixture further added iron (II) chloride (0.024 g, 0.19 mmol) and this was heated at 100° C. for 2 h. The reaction mixture was cooled and concentrated, diluted with water and extracted with ethyl acetate. The organic layer was washed with brine solution and dried over anhydrous Na₂SO₄, filtered and evaporated under reduced pressure to obtain crude compound, which was purified by column chromatography using 30% ethyl acetate in hexane to afford the title compound as an off white solid (169; 0.05 g; 23% yield). ¹H NMR (400 MHz, DMSO-d6): δ 8.55 (s, 1H), 7.82-7.80 (d, J=8.4 Hz, 2H), 7.68 (s, 1H), 7.54-7.52 (d, J=8.4 Hz, 2H), 7.45-7.43 (d, J=7.6 Hz, 1H), 7.04 (s, 1H), 6.78-6.76 (d, J=8.0 Hz, 1H), 1.19 (s, 9H). MS (M+1): 431.35 (LCMS Purity 97.44%, Rt=5.67 min) (1).

### Synthesis of Compound 170; 4-(tert-butyl)-N-(4-cyano-2-(oxazol-5-yl)pyrazolo[1,5-a]pyridin-7-yl)benzene sulfonamide

To a stirred solution of compound 169, (0.07 g, 0.16 mmol) in dimethylacetamide (5 ml) was added Zn(CN)₂ (0.025 g, 0.20 mmol). The reaction mixture was purged with argon for 20 min. To the reaction mixture was further added 1,1′-Bis (diphenylphosphino)ferrocene (0.08 g, 0.14 mmol), Pd₂dba₃ (0.12 g, 0.14 mmol) and a catalytic amount of Zn dust. The reaction mixture was heated at 120° C. for 2 h in a microwave reactor. The reaction mixture was cooled and filtered through a celite bed. The filtrate was concentrated, diluted with water and extracted with dichloromethane. The organic layer was washed with brine solution and dried over anhydrous Na₂SO₄, filtered and evaporated under reduced pressure to obtain the crude compound, which was purified by column chromatography using 2% methanol in dichloromethane to afford the title compound (170; 0.013 g, 20% yield). ¹H NMR (400 MHz, DMSO-d6): δ 8.50 (s, 1H), 7.76-7.72 (m, 3H), 7.50-7.46 (m, 3H), 6.72 (s, 1H), 6.43-6.41 (d, J=8.0 Hz, 1H), 1.25 (s, 9H). MS (M+1): 422.46 (LCMS Purity 99.60%, Rt=5.16 min) (1).

### Example 25

### Synthesis of Compound 171 [methyl 3-(7-((4-(tert-butyl)phenyl)sulfonamido)-4-chloropyrazolo[1,5-a]pyridin-2-yl)thiophene-2-carboxylate] and Compound 172 [3-(7-((4-(tert-butyl)phenyl)sulfonamido)-4-chloropyrazolo[1,5-a]pyridin-2-yl)thiophene-2-carboxylic acid]

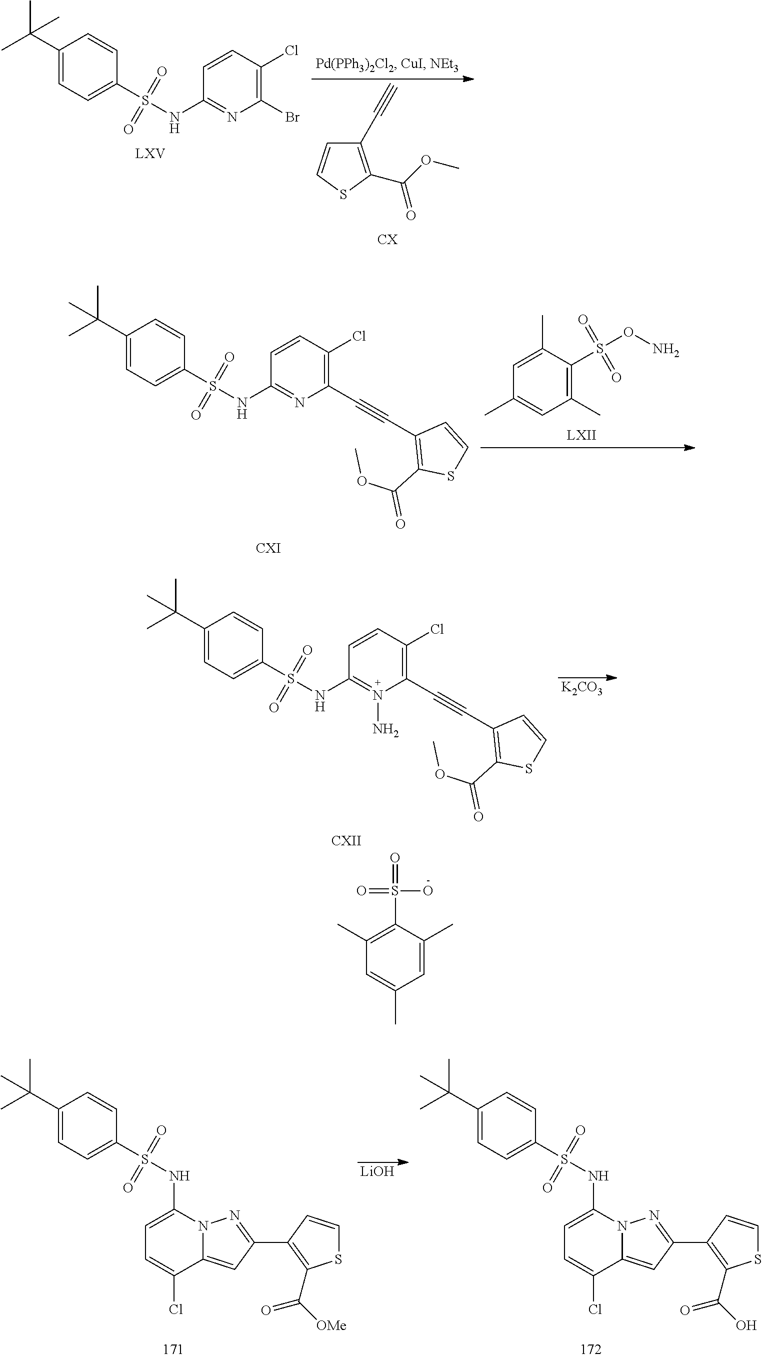

Synthesis of CXI:

A stirred solution of compound LXV (1 g, 2.48 mmol) in dimethylformide (40 ml) was placed in a sealed tube which was purged with argon for 20 minutes. To the reaction mixture was added Bis(triphenylphosphine)palladium(II) chloride (0.26 g, 0.37 mmol), copper(I)iodide (0.07 g, 0.37 mmol) and triethylamine (0.72 g, 7.19 mmol). The reaction mixture was cooled to 0° C., followed by addition of methyl 3-ethynylthiophene-2-carboxylate (CX; 2 g, 12.0 mmol). The reaction mixture was re-sealed and heated at 100° C. for 24 h. The reaction mixture was cooled and filtered through a celite bed. The collected filtrate was concentrated, diluted with water and extracted with ethyl acetate. The organic layer was washed with brine solution and dried over anhydrous Na₂SO₄, filtered and evaporated under reduced pressure to obtain crude compound CXI, methyl 3-((6-((4-(tert-butyl)phenyl)sulfonamido)-3-chloropyridin-2-yl)ethynyl)thiophene-2-carboxylate. MS (M+1): 489.16.

Synthesis of CXII:

To a stirred solution of CXI (0.2 g, 0.41 mmol) in dichloromethane (5 ml) was added O-(mesitylsulfonyl) hydroxylamine (LXII; 1 g). The reaction mixture was stirred for 24 h at room temperature, diluted with water and extracted with dichloromethane which was washed with a saturated aqueous solution of sodium bicarbonate. The organic layer was further washed with brine solution, dried over anhydrous Na₂SO₄, filtered and evaporated under reduced pressure to obtain crude compound CXII, 1-amino-6-((4-(tert-butyl)phenyl)sulfonamido)-3-chloro-2-((2-(methoxycarbonyl)thiophen-3-yl)ethynyl)pyridin-1-ium 2,4,0-trimethylbenzenesulfonate. MS (M+1): 505.12. The crude material was carried forward to next step without purification.

### Synthesis of Compound 171: methyl 3-(7-((4-(tert-butyl)phenyl)sulfonamido)-4-chloropyrazolo[1,5-a]pyridin-2-yl)thiophene-2-carboxylate

To a stirred solution of CXII (0.2 g, crude) in dimethylformide (3 ml) was added potassium carbonate (0.27 g, 1.98 mmol). The reaction mixture was stirred at 60° C. for 1 h. The reaction mixture was concentrated in vacuo and the residue was diluted with water and extracted with ethyl acetate. The organic layer was washed with brine solution and dried over anhydrous Na₂SO₄, filtered and evaporated under reduced pressure to obtain crude compound, which was purified by preparative HPLC to afford the title compound (171; 0.019 g). ¹H NMR (400 MHz, DMSO-d6): δ 11.34 (bs, 1H), 7.97-7.96 (d, J=5.2 Hz, 1H), 7.82-7.80 (d, J=8.8 Hz, 2H), 7.70-7.69 (d, J=5.2 Hz, 1H), 7.54-7.52 (d, J=8.4 Hz, 2H), 7.42-7.40 (m, 2H), 6.84-6.82 (d, J=8.4 Hz, 1H), 3.81 (s, 3H), 1.19 (s, 9H). MS (M+1): 504.12. (LCMS Purity 97.74%, Rt=5.34 min) (2).

### Synthesis of Compound 172; 3-(7-((4-(tert-butyl)phenyl)sulfonamido)-4-chloropyrazolo[1,5-a]pyridin-2-yl)thiophene-2-carboxylic acid

To a stirred solution of 171 (0.09 g, 0.17 mmol) in a mixture of methanol, tetrahydrofuran and water (1:1:0.5) (2.5 ml) was added lithium hydroxide (0.013 g, 0.53 mmol). The reaction mixture was stirred at room temperature for 12 h. This was concentrated under reduced pressure, diluted with water and acidified with an aqueous solution of potassium bisulphate to pH 1-2. The aqueous layer was extracted with ethyl acetate, which was washed with brine solution, dried over anhydrous Na₂SO₄, filtered and evaporated under reduced pressure to afford the title compound as an off white solid (172; 0.021 g; 20% yield). ¹H NMR (400 MHz, DMSO-d6): δ 7.90-7.89 (d, J=4.8 Hz, 1H), 7.82-7.80 (d, J=8.4 Hz, 2H), 7.69-7.68 (d, J=5.2 Hz, 1H), 7.53-7.51 (d, J=8.8 Hz, 2H), 7.40-7.38 (m, 2H), 6.81-6.79 (d, J=8.0 Hz, 1H), 1.19 (s, 9H). MS (M+1): 490.11. (LCMS Purity 98.97%, Rt=6.90 min) (2).

### Example 26

### Synthesis of Compound 173 [4-(tert-butyl)-N-(4-methoxy-2-(pyridin-3-yl)pyrazolo[1,5-a]pyridin-7-yl)benzenesulfonamide]; and Compounds 174-175

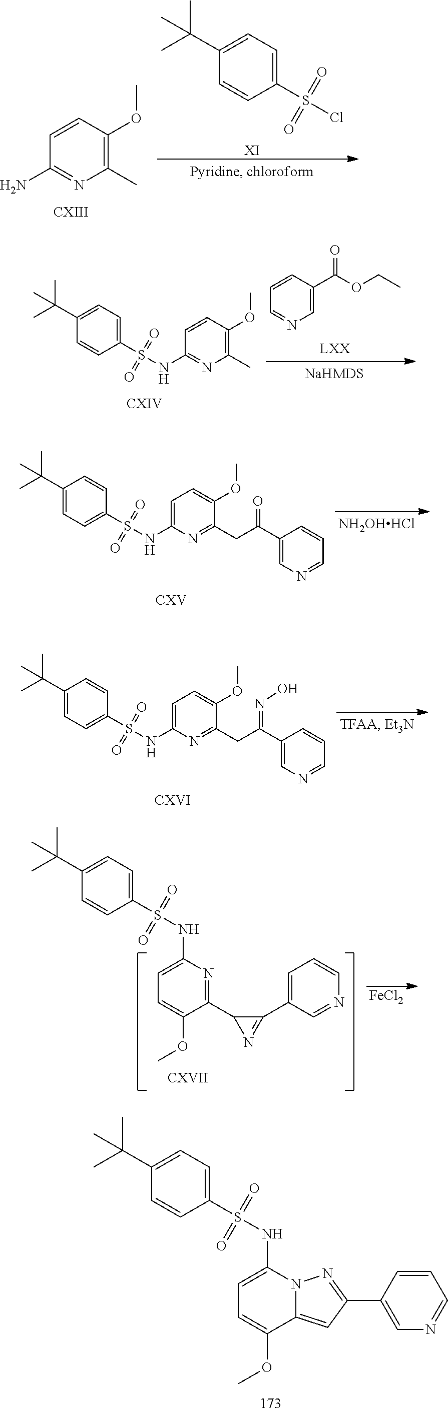

Synthesis of CXIV:

To a stirred solution of compound CXIII (0.38 g, 2.75 mmol) in chloroform (2 ml) was added pyridine (7.6 ml) at 0° C. followed by addition of 4-tert-butylbenzenesulfonyl chloride (XI, 0.76 g, 3.3 mmol). The reaction mixture was heated at 100° C. for 12 h and then cooled to room temperature and concentrated under reduced pressure. The crude mass was diluted with a saturated ammonium chloride solution and extracted with ethyl acetate. The organic layer was washed with brine solution, dried over anhydrous Na₂SO₄ and evaporated under reduced pressure to afford 4-(tert-butyl)-N-(5-methoxy-6-methylpyridin-2-yl)benzenesulfonamide (CXIV; 0.89 g, 97% yield). ¹H NMR (400 MHz, CDCl3): δ 7.75-7.73 (d, J=8.4 Hz, 2H), 7.44-7.42 (d, J=8.4 Hz, 2H), 7.24 (m, 1H), 7.09-7.07 (d, J=8.4 Hz, 1H), 3.78 (s, 3H), 2.28 (s, 3H), 1.29 (s, 9H). MS (M+1): 335.2.

Synthesis of CXV:

To a stirred solution of compound CXIV (0.89 g; 2.66 mmol) and ethyl nicotinate (LXX; 0.44 g; 2.92 mmol) in THF (30 ml) was added sodium bis(trimethylsilyl)amide (8 ml, 1.0 M in THF, 7.98 mmol) dropwise at 0° C. The resultant solution was stirred at ambient temperature for 3 h.

The reaction mixture was diluted with a saturated solution of ammonium chloride and extracted with ethyl acetate. The organic layer was washed with brine solution and dried over anhydrous Na₂SO₄, filtered and evaporated under reduced pressure to obtain 4-(tert-butyl)-N-(5-methoxy-6-(2-oxo-2-(pyridin-3-yl)ethyl)pyridin-2-yl)benzenesulfonamide CXV, as a keto-enol tautomeric mixture. MS (M+1): 440.2. The crude material was carried forward to next step without purification.

Synthesis of CXVI:

To a stirred solution of compound 275 (1 g, tautomeric mixture) in methanol (100 ml) was added hydroxylamine hydrochloride (0.79 g; 11.38 mmol) followed by a 10% aqueous solution of sodium hydroxide (10 ml). The resultant suspension was heated at 100° C. for 12 h. The reaction mixture was cooled and concentrated in vacuo. The residue was diluted with water and extracted with ethyl acetate. The organic layer was washed with brine solution and dried over anhydrous Na₂SO₄, filtered and evaporated under reduced pressure to obtain crude compound, which was purified by column chromatography using 60% ethyl acetate in hexane to afford desired product 4-(tert-butyl)-N-(6-(2-(hydroxyimino)-2-(pyridin-3-yl)ethyl)-5-methoxypyridin-2-yl)benzene sulfonamide as off white solid (CXVI; 0.8 g; 79% yield). MS (M+1): 455.1.

### Synthesis of 173; 4-(tert-butyl)-N-(4-methoxy-2-(pyridin-3-yl)pyrazolo[1,5-a]pyridin-7-yl)benzenesulfonamide

To a stirred solution of CXVI (0.82 g, 1.80 mmol) in 1,2-dimethoxyethane (15 ml) at 0° C. was added trifluoroacetic anhydride (0.75 g, 3.6 mmol). The reaction mixture was allowed to stir at 0° C. for 20 minutes, followed by dropwise addition of triethylamine (0.91 g, 9 mmol) in 1,2-dimethoxyethane (2 ml). The reaction mixture was stirred at room temperature for 2 h to leave CXVII in situ. To the reaction mixture was further added iron (II) chloride (0.09 g, 0.72 mmol) and this was heated at 90° C. for 2 h. The reaction mixture was cooled, concentrated, diluted with water and extracted with ethyl acetate. The organic layer was washed with brine solution and dried over anhydrous Na₂SO₄, filtered and evaporated under reduced pressure to obtain crude compound, which was purified by column chromatography using 30% ethyl acetate in hexane to afford the title compound as off white solid (173; 0.04 g). ¹H NMR (400 MHz, DMSO-d6): δ 10.74 (bs, 1H), 8.99 (d, J=1.6 Hz, 1H), 8.54-8.53 (m, 1H), 8.16-8.14 (d, J=7.6 Hz, 1H), 7.65-7.62 (d, J=8.4 Hz, 2H), 7.46-7.39 (m, 3H), 7.17 (s, 1H), 6.79-6.77 (d, J=8 Hz, 1H), 6.69-6.67 (d, J=8.4 Hz, 1H), 3.94 (s, 3H), 1.05 (s, 9H) MS (M+1): 437.39. (LCMS Purity 97.17%, Rt=5.95 min) (1).

The following compounds were prepared in a similar manner using the appropriate sulfonyl chloride.

| | | LCMS | Purity | |
|---|---|---|---|---|
| CPD | Structure | (M + 1) | (LCMS) | ¹H NMR |
| 174 | 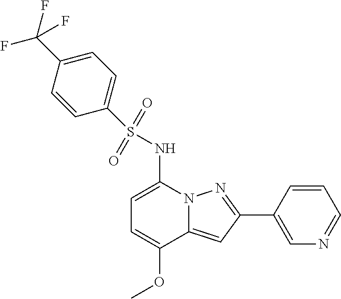 [SMILES: COc1ccc(NS(=O)(=O)c2ccc(C(F)(F)F)cc2)n2nc(-c3cccnc3)cc12] | 449.13 | 96.03%, Rt = 5.04 min (1) | ¹H NMR (400 MHz, DMSO- d6): δ 11.16 (bs, 1H), 8.93- 8.93 (d, J = 1.2 Hz, 1H), 8.53- 8.53 (d, J = 3.6 Hz, 1H), 7.98- 7.96 (d, J = 8.4 Hz, 1H), 7.93- 7.91 (d, J = 8.0 Hz, 2H), 7.81- 7.79 (d, J = 8.4, 2H), 7.41- 7.38 (m, 1H), 7.20 (s, 1H), 6.83-6.81 (d, J = 8.0 Hz, 1H), 6.70-6.68 (d, J = 8.0 Hz 1H), 3.95 (s, 3H) |
| 175 | 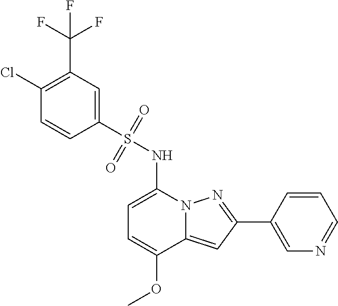 [SMILES: COc1ccc(NS(=O)(=O)c2ccc(Cl)c(C(F)(F)F)c2)n2nc(-c3cccnc3)cc12] | 483.06 | 97.45%, Rt = 5.53 min (2) | ¹H NMR (400 MHz, DMSO- d6): δ 11.28 (bs, 1H), 9.02 (s, 1H), 8.63 (m, 1H), 8.12 (m, 1H), 8.00-7.97 (m, 2H), 7.79 (m, 1H), 7.56 (m, 1H), 7.28 (s, 1H), 6.89 (m, 1H), 6.73 (m, 1H), 3.96 (s, 3H). |

### Example 27

### Synthesis of Compound 176 [N-(4-bromo-2-(pyridin-3-yl)pyrazolo[1,5-a]pyridin-7-yl)-4-(tert-butyl)benzenesulfonamide]; and Compound 177 [4-(tert-butyl)-N-(4-(methylsulfonyl)-2-(pyridin-3-yl)pyrazolo[1,5-a]pyridin-7-yl)benzenesulfonamide]

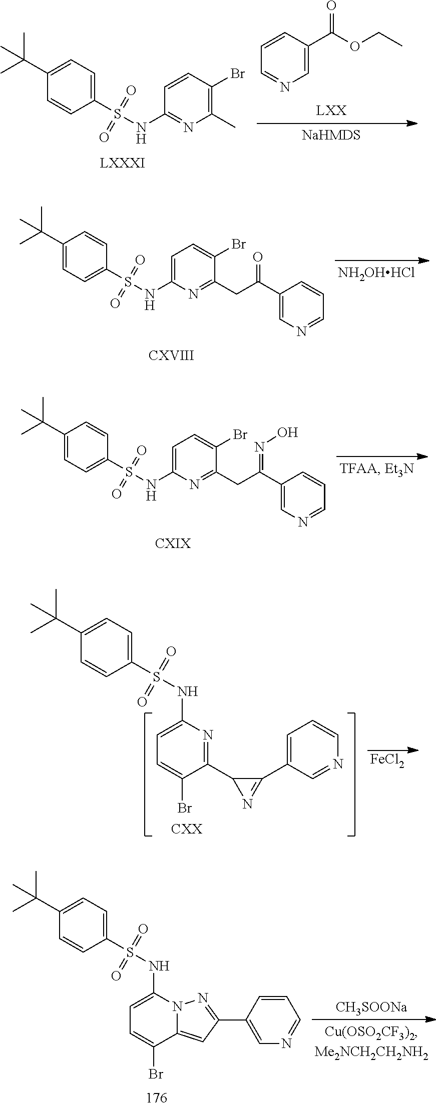

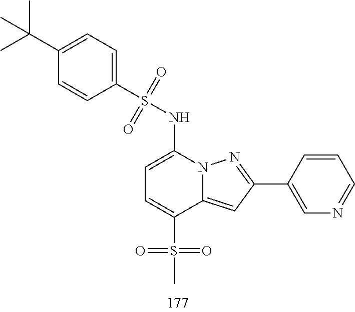 [SMILES: CC(C)(C)c1ccc(S(=O)(=O)Nc2ccc(S(C)(=O)=O)c3cc(-c4cccnc4)nn23)cc1]

Synthesis of CXVIII:

To a stirred solution of compound LXXXI (5 g; 13.08 mmol) and ethyl nicotinate (LXX; 5.96 g; 39.24 mmol) in THF (60 ml) was added sodium bis(trimethylsilyl)amide (59 ml, 1.0 M in THF, 58.86 mmol) dropwise at 0° C. Upon complete addition, the resultant solution was stirred at ambient temperature for 6 h. The reaction mixture was diluted with a saturated solution of ammonium chloride and extracted with ethyl acetate. The organic layer was washed with brine solution and dried over anhydrous Na₂SO₄, filtered and evaporated under reduced pressure to obtain CXVIII, N-(5-bromo-6-(2-oxo-2-(pyridin-3-yl)ethyl)pyridin-2-yl)-4-(tert-butyl)benzene sulfonamide as a keto-enol tautomeric mixture. MS (M+1):488.2. The crude material was carried forward to next step without purification.

Synthesis of CXIX:

To a stirred solution of compound CXVIII (15 g, tautomeric mixture) in methanol (100 ml) was added hydroxylamine hydrochloride (15 g; 215 mmol) followed by a 10% aqueous solution of sodium hydroxide (40 ml). The resultant suspension was heated at 90° C. for 10 h and then cooled to room temperature. The reaction mixture was concentrated in vacuo and the residue was diluted with water and extracted with ethyl acetate. The organic layer was washed with brine solution and dried over anhydrous Na₂SO₄, filtered and evaporated under reduced pressure to obtain crude compound, which was purified by column chromatography using 40% ethyl acetate in hexane to afford the desired product N-(5-bromo-6-(2-(hydroxyimino)-2-(pyridin-3-yl)ethyl)pyridin-2-yl)-4-(tert-butyl)benzenesulfonamide (CXIX; 9 g; 58% yield). ¹H NMR (400 MHz, DMSO-d6) δ 11.64 (s, 1H), 11.03 (bs, 1H), 8.66 (s, 1H), 8.47-8.46 (d, J=3.6 Hz, 1H), 7.82-7.74 (m, 4H), 7.50-7.47 (d, J=8.8 Hz, 2H), 7.28-7.25 (m, 1H), 6.72-6.70 (d, J=8.4 Hz, 1H), 4.23 (s, 2H), 1.25 (s, 9H). MS (M+1): 505.32 (LCMS Purity 95.64%).

### Synthesis of Compound 176; N-(4-bromo-2-(pyridin-3-yl)pyrazolo[1,5-a]pyridin-7-yl)-4-(tert-butyl)benzenesulfonamide

To a stirred solution of compound CXIX (1 g, 1.99 mmol) in 1,2-dimethoxyethane (15 ml) at 0° C. was added trifluoroacetic anhydride (0.83 g, 3.98 mmol). The reaction mixture was allowed to stir at 20° C. for 20 minutes, followed by dropwise addition of triethylamine (2.01 g, 19.9 mmol) in 1,2-dimethoxyethane (10 ml). The reaction mixture was stirred at room temperature for 2 h, forming CXX in situ. To the reaction mixture was further added iron (II) chloride (0.1 g, 0.79 mmol) and heated at 100° C. for 2 h. The reaction mixture was cooled and concentrated, diluted with water and extracted with ethyl acetate. The organic layer was washed with brine solution and dried over anhydrous Na₂SO₄, filtered and evaporated under reduced pressure to obtain the crude compound, which was purified by column chromatography using 25% ethyl acetate in hexane to afford the title compound as an off white solid (176; 0.2 g; 20% yield). ¹H NMR (400 MHz, DMSO-d6): δ 11.32 (bs, 1H), 9.23 (s, 1H), 8.60-8.59 (d, J=4.8 Hz, 1H), 8.38-8.36 (d, J=8.0 Hz, 1H), 7.83-7.81 (d, J=8.4 Hz, 2H), 7.56-7.49 (m, 4H), 7.25 (s, 1H), 6.80-6.78 (d, J=8.0 Hz, 1H), 1.15 (s, 9H). MS (M+1): 487.09.1 (LCMS Purity 99.12%, Rt=6.21 min) (2).

### Synthesis of Compound 177; 4-(tert-butyl)-N-(4-(methylsulfonyl)-2-(pyridin-3-yl)pyrazolo[1,5-a]pyridin-7-yl)benzenesulfonamide

To a stirred solution of 176 (0.25 g, 0.51 mmol) in dimethylsulfoxide (10 ml) was added sodium methanesulfinate (0.26 g, 2.55 mmol), copper (II) triflate (0.22 g, 0.61 mmol) and N, N-dimethylethylene diamine (0.05 g, 0.51 mmol). The reaction mixture was heated at 120° C. for 1 h in a microwave reactor. The reaction mixture was cooled and concentrated, diluted with water and extracted with ethyl acetate. The organic layer was washed with brine solution and dried over anhydrous Na₂SO₄, filtered and evaporated under reduced pressure to obtain the crude compound, which was purified by preparative HPLC to afford the title compound as an off white solid (177; 0.03 g). ¹H NMR (400 MHz, DMSO-d6): δ 9.41 (s, 1H), 8.71 (m, 2H), 7.95-7.93 (d, J=8.4 Hz, 2H), 7.75-7.69 (m, 2H), 7.60-7.58 (d, J=8.4 Hz, 2H), 7.46 (s, 1H), 6.93-6.91 (m, 1H), 3.26 (s, 3H), 1.24 (s, 9H). MS (M+1): 485.16. (LCMS Purity 99.22%, Rt=5.64 min) (2).

### Example 28

### Synthesis of Compound 178 [4-(tert-butyl)-N-(4-cyclopropyl-2-(pyridin-3-yl)pyrazolo[1,5-a]pyridin-7-yl)benzenesulfonamidel]

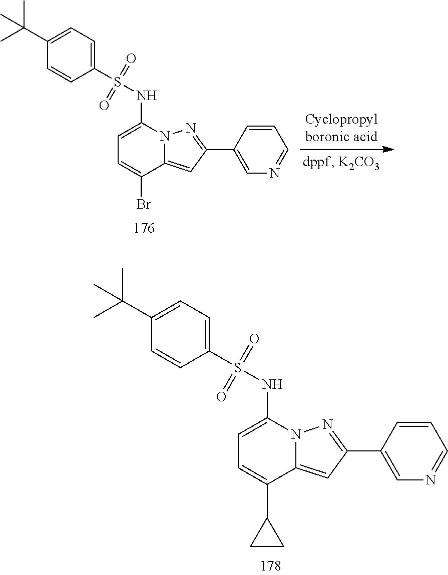 [SMILES: CC(C)(C)c1ccc(S(=O)(=O)Nc2ccc(Br)c3cc(-c4cccnc4)nn23)cc1 ; CC(C)(C)c1ccc(S(=O)(=O)Nc2ccc(C3CC3)c3cc(-c4cccnc4)nn23)cc1]

A stirred solution of compound 176 (0.15 g, 0.31 mmol) in 1,4-dioxane (8 ml) was purged with argon for 20 minutes, followed by addition of cyclopropylboronic acid (0.16 g, 1.86 mmol), [1,1′-Bis(diphenylphosphino)ferrocene] dichloropalladium(II), complex with dichloromethane (0.05 g, 0.06 mmol) and potassium carbonate (0.13 g, 0.9 mmol). The reaction mixture was heated at 120° C. for 12 h. The reaction mixture was cooled and filtered through a celite bed. The filtrate was concentrated, diluted with water and extracted with dichloromethane. The organic layer was washed with brine solution and dried over anhydrous Na₂SO₄, filtered and evaporated under reduced pressure to obtain crude compound, which was purified by column chromatography using 2% methanol in dichloromethane to afford the title compound (178; 0.01 g). ¹H NMR (400 MHz, DMSO-d6): δ 9.18 (s, 1H), 8.53-8.53 (d, J=3.6 Hz, 1H), 8.36-8.34 (d, J=7.6 Hz, 1H), 7.71-7.69 (d, J=8.4 Hz, 2H), 7.48-7.45 (m, 1H), 7.40-7.38 (d, J=8.4 Hz, 2H), 6.99 (s, 1H), 6.53-6.51 (d, J=8 Hz, 1H), 6.17-6.15 (d, J=7.6 Hz, 1H), 1.93 (m, 1H), 1.23 (s, 9H), 0.84-0.81 (m, 2H), 0.58-0.57 (m, 2H). MS (M+1): 447.23. (LCMS Purity 96.87%, Rt=6.14 min) (2).

### Example 29

### Synthesis of Compound 179 [N-(6-bromo-2-(pyridin-3-yl)pyrazolo[1,5-a]pyridin-7-yl)-4-(tert-butyl)benzenesulfonamide] and Compound 180 [4-(tert-butyl)-N-(6-cyano-2-(pyridin-3-yl)pyrazolo[1,5-a]pyridin-7-yl)benzenesulfonamide]

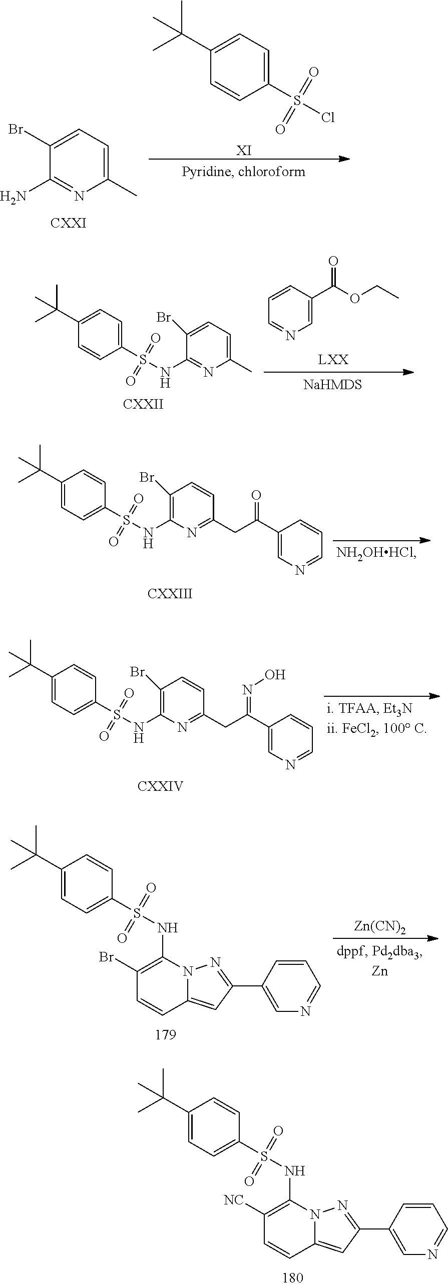

Synthesis of CXXII:

To a stirred solution of compound CXXI (5 g, 26.88 mmol) in chloroform (60 ml) was added pyridine (20 ml) at 0° C. followed by addition of 4-tert-butylbenzenesulfonyl chloride (XI, 12.4 g, 53.76 mmol). The reaction mixture was heated at 100° C. for 12 h. The reaction mixture was cooled to room temperature and concentrated under reduced pressure. The crude mass was diluted with saturated ammonium chloride solution and extracted with ethyl acetate. An organic layer was washed with brine solution, dried over anhydrous Na₂SO₄ and evaporated under reduced pressure to afford N-(3-bromo-6-methylpyridin-2-yl)-4-(tert-butyl)benzenesulfonamide (CXXII; 9 g, 90% yield). ¹H NMR (400 MHz, DMSO-d6) δ 11.18 (bs, 1H), 7.86-7.83 (m, 2H), 7.60-7.58 (d, J=8.4 Hz, 2H), 6.88-6.86 (d, J=8.4 Hz, 2H), 2.39 (s, 3H), 1.27 (s, 9H). MS (M+1): 381.22. (LCMS Purity 97.01%).

Synthesis of CXXIII:

To a stirred solution of compound CXXII (2.5 g; 6.53 mmol) and ethyl nicotinate (LXX; 1.97 g; 13.05 mmol) in THF (20 ml) was added sodium bis(trimethylsilyl)amide (35 ml, 1.0 M in THF, 32.63 mmol) dropwise at 0° C. The resultant solution was stirred at ambient temperature for 2 h. The reaction mixture was diluted with saturated solution of ammonium chloride and extracted with ethyl acetate. The organic layer was washed with brine solution and dried over anhydrous Na₂SO₄, filtered and evaporated under reduced pressure to N-(3-bromo-6-(2-oxo-2-(pyridin-3-yl)ethyl)pyridin-2-yl)-4-(tert-butyl)benzenesulfonamide CXXIII, as a keto-enol tautomeric mixture. MS (M+1): 491.12. The crude was carried forward to next step without purification.

Synthesis of CXXIV:

To a stirred solution of compound CXXIII (6 g, tautomeric mixture) in methanol (120 ml) was added hydroxylamine hydrochloride (4.28 g; 61.6 mmol) followed by a 10% aqueous solution of sodium hydroxide (50 ml). The resultant suspension was heated at 100° C. for 12 h and then cooled to room temperature. The reaction mixture was concentrated in vacuo and the residue was diluted with water and extracted with ethyl acetate. The organic layer was washed with brine solution and dried over anhydrous Na₂SO₄, filtered and evaporated under reduced pressure to obtain desired product N-(3-bromo-6-(2-(hydroxyimino)-2-(pyridin-3-yl)ethyl)pyridin-2-yl)-4-(tert-butyl) benzenesulfonamide as off white solid (CXXIV; 4 g; 64% yield). ¹H NMR (400 MHz, DMSO-d6) δ 11.64 (bs, 1H), 11.04 (bs, 1H), 8.67 (s, 1H), 8.47-8.46 (d, J=3.6 Hz, 1H), 7.88-7.74 (m, 4H), 7.60-7.58 (d, J=8.8 Hz, 2H), 7.28-7.25 (m, 1H), 6.72-6.70 (d, J=8.4 Hz, 1H), 4.23 (s, 2H), 1.25 (s, 9H). MS (M+1): 503.23.

### Synthesis of Compound 179; N-(6-bromo-2-(pyridin-3-yl)pyrazolo[1,5-a]pyridin-7-yl)-4-(tert-butyl)benzenesulfonamide

To a stirred solution of compound CXXIV (1.5 g, 2.98 mmol) in 1,2-dimethoxyethane (26 ml) at 0° C. was added trifluoroacetic anhydride (0.84 g, 5.99 mmol). The reaction mixture was allowed to stir at 0° C. for 20 minutes, followed by drop wise addition of triethylamine (4.1 g, 2.99 mmol) in 1,2-dimethoxyethane (5 ml). The reaction mixture was stirred at room temperature for 1 h. To the reaction mixture was further added iron (II) chloride (0.15 g, 1.19 mmol) and the resulting mixture heated at 100° C. for 3 h. The reaction mixture was cooled and concentrated, diluted with water and extracted with ethyl acetate. The organic layer was washed with brine solution and dried over anhydrous Na₂SO₄, filtered and evaporated under reduced pressure to obtain crude compound, which was purified by column chromatography using 21% ethyl acetate in hexane to afford the title compound as a white solid (179; 0.5 g; 30% yield). ¹H NMR (400 MHz, DMSO-d6): δ 9.21 (s, 1H), 8.61-8.60 (d, J=4.8 Hz, 1H), 8.40-8.38 (d, J=7.6 Hz, 1H), 7.83-7.81 (d, J=8.0 Hz, 2H), 7.57-7.51 (m, 4H), 7.27 (s, 1H), 6.81-6.79 (d, J=7.6 Hz, 1H), 1.15 (s, 9H). MS (M+1): 485.11 (LCMS purity 98.72%, Rt=6.22 min) (2).

### Synthesis of Compound 180; 4-(tert-butyl)-N-(6-cyano-2-(pyridin-3-yl)pyrazolo[1,5-a]pyridin-7-yl)benzenesulfonamide

To a stirred solution of compound 179, (0.25 g, 0.52 mmol) in dimethylacetamide (10 ml) was added Zn(CN)₂ (0.12 g, 1.03 mmol). The reaction mixture was purged with argon for 20 minutes before 1,1′-Bis (diphenylphosphino)ferrocene (0.056 g, 0.103 mmol), Pd₂dba₃ (0.094 g, 0.103 mmol) and a catalytic amount of Zn dust were added. The reaction mixture was heated at 120° C. for 2 h in a microwave reactor. The reaction mixture was cooled and filtered through a celite bed. The filtrate was concentrated, diluted with water and extracted with dichloromethane. The organic layer was washed with brine solution and dried over anhydrous Na₂SO₄, filtered and evaporated under reduced pressure to obtain the crude compound, which was purified by column chromatography using 2% methanol in 4% ammoniated dichloromethane to 4-(tert-butyl)-N-(6-cyano-2-(pyridin-3-yl)pyrazolo[1,5-a]pyridin-7-yl)benzenesulfonamide (180; 0.08 g, 36% yield). ¹H NMR (400 MHz, DMSO-d6): δ 8.32 (s, 1H), 8.67 (m, 1H), 8.61-8.59 (d, J=7.6 Hz, 1H), 7.84-7.82 (d, J=8.4 Hz, 2H), 7.66-7.63 (d, J=8.4 Hz, 2H), 7.54-7.51 (d, J=8.4 Hz, 2H), 7.23 (s, 1H), 6.61-6.59 (d, J=8 Hz, 1H), 1.24 (s, 9H). MS (M+1): 432.15 (LCMS purity 99.17%, Rt=5.19 min) (1).

### Example 30

### Synthesis of Compound 165 4-(tert-butyl)-N-(4-bromo-2-(1-methyl-1H-pyrazol-4-yl)pyrazolo[1,5-a]pyridin-7-yl)benzenesulfonamide; Compound 166 4-(tert-butyl)-N-(4-cyano-2-(1-methyl-1H-pyrazol-4-yl)pyrazolo[1,5-a]pyridin-7-yl)benzenesulfonamide and Compounds 181 to 193

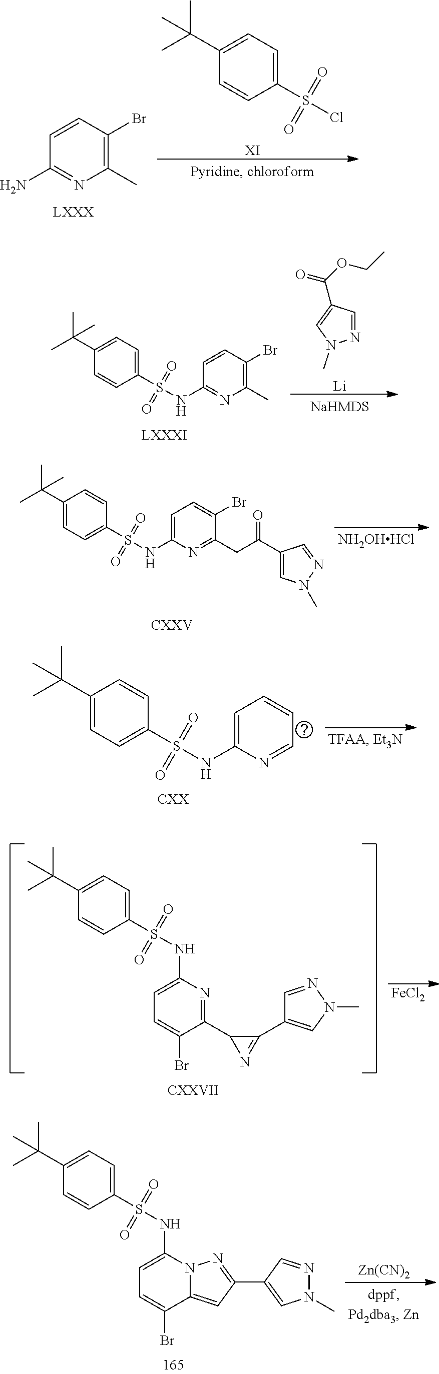

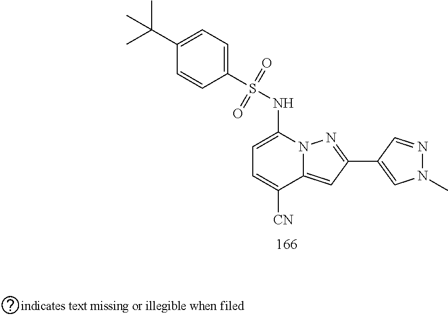 [SMILES: Cn1cc(-c2cc3c(C#N)ccc(NS(=O)(=O)c4ccc(C(C)(C)C)cc4)n3n2)cn1]

Synthesis of LXXXI:

To a stirred solution of compound LXXX (200 g, 1.07 mol) in chloroform (1 L) was added pyridine (600 ml) at 0° C. followed by addition of 4-tert-butylbenzenesulphonyl chloride (XI, 299 g, 1.28 mol). The reaction mixture was heated at 100° C. for 4 h, cooled to room temperature and concentrated under reduced pressure. The crude mass was diluted with a saturated ammonium chloride solution and extracted with ethyl acetate. The organic layer was washed with brine solution, dried over anhydrous Na₂SO₄ and evaporated under reduced pressure to afford N-(5-bromo-6-methylpyridin-2-yl)-4-(tert-butyl)benzenesulfonamide (LXXXI, 320 g, 78% yield). H NMR (400 MHz, DMSO-d6) δ 11.14 (bs, 1H), 7.86-7.82 (m, 3H), 7.60-7.58 (d, J=8.4 Hz, 2H), 6.87-6.85 (d, J=10.4 Hz, 1H), 2.39 (s, 3H), 1.27 (s, 9H). MS (M+1): 383.2.

Synthesis of CXXV:

To a stirred solution of compound LXXXI (250 g; 0.65 mol) and ethyl 1-methyl-1H-pyrazole-4-carboxylate (LI; 151 g; 0.98 mol) in THF (500 ml) was added sodium bis(trimethylsilyl)amide (2.6 L, 1.0 M in THF, 2.61 mol) dropwise at 0° C. Upon complete addition, the resultant solution was stirred at ambient temperature for 12 h. The reaction mixture was diluted with a saturated solution of ammonium chloride and extracted with ethyl acetate. The separated organic layer was washed with brine solution and dried over anhydrous Na₂SO₄, filtered and evaporated under reduced pressure to obtain CXXV, N-(5-bromo-6-(2-(1-methyl-1H-pyrazol-4-yl)-2-oxoethyl)pyridin-2-yl)-4-(tert-butyl)benzenesulfonamide as a keto-enol tautomeric mixture. MS (M+1): 491.17. The crude material was carried forward to the next step without purification.

Synthesis of CXXVI:

To a stirred solution of compound CXXV, (300 g, tautomeric mixture) in methanol (1.5 L) was added hydroxylamine hydrochloride (212 g; 3.05 mmol) followed by a 10% aqueous solution of sodium hydroxide (1.5 L). The resultant suspension was heated at 100° C. for 12 h and then cooled to room temperature. The reaction mixture was concentrated in vacuo and the residue was diluted with water and extracted with ethyl acetate. The organic layer was washed with brine solution and dried over anhydrous Na₂SO₄, filtered and was evaporated under reduced pressure to obtain the crude compound, which was triturated with diethyl ether and hexane to afford desired product, N-(5-bromo-6-(2-(hydroxyimino)-2-(1-methyl-1H-pyrazol-4-yl)ethyl)pyridin-2-yl)-4-(tert-butyl)benzenesulfonamide as an off white solid (CXXVI; 180 g; 58% yield). MS (M+1): 506.1 (LCMS Purity 96%).

### Synthesis of 165: N-(4-bromo-2-(1-methyl-1H-pyrazol-4-yl) pyrazolo[1,5-a]pyridin-7-yl)-4-(tert-butyl)benzenesulfonamide

To a stirred solution of CXXVI, (25 g, 0.049 mol) in dichloromethane (375 ml) at 0° C. was added trifluoroacetic anhydride (41.58 g, 0.198 mol). The reaction mixture was allowed to stir at 0° C. for 45 minutes, followed by the drop wise addition of triethylamine (60.11 g, 0.59 mol) in dichloromethane (80 ml). The reaction mixture was stirred at room temperature for 2 h. The reaction mixture was diluted with water and extracted with dichloromethane. The organic layer was washed with brine solution and dried over anhydrous Na₂SO₄, filtered and evaporated under reduced pressure to obtain crude product CXXVII. To this material, was added iron (II) chloride (2.5 g, 0.02 mol) and the mixture heated at 100° C. for 3 h. The reaction mixture was cooled and concentrated, diluted with water and extracted with ethyl acetate. The organic layer was washed with brine solution and dried over anhydrous Na₂SO₄, filtered and evaporated under reduced pressure to obtain crude compound, which was purified by column chromatography using 30% ethyl acetate in hexane to afford the title compound as an off white solid. (165; 10 g; 40% yield). ¹H NMR (400 MHz, DMSO-d6): δ 8.17 (s, 1H), 7.87-7.83 (m, 3H), 7.57-7.55 (d, J=8.0 Hz, 2H), 7.46-7.44 (d, J=8.0 Hz, 1H), 6.80 (s, 1H), 6.63-6.61 (d, J=8.4 Hz, 1H), 3.89 (s, 3H), 1.20 (s, 9H). MS (M+1): 488.11

### Synthesis of 166: 4-(tert-butyl)-N-(4-cyano-2-(1-methyl-1H-pyrazol-4-yl)pyrazolo[1,5-a]pyridin-7-yl)benzenesulfonamide

To a stirred solution of Compound 165, (10 g, 0.02 mol) in dimethylacetamide (100 ml) was added Zn(CN)₂ (11.8 g, 0.10 mol). The reaction mixture was purged with argon for 20 min, whereupon 1, 1′-Bis (diphenylphosphino)ferrocene (0.9 g, 1.6 mmol), Pd₂dba₃ (1.5 g, 1.6 mmol) and a catalytic amount of zinc dust were added. The reaction mixture was heated at 120° C. for 2 h. The reaction mixture was cooled and concentrated, diluted with water and extracted with dichloromethane. The organic layer was washed with brine solution and dried over anhydrous Na₂SO₄, filtered and evaporated under reduced pressure to obtain crude compound, which was purified by column chromatography using 5% methanol in dichloromethane and 10% ammonia hydroxide to afford the title compound (166; 7.5 g, 71% yield). ¹H NMR (400 MHz, DMSO-d6): δ 8.26 (s, 1H), 7.91 (s, 1H), 7.73-7.71 (d, J=8.0 Hz, 2H), 7.46-7.44 (d, J=8.0 Hz, 2H), 7.39-7.37 (d, J=8.4 Hz, 1H), 6.57 (s, 1H), 6.32-6.30 (d, J=8.4 Hz, 1H), 3.87 (s, 3H), 1.25 (s, 9H). MS (M+1): 435.43. (LCMS Purity 99.12%, Rt=6.69 min) (2), Melting point−269° C.-270° C.

The following nitrile derivatives were prepared in a similar manner, using the appropriate esters instead of ethyl 1-methyl-1H-pyrazole-4-carboxylate (LI) in Step 2. Chloro compounds were prepared by reacting the appropriate esters with LXXIX, prepared as in Example 14, instead of LXXXI, in step 2 and without the final step described above.

| | | LCMS | Purity | |
|---|---|---|---|---|
| S. No. | Structure | (M + 1) | (LCMS) | ¹H NMR |
| 181 | 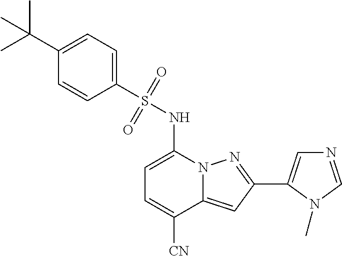 [SMILES: Cn1cncc1-c1cc2c(C#N)ccc(NS(=O)(=O)c3ccc(C(C)(C)C)cc3)n2n1] | 435.13 | 97.79% Rt = 5.08 min (1) | ¹H NMR (400 MHz, d-TFA): δ 9.22 (s, 1H), 8.33 (s, 1H), 7.95-7.91 (m, 3H), 7.58-7.56 (d, J = 8 Hz, 2H), 7.37 (s, 1H), 7.02-7.00 (d, J = 8 Hz, 1H), 4.21 (s, 3H), 1.20 (s, 9H). |
| 182 | 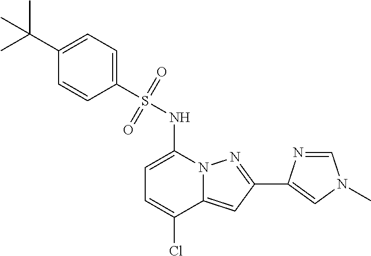 [SMILES: Cn1cnc(-c2cc3c(Cl)ccc(NS(=O)(=O)c4ccc(C(C)(C)C)cc4)n3n2)c1] | 444.10 | 94.16% Rt = 5.79 min (2) | ¹H NMR (400 MHz, DMSO- d6): δ 7.96 (s, 1H), 7.74-7.72 (m, 3H), 7.45-7.43 (m, 2H), 7.05-7.03 (m, 1H), 6.62 (s, 1H), 6.33-6.28 (m, 1H), 3.74 (s, 3H), 1.23 (s, 9H). |
| 183 | 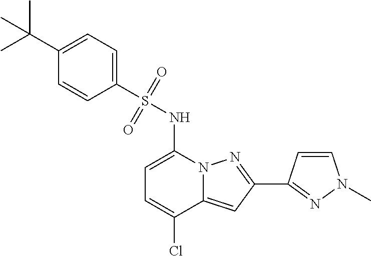 [SMILES: Cn1ccc(-c2cc3c(Cl)ccc(NS(=O)(=O)c4ccc(C(C)(C)C)cc4)n3n2)n1] | 444.13 | 98.85% Rt = 4.74 min (2) | ¹H NMR (400 MHz, DMSO- d6): δ 7.87-7.85 (d, J = 8 Hz, 2H), 7.78 (s, 1H), 7.56- 7.54 (d, J = 8 Hz, 2H), 7.36- 7.34 (d, J = 8 Hz, 1H), 6.86 (s, 1H), 6.68-6.66 (m, 2H), 3.90 (s, 3H), 1.21 (s, 9H). |
| 184 | 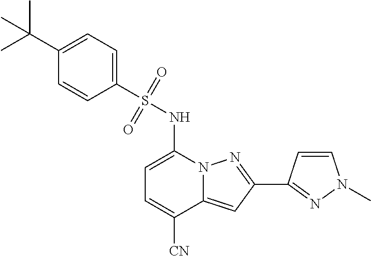 [SMILES: Cn1ccc(-c2cc3c(C#N)ccc(NS(=O)(=O)c4ccc(C(C)(C)C)cc4)n3n2)n1] | 435.14 | 95.53% Rt = 6.75 min (2) | ¹H NMR (400 MHz, CDCl₃): δ 8.02-8.00 (d, J = 8 Hz, 2H), 7.70-7.68 (d, J = 8 Hz, 1H), 7.33-7.31 (d, J = 8.4 Hz, 2H), 7.14 (s, 1H), 6.92 (s, 1H), 6.87-6.85 (d, J = 8.4 Hz, 1H), 6.53 (s, 1H), 2.93 (s, 3H), 1.25 (s, 9H). |
| 185 | 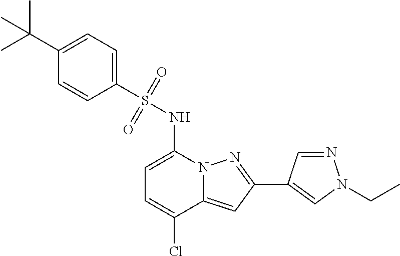 [SMILES: CCn1cc(-c2cc3c(Cl)ccc(NS(=O)(=O)c4ccc(C(C)(C)C)cc4)n3n2)cn1] | 458.17 | 99.56% Rt = 7.29 min (2) | ¹H NMR (400 MHz, DMSO- d6): δ 11.18 (bs, 1H), 8.22 (s, 1H), 7.87 (s, 1H), 7.84- 7.82 (d, J = 8.4 Hz, 2H), 7.55-7.53 (d, J = 8.4 Hz, 2H), 7.34-7.32 (d, J = 8 Hz, 1H), 6.86 (s, 1H), 6.68-6.66 (d, J = 8 Hz, 1H), 4.21-4.16 (q, J = 7.2 Hz, 2H), 1.43- 1.42 (t, J = 7.2 Hz, 3H), 1.19 (s, 9H). |
| 186 | 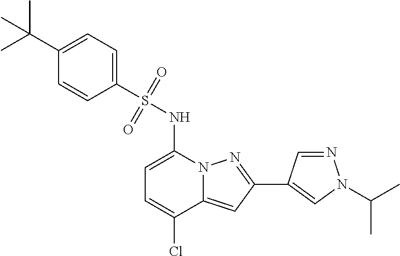 [SMILES: CC(C)n1cc(-c2cc3c(Cl)ccc(NS(=O)(=O)c4ccc(C(C)(C)C)cc4)n3n2)cn1] | 472.16 | 99.81% Rt = 7.53 min (2) | ¹H NMR (400 MHz, DMSO- d6): δ 11.07 (bs, 1H), 8.26 (s, 1H), 7.87 (s, 1H), 7.84- 7.82 (d, J = 8.4 Hz, 2H), 7.54-7.52 (d, J = 8.4 Hz, 2H), 7.34-7.32 (d, J = 8 Hz, 1H), 6.87 (s, 1H), 6.68-6.66 (d, J = 8 Hz, 1H), 4.58-4.51 (m, 1H), 1.46-1.45 (d, J = 6.8 Hz, 6H), 1.18 (s, 9H). |
| 187 | 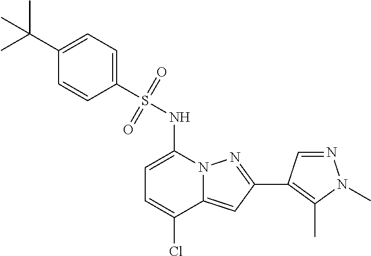 [SMILES: Cc1c(-c2cc3c(Cl)ccc(NS(=O)(=O)c4ccc(C(C)(C)C)cc4)n3n2)cnn1C] | 458.47 | 99.65% Rt = 7.23 min (2) | ¹H NMR (400 MHz, DMSO- d6): δ 11.03 (bs, 1H), 8.08 (s, 1H), 7.81-7.79 (d, J = 8 Hz, 2H), 7.54-7.52 (d, J = 8 Hz, 2H), 7.35-7.33 (d, J = 8 Hz, 1H), 6.73-6.68 (m, 2H), 3.80 (s, 3H), 2.38 (s, 3H), 1.19 (s, 9H). |
| 188 | 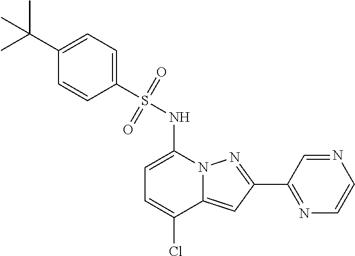 [SMILES: CC(C)(C)c1ccc(S(=O)(=O)Nc2ccc(Cl)c3cc(-c4cnccn4)nn23)cc1] | 442.45 | 97.54% Rt = 5.03 min (2) | ¹H NMR (400 MHz, DMSO- d6): δ 11.41 (bs, 1H), 9.24 (s, 1H), 8.72-8.68 (d, J = 8 Hz, 2H), 7.81-7.79 (d, J = 8 Hz, 2H), 7.51-7.47 (m, 3H), 7.20 (s, 1H), 6.92-6.91 (d, J = 6.8 Hz, 1H), 1.25 (s, 9H). |
| 189 | 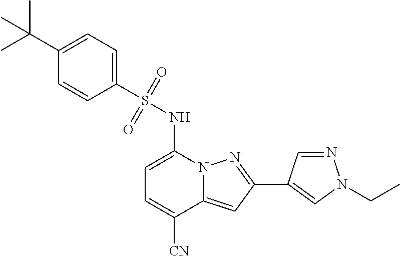 [SMILES: CCn1cc(-c2cc3c(C#N)ccc(NS(=O)(=O)c4ccc(C(C)(C)C)cc4)n3n2)cn1] | 449.19 | 99.29% Rt = 6.91 min (2) | ¹H NMR (400 MHz, DMSO with d-TFA): δ 8.32 (s, 1H), 8.01-7.97 (m, 3H), 7.81-7.79 (d, J = 7.6 Hz, 1H), 7.60- 7.59 (d, J = 5.2 Hz, 2H), 7.01 (s, 1H), 6.82-6.80 (d, J = 8 Hz, 1H), 4.21-4.16 (q, J = 7.2 Hz, 2H), 1.43-1.39 (t, J = 8 Hz, 3H), 1.22 (s, 9H). |
| 190 | 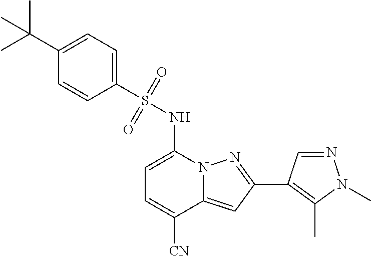 [SMILES: Cc1c(-c2cc3c(C#N)ccc(NS(=O)(=O)c4ccc(C(C)(C)C)cc4)n3n2)cnn1C] | 449.21 | 98.59% Rt = 6.88 min (2) | ¹H NMR (400 MHz, DMSO- d6): δ 8.19 (s, 1H), 7.94- 7.92 (d, J = 7.6 Hz, 2H), 7.79-7.80 (d, J = 7.6 Hz, 1H), 7.61-7.59 (d, J = 7.6 Hz, 2H), 6.82 (s, 1H), 6.75- 6.73 (d, J = 8 Hz, 1H), 3.81 (s, 3H), 2.45 (s, 3H), 1.24 (s, 9H). |
| 191 | 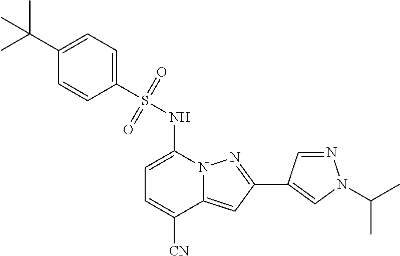 [SMILES: CC(C)n1cc(-c2cc3c(C#N)ccc(NS(=O)(=O)c4ccc(C(C)(C)C)cc4)n3n2)cn1] | 463.51 | 99.74% Rt = 7.07 min (2) | ¹H NMR (400 MHz, DMSO- d6): δ 8.32 (s, 1H), 7.92 (s, 1H), 7.73-7.71 (d, J = 8.4 Hz, 2H), 7.46-7.44 (d, J = 8.4 Hz, 2H), 7.38-7.36 (d, J = 8.4 Hz, 1H), 6.59 (s, 1H), 6.31-6.29 (d, J = 8.4 Hz, 1H), 4.52 (m, 1H), 1.47-1.45 (d, J = 6.4 Hz, 6H), 1.25 (s, 9H). |
| 192 | 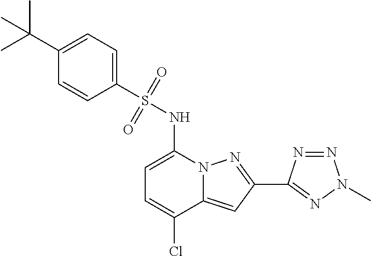 [SMILES: Cn1nnc(-c2cc3c(Cl)ccc(NS(=O)(=O)c4ccc(C(C)(C)C)cc4)n3n2)n1] | 446.37 | 95.80% Rt = 5.65 min (1) | ¹H NMR (400 MHz, DMSO- d6): δ 7.88-7.86 (d, J = 8 Hz, 2H), 7.55-7.53 (d, J = 8 Hz, 2H), 7.45-7.43 (d, J = 8 Hz, 1H), 7.15 (s, 1H), 6.76-6.74 (d, J = 8 Hz, 1H), 4.47 (s, 3H), 1.20 (s, 9H). |
| 193 | 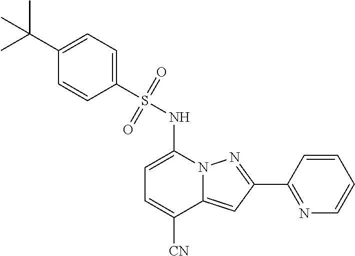 [SMILES: CC(C)(C)c1ccc(S(=O)(=O)Nc2ccc(C#N)c3cc(-c4ccccn4)nn23)cc1] | 432.11 | 99.35% Rt = 5.38 min (1) | ¹H NMR (400 MHz, DMSO- d6): δ 8.65-8.64 (d, J = 4 Hz, 1H), 8.19-8.17 (d, J = 7.6 Hz, 1H), 7.91-7.88 (t, J = 7.2 Hz, 1H), 7.77-7.74 (d, J = 8.4 Hz, 2H), 7.48-7.46 (m, 3H), 7.40-7.37 (m, 1H), 6.89 (s, 1H), 6.40-6.38 (d, J = 8.4 Hz, 1H), 1.25 (s, 9H). |

### Example 31

### Synthesis of Compounds 194 to 210

The following chloro compounds were prepared essentially as in Example 14 using the appropriate ester in step 2 not including the final oxidation described. Any pyridine N-oxides were prepared from the corresponding pyridines using the oxidation conditions described in the final step of Example 14. Nitriles were prepared from the corresponding chloro compound using the methodology described in Example 16.

| | | LCMS | Purity | |
|---|---|---|---|---|
| S. No. | Structure | (M + 1) | (LCMS) | ¹H NMR |
| 194 | 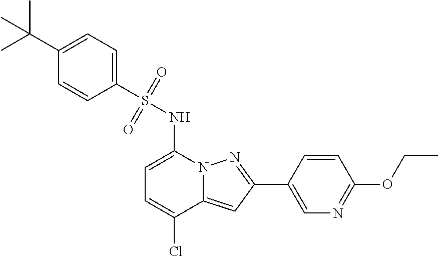 [SMILES: CCOc1ccc(-c2cc3c(Cl)ccc(NS(=O)(=O)c4ccc(C(C)(C)C)cc4)n3n2)cn1] | 485.14 | 99.68% Rt = 5.86 min (2) | ¹H NMR (400 MHz, DMSO- d6): δ 11.31 (bs, 1H), 8.71 (s, 1H), 8.27-8.25 (d, J = 7.6 Hz, 1H), 7.81-7.79 (d, J = 8.4 Hz, 2H), 7.53-7.51 (d, J = 8.4 Hz, 2H), 7.40-7.38 (d, J = 8.4 Hz, 1H), 7.17 (s, 1H), 6.90-6.88 (d, J = 8.8 Hz, 1H), 6.81-6.79 (d, J = 8 Hz, 1H), 4.40-4.33 (q, J = 6.8 Hz, 2H), 1.36-1.32 (t, J = 6.8 Hz, 3H), 1.16 (s, 9H). |
| 195 | 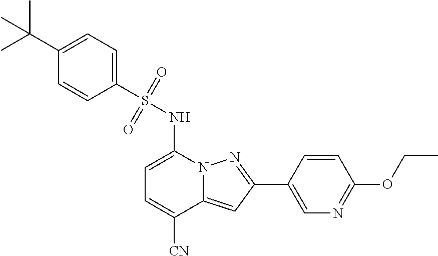 [SMILES: CCOc1ccc(-c2cc3c(C#N)ccc(NS(=O)(=O)c4ccc(C(C)(C)C)cc4)n3n2)cn1] | 476.20 | 98.91% Rt = 5.13 min (2) | ¹H NMR (400 MHz, DMSO- d6): δ 8.83 (s, 1H), 8.38- 8.36 (d, J = 8 Hz, 1H), 7.88- 7.87 (d, J = 6.8 Hz, 2H), 7.71-7.70 (m, 1H), 7.57-7.55 (d, J = 8 Hz, 2H), 7.14 (s, 1H), 6.92-6.90 (d, J = 8 Hz, 1H), 6.68-6.66 (d, J = 7.6 Hz, 1H), 4.37-4.36 (q, 2H), 1.36-1.33 (t, J = 6.8 Hz, 3H), 1.24 (s, 9H). |
| 196 | 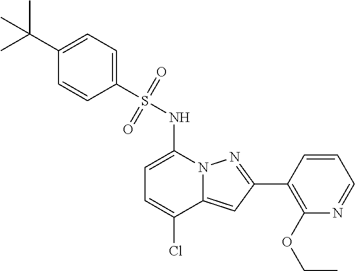 [SMILES: CCOc1ncccc1-c1cc2c(Cl)ccc(NS(=O)(=O)c3ccc(C(C)(C)C)cc3)n2n1] | 485.12 | 99.57% Rt = 6.22 min (2) | ¹H NMR (400 MHz, DMSO- d6): δ 11.28 (bs, 1H), 8.49- 8.47 (d, J = 6 Hz, 1H), 8.23- 8.22 (m, 1H), 7.81-7.79 (d, J = 8.4 Hz, 2H), 7.52-7.49 (d, J = 8.4 Hz, 2H), 7.42-7.40 (d, J = 8 Hz, 1H), 7.16-7.13 (m, 2H), 6.86-6.84 (d, J = 8 Hz, 1H), 4.50-4.45 (q, J = 6.8 Hz, 7.2 Hz, 2H), 1.42- 1.39 (t, J = 7.2 Hz, 3H), 1.14 (s, 9H). |
| 197 | 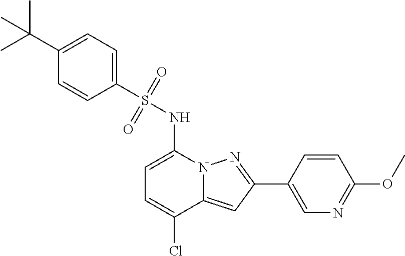 [SMILES: COc1ccc(-c2cc3c(Cl)ccc(NS(=O)(=O)c4ccc(C(C)(C)C)cc4)n3n2)cn1] | 471.07 | 97.34% Rt = 6.00 min (1) | ¹H NMR (400 MHz, DMSO- d6): δ 11.24 (bs, 1H), 8.73 (s, 1H), 8.28-8.26 (d, J = 8 Hz, 1H), 7.81-7.79 (d, J = 8.4 Hz, 2H), 7.53-7.51 (d, J = 8.4 Hz, 2H), 7.40-7.39 (d, J = 8.4 Hz, 1H), 7.18 (s, 1H), 6.94-6.91 (d, J = 8.4 Hz, 1H), 6.80-6.79 (d, J = 7.2 Hz, 1H), 3.90 (s, 3H), 1.16 (s, 9H). |
| 198 | 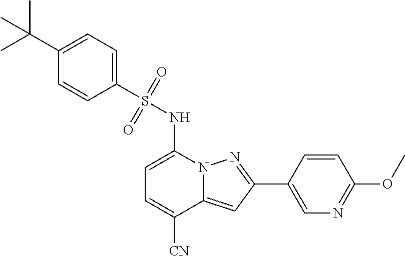 [SMILES: COc1ccc(-c2cc3c(C#N)ccc(NS(=O)(=O)c4ccc(C(C)(C)C)cc4)n3n2)cn1] | 462.11 | 97.33% Rt = 5.51 min (1) | ¹H NMR (400 MHz, DMSO- d6): δ 8.85 (s, 1H), 8.40- 8.38 (d, J = 8 Hz, 1H), 7.88- 7.85 (d, J = 8.4 Hz, 2H), 7.69-7.67 (d, J = 8 Hz, 1H), 7.56-7.54 (d, J = 8 Hz, 2H), 7.13 (s, 1H), 6.95-6.92 (d, J = 8.4 Hz, 1H), 6.66-6.65 (d, J = 7.2 Hz, 1H), 3.91 (s, 3H), 1.24 (s, 9H). |
| 199 | 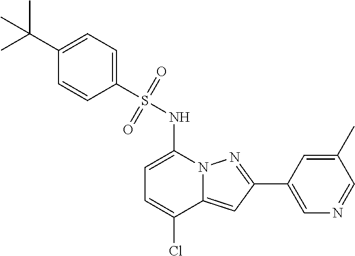 [SMILES: Cc1cncc(-c2cc3c(Cl)ccc(NS(=O)(=O)c4ccc(C(C)(C)C)cc4)n3n2)c1] | 455.08 | 99.73% Rt = 6.23 min (2) | ¹H NMR (400 MHz, DMSO- d6): δ 11.56-11.52 (bs, 1H), 9.00 (s, 1H), 8.45 (s, 1H), 8.23 (s, 1H), 7.83-7.81 (d, J = 8.4 Hz, 2H), 7.53-7.51 (d, J = 8.4 Hz, 2H), 7.42-7.40 (d, J = 8 Hz, 1H), 7.29 (s, 1H), 6.83-6.81 (d, J = 8 Hz, 1H), 2.39 (s, 3H), 1.15 (s, 9H). |
| 200 | 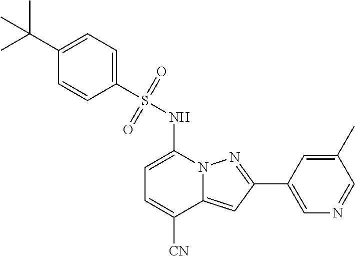 [SMILES: Cc1cncc(-c2cc3c(C#N)ccc(NS(=O)(=O)c4ccc(C(C)(C)C)cc4)n3n2)c1] | 446.11 | 99.49% Rt = 5.97 min (2) | ¹H NMR (400 MHz, DMSO- d6): δ 9.20 (s, 1H), 8.63-8.58 (m, 2H), 7.83-7.81 (d, J = 8.0 Hz, 2H), 7.65-7.63 (d, J = 7.6 Hz, 1H), 7.53-7.51 (d, J = 8.0 Hz, 2H), 7.26 (s, 1H), 6.60-6.58 (d, J = 8.0 Hz, 1H)), 2.43 (s, 3H), 1.25 (s, 9H). |
| 201 | 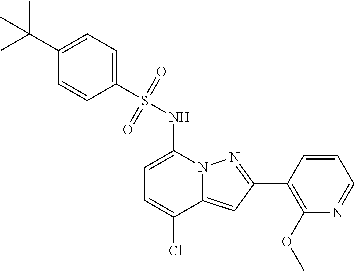 [SMILES: COc1ncccc1-c1cc2c(Cl)ccc(NS(=O)(=O)c3ccc(C(C)(C)C)cc3)n2n1] | 471.28 | 96.31% Rt = 5.84 min (2) | ¹H NMR (400 MHz, DMSO- d6): δ 11.31 (bs, 1H), 8.49- 8.47 (d, J = 7.6 Hz, 1H), 8.25-8.24 (d, J = 4 Hz, 1H), 7.81-7.79 (d, J = 8 Hz, 2H), 7.52-7.50 (d, J = 8 Hz, 2H), 7.44-7.40 (m, 1H), 7.18-7.12 (m, 2H), 6.86-6.84 (d, J = 8 Hz, 1H), 4.02 (s, 3H), 1.15 (s, 9H). |
| 202 | 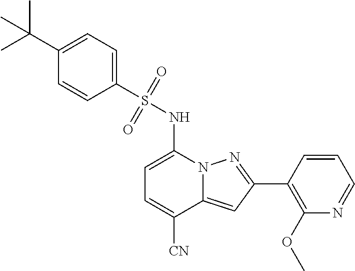 [SMILES: COc1ncccc1-c1cc2c(C#N)ccc(NS(=O)(=O)c3ccc(C(C)(C)C)cc3)n2n1] | 462.35 | 97.25% Rt = 5.07 min (2) | ¹H NMR (400 MHz, DMSO- d6): δ 8.50-8.42 (d, J = 7.6 Hz, 1H), 8.22-8.21 (d, J = 3.2 Hz, 1H), 7.75-7.73 (d, J = 8.4 Hz, 2H), 7.47-7.45 (m, 3H), 7.15-7.12 (m, 1H), 6.89 (s, 1H), 6.40-6.38 (d, J = 8.4 Hz, 1H), 4.02 (s, 3H), 1.25 (s, 9H). |
| 203 | 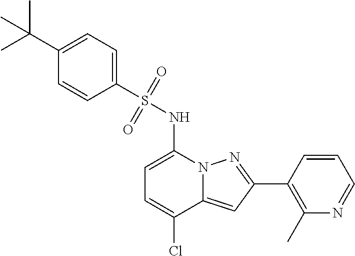 [SMILES: Cc1ncccc1-c1cc2c(Cl)ccc(NS(=O)(=O)c3ccc(C(C)(C)C)cc3)n2n1] | 455.14 | 99.63% Rt = 6.00 min (2) | ¹H NMR (400 MHz, DMSO- d6): δ 11.50 (bs, 1H), 8.51- 8.50 (d, J = 3.6 Hz, 1H), 8.06-8.04 (d, J = 8 Hz, 1H), 7.81-7.79 (d, J = 8 Hz, 2H), 7.54-7.52 (d, J = 8 Hz, 2H), 7.44-7.42 (d, J = 8 Hz, 1H), 7.38-7.35 (m, 1H), 7.02 (s, 1H), 6.84-6.82 (d, J = 8 Hz, 1H), 2.63 (s, 3H), 1.20 (s, 9H). |
| 204 | 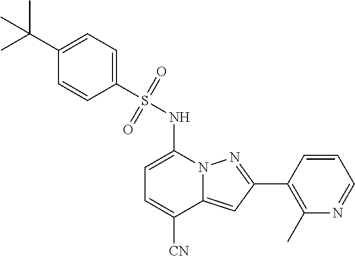 [SMILES: Cc1ncccc1-c1cc2c(C#N)ccc(NS(=O)(=O)c3ccc(C(C)(C)C)cc3)n2n1] | 444.18 (M − 1) | 99.88% Rt = 5.35 min (1) | ¹H NMR (400 MHz, DMSO- d6): δ 8.48-8.47 (d, J = 4 Hz, 1H), 8.05-8.03 (d, J = 6.8 Hz, 1H), 7.75-7.73 (d, J = 8.4 Hz, 2H), 7.49-7.45 (m, 3H), 7.34-7.31 (m, 1H), 6.67 (s, 1 H), 6.42-6.40 (d, J = 8.4 Hz, 1H), 2.69 (s, 3H), 1.25 (s, 9H). |
| 205 | 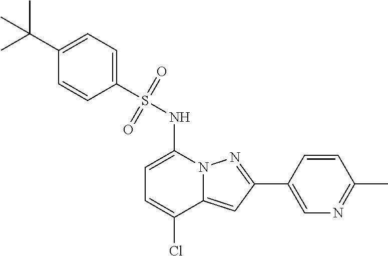 [SMILES: Cc1ccc(-c2cc3c(Cl)ccc(NS(=O)(=O)c4ccc(C(C)(C)C)cc4)n3n2)cn1] | 455.51 | 99.74% Rt = 6.05 min (2) | ¹H NMR (400 MHz, DMSO- d6): δ 11.43 (bs, 1H), 9.07 (s, 1H), 8.28-8.26 (m, 1H), 7.83-7.80 (d, J = 8.8 Hz, 2H), 7.53-7.50 (d, J = 8.4 Hz, 2H), 7.41-7.37 (m, 2H), 7.25 (s, 1H), 6.81-6.79 (d, J = 8 Hz, 1H), 2.52 (s, 3H), 1.15 (s, 9H). |
| 206 | 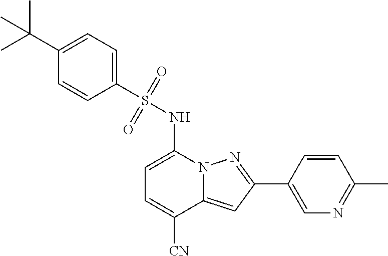 [SMILES: Cc1ccc(-c2cc3c(C#N)ccc(NS(=O)(=O)c4ccc(C(C)(C)C)cc4)n3n2)cn1] | 446.48 | 99.11% Rt = 5.84 min (2) | ¹H NMR (400 MHz, DMSO- d6): δ 9.10 (s, 1H), 8.36- 8.34 (d, J = 7.2 Hz, 1H), 7.77-7.75 (d, J = 8.4 Hz, 2H), 7.48-7.46 (m, 3H), 7.41-7.39 (d, J = 7.6 Hz, 1H), 6.99 (s, 1H), 6.42-6.40 (d, J = 8.4 Hz, 1H), 2.54 (s, 3H), 1.25 (s, 9H). |
| 207 | 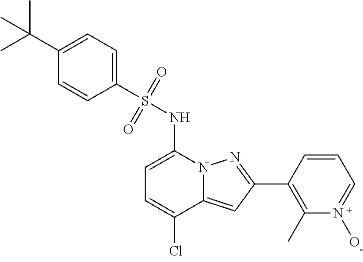 | 471.39 | 95.59% Rt = 6.57 min (2) | ¹H NMR (400 MHz, DMSO- d6 with D₂O & TFA): δ 8.73-8.72 (d, J = 6 Hz, 1H), 8.20-8.18 (d, J = 6.8 Hz, 1H), 7.77-7.72 (m, 3H), 7.47-7.41 (m, 3H), 7.08 (m, 1H), 6.98 (m, 1H), 2.69 (s, 3H), 1.14 (s, 9H). |
| 208 | 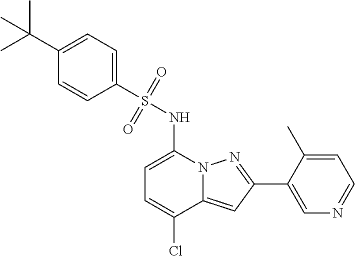 [SMILES: Cc1ccncc1-c1cc2c(Cl)ccc(NS(=O)(=O)c3ccc(C(C)(C)C)cc3)n2n1] | 455.32 | 98.30% Rt = 6.07 min (2) | ¹H NMR (400 MHz, DMSO- d6): δ 11.40 (bs, 1H), 8.76 (s, 1H), 8.47-8.46 (d, J = 5.6 Hz, 1H), 7.81-7.78 (d, J = 8 Hz, 2H), 7.54-7.52 (d, J = 8 Hz, 2H), 7.45-7.43 (d, J = 8 Hz, 1H), 7.37-7.36 (d, J = 8 Hz, 1H), 7.05 (s, 1H), 6.85- 6.83 (d, J = 8 Hz, 1H), 2.46 (s, 3H), 1.20 (s, 9H). |
| 209 | 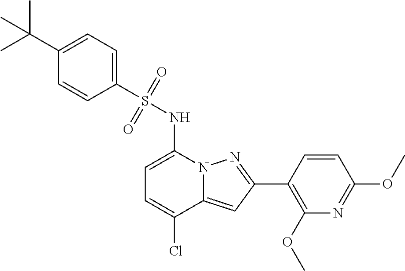 [SMILES: COc1ccc(-c2cc3c(Cl)ccc(NS(=O)(=O)c4ccc(C(C)(C)C)cc4)n3n2)c(OC)n1] | 500.77 | 99.00% Rt = 6.53 min (1) | ¹H NMR (400 MHz, DMSO- d6): δ 11.02 (bs, 1H), 8.44- 8.42 (d, J = 8.4 Hz, 1H), 7.82-7.80 (d, J = 8.4 Hz, 2H), 7.53-7.51 (d, J = 8.4 Hz, 2H), 7.38-7.35 (d, J = 8.4 Hz, 1H), 7.00 (s, 1H), 6.81-6.79 (d, J = 8 Hz, 1H), 6.57-6.55 (d, J = 8 Hz, 1H), 4.04 (s, 3H), 3.93 (s, 3H), 1.16 (s, 9H). |
| 210 | 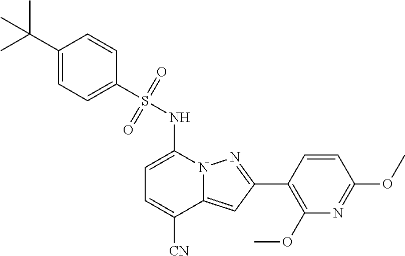 [SMILES: COc1ccc(-c2cc3c(C#N)ccc(NS(=O)(=O)c4ccc(C(C)(C)C)cc4)n3n2)c(OC)n1] | 492.14 | 97.16% Rt = 5.50 min (2) | ¹H NMR (400 MHz, DMSO- d6): δ 8.58-8.57 (d, J = 5.6 Hz, 1H), 7.90-7.88 (d, J = 7.6 Hz, 2H), 7.73 (m, 1H), 7.57-7.55 (d, J = 7.6 Hz, 2H), 7.00 (s, 1H), 6.73-6.71 (d, J = 8 Hz, 1H), 6.58-6.56 (d, J = 8 Hz, 1H), 4.06 (s, 3H), 3.94 (s, 3H), 1.24 (s, 9H). |

### Example 32

### Synthesis of Compound 211; 4-(tert-butyl)-N-(4-chloro-3-(pyridin-3-yl)pyrazolo[1,5-a]pyridin-7-yl)benzenesulfonamide

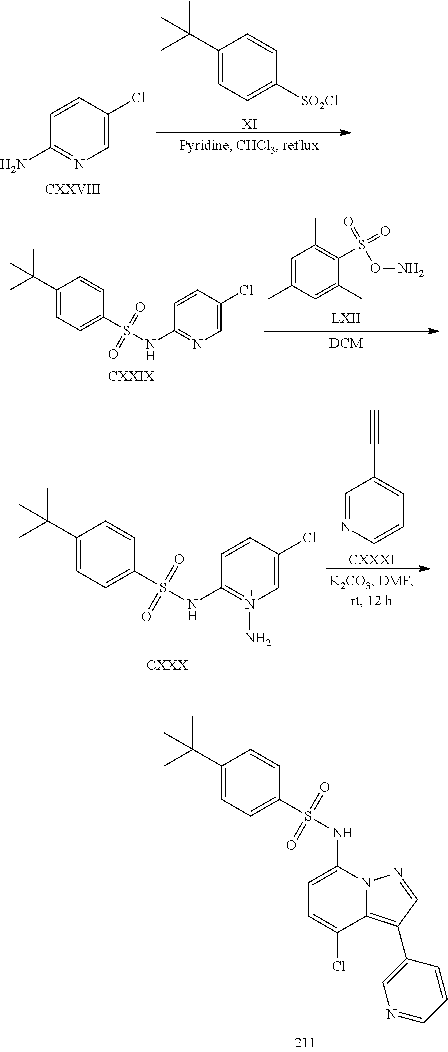

Synthesis of CXXIX:

To a stirred solution of compound CXXVIII (5 g, 39.06 mmol) in chloroform (50 ml) was added pyridine (15 ml) at 0° C. followed by 4-tert-butylbenzenesulphonyl chloride (XI, 10.8 g, 46.41 mmol). The reaction mixture was heated at 100° C. for 12 h, cooled to room temperature and concentrated under reduced pressure. The crude mass was diluted with saturated ammonium chloride solution and extracted with ethyl acetate. The organic layer was washed with brine solution and dried over anhydrous Na₂SO₄, filtered and evaporated under reduced pressure to afford 4-(tert-butyl)-N-(5-chloropyridin-2-yl)benzenesulfonamide (CXXIX; 11 g, 87% yield). ¹H NMR (400 MHz, DMSO d6) δ 11.25 (bs, 1H), 8.22 (s, 1H), 7.84-7.78 (m, 3H), 7.60-7.58 (d, J=8.4 Hz, 2H), 7.11-7.08 (d, J=8.8 Hz, 1H), 1.26 (s, 9H). MS (M+1): 324.98 (LCMS Purity 94.17%).

Synthesis of CXXX:

To a stirred solution of CXXIX (5 g, 15.39 mmol) in dichloromethane (50 ml) was added O-(mesitylsulfonyl) hydroxylamine (LXII; 10 g, 46.45 mmol). The reaction mixture was stirred for 12 h at room temperature and then diluted with water and extracted with dichloromethane. The organic layer was washed with a saturated aqueous solution of sodium bicarbonate and brine solution before being dried over anhydrous Na₂SO₄, filtered and evaporated under reduced pressure to obtain crude compound. This was purified by using by flash chromatography using 10% methanol in dichloromethane to afford the title compound as an off white solid (CXXX, 1.8 g; 34% yield. ¹H NMR (400 MHz, DMSO-d6): δ 8.38 (s, 1H), 7.78-7.76 (d, J=8.4 Hz, 2H), 7.74-7.71 (dd, J=2.4 and 7.2 Hz, 1H), 7.53-7.51 (d, J=8.4 Hz, 2H), 7.44-7.42 (d, J=9.6 Hz, 1H), 6.99 (bs, 2H), 1.27 (s, 9H).

### Synthesis of Compound 211: 4-(tert-butyl)-N-(4-chloro-3-(pyridin-3-yl)pyrazolo[1,5-a]pyridin-7-yl)benzenesulfonamide

To a stirred solution of CXXX (0.5 g, crude) in dimethylformamide (7.37 ml) was added potassium carbonate (0.717 g, 5.19 mmol), followed by addition of 3-ethynylpyridine (CXXXI, 0.45 g, 3.98 mmol). The reaction mixture was stirred at 60° C. for 24 h and then concentrated in vacuo. The residue was diluted with water and extracted with ethyl acetate. The organic layer was washed with brine solution and dried over anhydrous Na₂SO₄, filtered and evaporated under reduced pressure to obtain the crude compound, which was purified by preparative HPLC to afford, the title compound (211; 0.050 g, 7.71% yield). ¹H NMR (400 MHz, DMSO-d6): δ 8.70 (s, 1H), 8.54 (m, 1H), 8.23 (s, 1H), 7.90-7.88 (m, 3H), 7.60-7.58 (d, J=8.0 Hz, 2H), 7.46 (m, 1H), 7.40-7.38 (d, J=8.0 Hz, 1H), 6.81-6.79 (d, J=8.0 Hz, 1H), 1.26 (s, 9H). MS (M+1): 441.40. (LCMS Purity 97.81%, Rt=5.78 min) (2).

### Example 33

### Biological Activity: FLIPR Assay Using hCCR9 Over Expressed Cells

A calcium flux assay was used to determine the ability of the compounds to interfere with the binding between CCR9 and its chemokine ligand (TECK) in Cheml-hCCR9 overexpressing cells. hCCR9 overexpressing cells were seeded (25,000 cells/well) into black Poly-D-Lysine coated clear bottom 96-well plates (BD Biosciences, Cat #356640) and incubated overnight at 37° C./5% CO₂ in a humidified incubator. Media was aspirated and cells washed twice with 100 μL assay buffer (1×HBSS, 20 mM HEPES) containing 2.5 mM Probenecid. A 0.3× Fluo-4 NW calcium dye was prepared in assay buffer containing 5 mM Probenecid and stored in the dark. Each well was loaded with 100 μL of 0.3× Fluo-4 NW calcium dye and incubated at 37° C./5% CO₂ for 60 minutes and then at room temperature for 30 minutes. A half-log serially diluted concentration response curve was prepared at a 3× final assay concentration for each compound (10 μM−0.1 nM final assay concentration) and 50 μL of the compound then transferred to the cells (150 μL final volume) for 60 minutes prior to stimulation (30 minutes at 37° C./5% CO₂ and 30 minutes at room temperature). TECK was diluted to 4× its ECso in assay buffer (containing 0.1% [w/v] bovine serum albumin[BSA]) and 50 μL dispensed through the fluorometric imaging plate reader (FLIPR) instrument to stimulate the cells (200 μL final volume). The increase in intracellular calcium levels was measured with the FLIPR instrument. The potency of the compound as a CCR9 antagonist was calculated as an IC₅₀ using GraphPad Prism software (variable slope four parameter). The Ki of the compound was determined from the IC₅₀ values using the following equation.

Ki calculation: IC₅₀/1+(Agonist (TECK) conc. used in assay/EC₅₀ of agonist (TECK) generated on day of experiment)

| Compound | | |
|---|---|---|
| number | Structure | Ki (nM) |
| 1 | 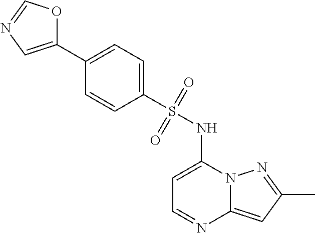 [SMILES: Cc1cc2nccc(NS(=O)(=O)c3ccc(-c4cnco4)cc3)n2n1] | 456 |
| 3 | 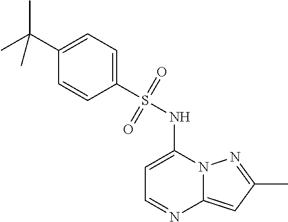 [SMILES: Cc1cc2nccc(NS(=O)(=O)c3ccc(C(C)(C)C)cc3)n2n1] | 1440 |
| 37 | 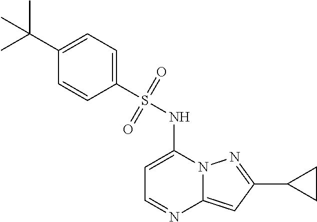 [SMILES: CC(C)(C)c1ccc(S(=O)(=O)Nc2ccnc3cc(C4CC4)nn23)cc1] | 1951 |
| 79 | 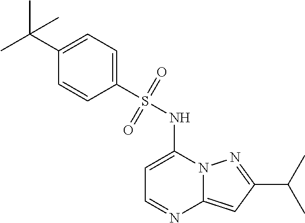 [SMILES: CC(C)c1cc2nccc(NS(=O)(=O)c3ccc(C(C)(C)C)cc3)n2n1] | 1515 |
| 85 | 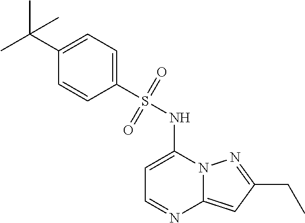 [SMILES: CCc1cc2nccc(NS(=O)(=O)c3ccc(C(C)(C)C)cc3)n2n1] | 1800 |
| 94 | 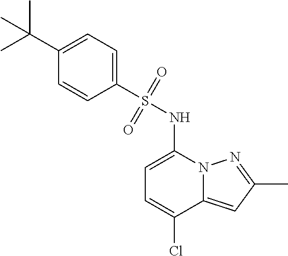 [SMILES: Cc1cc2c(Cl)ccc(NS(=O)(=O)c3ccc(C(C)(C)C)cc3)n2n1] | 181 |
| 95 | 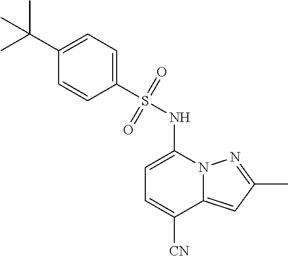 [SMILES: Cc1cc2c(C#N)ccc(NS(=O)(=O)c3ccc(C(C)(C)C)cc3)n2n1] | 110 |
| 98 | 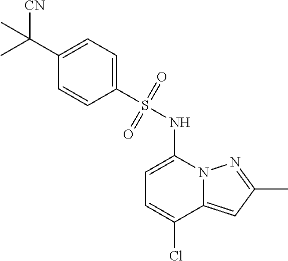 [SMILES: Cc1cc2c(Cl)ccc(NS(=O)(=O)c3ccc(C(C)(C)C#N)cc3)n2n1] | 512 |
| 105 | 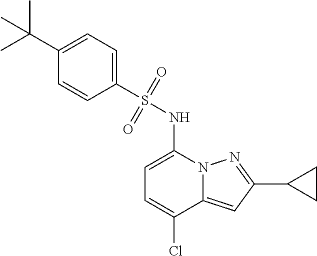 [SMILES: CC(C)(C)c1ccc(S(=O)(=O)Nc2ccc(Cl)c3cc(C4CC4)nn23)cc1] | 56 |
| 110 | 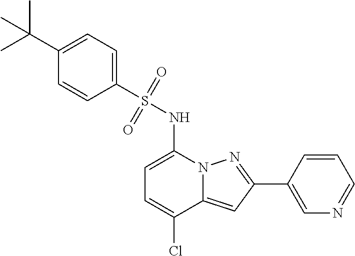 [SMILES: CC(C)(C)c1ccc(S(=O)(=O)Nc2ccc(Cl)c3cc(-c4cccnc4)nn23)cc1] | 394 |
| 111 | 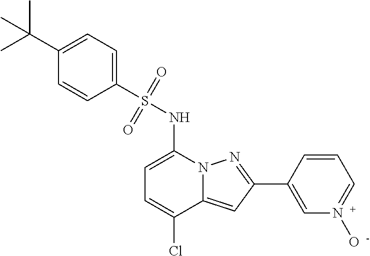 | 190 |
| 113 | 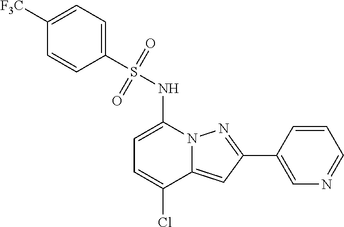 [SMILES: O=S(=O)(Nc1ccc(Cl)c2cc(-c3cccnc3)nn12)c1ccc(C(F)(F)F)cc1] | 756 |
| 149 | 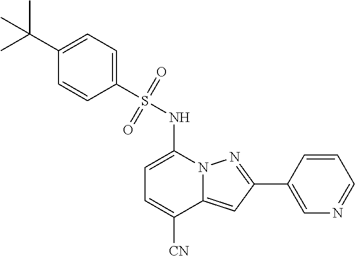 [SMILES: CC(C)(C)c1ccc(S(=O)(=O)Nc2ccc(C#N)c3cc(-c4cccnc4)nn23)cc1] | 159 |
| 152 | 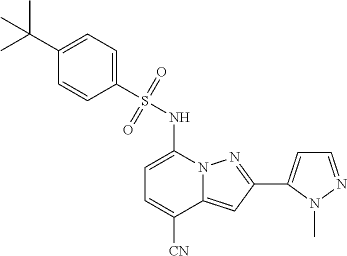 [SMILES: Cn1nccc1-c1cc2c(C#N)ccc(NS(=O)(=O)c3ccc(C(C)(C)C)cc3)n2n1] | 125 |
| 156 | 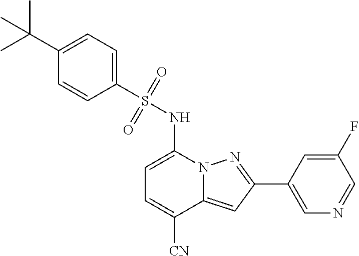 [SMILES: CC(C)(C)c1ccc(S(=O)(=O)Nc2ccc(C#N)c3cc(-c4cncc(F)c4)nn23)cc1] | 107 |
| 158 | 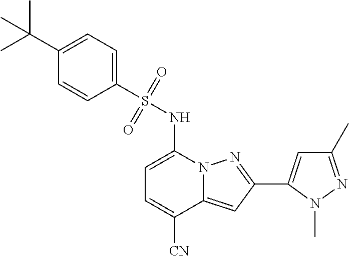 [SMILES: Cc1cc(-c2cc3c(C#N)ccc(NS(=O)(=O)c4ccc(C(C)(C)C)cc4)n3n2)n(C)n1] | 186 |
| 159 | 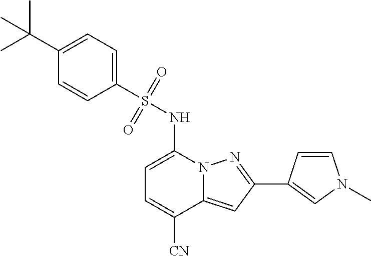 [SMILES: Cn1ccc(-c2cc3c(C#N)ccc(NS(=O)(=O)c4ccc(C(C)(C)C)cc4)n3n2)c1] | 136 |
| 161 | 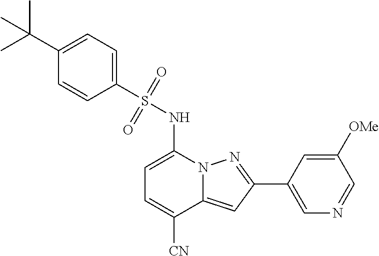 [SMILES: COc1cncc(-c2cc3c(C#N)ccc(NS(=O)(=O)c4ccc(C(C)(C)C)cc4)n3n2)c1] | 127 |
| 164 | 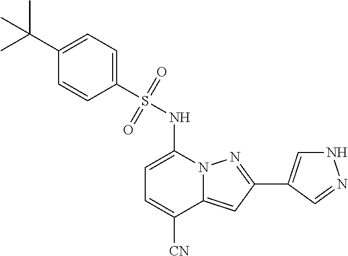 [SMILES: CC(C)(C)c1ccc(S(=O)(=O)Nc2ccc(C#N)c3cc(-c4cn[nH]c4)nn23)cc1] | 112 |
| 166 | 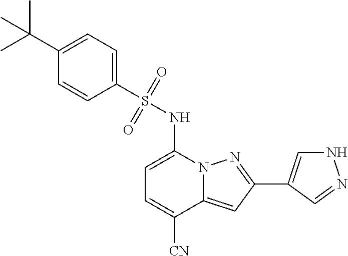 [SMILES: CC(C)(C)c1ccc(S(=O)(=O)Nc2ccc(C#N)c3cc(-c4cn[nH]c4)nn23)cc1] | 101 |
| 169 | 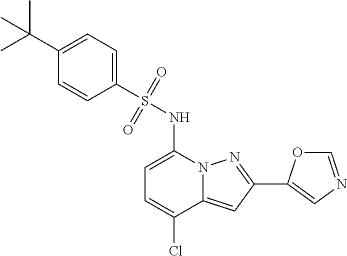 [SMILES: CC(C)(C)c1ccc(S(=O)(=O)Nc2ccc(Cl)c3cc(-c4cnco4)nn23)cc1] | 343 |
| 172 | 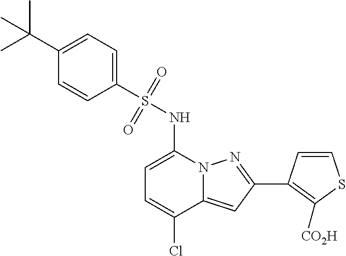 [SMILES: CC(C)(C)c1ccc(S(=O)(=O)Nc2ccc(Cl)c3cc(-c4ccsc4C(=O)O)nn23)cc1] | 129 |
| 180 | 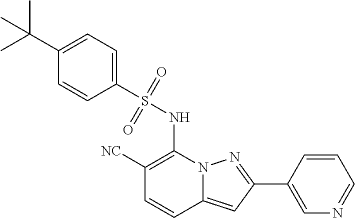 [SMILES: CC(C)(C)c1ccc(S(=O)(=O)Nc2c(C#N)ccc3cc(-c4cccnc4)nn23)cc1] | 133 |
| 182 | 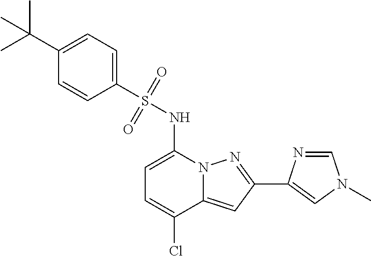 [SMILES: Cn1cnc(-c2cc3c(Cl)ccc(NS(=O)(=O)c4ccc(C(C)(C)C)cc4)n3n2)c1] | 168 |
| 183 | 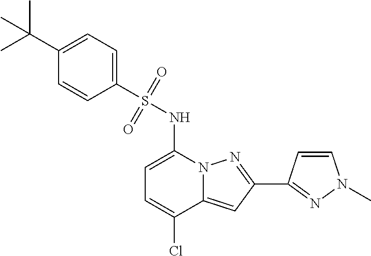 [SMILES: Cn1ccc(-c2cc3c(Cl)ccc(NS(=O)(=O)c4ccc(C(C)(C)C)cc4)n3n2)n1] | 94 |
| 184 | 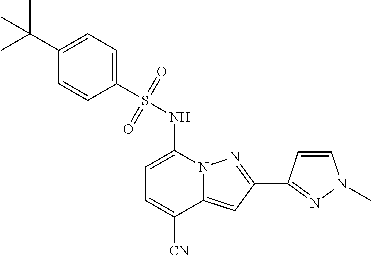 [SMILES: Cn1ccc(-c2cc3c(C#N)ccc(NS(=O)(=O)c4ccc(C(C)(C)C)cc4)n3n2)n1] | 17 |
| 186 | 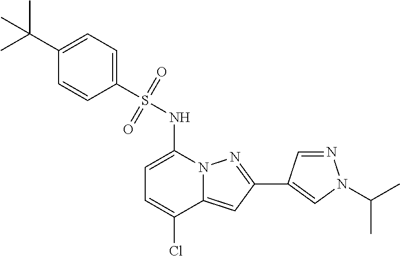 [SMILES: CC(C)n1cc(-c2cc3c(Cl)ccc(NS(=O)(=O)c4ccc(C(C)(C)C)cc4)n3n2)cn1] | 67 |
| 189 | 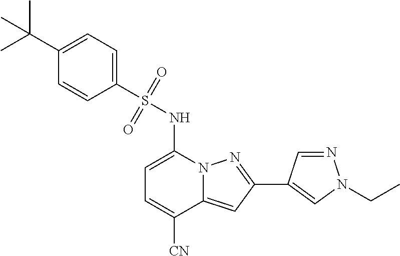 [SMILES: CCn1cc(-c2cc3c(C#N)ccc(NS(=O)(=O)c4ccc(C(C)(C)C)cc4)n3n2)cn1] | 22 |
| 190 | 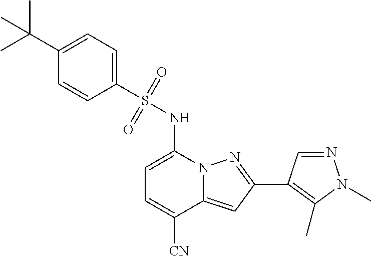 [SMILES: Cc1c(-c2cc3c(C#N)ccc(NS(=O)(=O)c4ccc(C(C)(C)C)cc4)n3n2)cnn1C] | 114 |
| 191 | 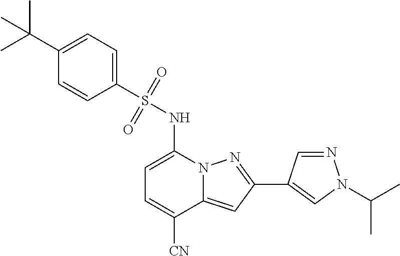 [SMILES: CC(C)n1cc(-c2cc3c(C#N)ccc(NS(=O)(=O)c4ccc(C(C)(C)C)cc4)n3n2)cn1] | 9 |
| 192 | 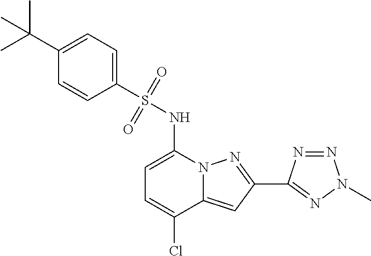 [SMILES: Cn1nnc(-c2cc3c(Cl)ccc(NS(=O)(=O)c4ccc(C(C)(C)C)cc4)n3n2)n1] | 183 |
| 198 | 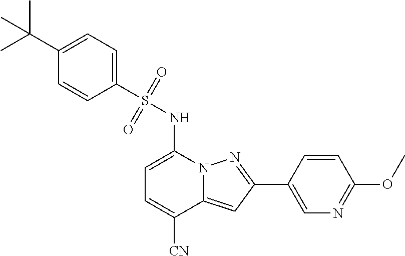 [SMILES: COc1ccc(-c2cc3c(C#N)ccc(NS(=O)(=O)c4ccc(C(C)(C)C)cc4)n3n2)cn1] | 54 |
| 199 | 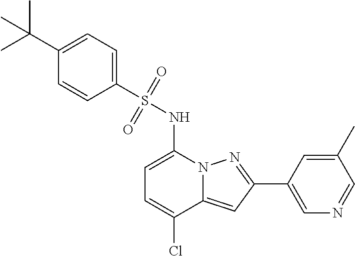 [SMILES: Cc1cncc(-c2cc3c(Cl)ccc(NS(=O)(=O)c4ccc(C(C)(C)C)cc4)n3n2)c1] | 63 |
| 200 | 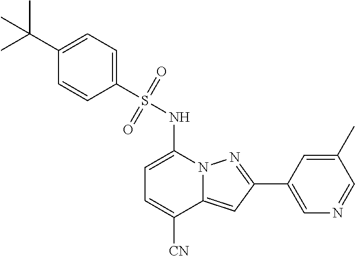 [SMILES: Cc1cncc(-c2cc3c(C#N)ccc(NS(=O)(=O)c4ccc(C(C)(C)C)cc4)n3n2)c1] | 12 |
| 202 | 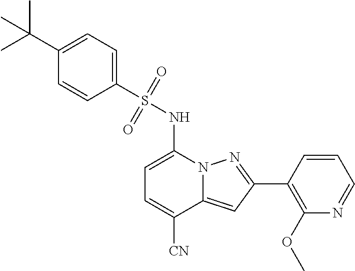 [SMILES: COc1ncccc1-c1cc2c(C#N)ccc(NS(=O)(=O)c3ccc(C(C)(C)C)cc3)n2n1] | 127 |
| 203 | 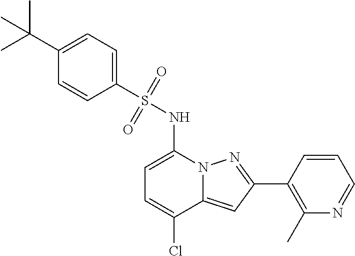 [SMILES: Cc1ncccc1-c1cc2c(Cl)ccc(NS(=O)(=O)c3ccc(C(C)(C)C)cc3)n2n1] | 149 |
| 204 | 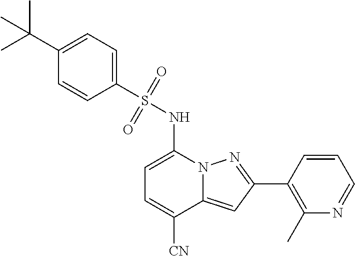 [SMILES: Cc1ncccc1-c1cc2c(C#N)ccc(NS(=O)(=O)c3ccc(C(C)(C)C)cc3)n2n1] | 121 |
| 206 | 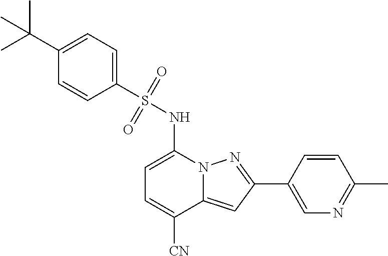 [SMILES: Cc1ccc(-c2cc3c(C#N)ccc(NS(=O)(=O)c4ccc(C(C)(C)C)cc4)n3n2)cn1] | 128 |

### Example 34

### Biological Activity: FLIPR Assay Using MOLT4 Cells

A calcium flux assay was used to determine the ability of the compounds to interfere with the binding between CCR9 and its chemokine ligand (TECK) in MOLT4 cells (a human T-cell line). MOLT4 cells were seeded (100,000 cells/well) in coming cell culture plates (Cat #3603) in assay buffer (lx HBSS, 20 mM HEPES) containing 2.5 mM Probenecid. The plate was centrifuged at 1200 rpm for 3 minutes and incubated at 37° C./5% CO₂ for 2 hours. A 0.3× Fluo-4 NW calcium dye was prepared in assay buffer containing 5 mM Probenecid and stored in the dark. Each well was loaded with 25 μL of 0.3× Fluo-4 NW calcium dye and incubated at 37 C/5% CO₂ for 60 minutes and then at room temperature for 30 minutes. A half-log serially diluted concentration response curve was prepared at a 4× concentration for each (10 μM-0.1 nM final assay concentration) and 25 μL of the compound then transferred to the cells (100 μL final volume) for 60 minutes prior to stimulation (30 minutes at 37° C./5% CO₂ and 30 minutes at room temperature). TECK was diluted to 5× its EC₅₀ in assay buffer (containing 0.1% [w/v] bovine serum albumin [BSA]) and 25 μL dispensed through the FLIPR instrument to stimulate the cells (125 μL final volume). The increased in intracellular calcium levels was measured with the FLIPR instrument. The potency of the compound as CCR9 antagonist was calculated as an IC₅₀ using GraphPad Prism software (variable slope four parameter). The Ki of the compound was determined from the IC₅₀ values using the following equation.

Ki calculation: IC₅₀/1+(Agonist (TECK) conc. used in assay/EC₅₀ of agonist (TECK) generated on day of experiment)

| Compound | | |
|---|---|---|
| number | Structure | Ki (nM) |
| 1 | 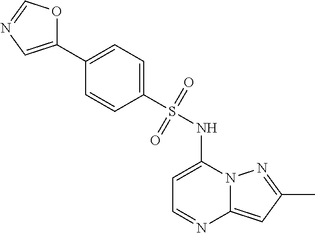 [SMILES: Cc1cc2nccc(NS(=O)(=O)c3ccc(-c4cnco4)cc3)n2n1] | 19 |
| 3 | 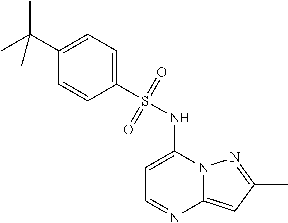 [SMILES: Cc1cc2nccc(NS(=O)(=O)c3ccc(C(C)(C)C)cc3)n2n1] | 315 |
| 19 | 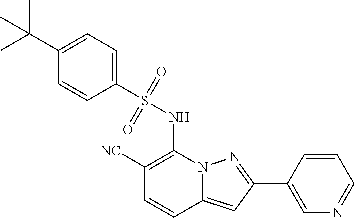 [SMILES: CC(C)(C)c1ccc(S(=O)(=O)Nc2c(C#N)ccc3cc(-c4cccnc4)nn23)cc1] | 1113 |
| 22 | 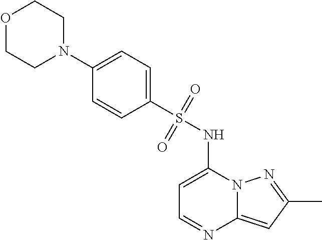 [SMILES: Cc1cc2nccc(NS(=O)(=O)c3ccc(N4CCOCC4)cc3)n2n1] | 4000 |
| 23 | 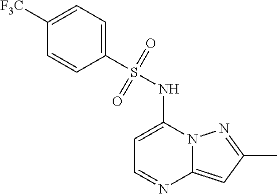 [SMILES: Cc1cc2nccc(NS(=O)(=O)c3ccc(C(F)(F)F)cc3)n2n1] | 1701 |
| 28 | 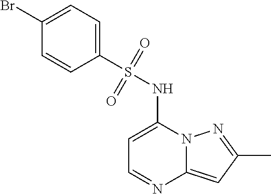 [SMILES: Cc1cc2nccc(NS(=O)(=O)c3ccc(Br)cc3)n2n1] | 6194 |
| 32 | 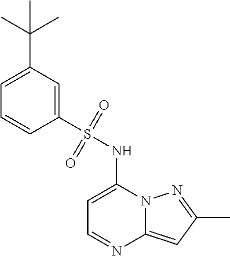 [SMILES: Cc1cc2nccc(NS(=O)(=O)c3cccc(C(C)(C)C)c3)n2n1] | 1783 |
| 33 | 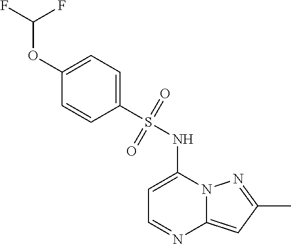 [SMILES: Cc1cc2nccc(NS(=O)(=O)c3ccc(OC(F)F)cc3)n2n1] | 1207 |
| 37 | 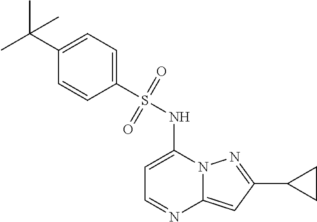 [SMILES: CC(C)(C)c1ccc(S(=O)(=O)Nc2ccnc3cc(C4CC4)nn23)cc1] | 703 |
| 39 | 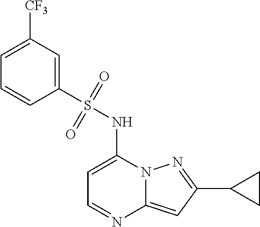 [SMILES: O=S(=O)(Nc1ccnc2cc(C3CC3)nn12)c1cccc(C(F)(F)F)c1] | 5886 |
| 41 | 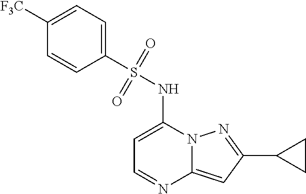 [SMILES: O=S(=O)(Nc1ccnc2cc(C3CC3)nn12)c1ccc(C(F)(F)F)cc1] | 1878 |
| 42 | 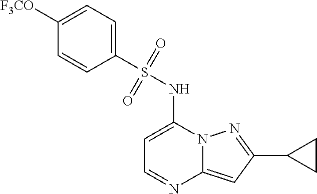 [SMILES: O=S(=O)(Nc1ccnc2cc(C3CC3)nn12)c1ccc(OC(F)(F)F)cc1] | 1593 |
| 44 | 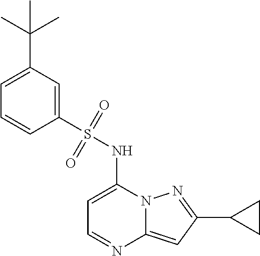 [SMILES: CC(C)(C)c1cccc(S(=O)(=O)Nc2ccnc3cc(C4CC4)nn23)c1] | 1424 |
| 50 | 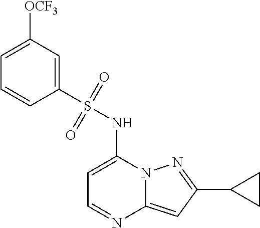 [SMILES: O=S(=O)(Nc1ccnc2cc(C3CC3)nn12)c1cccc(OC(F)(F)F)c1] | 3752 |
| 51 | 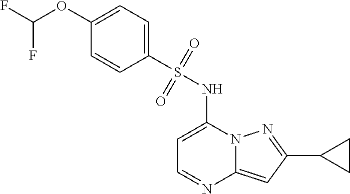 [SMILES: O=S(=O)(Nc1ccnc2cc(C3CC3)nn12)c1ccc(OC(F)F)cc1] | 3202 |
| 62 | 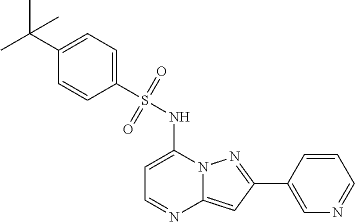 [SMILES: CC(C)(C)c1ccc(S(=O)(=O)Nc2ccnc3cc(-c4cccnc4)nn23)cc1] | 253 |
| 75 | 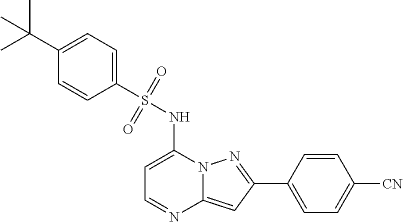 [SMILES: CC(C)(C)c1ccc(S(=O)(=O)Nc2ccnc3cc(-c4ccc(C#N)cc4)nn23)cc1] | 1555 |
| 79 | 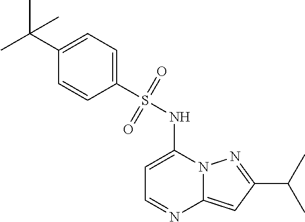 [SMILES: CC(C)c1cc2nccc(NS(=O)(=O)c3ccc(C(C)(C)C)cc3)n2n1] | 295 |
| 85 | 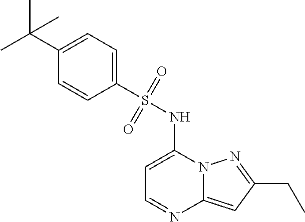 [SMILES: CCc1cc2nccc(NS(=O)(=O)c3ccc(C(C)(C)C)cc3)n2n1] | 111 |
| 87 | 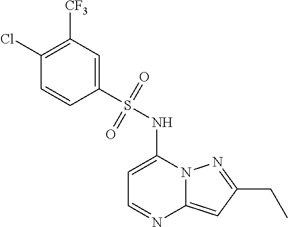 [SMILES: CCc1cc2nccc(NS(=O)(=O)c3ccc(Cl)c(C(F)(F)F)c3)n2n1] | 3234 |
| 90 | 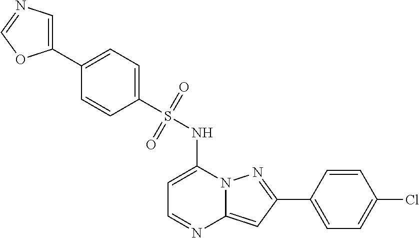 [SMILES: O=S(=O)(Nc1ccnc2cc(-c3ccc(Cl)cc3)nn12)c1ccc(-c2cnco2)cc1] | 1375 |
| 94 | 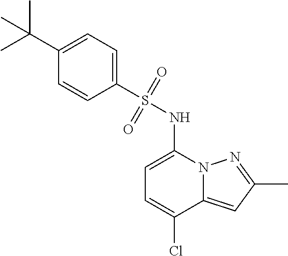 [SMILES: Cc1cc2c(Cl)ccc(NS(=O)(=O)c3ccc(C(C)(C)C)cc3)n2n1] | 162 |
| 95 | 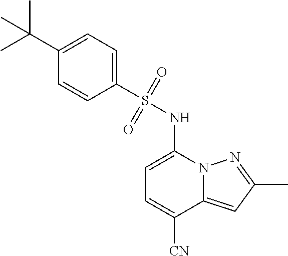 [SMILES: Cc1cc2c(C#N)ccc(NS(=O)(=O)c3ccc(C(C)(C)C)cc3)n2n1] | 2 |
| 98 | 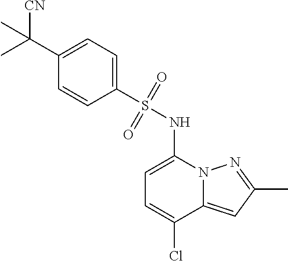 [SMILES: Cc1cc2c(Cl)ccc(NS(=O)(=O)c3ccc(C(C)(C)C#N)cc3)n2n1] | 109 |
| 101 | 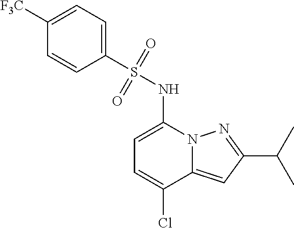 [SMILES: CC(C)c1cc2c(Cl)ccc(NS(=O)(=O)c3ccc(C(F)(F)F)cc3)n2n1] | 492 |
| 105 | 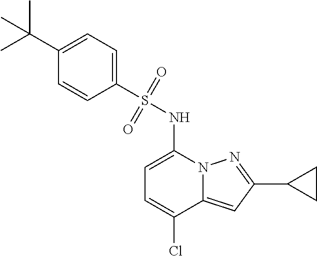 [SMILES: CC(C)(C)c1ccc(S(=O)(=O)Nc2ccc(Cl)c3cc(C4CC4)nn23)cc1] | 285 |
| 106 | 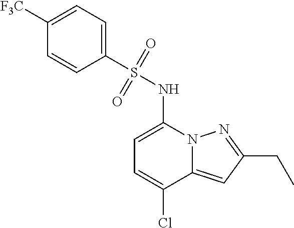 [SMILES: CCc1cc2c(Cl)ccc(NS(=O)(=O)c3ccc(C(F)(F)F)cc3)n2n1] | 177 |
| 108 | 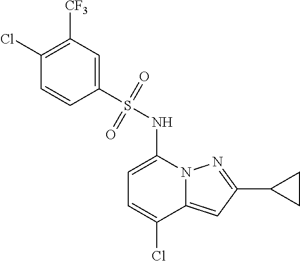 [SMILES: O=S(=O)(Nc1ccc(Cl)c2cc(C3CC3)nn12)c1ccc(Cl)c(C(F)(F)F)c1] | 456 |
| 110 | 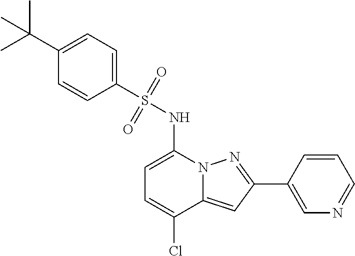 [SMILES: CC(C)(C)c1ccc(S(=O)(=O)Nc2ccc(Cl)c3cc(-c4cccnc4)nn23)cc1] | 115 |
| 111 | 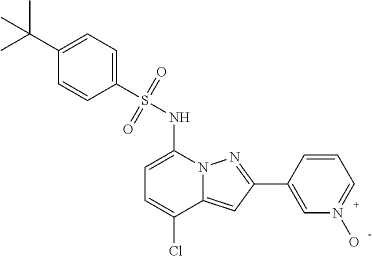 | 56 |
| 112 | 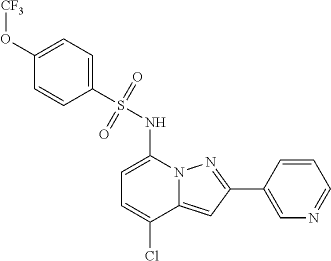 [SMILES: O=S(=O)(Nc1ccc(Cl)c2cc(-c3cccnc3)nn12)c1ccc(OC(F)(F)F)cc1] | 92 |
| 113 | 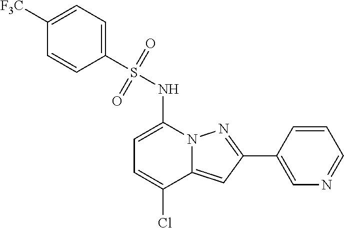 [SMILES: O=S(=O)(Nc1ccc(Cl)c2cc(-c3cccnc3)nn12)c1ccc(C(F)(F)F)cc1] | 187 |
| 115 | 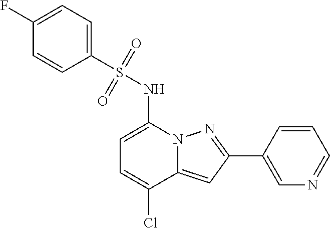 [SMILES: O=S(=O)(Nc1ccc(Cl)c2cc(-c3cccnc3)nn12)c1ccc(F)cc1] | 640 |
| 116 | 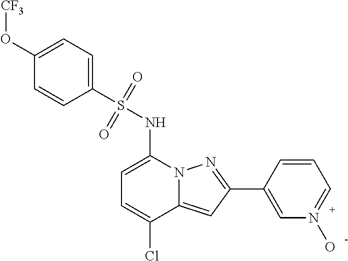 | 1030 |
| 123 | 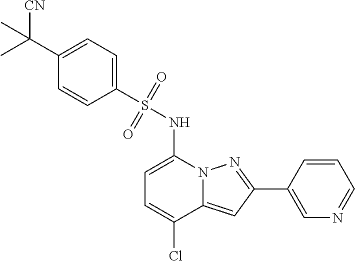 [SMILES: CC(C)(C#N)c1ccc(S(=O)(=O)Nc2ccc(Cl)c3cc(-c4cccnc4)nn23)cc1] | 219 |
| 126 | 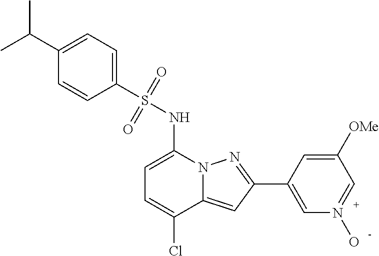 | 73 |
| 127 | 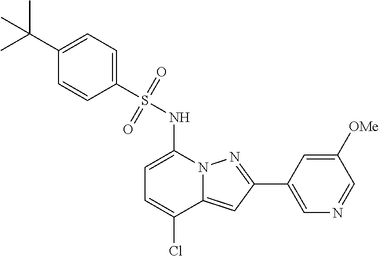 [SMILES: COc1cncc(-c2cc3c(Cl)ccc(NS(=O)(=O)c4ccc(C(C)(C)C)cc4)n3n2)c1] | 192 |
| 128 | 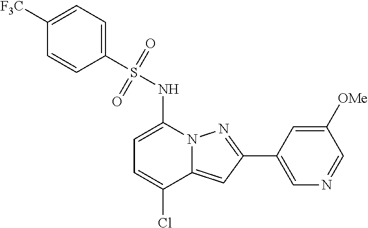 [SMILES: COc1cncc(-c2cc3c(Cl)ccc(NS(=O)(=O)c4ccc(C(F)(F)F)cc4)n3n2)c1] | 170 |
| 132 | 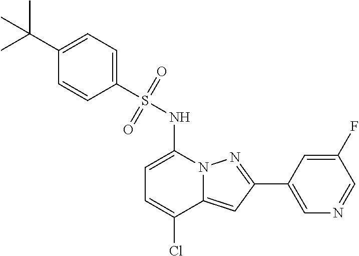 [SMILES: CC(C)(C)c1ccc(S(=O)(=O)Nc2ccc(Cl)c3cc(-c4cncc(F)c4)nn23)cc1] | 171 |
| 133 | 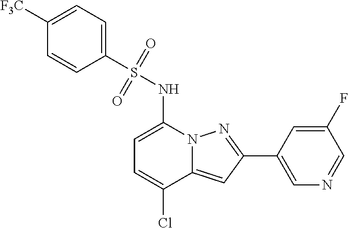 [SMILES: O=S(=O)(Nc1ccc(Cl)c2cc(-c3cncc(F)c3)nn12)c1ccc(C(F)(F)F)cc1] | 182 |
| 139 | 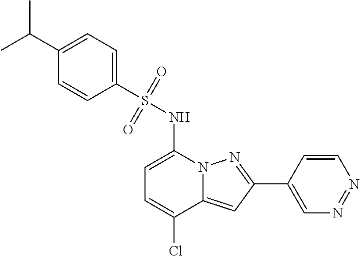 [SMILES: CC(C)c1ccc(S(=O)(=O)Nc2ccc(Cl)c3cc(-c4ccnnc4)nn23)cc1] | 93 |
| 143 | 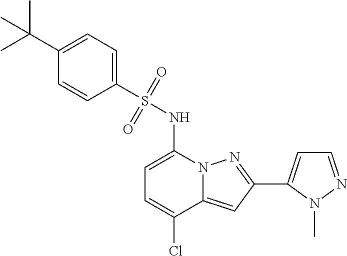 [SMILES: Cn1nccc1-c1cc2c(Cl)ccc(NS(=O)(=O)c3ccc(C(C)(C)C)cc3)n2n1] | 105 |
| 149 | 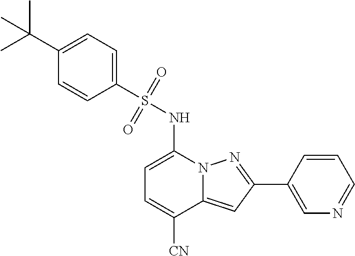 [SMILES: CC(C)(C)c1ccc(S(=O)(=O)Nc2ccc(C#N)c3cc(-c4cccnc4)nn23)cc1] | 87 |
| 150 | 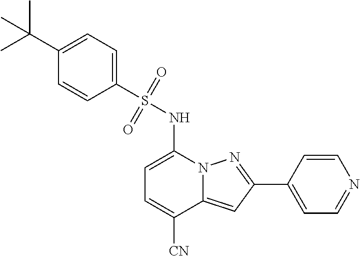 [SMILES: CC(C)(C)c1ccc(S(=O)(=O)Nc2ccc(C#N)c3cc(-c4ccncc4)nn23)cc1] | 39 |
| 152 | 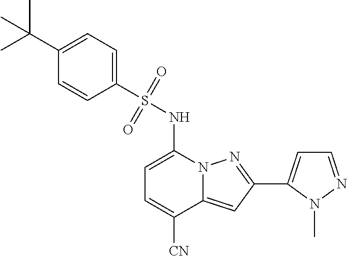 [SMILES: Cn1nccc1-c1cc2c(C#N)ccc(NS(=O)(=O)c3ccc(C(C)(C)C)cc3)n2n1] | 41 |
| 154 | 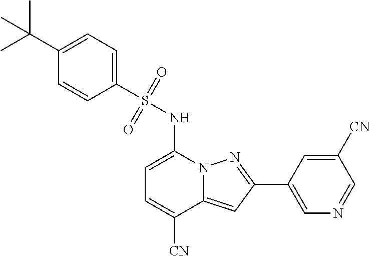 [SMILES: CC(C)(C)c1ccc(S(=O)(=O)Nc2ccc(C#N)c3cc(-c4cncc(C#N)c4)nn23)cc1] | 76 |
| 156 | 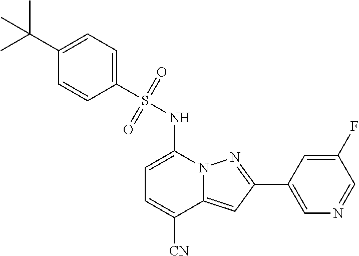 [SMILES: CC(C)(C)c1ccc(S(=O)(=O)Nc2ccc(C#N)c3cc(-c4cncc(F)c4)nn23)cc1] | 51 |
| 157 | 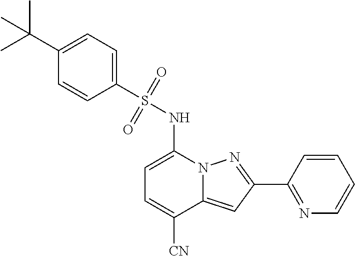 [SMILES: CC(C)(C)c1ccc(S(=O)(=O)Nc2ccc(C#N)c3cc(-c4ccccn4)nn23)cc1] | 81 |
| 158 | 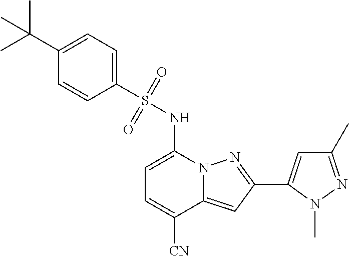 [SMILES: Cc1cc(-c2cc3c(C#N)ccc(NS(=O)(=O)c4ccc(C(C)(C)C)cc4)n3n2)n(C)n1] | 48 |
| 159 | 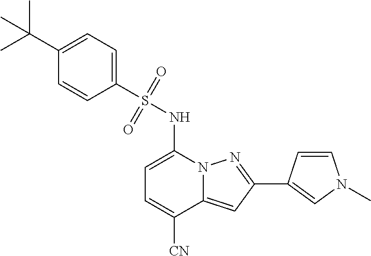 [SMILES: Cn1ccc(-c2cc3c(C#N)ccc(NS(=O)(=O)c4ccc(C(C)(C)C)cc4)n3n2)c1] | 96 |
| 160 | 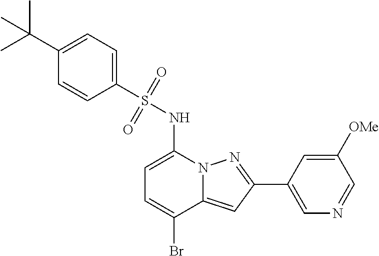 [SMILES: COc1cncc(-c2cc3c(Br)ccc(NS(=O)(=O)c4ccc(C(C)(C)C)cc4)n3n2)c1] | 170 |
| 161 | 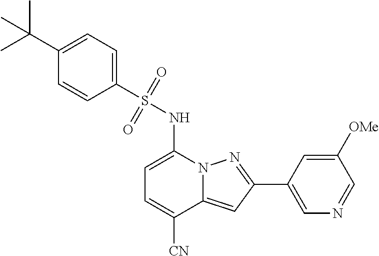 [SMILES: COc1cncc(-c2cc3c(C#N)ccc(NS(=O)(=O)c4ccc(C(C)(C)C)cc4)n3n2)c1] | 62 |
| 164 | 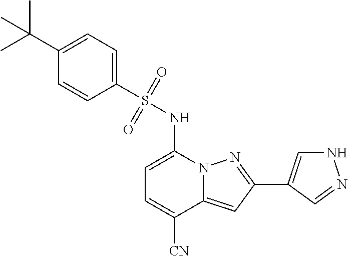 [SMILES: CC(C)(C)c1ccc(S(=O)(=O)Nc2ccc(C#N)c3cc(-c4cn[nH]c4)nn23)cc1] | 34 |
| 165 | 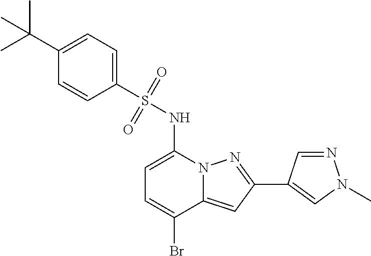 [SMILES: Cn1cc(-c2cc3c(Br)ccc(NS(=O)(=O)c4ccc(C(C)(C)C)cc4)n3n2)cn1] | 129 |
| 166 | 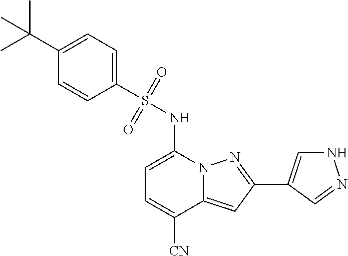 [SMILES: CC(C)(C)c1ccc(S(=O)(=O)Nc2ccc(C#N)c3cc(-c4cn[nH]c4)nn23)cc1] | 17 |
| 169 | 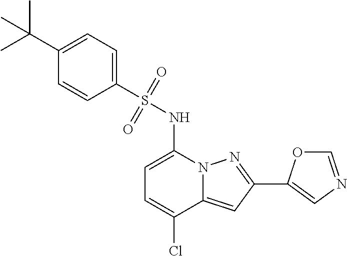 [SMILES: CC(C)(C)c1ccc(S(=O)(=O)Nc2ccc(Cl)c3cc(-c4cnco4)nn23)cc1] | 164 |
| 170 | 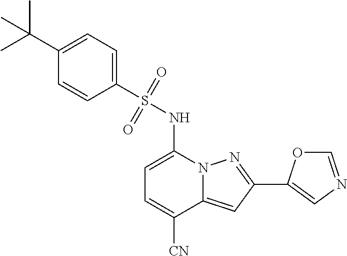 [SMILES: CC(C)(C)c1ccc(S(=O)(=O)Nc2ccc(C#N)c3cc(-c4cnco4)nn23)cc1] | 27 |
| 172 | 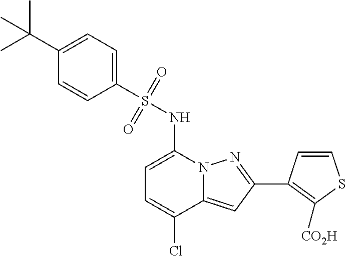 [SMILES: CC(C)(C)c1ccc(S(=O)(=O)Nc2ccc(Cl)c3cc(-c4ccsc4C(=O)O)nn23)cc1] | 79 |
| 173 | 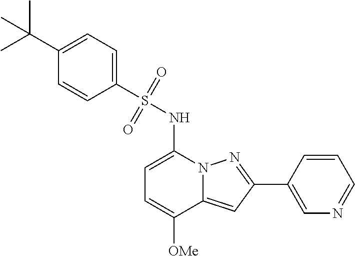 [SMILES: COc1ccc(NS(=O)(=O)c2ccc(C(C)(C)C)cc2)n2nc(-c3cccnc3)cc12] | 377 |
| 180 | 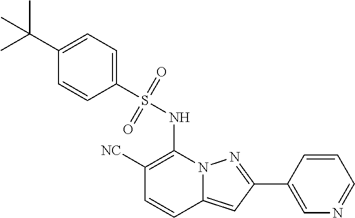 [SMILES: CC(C)(C)c1ccc(S(=O)(=O)Nc2c(C#N)ccc3cc(-c4cccnc4)nn23)cc1] | 30 |
| 182 | 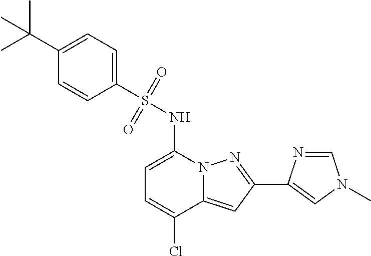 [SMILES: Cn1cnc(-c2cc3c(Cl)ccc(NS(=O)(=O)c4ccc(C(C)(C)C)cc4)n3n2)c1] | 80 |
| 183 | 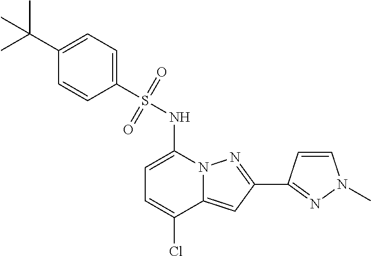 [SMILES: Cn1ccc(-c2cc3c(Cl)ccc(NS(=O)(=O)c4ccc(C(C)(C)C)cc4)n3n2)n1] | 95 |
| 184 | 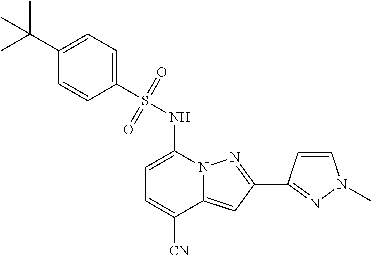 [SMILES: Cn1ccc(-c2cc3c(C#N)ccc(NS(=O)(=O)c4ccc(C(C)(C)C)cc4)n3n2)n1] | 22 |
| 185 | 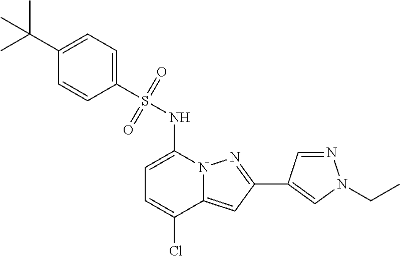 [SMILES: CCn1cc(-c2cc3c(Cl)ccc(NS(=O)(=O)c4ccc(C(C)(C)C)cc4)n3n2)cn1] | 116 |
| 186 | 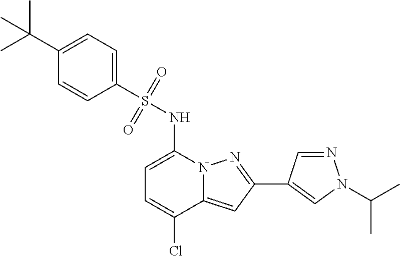 [SMILES: CC(C)n1cc(-c2cc3c(Cl)ccc(NS(=O)(=O)c4ccc(C(C)(C)C)cc4)n3n2)cn1] | 31 |
| 187 | 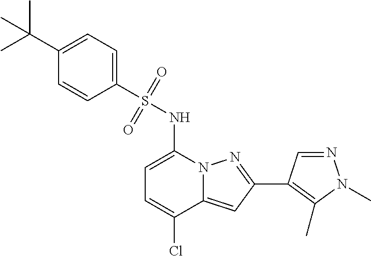 [SMILES: Cc1c(-c2cc3c(Cl)ccc(NS(=O)(=O)c4ccc(C(C)(C)C)cc4)n3n2)cnn1C] | 318 |
| 188 | 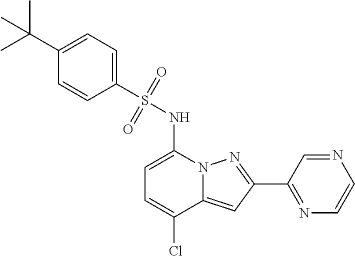 [SMILES: CC(C)(C)c1ccc(S(=O)(=O)Nc2ccc(Cl)c3cc(-c4cnccn4)nn23)cc1] | 194 |
| 189 | 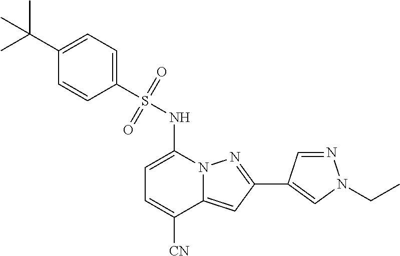 [SMILES: CCn1cc(-c2cc3c(C#N)ccc(NS(=O)(=O)c4ccc(C(C)(C)C)cc4)n3n2)cn1] | 10 |
| 190 | 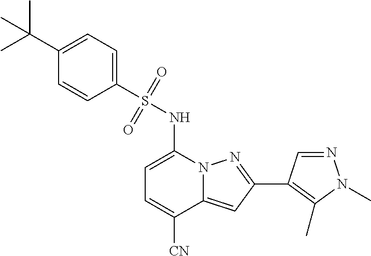 [SMILES: Cc1c(-c2cc3c(C#N)ccc(NS(=O)(=O)c4ccc(C(C)(C)C)cc4)n3n2)cnn1C] | 54 |
| 191 | 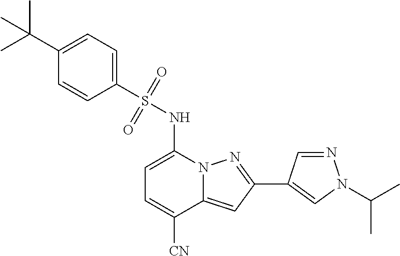 [SMILES: CC(C)n1cc(-c2cc3c(C#N)ccc(NS(=O)(=O)c4ccc(C(C)(C)C)cc4)n3n2)cn1] | 19 |
| 192 | 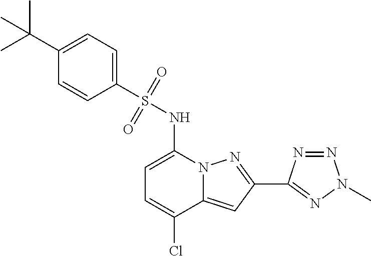 [SMILES: Cn1nnc(-c2cc3c(Cl)ccc(NS(=O)(=O)c4ccc(C(C)(C)C)cc4)n3n2)n1] | 161 |
| 193 | 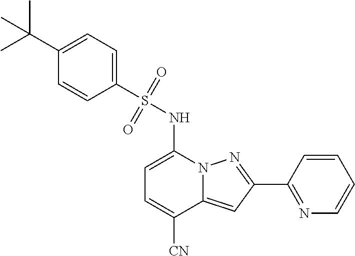 [SMILES: CC(C)(C)c1ccc(S(=O)(=O)Nc2ccc(C#N)c3cc(-c4ccccn4)nn23)cc1] | 81 |
| 194 | 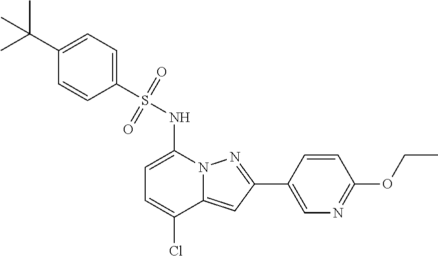 [SMILES: CCOc1ccc(-c2cc3c(Cl)ccc(NS(=O)(=O)c4ccc(C(C)(C)C)cc4)n3n2)cn1] | 110 |
| 195 | 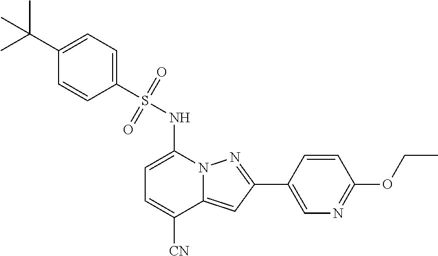 [SMILES: CCOc1ccc(-c2cc3c(C#N)ccc(NS(=O)(=O)c4ccc(C(C)(C)C)cc4)n3n2)cn1] | 34 |
| 197 | 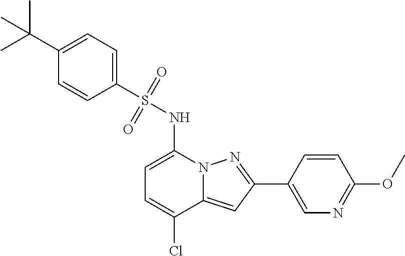 [SMILES: COc1ccc(-c2cc3c(Cl)ccc(NS(=O)(=O)c4ccc(C(C)(C)C)cc4)n3n2)cn1] | 189 |
| 198 | 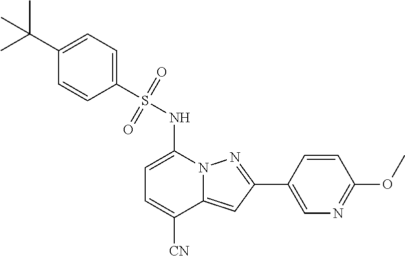 [SMILES: COc1ccc(-c2cc3c(C#N)ccc(NS(=O)(=O)c4ccc(C(C)(C)C)cc4)n3n2)cn1] | 26 |
| 199 | 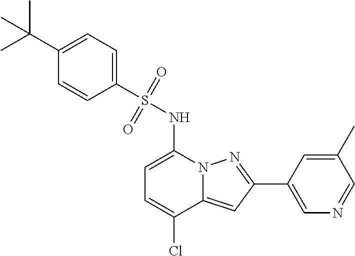 [SMILES: Cc1cncc(-c2cc3c(Cl)ccc(NS(=O)(=O)c4ccc(C(C)(C)C)cc4)n3n2)c1] | 105 |
| 200 | 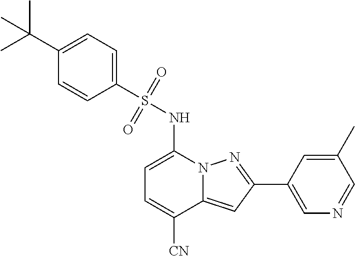 [SMILES: Cc1cncc(-c2cc3c(C#N)ccc(NS(=O)(=O)c4ccc(C(C)(C)C)cc4)n3n2)c1] | 20 |
| 201 | 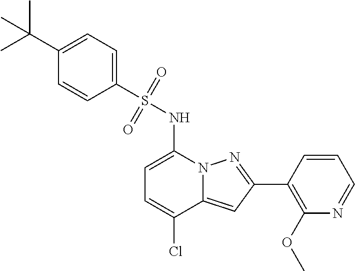 [SMILES: COc1ncccc1-c1cc2c(Cl)ccc(NS(=O)(=O)c3ccc(C(C)(C)C)cc3)n2n1] | 192 |
| 202 | 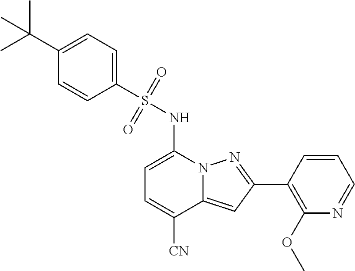 [SMILES: COc1ncccc1-c1cc2c(C#N)ccc(NS(=O)(=O)c3ccc(C(C)(C)C)cc3)n2n1] | 107 |
| 203 | 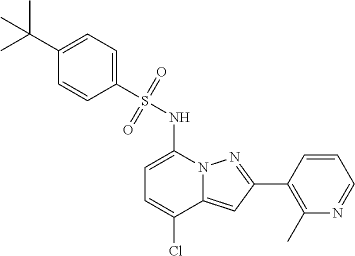 [SMILES: Cc1ncccc1-c1cc2c(Cl)ccc(NS(=O)(=O)c3ccc(C(C)(C)C)cc3)n2n1] | 85 |
| 204 | 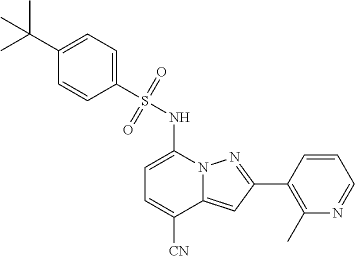 [SMILES: Cc1ncccc1-c1cc2c(C#N)ccc(NS(=O)(=O)c3ccc(C(C)(C)C)cc3)n2n1] | 37 |
| 206 | 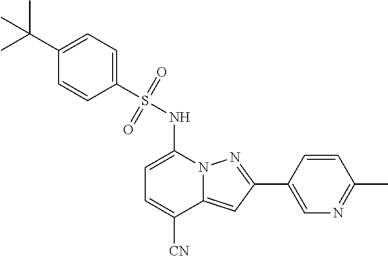 [SMILES: Cc1ccc(-c2cc3c(C#N)ccc(NS(=O)(=O)c4ccc(C(C)(C)C)cc4)n3n2)cn1] | 64 |
| 208 | 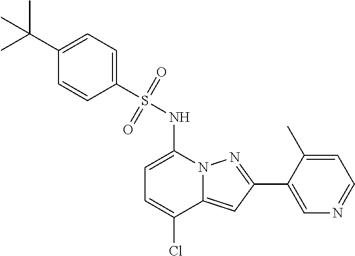 [SMILES: Cc1ccncc1-c1cc2c(Cl)ccc(NS(=O)(=O)c3ccc(C(C)(C)C)cc3)n2n1] | 88 |
| 210 | 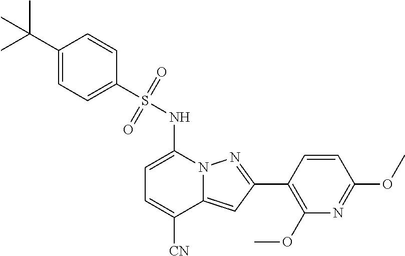 [SMILES: COc1ccc(-c2cc3c(C#N)ccc(NS(=O)(=O)c4ccc(C(C)(C)C)cc4)n3n2)c(OC)n1] | 149 |

What we claim is:

1. A compound of Formula (I) or a salt or solvate thereof, including a solvate of such a salt: 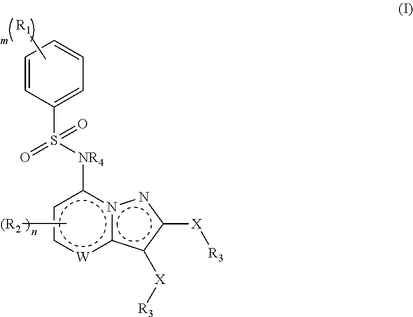
in which:
each R₁ is Z_{q1}B;
m is 0, 1,2 or 3;
q₁ is 0, 1, 2, 3, 4, 5 or 6;
each Z is independently selected from CR₅R₆, O, C═O, SO₂, and NR₇;
each R₅ is independently selected from hydrogen, methyl, ethyl, and halo;
each R₆ is independently selected from hydrogen, methyl, ethyl, and halo;
each R₇ is independently selected from hydrogen, methyl, and ethyl;
each B is independently selected from hydrogen, halo, cyano (CN), optionally substituted aryl, optionally substituted heteroaryl, optionally substituted cycloalkyl, and A;
A is 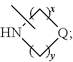
Q is selected from CH₂, O, NH, and NCH₃;
x is 0, 1, 2, 3 or 4, and y is 1, 2, 3, 4 or 5, the total of x and y being greater or equal to 1 and less than or equal to 5 (1≦x+y≦5);
each R₂ is independently selected from halo, cyano (CN), C₁₋₆alkyl, C₁₋₆alkoxy, haloalkyl, haloalkoxy, and C₃₋₇cycloalkyl;
n is 0, 1 or 2;
each X is independently selected from a direct bond and (CR₈R₉)ₚ;
each R₈ is independently selected from hydrogen, methyl, and fluoro;
each R₉ is independently selected from hydrogen, methyl, and fluoro;
p is 1, 2, 3, 4, or 5;
each R₃ is independently selected from hydrogen, cyano (CN), C₃₋₇cycloalkyl, optionally substituted C₅₋₆heterocycloalkyl, optionally substituted aryl, and optionally substituted heteroaryl;
R₄ is selected from hydrogen, methyl, and ethyl;
W is selected from N, and CR₁₀;
R₁₀ is selected from hydrogen, halo, cyano (CN), methyl sulfonyl (SO₂CH₃), C₁₋₆alkyl, C₁₋₆alkoxy, haloalkyl, haloalkoxy, and C₃₋₇cycloalkyl; provided that when W is N and n is 1 and R₂ is butyl, at least one of the XR₃ groups is not hydrogen.

2. A compound of Formula (I) as claimed in claim 1, or a salt or solvate thereof, including a solvate of such a salt, wherein n is 0 or 1.

3. A compound of Formula (I) as claimed in claim 2, or a salt or solvate thereof, including a solvate of such a salt, wherein n is 0.

4. A compound of Formula (I) as claimed in any of the preceding claims, or a salt or solvate thereof, including a solvate of such a salt, wherein at least one of the XR₃ groups is not hydrogen.

5. A compound of Formula (I) as claimed in claim 4, or a salt or solvate thereof, including a solvate of such a salt, wherein either one of the XR₃ groups is not hydrogen and the other XR₃ group is hydrogen.

6. A compound of Formula (I) as claimed in claim 1, or a salt or solvate thereof, including a solvate of such a salt, which is a compound of Formula (II):

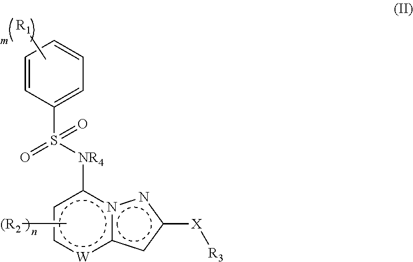

7. A compound of Formula (II) as claimed in claim 6, or a salt or solvate thereof, including a solvate of such a salt, wherein n is 0 or 1.

8. A compound of Formula (II) as claimed in claim 7, or a salt or solvate thereof, including a solvate of such a salt, wherein n is 0 and W is C-halo or C-cyano.

9. A compound of Formula (II) as claimed in any of claims 6 to 8, or a salt or solvate thereof, including a solvate of such a salt, wherein the XR₃ group is not hydrogen.

10. A compound of Formula (I) or Formula (II) as claimed in any of the preceding claims, or a salt or solvate thereof, including a solvate of such a salt, wherein R₁ is Z_{q1}B and q₁ is 0, and each B is independently selected from halo, CN, optionally substituted aryl, optionally substituted heteroaryl, and A.

11. A compound of Formula (I) or Formula (II) as claimed in any of the preceding claims, or a salt or solvate thereof, including a solvate of such a salt, wherein R₁ is Z_{q1}B and q₁ is 1, 2 or 3, each Z is independently selected from C₁₋₃alkyl, and each B is independently selected from halo, CN, optionally substituted aryl, optionally substituted heteroaryl, and A.

12. A compound of Formula (I) or Formula (II) as claimed in claim 10 or claim 11, or a salt or solvate thereof, including a solvate of such a salt, wherein each B is independently selected from halo, optionally substituted C₅₋₆heteroaryl, and C₅₋₆heterocycloalkyl.

13. A compound of Formula (I) or Formula (II) as claimed in claim 12, or a salt or solvate thereof, including a solvate of such a salt, wherein each B is independently selected from bromo, chloro, fluoro, pyridyl, pyrazolyl, methyl-pyrazolyl, oxazolyl, isoxazolyl, dimethyl-isoxazolyl, imidazolyl, thiophenyl, pyrrolyl, piperidinyl, pyrrolidinyl, and morpholinyl.

14. A compound of Formula (I) or Formula (II) as claimed in any of the preceding claims, or a salt or solvate thereof, including a solvate of such a salt, wherein R₁ is Z_{q1}B and q₁ is 1, 2, 3, 4, 5 or 6, each Z is independently selected from CR₅R₆, O, C═O, and SO₂, each R₅ is independently selected from hydrogen, methyl, and halo, each R₆ is independently selected from hydrogen, methyl, and halo, and each B is selected from hydrogen, halo, and cyano.

15. A compound of Formula (I) or Formula (II) as claimed in claim 14, or a salt or solvate thereof, including a solvate of such a salt, wherein each R₁ is independently selected from butyl (including tert-butyl), propyl (including isopropyl), methyl, trifluoromethyl, trifluoromethoxy, difluoromethoxy, methoxy, carboxy-methyl, (CO)CH₃, methyl sulfonyl (SO₂CH₃), (CH₂)₃OCH₃, and C(CH₃)(CH₃)CN.

16. A compound of Formula (I) or Formula (II) as claimed in any of the preceding claims, or a salt or solvate thereof, including a solvate of such a salt, wherein m is 0, 1 or 2.

17. A compound of Formula (I) or Formula (II) as claimed in claim 16, or a salt or solvate thereof, including a solvate of such a salt, wherein m is 1 and R₁ is para to the sulfonamide, or m is 2 and one R₁ group is meta to the sulfonamide and the other R₁ group is para to the sulfonamide.

18. A compound of Formula (I) or Formula (II) as claimed in any of the preceding claims, or a salt or solvate thereof, including a solvate of such a salt, wherein each R₂ is independently selected from halo, cyano (CN), C₁₋₃alkyl, C₁₋₃alkoxy, C₁₋₃haloalkyl, and cyclopropyl.

19. A compound of Formula (I) or Formula (II) as claimed in claim 18, or a salt or solvate thereof, including a solvate of such a salt, wherein each R₂ is independently selected from bromo, chloro, cyano, methyl, methoxy (CH₃O), propoxy (including isopropoxy), trifluoromethyl, and cyclopropyl.

20. A compound of Formula (I) or Formula (II) as claimed in any of the preceding claims, or a salt or solvate thereof, including a solvate of such a salt, wherein each X is independently selected from a direct bond, CH₂, CH₂CH₂, C(CH₃)(CH₃) and C(CH₃)(CH₃)CH₂.

21. A compound of Formula (I) or Formula (II) as claimed in claim 20, or a salt or solvate thereof, including a solvate of such a salt, wherein X is selected from a direct bond, CH₂, and CH₂CH₂.

22. A compound of Formula (I) or Formula (II) as claimed in any of the preceding claims, or a salt or solvate thereof, including a solvate of such a salt, wherein each R₃ is independently selected from hydrogen, C₃₋₇cycloalkyl, optionally substituted C5-6heterocycloalkyl, optionally substituted aryl, and optionally substituted heteroaryl.

23. A compound of Formula (I) or Formula (II) as claimed in claim 22, or a salt or solvate thereof, including a solvate of such a salt, wherein each R₃ is selected from hydrogen, cyclopropyl, optionally substituted piperidinyl, optionally substituted phenyl, optionally substituted pyridyl, optionally substituted thiophenyl, optionally substituted pyrazolyl, optionally substituted pyridonyl, optionally substituted pyrimidinyl, optionally substituted pyrazinyl, optionally substituted imidazolyl, optionally substituted pyridazinyl, optionally substituted thiazolyl, optionally substituted oxazolyl, optionally substituted pyrrolyl, and optionally substituted isoquinoline.

24. A compound of Formula (I) or Formula (II) as claimed in claim 23, or a salt or solvate thereof, including a solvate of such a salt, wherein each R₃ is selected from hydrogen, cyclopropyl, optionally substituted pyridyl, optionally substituted thiophenyl, optionally substituted pyrazolyl, optionally substituted pyridazinyl, optionally substituted oxazolyl, and optionally substituted pyrrolyl.

25. A compound of Formula (I) or Formula (II) as claimed in claim 24, or a salt or solvate thereof, including a solvate of such a salt, wherein each R₃ is selected from hydrogen, cyclopropyl, pyridyl, cyano-pyridyl, fluoro-pyridyl, methoxy-pyridyl, pyridine-N oxide, methoxy-pyridine-N oxide, ethoxy-pyridyl, ethoxy-pyridyl N-oxide, methyl-pyridyl and methyl-pyridyl N-oxide, thiophenyl-CO₂H, pyrazolyl, methyl-pyrazolyl, dimethyl-pyrazolyl, pyridazinyl, oxazolyl, and methyl-pyrrolyl.

26. A compound of Formula (I) or Formula (II) as claimed in any of the preceding claims, or a salt or solvate thereof, including a solvate of such a salt, wherein R₄ is hydrogen.

27. A compound of Formula (I) or Formula (II) as claimed in any of the preceding claims, or a salt or solvate thereof, including a solvate of such a salt, wherein W is selected from N, CH, C-halo, and C-cyano.

28. A compound of Formula (I) as claimed in claim 1 or a compound of Formula (II) as claimed in claim 6, or a salt or solvate thereof, including a solvate of such a salt, wherein m is 2, one R₁ group is halo and the other R₁ group is trifluoromethyl, n is 0, X is CH₂CH₂, R₃ is hydrogen, R₄ is hydrogen, and W is N.

29. A compound of Formula (I) as claimed in claim 1 or a compound of Formula (II) as claimed in claim 6, or a salt or solvate thereof, including a solvate of such a salt, wherein R₁ is C₅₋₆heterocycloalkyl, m is 1, n is 0, X is CH₂, R₃ is hydrogen, R₄ is hydrogen, and W is N

30. A compound of Formula (I) as claimed in claim 1 or a compound of Formula (II) as claimed in claim 6, or a salt or solvate thereof, including a solvate of such a salt, wherein R₁ is optionally substituted heteroaryl, m is 1, n is 0, X is CH₂, R₃ is hydrogen, R₄ is hydrogen, and W is N.

31. A compound of Formula (I) as claimed in claim 1 or a compound of Formula (II) as claimed in claim 6, or a salt or solvate thereof, including a solvate of such a salt, wherein R₁ is butyl (including tert-butyl), m is 1, n is 0, X is a direct bond, R₃ is optionally substituted heteroaryl, R₄ is hydrogen, and W is N.

32. A compound of Formula (I) as claimed in claim 1 or a compound of Formula (II) as claimed in claim 6, or a salt or solvate thereof, including a solvate of such a salt, wherein R₁ is selected from tert-butyl, trifluoromethyl, trifluoromethoxy, difluoromethoxy, and methoxy, m is 1, n is 0, X is a direct bond, R₃ is cyclopropyl, R₄ is hydrogen, and W is N.

33. A compound of Formula (I) as claimed in claim 1 or a compound of Formula (II) as claimed in claim 6, or a salt or solvate thereof, including a solvate of such a salt, wherein R₁ is selected from halo, tert-butyl, trifluoromethyl, trifluoromethoxy, or difluoromethoxy, m is 1, n is 0, X is selected from CH₂, CH₂CH₂, and C(CH₃)(CH₃), R₃ is hydrogen, R₄ is hydrogen, and W is N.

34. A compound of Formula (II) as claimed in claim 6, or a salt or solvate thereof, including a solvate of such a salt, wherein m is 1, R₁ is butyl (including tert-butyl), n is 0, XR₃ is selected from methyl, cyclopropyl, optionally substituted pyridyl, optionally substituted thiophenyl, optionally substituted pyrazolyl, optionally substituted pyridazinyl, optionally substituted oxazolyl, and optionally substituted pyrrolyl, R₄ is hydrogen, and W is C-chloro or C-cyano.

35. A compound of Formula (II) as claimed in claim 6, or a salt or solvate thereof, including a solvate of such a salt, wherein m is 1, R₁ is butyl (including tert-butyl), n is 1, R₂ is chloro or cyano, XR₃ is selected from methyl, cyclopropyl, optionally substituted pyridyl, optionally substituted thiophenyl, optionally substituted pyrazolyl, optionally substituted pyridazinyl, optionally substituted oxazolyl, and optionally substituted pyrrolyl, R₄ is hydrogen, and W is CH.

36. A compound of Formula (I) as claimed in claim 1 or a compound of Formula (II) as claimed in claim 6, or a salt or solvate thereof, including a solvate of such a salt, which is any one of Compounds 1 to 211 as listed in Table 1.

37. A compound of Formula (I) as claimed in claim 1 or a compound of Formula (II) as claimed in claim 6, or a salt or solvate thereof, including a solvate of such a salt, for use in therapy.

38. A compound of Formula (I) or Formula (II) as claimed in claim 37, or a salt or solvate thereof, including a solvate of such a salt, for use in the treatment, prevention or amelioration of a disease or condition associated with CCR9 activation.

39. A compound of Formula (I) or Formula (II) as claimed in claim 38, or a salt or solvate thereof, including a solvate of such a salt, for use in the treatment, prevention or amelioration of an inflammatory disease or condition, or an immune disorder.

40. A compound of Formula (I) or Formula (II) as claimed in claim 39, or a salt or solvate thereof, including a solvate of such a salt, for use in the treatment, prevention or amelioration of Crohn's disease or ulcerative colitis.

41. A compound of Formula (I) or Formula (II) as claimed in claim 40, or a salt or solvate thereof, including a solvate of such a salt, for use in the treatment, prevention or amelioration of Crohn's disease.

42. Use of a compound of Formula (I) as claimed in claim 1 or a compound of Formula (II) as claimed in claim 6, or a salt or solvate thereof, including a solvate of such a salt, in the treatment, prevention or amelioration of a disease or condition associated with CCR9 activation.

43. A method of treating, preventing or ameliorating a disease or condition associated with CCR9 activation in a subject, which comprises administering an effective amount of a compound of Formula (I) as claimed in claim 1 or a compound of Formula (II) as claimed in claim 6, or a salt or solvate thereof, including a solvate of such a salt, to the subject.

44. A composition comprising a compound of Formula (I) or Formula (II) as claimed in any of claims 1 to 41, or a salt or solvate thereof, including a solvate of such a salt, together with an acceptable carrier.

45. Use of a composition as claimed in claim 44 in the treatment, prevention or amelioration of a disease or condition associated with CCR9 activation.

46. A method of treating, preventing or ameliorating a disease or condition associated with CCR9 activation in a subject, which comprises administering an effective amount of a composition as claimed in claim 44 to the subject.

47. A process for the preparation of a compound of Formula (I) as claimed in claim 1, wherein the process is selected from the processes shown in Scheme 1, Scheme 2, Scheme 3, and Scheme 4.
